SEQUENCE LISTING (51 sequences)

SEQ ID NO: 1 - DNA, 1006 bp, synthetic construct
  source 1..1006: mol_type = other DNA; note = Oncomodulin promoter
gtgcaatttatggtatagctgggaaacgtcaaagtcaagagttttgtaggaaagtcacgtcacttagccctgtctcctgtgccgggtgagacctgtgtgtgcacttggtgacaatggctttgagtctgtcaactccagactgaggtcagccttacacacccatagttcccaaagctgaaaacaggcctgcctccaacggtacctgctaatatcaggggagccttttcagcttacagagcaccctgtatgtgtttgtcttagttcaggccaccatctccaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaagtgcaatttatggtatagctgggaaacgtcaaagtcaagagttttgtaggaaagtcacgtcacttagccctgtctcctgtgccgggtgagacctgtgtgtgcacttggtgacaatggctttgagtctgtcaactccagactgaggtcagccttacacacccatagttcccaaagctgaaaacaggcctgcctccaacggtacctgctaatatcaggggagccttttcagcttacagagcaccctgtatgtgtttgtcttagttcaggccaccatctccaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaa

SEQ ID NO: 2 - DNA, 1454 bp, synthetic construct
  source 1..1454: mol_type = other DNA; note = Oncomodulin promoter
gtgcaatttatggtatagctgggaaacgtcaaagtcaagagttttgtaggaaagtcacgtcacttagccctgtctcctgtgccgggtgagacctgtgtgtgcacttggtgacaatggctttgagtctgtcaactccagactgaggtcagccttacacacccatagttcccaaagctgaaaacaggcctgcctccaacggtacctgctaatatcaggggagccttttcagcttacagagcaccctgtatgtgtttgtcttagttcaggccaccatctccaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaaggaaatctaagaggttgtaactatcactttttctagcctatataaggtaggtcagtaaggtagcaaaaacacatctgttgttttgctccttcaactctttttcctgattcttcctggggggaaaccgaaaacggtgagtaactggtggacacatcagaccccagactcttttcttcactgcatgcattcatattaggctcaggtgcttagactcctgttttccggtggctctgacacctggaaggattttaatctctgggagatgggcttttcatccatctgcttcccacctttcaggacaggtgcatgccttcttccacagaatgtctgcaagcagcccaaactgtatcctttcccacgtggaatttgcaacattgcatctctcgggctgctgtaggaaaatgccagtgcatgtgtaacatggtttacggctgcctatgcaaatgactgattatgtcagtataatttttataagaaaacaattgaatccttctttgggtcattttttttttccatttttggcatgtattcaaaagaaggctctgagacaaaaaaggctggggtgttttccgtatctggttttaatttggatattctgtcccgtcacttaatacaaaaccatgcttatcacattttaaaaattctagacaggcctggctcggtggcttgcatctgtcatcccagcactttgtgaggccaaggcaggcagatcacctgaggtcaggagctcaagaccagcctggccaacatggcaaaaccccgtctctactaaaaacacaaaaattagccaggcatggtagtgcgcacctgtaatcccagctactgggaaggcttaggcaggagaatcacttgagcccaggaggcggaggttgcggtgagccgagatcacgctcttgcactccagcctgggtgacagagtgagactccgtcttaatttaaaaaaaaaaataa

SEQ ID NO: 3 - DNA, 1511 bp, synthetic construct
  source 1..1511: mol_type = other DNA; note = Prestin promoter
aaagcaaactcatctctaaaccagaaataatagcaatatctatacaagtaaatacatgtactcagaacagtgcctactacatgtaaacactgaacaggtgttagcaacattgccattattgtgttagtatattaggtacctggtgctaccggcaaaaccagtttatcatccaactgtctccagtgttgctactcaaagtttggtcctccagtagcctatcaggatcacccaggggcctgttagaaaggcacatctcagaccccaccccagacctactgaatcagaatctgcgtttttaacgggatccgcaggtgattcctatgcacattaaagtgtaagaagtactgggctacagacaggtatgtgacaaaataatttcataggatggcaaaggccaagtggcaaatgaaggacaccagaaatgcacgtcccaggagcccaactcctccttagtaaattaccctattaagatttgtttagagatgttcaaaagcgtggagaaaagcaaatttggtttccctggtgcccttgaagagatcgccctcgtgtggagtagggagggaatctctagcctttcctctcggatgaagaacagcaccagcgctcccagccaaaggcctggcccaggttctggaggtggggtctccttggcagaagcctctggtgtctgcaggcgtgcatttacagctttaagaccaaacagctagtccgccacgtgtcactacagtgtgcacgcgcagaaatgcacaaagcaaaaaaaaaaaaaaagatgctcttaatgaaccaactataatccttgctaaggcataaagccagagggaagtatgtatctgaaatcattttctacccctcaccctcttggagcccggcactctggctgcggtgctctcttgtatcccagttgctagatgcaaaacaagctatttcctatctaattttttttttgttttataaattctaacttaaatgcccagaaaataactactcatactcacattgtcctctaattgaaaagataagtcaggttttttgtgtttttttttcattttaaaatcataatacgcaatgttttccacttgaacgctataccttgtgtattgtgcttgcttcagcctcgagcctctactgatgttccacctcaaggcgacaggaatgccacctggagaaactcctgggcggtatgggaagaaagccggtctcatcagagtatatttgcggggatcgacgaccaaggtgttaaattccaagcacgctttggaaagttctaggtgcttgggaagagatccgtaggcggcagggatgcccgcgccccggcgtcccagcgcggagggtggcggcggggcctggccctagcggggcggggcgggctcgggttaccgggagtcgcggggcgcggccggcactgcccgcggcgcctcctcctagagccgcacctggaggcagcgcgcgcgtcgaagaggcagcggctgtggagcgcggcggggcggctccgcccagggcagcccgggctg

SEQ ID NO: 4 - DNA, 740 bp, synthetic construct
  source 1..740: mol_type = other DNA; note = CHRNA10 promoter
ttcagatgccatcattaatgagaactatgactacctgaaggggttcttggaagacctggcaaggaactccccttggattaattggcttctctgcttctttgtaggtggattgctcaggtaatgacctggagcagttacacatcaaagtgacttcactgtgcagtcggatagagcagattcagtgtctggtattggctttccctttgtatttttgaatagaatataccattcaaagcctcctcgctcttctactatagtggttttgtttttaaaccctgagtgacgcttcacctttctaaatcagattcccttttgtaaaggggataatgattgctgatgttacttcacacagggctattttcaagaggaatcaattgagtagcatgagtactattccagatcttattttgatctgtcaagctgaagatgtgagcaaattccaattaagattagaccaaagacttctgagactttcaggaattcagggatgagaaagcagagtgggtcagctctgttgtctggaacttccatttaacttagatgcctcaggataggggttactcagctggaatcccctccactactgactcactatgtgaacctgagtgagtcacaaaacatagttggacttccagcaaagaacacctgacctggtttccttaccagaggaatgtttcagaaagtgagtatgctatagaaatggttagctcttagcagtgttcggaattgtgggccaggag

SEQ ID NO: 5 - DNA, 918 bp, synthetic construct
  source 1..918: mol_type = other DNA; note = DNM3 promoter
ccttgattcagagttaaagctatgggaaagtcctcaggcagaggacaaacattagacaagaaaatgcccatatatgaaaccctgcgaagcatcagtatttgaggagcagactaaaaaggaaccgtctgtggaggctaagagaagcatggccatttatctttgtgtcccgatcatcaggcacaggaccccacacacagtcacttctcaatgtgctaaatttcacagaatgcgtccagggtacctggttctggatagatccggtagaaggagatagaccgggagggcaaatggcatgaggagtctcacaggccagagtgattaaaggggtgtatcggggcggtaaaccctacagactctacctgtgcttatgcggggctggggaggacgagtcattacagatgaagaattaagtaaggtcagaccactcagggccttagatggatgtcacattgaagaatttagactccaacaggcctgccaccctgggaggagtcatcgcggattctggagaagggcgtgacagaggagatttcctttcgggaagtgtagtctggcagcggtgccccggtggtggcggcggcggtgctgctgttgctggtgatcgtgtggtggtgttagcggcgatagtgctttccactgggctttggcttggtagccgctgaaagagaacaacgctgccgctgctgctgatttcatgccatttcctgacccggcgctgtaacttggcctctgagccttggccacagaacgcagaggccgtggcatctggccgcagctgggctgcagtgcgtgcgcgcctggcctggtggtccgatgggaagcccggggcggggcagccgcggggcgggggcggggcgtcgcggagataggccacgcccctgcccgcccgcgcaggcgcgctgcgggtcgttagctgtc

SEQ ID NO: 6 - DNA, 1631 bp, synthetic construct
  source 1..1631: mol_type = other DNA; note = MUC15 promoter
tttctcctaattcagcacaaaaattgagttccttttctgtagctaaagagcttgtatgaactgtcagcttagctaaccatatgttttcaatgttccctgcaaattgtttaaggtatgtatagtcctttcaatggatgagtaagtcttttgtcattgttatttgctgcctgtggacttgatttcaaaatcttcttcaggtcatgaataaatttccttttccttctgtccctacttttgagccaaggaacaaatcaagattcttcctcagagtgtacacaccttcccaggcatctcactctctcctcactctatctgcttcaagttatggctcgttggtgagaacactctgctgctgaggttattatttagctataataactttttctaactagacagaaacaaattagatatgccaggattttctaattacctgccttaagtgcttttttagaaagcattaataaatcatgtggatcttttcctagcagtggtaagataagttataatattatcaaactgtcagttttgccacttcaatatatgtatgcctggttgtaacctcacttaataagttaagtccatgtaaaaatagttgatagttaataaattgggcaagagttgcttaaacagattagactatataacaaaattagggttttaaaagaataaagctgctataacagtacgcttcatctcacaggaattaatcagttatggtatctccacaaaacagaatatcacgtattgttgaagagagccgtctcatttccccggggttggtttaatttctaatcaaactctgaaggggcctttgggcttcagaaaatttaaaactatagaattaccttgttctttcctcgggccaattaactgggcagattctttgcattccatttgaagcttactagctcctgcattttagctaaagtttcgtttctcgctcagcagttgaaaacctatctccttgtgcagcagaaaccaagtatgaacctcaggcatattgagctgaacggcccttggcgccatccccaaacgctgatgtgcggaagatcccagtttcactcttctccctttcataagctctgaaaggaagtgtaggaagtatgccaagttgttattcaactctagtatttaatcaagcattacctgggcacttctgaaattctccagcttctaaagtgagagtaaaccagagagaacacagggtggaaactacttaatcgagaaggctcctaggataagtgaggatcacatggccattctcaggccccagttcctctccaaactcctgaaagtcagcaagaaaccgaatctcagtcatgatgattatttttcatgtaacacctcacagcgttctcagggatcccaatatatgctactaattcactttgtgttaagtaggagtttcttaaaaaaacaatttcagtggagaaattcctgctataccagatgactttgccaaaatctttgtcctttttttcacttagggtgaaaaaaaaaattgatgacccgtgttttgctaccactgacgagagtaataccttgtcccaaagctaaaacgatcaacctatgaaaactggagggttgggcttttgttgttgttgttaaaggcctgaatgaggtgatatctt

SEQ ID NO: 7 - DNA, 984 bp, synthetic construct
  source 1..984: mol_type = other DNA; note = PLBD1 promoter
gacccattattcaatgggagttgtcaggatgtcagcaatgtacaaaatcattgcttaatttgtttgacaatggaaatggccattatggtttttatgtaactttgcttctgttacataattcttgctgacacggtgtttcaaccaaggtacttggtagcaagtgttgtacagaaaaggatctgtaagtggtttatgtggtcatcaaccacagcaaagatttcatctgagctgtgctatgaagaatgtagcttgagaaacacaaaatgtatcactgggcaaaaaggaagcagaagaaaaatacagttctgctaatgagagctctgactggtatctggagtataagatgggcccagccaatgctgagtgaatgaatgaaatgccttttgcctacttcacaatgtcacctagggcacccggtgccaacttcacaatatcacccaaggcataacttttgactacttcacaatgtcacttttaactgaccccacacagaaatggggactccacagaaacgtaggagtgtgtctagtgtcagccccgtctgaatcactctcctgtggtggctccagccaacgaagaggaagcaaaaaggataaaaaatctgagctacagcgcatggatttaggttaaacagcctgggaatgaggggtacgctaatcgctgaggaaaacgcacctgtggaggcctctccagaaacagcagaggatccgagctgcgtgtaggcagggcgcgcatgtcaccctggcccgggcgcctggtccgctgctggagataaatggtcgaccccggagggagaggctagtaggggtgttgatgtgaactgattcgcccaagccttgggccgcaaaactgcgaaagaaagcggcaggcagcctctgcatttcccagaagtgcagctggggaactttcccagaccggcccaggggttgctagagggtcagacgtaaaggatccgcctttcctaggcggggtgggccccaggcc

SEQ ID NO: 8 - DNA, 1416 bp, synthetic construct
  source 1..1416: mol_type = other DNA; note = RORB promoter
ccaataaatgttggctcttgttttttctgacctgtatgttttgtctttgttccaaagctagccttacctctcccacatactggggttaattcatgctttggcccttatcaccttttccaatttatttcaaaattacatgctctattttaatatttgctttcttttttttattttgaaaacttattgaacttgcatctacactttaaaatgaagcagaacttaaagaactcaaagattatgaagaagactcagtacctgggaataaaattgagaataggttccttttatgactatataaccaatctcaaccattattttttgcttccccaaattaggagagtttaaaatgcagattctccccactctcctcttcccattcaatagaaactgagaaagaaggatcttattcaggtcttcactccatttgtgattcatattcagtggctgaaaggttagaaagcattcactccaccaataatgatcaagcacccataaagtaccaggagctcttacaaactctagggaaatcctggctcctgttgtcatgaattttgcattctcaggtaggaaatgtggctctgatgcctgctggggcagtgtacacttagagctacagaggatcttggaggtaatctaaaacctttctaaagagcaccctgcaatcacaccttctagcaacagccatttctcttgaattagtaaggtggctacaccgccaatttgagctgttctccttcagtcctgtagtccatcgccaggggagtctccaaatgctaataaaaatcaatttcccagacaaaagaacatagagggtcagggagcatctgacggacgtttttaaaggaaggggacagctacttccatgggactgcattttagttgtgctaaaagtgatgaaagtgggtttgcattattctaccaccaacacccaaaccacctgcccacggaaacccccgccggagaccgaagtttacccaaatagcgctcggcaaagcgctgccataaattcaaaactaactctgccgggcccgcgggggttgcgagacagggaccgaacgtgaaacccggggagccccgcgtctcttgcctccgaaggttttccgtgatcagtgtccccttctctgctggagtcggaagtgcctgtcacctgcggatctgcccgactctcccggtcggcccttcttctctgcccagttcggacagtctcgaattccccgtcgcagccccggccacctcggactccctggtccccagcccccgccccaccccccgcctccaccacgtcccctccccgcggtcccagcctctccaggcgctgctgggctctgattggcggctgcgctgacagcaggcggggcctggaagtcgcggccaagcccgccctcgcgtataagcccctctcagcgctctctctcc

SEQ ID NO: 9 - DNA, 1297 bp, synthetic construct
  source 1..1297: mol_type = other DNA; note = STRIP2 promoter
ccttgaatatttatgtccattttaacacttcctggttgcaagagggatgtgcctccattatttcctccacagttttggtatttgtcagacatttgttctgctgtctttctaatccagccaacgtctgctcaggaagtggggccagctccactgggacccatagttttacttccttgtcatttgattggatagtttccaaggaagcccctccagattggcactatctcagaaaaggagagcttgttgtgaaacactgcttcctgaaacttcctgctattgcctaaagctacgtctgaaactgagtagggaaaggcatacttttccagggacttagggggataggctttgaggtctctcctcgtgttgactctatgcaatcttcatagcaccagttttacacattccttctctgaaattaaagccagatggagcctctaggcttagcaagtggctttagatagccaccagaggggacttgcaagctgtcctctatcctactcccaagatcagtctgccctttcccctaggaataggcaggaaaagaataaaggaaagaaaggactggcgagcaggtgagggtgggggcttgctctaccctcaacatttacacaccatgaggaagaggccccctacagcagagaagggcagatgacaggagcagccctcgagggcaccaccaatttcagtgatggaaaaactcccccatcccacccttagacctccagtctcccagccaagccctagctccgggcgagatgcgttctcttcagaaaaacgctgagaattctcagcttccagagacagcgagtccctcgtttcgggcgatgtccctggccacctggcggtgccatccctcccctgagactaagcgggatatgggacgtgtgcaggagccgggatatggggggccgggtcggtggtaacagggaaacggagactgctgtggagcagtaggcggagactagagctccggaaaaggtcgctacagggacgggggtgagagctgagagacaccgagtgaggagcacagagataacccgcctgatctcaaggcccagctttcgcgaggtgtggagcctgtagctaacctaggagtctccgtccgccagcaatgccgcaggactaaaaagatcccctcaaaaatctcttcattgagcccccacctcctcgagtcccgctccggccggtcgagcagccaatcgcctcgcggggcggggttgcggcgagctgccgtaaccaatagaggtggagggggcggggcctggctcccggcgcgcggcggtagggtcgcctccgg

SEQ ID NO: 10 - DNA, 1283 bp, synthetic construct
  source 1..1283: mol_type = other DNA; note = AQP11 promoter
aggcatgagccactatgcccaaatgagaaataattttgtatgaaaaataatcttgtatggtaaatttagaccaagaataaaatgagtggttgtataagaaagaaagatgttcagaacaaaccaaaaagtccaagcatgtcacgaatggtctgtgtaagtcataataaaaggatttatctaaaaaaaccaaaaacttttatatgatcaagtcgtctataattaaaggaaaattataatgggtttttctagacattgggtgtgatgtaatgaaacgtacacactaaagaattcattacaaggctttcatgttttgttttttgtttgtttgactggtttgttgttgttgttgttgttgttgttgttgtttttgagacggagtttcgctcttgttgcccaggctggagtacaatggcgcgatctaggctcaccacaacctctgcctcccgggttcaagcgattctcctgcatcatcctcccgagtagctgggatgacaggcatgcgccaccatgcccggctaattttgtatttttagtagagacgggggtttctcatgttggtcaggctagtctcgaactcccgacctccggtgatccgcccgcctcggcctcccaaagtgctgggattataggcgtgagccaccgcgcccagccgcgcccggtttttgttgttgtttttgtttctaaaaacagcgtctcgctctgtggcccaggcaggggtgcagtggcgcgatctcagctcactgcagcctggaactcctggggtcaagcggtcttcccacctaagcctctccgtgctgggactccggacgcgctccacctcacgcagccgtattcctgctttcaaagcagatggaagaggtgcgccaggacccccagttcttggaaacagacctctccagttacctgttgtttcctcttcacgaagagtgcatgtaacagtaagacacaactgtttcatattatacgtaaagagttcatgccaaaggttatagacagtcacatgctaaaactaggctacactttgaagaatcaccgctcaagttctggaaaaaagaggtgactgttgaacaacactgtgagggtaatcgatgccactgaaatatacacttaaattgattaaagtggcgaattttatctggcatatattaccaccatttttagaaatgttttttggcaggtgaagaaaagcaaggctccaggaggccctgcgcaccggtctacgcccactaactcacccgccccctgcgccgcgtctcccctctcaatttcagtcgcccattgat

SEQ ID NO: 11 - DNA, 1555 bp, synthetic construct
  source 1..1555: mol_type = other DNA; note = KCNQ4 promoter
agggcccatcctggtgtaaacaaaccctttggcgcagcccaagagagcccctatctaatacccagcacgatccccttacatccggagcactctttaaacatttttcctagctgatcttcacagtgaccctgcaggggagacaggaagaggtatcatgatccctgtgttagcgtgggagggctagtgaagtgcagtgacttgcccaaggtcactccatgaattgagggtggaattgaaaccaaaacactgatctcctgaccccctgtgcatacacagttgctcttggagattggagacccctggaatctggagcagacaatctggctggcttccttgcagctcaggtctgcggaggccacaagggggcagcatgcagccctcacctgtgtctctgggacctttgaagggagggtcctccctaggataacagtgagagctggaaactctaccctctccagagtattgcctcaagatccctgaacttagctccatgttttcagaatgtgctagctacaattcctgaaatgcccttttacttcccttttcacttattgagctcctatacatccatcaaggcccaatttaaatggccctttcagcagctatttctttggcaccttctgtgtgtcagacgttgttttaaacattgtgaatacagcttaaaacaagtctgacgggtggaaaggaaactgctgagggtggggtcaggggaacaggtgggagagggaccagtcccctccagcagaggggccaattgagggagcctgagacagctgtttgctcagaaaagtgtcttagtcactaaaggttgtggtggggaaagtccgtcctcccagtcatgtcctgggaatccggatggcgcaggaaggccacccggtgaccctaagagtggccacctgtcctctctgaactggactttctcttctggcccttcccctccctccctccctcactggcgctcagcagatcaatgctgcctttgctgacagctgagaatcgagctcgccttcccgccccttcccccgcccctcccgctcggcttcgtccctcgagatcctcccggaggaaccgggaagagtttgctgcggaaggctcaccctggggcagggcctgcggagggagcggctggtgtggccgcagctttccgtggaggaagagggaaagaggatcgggaaacccaagttaccaaccctgtgcaggggagatggaggtcggggactaagaaaaactgctgcccacccagccacacacagcactgggcacactttaagcacccgcaccaggcacacagtgctcgaccccaacggacacacctcatcctgccgcccgcggccacaactccacattcacttgcacgcgtccggcttcccggccccgcgcgctgcccccgccacgcggttcggcccaggcaccaactcggccgcccgtgcgccctgccccgccgcctgctccgcgcgttccctccctccgcctcgcctcgcttgctcgctcgctccctcccgatttgggaaggcggccgcggggcgggcgggggaggggcggggcgggggagggt

SEQ ID NO: 12 - DNA, 1214 bp, synthetic construct
  source 1..1214: mol_type = other DNA; note = LBH promoter
gtgaattcgatgatgtgcttgtgtggacatgtggaggtctcaagaacaaaagaagagctgggcctggcacacagtgggtgctaatgcctgttagaattgttgttgagagggcaggagggtgtaacatggacccagctatctgatcctgaggctgggcgccatgtgggtgtgaagtacaccaggggctccaaccagcaagtgctagctcaggttacagtcagctgcccctggaggaagctagcagacatcctgtgtacttgaaaagaaaactgaaagtgctatctgcatcctggtgatagtaacctctcttttctggctgttgaagtgcattcctgtgcggatgtggaaagagagaaagcaagatacagccagggctaggacaggaatgtgagtatttccttaattggacatgagagccttgaactgattccagttggagtgttttcttttagggcctggaccctaaagatttcatacagttttctttgtcagaaaaatccctttggttcaaaggcccctcgatagaaataaagaaaaagccagggctgaatttctttgatatgtgggaaggcaagagtttatgagctgccagatctcaggcttcttttggggtggaggattttgtctggtgggttcgggttgctttgtgttgttgactgctaattcactgatgaccaagtttctcaaataccttaaaaacaagccctacgtctgctcagtgctttccaatttaccaagtgttttcataacatttcttatgtacgcaaatgagtttcaccgaaaaattggctagaaacttcccttctcctactcacgttcatagtgtagctgtgaaaacaaacaaaaccacagaggcatggtaagtgtggtatggtggggaaaacaaagccattttacaggcgtgattgaagcggaggccacagagcggcagcgctgggtcccgagtgagactcccatcatgtggctcaatggaaaaatcctacccaggacgacaccacatccttgctcccacaaataaaaccttccacggaactcagggctgcagacgcagagccgagcgcgcccccgagccgccgcccgccggagctgcgagcgctgaagccattcatgattttggtgacgttattccaggagtgggcgagggagggcggggcctctcggggccaagccccgcccccgcccctataaatacggcttcccgggctctttgtggg

SEQ ID NO: 13 - DNA, 1130 bp, synthetic construct
  source 1..1130: mol_type = other DNA; note = STRC promoter
cactgccatctcaacatgtggtttctaggttgcctcaacagggaagagattgttggaggctcatttgctagctcttaaattctttggtcaaccacagtccattgaacagaactaatcacctgactgcaagagatctgggaaatgtgagaaacacctagatatctagtaagcaataaatatttcagttaccaaagccaaaccaaaaaaagagaaaaataattgtactttacaaagggaggcatctgggtcctgtgggagttttggggagtgaggatgtttcagagttctcaactcctgtggctatccatttcattttagcaggacatatgattaatttcttgttctggacctttgtaatttaaagtctgaatccttagcggcaagagaattgcttaatcaatggcttacaacagcagaacgtggactgccaggaaaatttccatcctgagttaagaaagaaggataatttattataagagggttgttacagaatgaagggcagaaattcagaaggattacaggatgggctggaaccacaaagcactgtctgctttttagactaggtgtggtatccttgatgggcaaagggaatattggtaaaattatttgtgacctgggttaagtcattccatttctctgggcttcaattcccctgtctataaaatgtttgagggagagaatggggaagggttctagggaaagaaggacagaataaaagtttgggtatatgaattactatttagagttggtataaagtgaaggcctttggggagatataccctgaccagaccagattattttgaatgaaatctctttctctgttccatgagcagttctgtgtgtagggagaacatttgaatggcctaatgagcaaatcacatttctctgggtctgtttccttatccataagttctgcatcactggctcctaactcaagcaatctccttgggtttctctgaggggcccctgggatcccctatcattagtccctctcacagaagcatacccttctccagagctaaaggatcagatattcagcggctcaggtaacaaacctgctgtcaggttacacatattgtttcctgaaagaccacactacagtgtcagtggagcctcaggttgcctgcagt

SEQ ID NO: 14 - DNA, 1806 bp, synthetic construct
  source 1..1806: mol_type = other DNA; note = TUBA8 promoter
gaagacatagttccagtctgagtctgaagcctgggacccatgagagctgaagacgtggtcccagtctgagtctgaagcctgagacccaggagagctgaagacgtggttccagtctgagtctgaagcctgagatccaggagagctaaggacatggttctagtctgagtctgaagcctgagacccaggagagctgatggtgtggttccagtctgagtctgaagcctgagacccaggagagctgaatacgtagttccagtctgagtctgaagcctgagttccaggagagctgaggacatggttccagtctgaatctgaagcctgagacccaggagagctgatggtgtggtctgaagacgtagttccagtctgagtctgaagcctgagacccaggagagctgaagatgtggtttcagtctgtctgaagcctgagacccaggagagctgatggtgtggttccagtctgagtctgaagtctgagacccaggagagctgaagatgtggttccagtctgagtctgaagcctgagacccagcagagctgaagacatggttccagtctgagtctgaagcctgagacccaggagagctgaagatgtggtttcagtctgtctgaagcctgagacccgggagagctgaagacgtagttccagtctgagtctgaagcctgagagacccaggagagctgatggtgtggttccagtctgagtctgaagcctgagacccaggagagctgaagatgtggtttcagtctgtctgaagcttgagacccaggggagctgaagatgtagttccagtctgagtctgaagcctgagacccaggagaggtgaagacgtggtttcagtctgagtcaaggcctgagaaccaggagagctgctggtgaaagttctagtgcaagggcagaagaccaatgtcctacctagctcaacagtcaggcaggcagaagttccctgtttctcagcctttttgttctattctgttcttcagttggttggatgaggcccctgcacattaaggatagacaaaaattcaacgcatgctttactaagtaccgtttgtatcagtgggtaaagcactgtgtttggtactctctcaaatgcaaagatgattacgacacatgtactatcgtttatgaatgggtggccaacagaacagattgccgcataggtaagcagaaatctgctctcattctctattggccacaagcaggcatgtcttaggagcagaagggtaggaagatctctaactgtgcttggaaacttggggagttaccacgtctggctaaagtggtattgtcttaaggaaaacctcttactactgggcagaggcaggggaaccctggtatgagttctggattacataggagatgtgacttggacacgtttggggcttaaaagtaggaagggatcaaggggggagatttgaaaatcccggtggaggtgcgaggtatccggggagaggtgggagcagaggccctgcagcttgccaagcacacacggccctagggcgcccagctgagacggcaccttggcacccgggcccgctgcagcccgctccggtcagctgcaccccagtcaggagcctttccagcgggtcggaggagaacggaagtttggggagacccgcgcgattcgcctggctgcattttacatttctttctccggcagctggggtcacgaaggctgctctcgccggcggtgttggaacgtggacacgtgcgctttggtaatagggcagcctcccccgcgggcgcagtccccgctgcgagcgcccccggctgctgaggcgggaccgaggacccggagattt

SEQ ID NO: 15 - DNA, 1905 bp, synthetic construct
  source 1..1905: mol_type = other DNA; note = Prestin promoter
taaacactgaacaggtgttagcaacattgccattattgtgttagtatattaggtacctggtgctaccggcaaaaccagtttatcatccaactgtctccagtgttgctactcaaagtttggtcctccagtagcctatcaggatcacccaggggcctgttagaaaggcacatctcagaccccaccccagacctactgaatcagaatctgcgtttttaacgggatccgcaggtgattcctatgcacattaaagtgtaagaagtactgggctacagacaggtatgtgacaaaataatttcataggatggcaaaggccaagtggcaaatgaaggacaccagaaatgcacgtcccaggagcccaactcctccttagtaaattaccctattaagatttgtttagagatgttcaaaagcgtggagaaaagcaaatttggtttcctcagctagggacgcggagagtggtctggtgcccttgaagagatcgccctcgtgtggagtagggagggaatctctagcctttcctctcggatgaagaacagcaccagcgctcccagccaaaggcctggcccaggttctggaggtggggtctccttggcagaagcctctggtgtctgcaggcgtgcatttacagctttaagaccaaacagctagtccgccacgtgtcactacagtgtgcacgcgcagaaatgcacaaagcaaaaaaaaaaaaaaagatgctcttaatgaaccaactataatccttgctaaggcataaagccagagggaagtatgtatctgaaatcattttctacccctcaccctcttggagcccggcactctggctgcggtgctctcttgtatcccagttgctagatgcaaaacaagctatttcctatctaatttttttttttaagagacggagtctcgctttgttgcccaggctggtctcaaactcctggactcaagcaattctcccagcttggggtaacgtgttacattattctacttaataaaaagcaaaagttgttttataaattctaacttaaatgcccagaaaataacttatcatgcattgccttgtcgtgcaatagtcaatatttgcaaaccaagtgttaaccaaaggcagttcatcaaagatttttgaaaattaaaaaaaaaaaaaaactcatactcacattgtcctcaggatttcctgttttcgaaatgttcctgtacgaatcggagtctctataatgattgtaattgaaaagataagtcaggttttttgtgtttttttttcattttaaaatcataatacgcaatgttttccacttgaacgctataccttgtgtattgtgcttgcttcagcctcgagcctctactgatgttccacctcaaggcgacaggaatgccacctggagaaactcctgggcggtatgggaagaaagccggtctcatcagagtatatttgcggggatcgacgaccaaggtgttaaattccaagcacgctttggaaagttctaggtgcttgggaagagatccgtaggcggcagggatgcccgcgccccggcgtcccagcgcggagggtggcggcggggcctggccctagcggggcggggcgggctcgggttaccgggagtcgcggggcgcggccggcactgcccgcggcgcctcctcctagagccgcacctggaggcagcgcgcgcgtcgaagaggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaattgcttgtctccggctgttaaggaataatttcagagtact

SEQ ID NO: 16 - DNA, 119 bp, synthetic construct
  source 1..119: mol_type = other DNA; note = 5' ITR
ctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 17 - DNA, 130 bp, synthetic construct
  source 1..130: mol_type = other DNA; note = 3' ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - DNA, 380 bp, synthetic construct
  source 1..380: mol_type = other DNA; note = CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 19 - DNA, 290 bp, synthetic construct
  source 1..290: mol_type = other DNA; note = KCNQ4 5' UTR
cgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgccc

SEQ ID NO: 20 - DNA, 2085 bp, synthetic construct
  source 1..2085: mol_type = other DNA; note = Human KCNQ4 codon optimized
atggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggac

SEQ ID NO: 21 - DNA, 66 bp, synthetic construct
  source 1..66: mol_type = other DNA; note = 3x flag
gactacaaagaccatgacggtgattataaagatcatgacatcgactacaaggatgacgatgacaag

SEQ ID NO: 22 - DNA, 224 bp, synthetic construct
  source 1..224: mol_type = other DNA; note = Bgh poly A
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatg

SEQ ID NO: 23 - DNA, 4407 bp, synthetic construct
  source 1..4407: mol_type = other DNA; note = pITR-CMVe.sOCMp.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgcaatttatggtatagctgggaaacgtcaaagtcaagagttttgtaggaaagtcacgtcacttagccctgtctcctgtgccgggtgagacctgtgtgtgcacttggtgacaatggctttgagtctgtcaactccagactgaggtcagccttacacacccatagttcccaaagctgaaaacaggcctgcctccaacggtacctgctaatatcaggggagccttttcagcttacagagcaccctgtatgtgtttgtcttagttcaggccaccatctccaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaagtgcaatttatggtatagctgggaaacgtcaaagtcaagagttttgtaggaaagtcacgtcacttagccctgtctcctgtgccgggtgagacctgtgtgtgcacttggtgacaatggctttgagtctgtcaactccagactgaggtcagccttacacacccatagttcccaaagctgaaaacaggcctgcctccaacggtacctgctaatatcaggggagccttttcagcttacagagcaccctgtatgtgtttgtcttagttcaggccaccatctccaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaacgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 4475 bp, synthetic construct
  source 1..4475: mol_type = other DNA; note = pITR-OCMp.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgcaatttatggtatagctgggaaacgtcaaagtcaagagttttgtaggaaagtcacgtcacttagccctgtctcctgtgccgggtgagacctgtgtgtgcacttggtgacaatggctttgagtctgtcaactccagactgaggtcagccttacacacccatagttcccaaagctgaaaacaggcctgcctccaacggtacctgctaatatcaggggagccttttcagcttacagagcaccctgtatgtgtttgtcttagttcaggccaccatctccaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaaggaaatctaagaggttgtaactatcactttttctagcctatataaggtaggtcagtaaggtagcaaaaacacatctgttgttttgctccttcaactctttttcctgattcttcctggggggaaaccgaaaacggtgagtaactggtggacacatcagaccccagactcttttcttcactgcatgcattcatattaggctcaggtgcttagactcctgttttccggtggctctgacacctggaaggattttaatctctgggagatgggcttttcatccatctgcttcccacctttcaggacaggtgcatgccttcttccacagaatgtctgcaagcagcccaaactgtatcctttcccacgtggaatttgcaacattgcatctctcgggctgctgtaggaaaatgccagtgcatgtgtaacatggtttacggctgcctatgcaaatgactgattatgtcagtataatttttataagaaaacaattgaatccttctttgggtcattttttttttccatttttggcatgtattcaaaagaaggctctgagacaaaaaaggctggggtgttttccgtatctggttttaatttggatattctgtcccgtcacttaatacaaaaccatgcttatcacattttaaaaattctagacaggcctggctcggtggcttgcatctgtcatcccagcactttgtgaggccaaggcaggcagatcacctgaggtcaggagctcaagaccagcctggccaacatggcaaaaccccgtctctactaaaaacacaaaaattagccaggcatggtagtgcgcacctgtaatcccagctactgggaaggcttaggcaggagaatcacttgagcccaggaggcggaggttgcggtgagccgagatcacgctcttgcactccagcctgggtgacagagtgagactccgtcttaatttaaaaaaaaaaataacgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 4027 bp, synthetic construct
  source 1..4027: mol_type = other DNA; note = pITR-sOCMp.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgcaatttatggtatagctgggaaacgtcaaagtcaagagttttgtaggaaagtcacgtcacttagccctgtctcctgtgccgggtgagacctgtgtgtgcacttggtgacaatggctttgagtctgtcaactccagactgaggtcagccttacacacccatagttcccaaagctgaaaacaggcctgcctccaacggtacctgctaatatcaggggagccttttcagcttacagagcaccctgtatgtgtttgtcttagttcaggccaccatctccaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaagtgcaatttatggtatagctgggaaacgtcaaagtcaagagttttgtaggaaagtcacgtcacttagccctgtctcctgtgccgggtgagacctgtgtgtgcacttggtgacaatggctttgagtctgtcaactccagactgaggtcagccttacacacccatagttcccaaagctgaaaacaggcctgcctccaacggtacctgctaatatcaggggagccttttcagcttacagagcaccctgtatgtgtttgtcttagttcaggccaccatctccaccttaccaggcatctagaaccttctccacactttgccaacagggttcgtttgcagaattgaaatcttagttaaggtttgttgaagtttgttgttgttttttttttttttttacaattggctgttcccacccacattcccttgagacataaatagaaaaaaaaaaaaaaagaggtttcatgagtaagacaagacatttgagctgcatccacttgatccttgaaaacgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 26 - DNA, 4532 bp, synthetic construct
  source 1..4532: mol_type = other DNA; note = pITR-sPrestinP.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaaagcaaactcatctctaaaccagaaataatagcaatatctatacaagtaaatacatgtactcagaacagtgcctactacatgtaaacactgaacaggtgttagcaacattgccattattgtgttagtatattaggtacctggtgctaccggcaaaaccagtttatcatccaactgtctccagtgttgctactcaaagtttggtcctccagtagcctatcaggatcacccaggggcctgttagaaaggcacatctcagaccccaccccagacctactgaatcagaatctgcgtttttaacgggatccgcaggtgattcctatgcacattaaagtgtaagaagtactgggctacagacaggtatgtgacaaaataatttcataggatggcaaaggccaagtggcaaatgaaggacaccagaaatgcacgtcccaggagcccaactcctccttagtaaattaccctattaagatttgtttagagatgttcaaaagcgtggagaaaagcaaatttggtttccctggtgcccttgaagagatcgccctcgtgtggagtagggagggaatctctagcctttcctctcggatgaagaacagcaccagcgctcccagccaaaggcctggcccaggttctggaggtggggtctccttggcagaagcctctggtgtctgcaggcgtgcatttacagctttaagaccaaacagctagtccgccacgtgtcactacagtgtgcacgcgcagaaatgcacaaagcaaaaaaaaaaaaaaagatgctcttaatgaaccaactataatccttgctaaggcataaagccagagggaagtatgtatctgaaatcattttctacccctcaccctcttggagcccggcactctggctgcggtgctctcttgtatcccagttgctagatgcaaaacaagctatttcctatctaattttttttttgttttataaattctaacttaaatgcccagaaaataactactcatactcacattgtcctctaattgaaaagataagtcaggttttttgtgtttttttttcattttaaaatcataatacgcaatgttttccacttgaacgctataccttgtgtattgtgcttgcttcagcctcgagcctctactgatgttccacctcaaggcgacaggaatgccacctggagaaactcctgggcggtatgggaagaaagccggtctcatcagagtatatttgcggggatcgacgaccaaggtgttaaattccaagcacgctttggaaagttctaggtgcttgggaagagatccgtaggcggcagggatgcccgcgccccggcgtcccagcgcggagggtggcggcggggcctggccctagcggggcggggcgggctcgggttaccgggagtcgcggggcgcggccggcactgcccgcggcgcctcctcctagagccgcacctggaggcagcgcgcgcgtcgaagaggcagcggctgtggagcgcggcggggcggctccgcccagggcagcccgggctgcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 27 - DNA, 3761 bp, synthetic construct
  source 1..3761: mol_type = other DNA; note = pITR-CHRNA10p.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtttcagatgccatcattaatgagaactatgactacctgaaggggttcttggaagacctggcaaggaactccccttggattaattggcttctctgcttctttgtaggtggattgctcaggtaatgacctggagcagttacacatcaaagtgacttcactgtgcagtcggatagagcagattcagtgtctggtattggctttccctttgtatttttgaatagaatataccattcaaagcctcctcgctcttctactatagtggttttgtttttaaaccctgagtgacgcttcacctttctaaatcagattcccttttgtaaaggggataatgattgctgatgttacttcacacagggctattttcaagaggaatcaattgagtagcatgagtactattccagatcttattttgatctgtcaagctgaagatgtgagcaaattccaattaagattagaccaaagacttctgagactttcaggaattcagggatgagaaagcagagtgggtcagctctgttgtctggaacttccatttaacttagatgcctcaggataggggttactcagctggaatcccctccactactgactcactatgtgaacctgagtgagtcacaaaacatagttggacttccagcaaagaacacctgacctggtttccttaccagaggaatgtttcagaaagtgagtatgctatagaaatggttagctcttagcagtgttcggaattgtgggccaggagcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 28 - DNA, 3939 bp, synthetic construct
  source 1..3939: mol_type = other DNA; note = pITR-DNM3p.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtccttgattcagagttaaagctatgggaaagtcctcaggcagaggacaaacattagacaagaaaatgcccatatatgaaaccctgcgaagcatcagtatttgaggagcagactaaaaaggaaccgtctgtggaggctaagagaagcatggccatttatctttgtgtcccgatcatcaggcacaggaccccacacacagtcacttctcaatgtgctaaatttcacagaatgcgtccagggtacctggttctggatagatccggtagaaggagatagaccgggagggcaaatggcatgaggagtctcacaggccagagtgattaaaggggtgtatcggggcggtaaaccctacagactctacctgtgcttatgcggggctggggaggacgagtcattacagatgaagaattaagtaaggtcagaccactcagggccttagatggatgtcacattgaagaatttagactccaacaggcctgccaccctgggaggagtcatcgcggattctggagaagggcgtgacagaggagatttcctttcgggaagtgtagtctggcagcggtgccccggtggtggcggcggcggtgctgctgttgctggtgatcgtgtggtggtgttagcggcgatagtgctttccactgggctttggcttggtagccgctgaaagagaacaacgctgccgctgctgctgatttcatgccatttcctgacccggcgctgtaacttggcctctgagccttggccacagaacgcagaggccgtggcatctggccgcagctgggctgcagtgcgtgcgcgcctggcctggtggtccgatgggaagcccggggcggggcagccgcggggcgggggcggggcgtcgcggagataggccacgcccctgcccgcccgcgcaggcgcgctgcgggtcgttagctgtccgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 29 - DNA, 4652 bp, synthetic construct
  source 1..4652: mol_type = other DNA; note = pITR-MUC15p.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttttctcctaattcagcacaaaaattgagttccttttctgtagctaaagagcttgtatgaactgtcagcttagctaaccatatgttttcaatgttccctgcaaattgtttaaggtatgtatagtcctttcaatggatgagtaagtcttttgtcattgttatttgctgcctgtggacttgatttcaaaatcttcttcaggtcatgaataaatttccttttccttctgtccctacttttgagccaaggaacaaatcaagattcttcctcagagtgtacacaccttcccaggcatctcactctctcctcactctatctgcttcaagttatggctcgttggtgagaacactctgctgctgaggttattatttagctataataactttttctaactagacagaaacaaattagatatgccaggattttctaattacctgccttaagtgcttttttagaaagcattaataaatcatgtggatcttttcctagcagtggtaagataagttataatattatcaaactgtcagttttgccacttcaatatatgtatgcctggttgtaacctcacttaataagttaagtccatgtaaaaatagttgatagttaataaattgggcaagagttgcttaaacagattagactatataacaaaattagggttttaaaagaataaagctgctataacagtacgcttcatctcacaggaattaatcagttatggtatctccacaaaacagaatatcacgtattgttgaagagagccgtctcatttccccggggttggtttaatttctaatcaaactctgaaggggcctttgggcttcagaaaatttaaaactatagaattaccttgttctttcctcgggccaattaactgggcagattctttgcattccatttgaagcttactagctcctgcattttagctaaagtttcgtttctcgctcagcagttgaaaacctatctccttgtgcagcagaaaccaagtatgaacctcaggcatattgagctgaacggcccttggcgccatccccaaacgctgatgtgcggaagatcccagtttcactcttctccctttcataagctctgaaaggaagtgtaggaagtatgccaagttgttattcaactctagtatttaatcaagcattacctgggcacttctgaaattctccagcttctaaagtgagagtaaaccagagagaacacagggtggaaactacttaatcgagaaggctcctaggataagtgaggatcacatggccattctcaggccccagttcctctccaaactcctgaaagtcagcaagaaaccgaatctcagtcatgatgattatttttcatgtaacacctcacagcgttctcagggatcccaatatatgctactaattcactttgtgttaagtaggagtttcttaaaaaaacaatttcagtggagaaattcctgctataccagatgactttgccaaaatctttgtcctttttttcacttagggtgaaaaaaaaaattgatgacccgtgttttgctaccactgacgagagtaataccttgtcccaaagctaaaacgatcaacctatgaaaactggagggttgggcttttgttgttgttgttaaaggcctgaatgaggtgatatcttcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - DNA, 4005 bp, synthetic construct
  source 1..4005: mol_type = other DNA; note = pITR-PLBD1p.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgacccattattcaatgggagttgtcaggatgtcagcaatgtacaaaatcattgcttaatttgtttgacaatggaaatggccattatggtttttatgtaactttgcttctgttacataattcttgctgacacggtgtttcaaccaaggtacttggtagcaagtgttgtacagaaaaggatctgtaagtggtttatgtggtcatcaaccacagcaaagatttcatctgagctgtgctatgaagaatgtagcttgagaaacacaaaatgtatcactgggcaaaaaggaagcagaagaaaaatacagttctgctaatgagagctctgactggtatctggagtataagatgggcccagccaatgctgagtgaatgaatgaaatgccttttgcctacttcacaatgtcacctagggcacccggtgccaacttcacaatatcacccaaggcataacttttgactacttcacaatgtcacttttaactgaccccacacagaaatggggactccacagaaacgtaggagtgtgtctagtgtcagccccgtctgaatcactctcctgtggtggctccagccaacgaagaggaagcaaaaaggataaaaaatctgagctacagcgcatggatttaggttaaacagcctgggaatgaggggtacgctaatcgctgaggaaaacgcacctgtggaggcctctccagaaacagcagaggatccgagctgcgtgtaggcagggcgcgcatgtcaccctggcccgggcgcctggtccgctgctggagataaatggtcgaccccggagggagaggctagtaggggtgttgatgtgaactgattcgcccaagccttgggccgcaaaactgcgaaagaaagcggcaggcagcctctgcatttcccagaagtgcagctggggaactttcccagaccggcccaggggttgctagagggtcagacgtaaaggatccgcctttcctaggcggggtgggccccaggcccgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 31 - DNA, 4437 bp, synthetic construct
  source 1..4437: mol_type = other DNA; note = pITR-RORBp.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtccaataaatgttggctcttgttttttctgacctgtatgttttgtctttgttccaaagctagccttacctctcccacatactggggttaattcatgctttggcccttatcaccttttccaatttatttcaaaattacatgctctattttaatatttgctttcttttttttattttgaaaacttattgaacttgcatctacactttaaaatgaagcagaacttaaagaactcaaagattatgaagaagactcagtacctgggaataaaattgagaataggttccttttatgactatataaccaatctcaaccattattttttgcttccccaaattaggagagtttaaaatgcagattctccccactctcctcttcccattcaatagaaactgagaaagaaggatcttattcaggtcttcactccatttgtgattcatattcagtggctgaaaggttagaaagcattcactccaccaataatgatcaagcacccataaagtaccaggagctcttacaaactctagggaaatcctggctcctgttgtcatgaattttgcattctcaggtaggaaatgtggctctgatgcctgctggggcagtgtacacttagagctacagaggatcttggaggtaatctaaaacctttctaaagagcaccctgcaatcacaccttctagcaacagccatttctcttgaattagtaaggtggctacaccgccaatttgagctgttctccttcagtcctgtagtccatcgccaggggagtctccaaatgctaataaaaatcaatttcccagacaaaagaacatagagggtcagggagcatctgacggacgtttttaaaggaaggggacagctacttccatgggactgcattttagttgtgctaaaagtgatgaaagtgggtttgcattattctaccaccaacacccaaaccacctgcccacggaaacccccgccggagaccgaagtttacccaaatagcgctcggcaaagcgctgccataaattcaaaactaactctgccgggcccgcgggggttgcgagacagggaccgaacgtgaaacccggggagccccgcgtctcttgcctccgaaggttttccgtgatcagtgtccccttctctgctggagtcggaagtgcctgtcacctgcggatctgcccgactctcccggtcggcccttcttctctgcccagttcggacagtctcgaattccccgtcgcagccccggccacctcggactccctggtccccagcccccgccccaccccccgcctccaccacgtcccctccccgcggtcccagcctctccaggcgctgctgggctctgattggcggctgcgctgacagcaggcggggcctggaagtcgcggccaagcccgccctcgcgtataagcccctctcagcgctctctctcccgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 32 - DNA, 4318 bp, synthetic construct
  source 1..4318: mol_type = other DNA; note = pITR-STRIP2p.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtccttgaatatttatgtccattttaacacttcctggttgcaagagggatgtgcctccattatttcctccacagttttggtatttgtcagacatttgttctgctgtctttctaatccagccaacgtctgctcaggaagtggggccagctccactgggacccatagttttacttccttgtcatttgattggatagtttccaaggaagcccctccagattggcactatctcagaaaaggagagcttgttgtgaaacactgcttcctgaaacttcctgctattgcctaaagctacgtctgaaactgagtagggaaaggcatacttttccagggacttagggggataggctttgaggtctctcctcgtgttgactctatgcaatcttcatagcaccagttttacacattccttctctgaaattaaagccagatggagcctctaggcttagcaagtggctttagatagccaccagaggggacttgcaagctgtcctctatcctactcccaagatcagtctgccctttcccctaggaataggcaggaaaagaataaaggaaagaaaggactggcgagcaggtgagggtgggggcttgctctaccctcaacatttacacaccatgaggaagaggccccctacagcagagaagggcagatgacaggagcagccctcgagggcaccaccaatttcagtgatggaaaaactcccccatcccacccttagacctccagtctcccagccaagccctagctccgggcgagatgcgttctcttcagaaaaacgctgagaattctcagcttccagagacagcgagtccctcgtttcgggcgatgtccctggccacctggcggtgccatccctcccctgagactaagcgggatatgggacgtgtgcaggagccgggatatggggggccgggtcggtggtaacagggaaacggagactgctgtggagcagtaggcggagactagagctccggaaaaggtcgctacagggacgggggtgagagctgagagacaccgagtgaggagcacagagataacccgcctgatctcaaggcccagctttcgcgaggtgtggagcctgtagctaacctaggagtctccgtccgccagcaatgccgcaggactaaaaagatcccctcaaaaatctcttcattgagcccccacctcctcgagtcccgctccggccggtcgagcagccaatcgcctcgcggggcggggttgcggcgagctgccgtaaccaatagaggtggagggggcggggcctggctcccggcgcgcggcggtagggtcgcctccggcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 4304 bp, synthetic construct
  source 1..4304: mol_type = other DNA; note = pITR-AQP11p.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtaggcatgagccactatgcccaaatgagaaataattttgtatgaaaaataatcttgtatggtaaatttagaccaagaataaaatgagtggttgtataagaaagaaagatgttcagaacaaaccaaaaagtccaagcatgtcacgaatggtctgtgtaagtcataataaaaggatttatctaaaaaaaccaaaaacttttatatgatcaagtcgtctataattaaaggaaaattataatgggtttttctagacattgggtgtgatgtaatgaaacgtacacactaaagaattcattacaaggctttcatgttttgttttttgtttgtttgactggtttgttgttgttgttgttgttgttgttgttgtttttgagacggagtttcgctcttgttgcccaggctggagtacaatggcgcgatctaggctcaccacaacctctgcctcccgggttcaagcgattctcctgcatcatcctcccgagtagctgggatgacaggcatgcgccaccatgcccggctaattttgtatttttagtagagacgggggtttctcatgttggtcaggctagtctcgaactcccgacctccggtgatccgcccgcctcggcctcccaaagtgctgggattataggcgtgagccaccgcgcccagccgcgcccggtttttgttgttgtttttgtttctaaaaacagcgtctcgctctgtggcccaggcaggggtgcagtggcgcgatctcagctcactgcagcctggaactcctggggtcaagcggtcttcccacctaagcctctccgtgctgggactccggacgcgctccacctcacgcagccgtattcctgctttcaaagcagatggaagaggtgcgccaggacccccagttcttggaaacagacctctccagttacctgttgtttcctcttcacgaagagtgcatgtaacagtaagacacaactgtttcatattatacgtaaagagttcatgccaaaggttatagacagtcacatgctaaaactaggctacactttgaagaatcaccgctcaagttctggaaaaaagaggtgactgttgaacaacactgtgagggtaatcgatgccactgaaatatacacttaaattgattaaagtggcgaattttatctggcatatattaccaccatttttagaaatgttttttggcaggtgaagaaaagcaaggctccaggaggccctgcgcaccggtctacgcccactaactcacccgccccctgcgccgcgtctcccctctcaatttcagtcgcccattgatcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 34 - DNA, 4576 bp, synthetic construct
  source 1..4576: mol_type = other DNA; note = pITR-KCNQ4p.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtagggcccatcctggtgtaaacaaaccctttggcgcagcccaagagagcccctatctaatacccagcacgatccccttacatccggagcactctttaaacatttttcctagctgatcttcacagtgaccctgcaggggagacaggaagaggtatcatgatccctgtgttagcgtgggagggctagtgaagtgcagtgacttgcccaaggtcactccatgaattgagggtggaattgaaaccaaaacactgatctcctgaccccctgtgcatacacagttgctcttggagattggagacccctggaatctggagcagacaatctggctggcttccttgcagctcaggtctgcggaggccacaagggggcagcatgcagccctcacctgtgtctctgggacctttgaagggagggtcctccctaggataacagtgagagctggaaactctaccctctccagagtattgcctcaagatccctgaacttagctccatgttttcagaatgtgctagctacaattcctgaaatgcccttttacttcccttttcacttattgagctcctatacatccatcaaggcccaatttaaatggccctttcagcagctatttctttggcaccttctgtgtgtcagacgttgttttaaacattgtgaatacagcttaaaacaagtctgacgggtggaaaggaaactgctgagggtggggtcaggggaacaggtgggagagggaccagtcccctccagcagaggggccaattgagggagcctgagacagctgtttgctcagaaaagtgtcttagtcactaaaggttgtggtggggaaagtccgtcctcccagtcatgtcctgggaatccggatggcgcaggaaggccacccggtgaccctaagagtggccacctgtcctctctgaactggactttctcttctggcccttcccctccctccctccctcactggcgctcagcagatcaatgctgcctttgctgacagctgagaatcgagctcgccttcccgccccttcccccgcccctcccgctcggcttcgtccctcgagatcctcccggaggaaccgggaagagtttgctgcggaaggctcaccctggggcagggcctgcggagggagcggctggtgtggccgcagctttccgtggaggaagagggaaagaggatcgggaaacccaagttaccaaccctgtgcaggggagatggaggtcggggactaagaaaaactgctgcccacccagccacacacagcactgggcacactttaagcacccgcaccaggcacacagtgctcgaccccaacggacacacctcatcctgccgcccgcggccacaactccacattcacttgcacgcgtccggcttcccggccccgcgcgctgcccccgccacgcggttcggcccaggcaccaactcggccgcccgtgcgccctgccccgccgcctgctccgcgcgttccctccctccgcctcgcctcgcttgctcgctcgctccctcccgatttgggaaggcggccgcggggcgggcgggggaggggcggggcgggggagggtcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 35 - DNA, 4235 bp, synthetic construct
  source 1..4235: mol_type = other DNA; note = pITR-LBHp.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgaattcgatgatgtgcttgtgtggacatgtggaggtctcaagaacaaaagaagagctgggcctggcacacagtgggtgctaatgcctgttagaattgttgttgagagggcaggagggtgtaacatggacccagctatctgatcctgaggctgggcgccatgtgggtgtgaagtacaccaggggctccaaccagcaagtgctagctcaggttacagtcagctgcccctggaggaagctagcagacatcctgtgtacttgaaaagaaaactgaaagtgctatctgcatcctggtgatagtaacctctcttttctggctgttgaagtgcattcctgtgcggatgtggaaagagagaaagcaagatacagccagggctaggacaggaatgtgagtatttccttaattggacatgagagccttgaactgattccagttggagtgttttcttttagggcctggaccctaaagatttcatacagttttctttgtcagaaaaatccctttggttcaaaggcccctcgatagaaataaagaaaaagccagggctgaatttctttgatatgtgggaaggcaagagtttatgagctgccagatctcaggcttcttttggggtggaggattttgtctggtgggttcgggttgctttgtgttgttgactgctaattcactgatgaccaagtttctcaaataccttaaaaacaagccctacgtctgctcagtgctttccaatttaccaagtgttttcataacatttcttatgtacgcaaatgagtttcaccgaaaaattggctagaaacttcccttctcctactcacgttcatagtgtagctgtgaaaacaaacaaaaccacagaggcatggtaagtgtggtatggtggggaaaacaaagccattttacaggcgtgattgaagcggaggccacagagcggcagcgctgggtcccgagtgagactcccatcatgtggctcaatggaaaaatcctacccaggacgacaccacatccttgctcccacaaataaaaccttccacggaactcagggctgcagacgcagagccgagcgcgcccccgagccgccgcccgccggagctgcgagcgctgaagccattcatgattttggtgacgttattccaggagtgggcgagggagggcggggcctctcggggccaagccccgcccccgcccctataaatacggcttcccgggctctttgtgggcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 36 - DNA, 4151 bp, synthetic construct
  source 1..4151: mol_type = other DNA; note = pITR-STRCp.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcactgccatctcaacatgtggtttctaggttgcctcaacagggaagagattgttggaggctcatttgctagctcttaaattctttggtcaaccacagtccattgaacagaactaatcacctgactgcaagagatctgggaaatgtgagaaacacctagatatctagtaagcaataaatatttcagttaccaaagccaaaccaaaaaaagagaaaaataattgtactttacaaagggaggcatctgggtcctgtgggagttttggggagtgaggatgtttcagagttctcaactcctgtggctatccatttcattttagcaggacatatgattaatttcttgttctggacctttgtaatttaaagtctgaatccttagcggcaagagaattgcttaatcaatggcttacaacagcagaacgtggactgccaggaaaatttccatcctgagttaagaaagaaggataatttattataagagggttgttacagaatgaagggcagaaattcagaaggattacaggatgggctggaaccacaaagcactgtctgctttttagactaggtgtggtatccttgatgggcaaagggaatattggtaaaattatttgtgacctgggttaagtcattccatttctctgggcttcaattcccctgtctataaaatgtttgagggagagaatggggaagggttctagggaaagaaggacagaataaaagtttgggtatatgaattactatttagagttggtataaagtgaaggcctttggggagatataccctgaccagaccagattattttgaatgaaatctctttctctgttccatgagcagttctgtgtgtagggagaacatttgaatggcctaatgagcaaatcacatttctctgggtctgtttccttatccataagttctgcatcactggctcctaactcaagcaatctccttgggtttctctgaggggcccctgggatcccctatcattagtccctctcacagaagcatacccttctccagagctaaaggatcagatattcagcggctcaggtaacaaacctgctgtcaggttacacatattgtttcctgaaagaccacactacagtgtcagtggagcctcaggttgcctgcagtcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 37 - DNA, 4827 bp, synthetic construct
  source 1..4827: mol_type = other DNA; note = pITR-TUBA8p.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgaagacatagttccagtctgagtctgaagcctgggacccatgagagctgaagacgtggtcccagtctgagtctgaagcctgagacccaggagagctgaagacgtggttccagtctgagtctgaagcctgagatccaggagagctaaggacatggttctagtctgagtctgaagcctgagacccaggagagctgatggtgtggttccagtctgagtctgaagcctgagacccaggagagctgaatacgtagttccagtctgagtctgaagcctgagttccaggagagctgaggacatggttccagtctgaatctgaagcctgagacccaggagagctgatggtgtggtctgaagacgtagttccagtctgagtctgaagcctgagacccaggagagctgaagatgtggtttcagtctgtctgaagcctgagacccaggagagctgatggtgtggttccagtctgagtctgaagtctgagacccaggagagctgaagatgtggttccagtctgagtctgaagcctgagacccagcagagctgaagacatggttccagtctgagtctgaagcctgagacccaggagagctgaagatgtggtttcagtctgtctgaagcctgagacccgggagagctgaagacgtagttccagtctgagtctgaagcctgagagacccaggagagctgatggtgtggttccagtctgagtctgaagcctgagacccaggagagctgaagatgtggtttcagtctgtctgaagcttgagacccaggggagctgaagatgtagttccagtctgagtctgaagcctgagacccaggagaggtgaagacgtggtttcagtctgagtcaaggcctgagaaccaggagagctgctggtgaaagttctagtgcaagggcagaagaccaatgtcctacctagctcaacagtcaggcaggcagaagttccctgtttctcagcctttttgttctattctgttcttcagttggttggatgaggcccctgcacattaaggatagacaaaaattcaacgcatgctttactaagtaccgtttgtatcagtgggtaaagcactgtgtttggtactctctcaaatgcaaagatgattacgacacatgtactatcgtttatgaatgggtggccaacagaacagattgccgcataggtaagcagaaatctgctctcattctctattggccacaagcaggcatgtcttaggagcagaagggtaggaagatctctaactgtgcttggaaacttggggagttaccacgtctggctaaagtggtattgtcttaaggaaaacctcttactactgggcagaggcaggggaaccctggtatgagttctggattacataggagatgtgacttggacacgtttggggcttaaaagtaggaagggatcaaggggggagatttgaaaatcccggtggaggtgcgaggtatccggggagaggtgggagcagaggccctgcagcttgccaagcacacacggccctagggcgcccagctgagacggcaccttggcacccgggcccgctgcagcccgctccggtcagctgcaccccagtcaggagcctttccagcgggtcggaggagaacggaagtttggggagacccgcgcgattcgcctggctgcattttacatttctttctccggcagctggggtcacgaaggctgctctcgccggcggtgttggaacgtggacacgtgcgctttggtaatagggcagcctcccccgcgggcgcagtccccgctgcgagcgcccccggctgctgaggcgggaccgaggacccggagatttcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 38 - DNA, 4926 bp, synthetic construct
  source 1..4926: mol_type = other DNA; note = pITR-prestinp.hKCNQ4.FLAG
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttaaacactgaacaggtgttagcaacattgccattattgtgttagtatattaggtacctggtgctaccggcaaaaccagtttatcatccaactgtctccagtgttgctactcaaagtttggtcctccagtagcctatcaggatcacccaggggcctgttagaaaggcacatctcagaccccaccccagacctactgaatcagaatctgcgtttttaacgggatccgcaggtgattcctatgcacattaaagtgtaagaagtactgggctacagacaggtatgtgacaaaataatttcataggatggcaaaggccaagtggcaaatgaaggacaccagaaatgcacgtcccaggagcccaactcctccttagtaaattaccctattaagatttgtttagagatgttcaaaagcgtggagaaaagcaaatttggtttcctcagctagggacgcggagagtggtctggtgcccttgaagagatcgccctcgtgtggagtagggagggaatctctagcctttcctctcggatgaagaacagcaccagcgctcccagccaaaggcctggcccaggttctggaggtggggtctccttggcagaagcctctggtgtctgcaggcgtgcatttacagctttaagaccaaacagctagtccgccacgtgtcactacagtgtgcacgcgcagaaatgcacaaagcaaaaaaaaaaaaaaagatgctcttaatgaaccaactataatccttgctaaggcataaagccagagggaagtatgtatctgaaatcattttctacccctcaccctcttggagcccggcactctggctgcggtgctctcttgtatcccagttgctagatgcaaaacaagctatttcctatctaatttttttttttaagagacggagtctcgctttgttgcccaggctggtctcaaactcctggactcaagcaattctcccagcttggggtaacgtgttacattattctacttaataaaaagcaaaagttgttttataaattctaacttaaatgcccagaaaataacttatcatgcattgccttgtcgtgcaatagtcaatatttgcaaaccaagtgttaaccaaaggcagttcatcaaagatttttgaaaattaaaaaaaaaaaaaaactcatactcacattgtcctcaggatttcctgttttcgaaatgttcctgtacgaatcggagtctctataatgattgtaattgaaaagataagtcaggttttttgtgtttttttttcattttaaaatcataatacgcaatgttttccacttgaacgctataccttgtgtattgtgcttgcttcagcctcgagcctctactgatgttccacctcaaggcgacaggaatgccacctggagaaactcctgggcggtatgggaagaaagccggtctcatcagagtatatttgcggggatcgacgaccaaggtgttaaattccaagcacgctttggaaagttctaggtgcttgggaagagatccgtaggcggcagggatgcccgcgccccggcgtcccagcgcggagggtggcggcggggcctggccctagcggggcggggcgggctcgggttaccgggagtcgcggggcgcggccggcactgcccgcggcgcctcctcctagagccgcacctggaggcagcgcgcgcgtcgaagaggcagcggctgtggagcgcggcggggcggctccgcccagggcagcccgggctgggccaaggagcgagctctcccttctcctgctctcagcctcagtgatcaaggcttcagtgaactgcactggagctcccagcgggggatcttgtcccctgtcccgacttttgtgctgcacattggatctggtgacactcaggaaattgcttgtctccggctgttaaggaataatttcagagtactcgccggtggcaggtggaaaggcgagcggcatggagcgcgtaataagagagttggagtcggaaagagcagccccagtcgccggggaagcgggaggtcagtgcgggctccggcggcccccaggctccgagcgcccgcccgcggccccggcccggcccctagcccccgccgcccgcgcccgccccgggtcgcccctctggccccgggtccgagccatgcgtctctgagcgccccgagcgcgcccccgccccggaccgtgcccgggccccggcgcccccagcccggcgccgcccaccggtcgctagccaccatggctgaagcccctcctagaaggcttggactgggacctcctcctggggatgctcctagagctgaactggtggctctgacagccgtgcagtctgaacaaggcgaagctggtggcggcggatctccacgtagacttggactgctgggaagccctcttcctcctggtgctccacttcctggacctggcagtggatctggatctgcctgtggccagagaagctctgccgctcacaagagataccggcggctgcagaactgggtgtacaacgtgctggaaagacccagaggctgggccttcgtgtaccacgtgttcatctttctgctggtgttcagctgcctggtgctgtccgtgctgagcaccatccaagaacatcaagagctggctaacgagtgcctgttaatactggagtttgtgatgattgtggtgttcggcctcgagtacatcgtccgcgtttggagcgccggctgctgctgcagatatagaggttggcaaggcagattccgcttcgccagaaagcccttctgcgtgatcgacttcatcgtgttcgtggccagcgtggccgtgattgctgctggcacacagggcaacatcttcgccacaagcgccctgcggagcatgcggtttctgcagatcctgagaatggtccgaatggacagaagaggcggcacctggaagctgctgggctctgtggtgtacgcccacagcaaagagctgatcaccgcctggtacatcggatttctggtgctgatcttcgcctccttcctggtgtacctggccgagaaggacgccaacagcgactttagcagctacgccgactctctttggtggggcaccatcacactgaccaccatcggctacggcgacaagacccctcacacatggctgggaagagtgctggccgctggatttgctctgctgggcatcagctttttcgccctgcctgccggaatcctcggatctggctttgccctgaaggtgcaagagcagcaccggcagaagcacttcgagaagagaagaatgcctgccgccaacctgattcaggccgcttggagactgtacagcaccgacatgagcagagcctacctgaccgccacgtggtattattacgactcgatcctgcctagcttccgcgaactggccctgctgtttgagcatgtgcagagagccagaaacggcggcctcagacctctggaagttcggagagcacctgtgcctgatggcgccccttctagatatcctccagtggccacctgtcacagacccggcagcacatctttttgccctggcgagtctagccggatgggcatcaaggacagaatcagaatgggcagcagccagcggagaacaggcccttctaaacagcatctggcccctccaaccatgcctacaagccctagctctgagcaagtgggcgaagccacctctcctaccaaggtgcagaagtcctggtccttcaacgaccggaccagattcagagccagcctgagactgaagcccagaacctctgccgaggatgccccttctgaagaggtggccgaagagaagtcctaccagtgcgagctgaccgtggacgacatcatgccagccgtgaaaaccgtgatacggtctatccggatcctgaagttcctggtggccaagcggaagttcaaagagacactgcggccctacgacgtgaaggacgtgatcgagcagtattctgccggccacctggacatgctgggcagaatcaagagcctgcagaccagagtggaccagatcgttggaagaggcccaggcgacagaaaggccagagagaagggcgataagggcccatctgatgccgaggttgtcgacgagatatcaatgatgggcagagtggtcaaggtggaaaaacaggtgcagagcatcgagcacaagctggacctgctgctgggattctacagccggtgtctgagaagcggcacatctgcatctctgggcgctgtgcaggtcccactgttcgaccctgatatcaccagcgactatcacagccccgtggaccacgaggacatctccgtttctgctcagaccctgagcatcagcagatccgtgtccaccaacatggacggatcccgggctgactacaaagaccatgacggtgattataaagatcatgacatcgactacaaggatgacgatgacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgttaattcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 39 - DNA, 69 bp, synthetic construct
  source 1..69: mol_type = other DNA; note = 3xFLAG tag sequence with stop codon
gactacaaagaccatgacggtgattataaagatcatgacatcgactacaaggatgacgatgacaagtaa

SEQ ID NO: 40 - DNA, 2208 bp, synthetic construct
  source 1..2208: mol_type = other DNA; note = AAV Anc80 Capsid DNA Sequence
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcaatcaccccaggaaccagactcctcttcgggcatcggcaagaaaggccagcagcccgcgaagaagagactcaactttgggcagacaggcgactcagagtcagtgcccgaccctcaaccactcggagaaccccccgcagccccctctggtgtgggatctaatacaatggcagcaggcggtggcgctccaatggcagacaataacgaaggcgccgacggagtgggtaacgcctcaggaaattggcattgcgattccacatggctgggcgacagagtcatcaccaccagcacccgaacctgggccctccccacctacaacaaccacctctacaagcaaatctccagccaatcgggagcaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgacgaatgatggcaccacgaccatcgccaataaccttaccagcacggttcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctacacgtttgaggacgtgccttttcacagcagctacgcgcacagccaaagcctggaccggctgatgaaccccctcatcgaccagtacctgtactacctgtctcggactcagaccacgagtggtaccgcaggaaatcggacgttgcaattttctcaggccgggcctagtagcatggcgaatcaggccaaaaactggctacccgggccctgctaccggcagcaacgcgtctccaagacagcgaatcaaaataacaacagcaactttgcctggaccggtgccaccaagtatcatctgaatggcagagactctctggtaaatcccggtcccgctatggcaacccacaaggacgacgaagacaaattttttccgatgagcggagtcttaatatttgggaaacagggagctggaaatagcaacgtggaccttgacaacgttatgataaccagtgaggaagaaattaaaaccaccaacccagtggccacagaacagtacggcacggtggccactaacctgcaatcgtcaaacaccgctcctgctacagggaccgtcaacagtcaaggagccttacctggcatggtctggcagaaccgggacgtgtacctgcagggtcctatctgggccaagattcctcacacggacggacactttcatccctcgccgctgatgggaggctttggactgaaacacccgcctcctcagatcctgattaagaatacacctgttcccgcgaatcctccaactaccttcagtccagctaagtttgcgtcgttcatcacgcagtacagcaccggacaggtcagcgtggaaattgaatgggagctgcagaaagaaaacagcaaacgctggaacccagagattcaatacacttccaactacaacaaatctacaaatgtggactttgctgttgacacaaatggcgtttattctgagcctcgccccatcggcacccgttacctcacccgtaatctg

SEQ ID NO: 41 - protein, 736 aa, synthetic construct
  source 1..736: note = AAV Anc80 Capsid Amino Acid Sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKKGQQPAKKRLNFGQTGDSESVPDPQPLGEPPAAPSGVGSNTMAAGGGAPMADNNEGADGVGNASGNWHCDSTWLGDRVITTSTRTWALPTYNNHLYKQISSQSGASTNDNTYFGYSTPWGYFDFNRFHCHFSPRDWQRLINNNWGFRPKRLNFKLFNIQVKEVTTNDGTTTIANNLTSTVQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQTTSGTAGNRTLQFSQAGPSSMANQAKNWLPGPCYRQQRVSKTANQNNNSNFAWTGATKYHLNGRDSLVNPGPAMATHKDDEDKFFPMSGVLIFGKQGAGNSNVDLDNVMITSEEEIKTTNPVATEQYGTVATNLQSSNTAPATGTVNSQGALPGMVWQNRDVYLQGPIWAKIPHTDGHFHPSPLMGGFGLKHPPPQILIKNTPVPANPPTTFSPAKFASFITQYSTGQVSVEIEWELQKENSKRWNPEIQYTSNYNKSTNVDFAVDTNGVYSEPRPIGTRYLTRNL

SEQ ID NO: 42 - DNA, 24 bp, synthetic construct
  source 1..24: mol_type = other DNA; note = Taqman probe for quantification of constructs
tctggctcaccgtcctcttcattt

SEQ ID NO: 43 - DNA, 20 bp, synthetic construct
  source 1..20: mol_type = other DNA; note = forward qPCR primer for quantification of constructs
caaacactccaccagcattg

SEQ ID NO: 44 - DNA, 20 bp, synthetic construct
  source 1..20: mol_type = other DNA; note = reverse qPCR primer for quantification of constructs
cagccacaacgaggatcata

SEQ ID NO: 45 - DNA, 1929 bp, synthetic construct
  source 1..1929: mol_type = other DNA; note = KCNQ4 3' UTR
gggacttctcagaggcagggcagcacacggccagccccgcggcctggcgctccgactgccctctgaggcctccggactcctctcgtacttgaactcactccctcacggggagagagaccacacgcagtattgagctgcctgagtgggcgtggtacctgctgtgggtgccagcgccccttccccacctcaggagcgtgagatgccaggtcgcacagagggcagcagcagcggccgtcccgcggcctctgggccccccagtgccctgcccactccatcaaggccctatgtggcccacctggcaggggcacagccccgggagtgggagcgggcgctggggccctgggccctgacccagcttccagctatgcaaggtgaggtctctggcccacccttcggacacagcagggaagccctcccgccaagtccccgccccacttgggggtgggccaaggtgcccccacaggtacccacaaagcacaggaccctgccacaaggcaggtggacaccatatatgcaaaccatgttaaatatgcaactttggggacccccatggggtctctctgtccctcccccattgggagctgggcccccagcagtagctggtctcaggctgcttggccaccaccctgtccctattctttggcttatcactccttcccctcccagcatggggcctgtttctcccctgccctctcctaagggcaatgcctgggcctttcttcccatttgcaagtgtcagctcccaggggctccctcctcctgctgggtggccactcccctccttggccctccagacaccactcatagtcagcacaggtttctgtatcctccccaaaactcccagacagtgcttcgtggacgatcgcacaaacatagccttttagtttctccagacaggaagaaagcctctcacacttaaacatgcaatgacgtgacacacttggagacatgagtgcagagccactcagccgctcctgggcctctgcagcagatgccagtggactggccttgcagggtgacgaccactaagaggaagacccccaactccatctgagcaggagaaggagctttgaagtaacccgagagctctccaggccccacccagacctttacccgctccccttcttcaagaagatctcctcctctctggtccaggagccctaacccactgcctctgcctgtccccaagggcccgcctccgtgtctccacagcacaactcgggcccaggcctgacaccactggagagaccccaggcccacttctagccaggcctgtgccttcctagtcactctaactcccagagagaataagaatgcatgtaatagctataccaaccgcgcatccggctttcacatgcactgtctcccctccctccacaccccacttcttcacttcaattggcagcgccacatccaggcgtcagcccccattcactccaggaacactttcttatccccacccctttgctcctcttctgcaaagccaatgcaggtggcaggaaggtgaggggtagtggaccaatggcaaccctctgtgggaacaaggggccgaggccacgctgcctgcatctcgtgctggggacctgcatgcgccagcaccagggcttggactggatcttactcagtccatggtgcccagcctctgccccaacatgccctctgcatgtgaccgtcatgccctggatggagccactcctggctcaccccacctgcactgcactgtccccagagagccacccctccacccactcagagacagctgtggagagggccaggagaatgggattaccctatgaccaaggagacatgggaagaagccctccttccttccacgatcgaggttccgccatcaactcggttctcggatatgcaagtacctcactttgttaacttattaacttattggtttcattaaagttttcaagagga

SEQ ID NO: 46 - DNA, 145 bp, synthetic construct
  source 1..145: mol_type = other DNA; note = 5' ITR
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 47 - DNA, 145 bp, synthetic construct
  source 1..145: mol_type = other DNA; note = 3' ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 48 - DNA, 122 bp, synthetic construct
  source 1..122: mol_type = other DNA; note = Poly A
aacttgtttattgcagcttataatggttacaaataaagcaatagcatcacaaatttcacaaataaagcatttttttcactgcattctagttgtggtttgtccaaactcatcaatgtatctta

SEQ ID NO: 49 - DNA, 6417 bp, synthetic construct
  source 1..6417: mol_type = other DNA; note = CMV.hsa.KCNQ4.mscarlet
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctagatcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgcaccggtgccaccatggccgaggcccccccgcgccgcctcggcctgggtcccccgcccggggacgccccccgcgcggagctagtggcgctcacggccgtgcagagcgaacagggcgaggcgggcgggggcggctccccgcgccgcctcggcctcctgggcagccccctgccgccgggcgcgcccctccctgggccgggctccggctcgggctccgcctgcggccagcgctcctcggccgcgcacaagcgctaccgccgcctgcagaactgggtctacaacgtgctggagcggccccgcggctgggccttcgtctaccacgtcttcatatttttgctggtcttcagctgcctggtgctgtctgtgctgtccactatccaggagcaccaggaacttgccaacgagtgtctcctcatcttggaattcgtgatgatcgtggttttcggcttggagtacatcgtccgggtctggtccgccggatgctgctgccgctaccgaggatggcagggtcgcttccgctttgccagaaagcccttctgtgtcatcgacttcatcgtgttcgtggcctcggtggccgtcatcgccgcgggtacccagggcaacatcttcgccacgtccgcgctgcgcagcatgcgcttcctgcagatcctgcgcatggtgcgcatggaccgccgcggcggcacctggaagctgctgggctcagtggtctacgcgcatagcaaggagctgatcaccgcctggtacatcgggttcctggtgctcatcttcgcctccttcctggtctacctggctgagaaggacgccaactccgacttctcctcctacgccgactcgctctggtgggggacgattacattgacaaccatcggctatggtgacaagacaccgcacacatggctgggcagggtcctggctgctggcttcgccttactgggcatctctttctttgccctgcctgccggcatcctaggctccggctttgccctgaaggtccaggagcagcaccggcagaagcacttcgagaagcggaggatgccggcagccaacctcatccaggctgcctggcgcctgtactccaccgatatgagccgggcctacctgacagccacctggtactactatgacagtatcctcccatccttcagagagctggccctcttgtttgagcacgtgcaacgggcccgcaatgggggcctacggcccctggaggtgcggcgggcgccggtacccgacggagcaccctcccgttacccgcccgttgccacctgccaccggccgggcagcacctccttctgccctggggaaagcagccggatgggcatcaaagaccgcatccgcatgggcagctcccagcggcggacgggtccttccaagcagcatctggcacctccaacaatgcccacctccccaagcagcgagcaggtgggtgaggccaccagccccaccaaggtgcaaaagagctggagcttcaatgaccgcacccgcttccgggcatctctgagactcaaaccccgcacctctgctgaggatgccccctcagaggaagtagcagaggagaagagctaccagtgtgagctcacggtggacgacatcatgcctgctgtgaagacagtcatccgctccatcaggattctcaagttcctggtggccaaaaggaaattcaaggagacactgcgaccgtacgacgtgaaggacgtcattgagcagtactcagcaggccacctggacatgctgggccggatcaagagcctgcaaactcgggtggaccaaattgtgggtcgggggcccggggacaggaaggcccgggagaagggcgacaaggggccctccgacgcggaggtggtggatgaaatcagcatgatgggacgcgtggtcaaggtggagaagcaggtgcagtccatcgagcacaagctggacctgctgttgggcttctattcgcgctgcctgcgctctggcacctcggccagcctgggcgccgtgcaagtgccgctgttcgaccccgacatcacctccgactaccacagccctgtggaccacgaggacatctccgtctccgcacagacgctcagcatctcccgctcggtcagcaccaacatggacggatcccgggctgactacaaagaccatgacggtgattataaagatcatgacatcgactacaaggatgacgatgacaagggctccggagagggcagaggaagtctgctaacatgcggtgacgtcgaggagaatcctggcccaatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagtaataagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 50 - DNA, 5555 bp, synthetic construct
  source 1..5555: mol_type = other DNA; note = CAG-GFP
tcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctttgtcgacgcggccgcacgcgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgaccggtcgctagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaagcttgaattcagctgacgtgcctcggaccgtcctaggaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacataggcgcgcctcgggccgtgcttgtaatcctgaggaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgattccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgacccgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccaggga

SEQ ID NO: 51 - DNA, 145 bp, synthetic construct
  source 1..145: mol_type = other DNA; note = 5' ITR
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

(19) United States
(12) Patent Application Publication
(10) Pub. No.: US 2025/0222137 A1
(43) Pub. Date: Jul. 10, 2025

(54) GENE THERAPY DELIVERY COMPOSITIONS AND METHODS FOR TREATING HEARING LOSS

(71) Applicant: Akouos, Inc., Boston, MA (US)

(72) Inventors: Katherine Diane GRIBBLE, Boston, MA (US); Danielle R. LENZ, Boston, MA (US); Robert NG, Boston, MA (US); Hao CHIANG, Boston, MA (US)

(21) Appl. No.: 18/697,425

(22) PCT Filed: Sep. 30, 2022
(86) PCT No.: PCT/US2022/077397

§ 371 Date: Mar. 29, 2024

Related U.S. Application Data
(60) Provisional application No. 63/251,017, filed on Sep. 30, 2021.

(51) Int. Cl. A61K 48/00; A61K 38/17; A61P 27/16; C12N 15/86

(52) U.S. Cl. CPC A61K 48/0058; A61K 38/1709; A61P 27/16; C12N 15/86; C12N 2750/14122; C12N 2750/14143; C12N 2830/008; C12N 2830/50

(57) ABSTRACT

The present disclosure provides constructs comprising a coding sequence operably linked to a promoter which expresses the polynucleotide in an outer hair cell, wherein the coding sequence encodes a polypeptide (e.g., a heterologous polypeptide). Exemplary constructs include AAV constructs. Also provided are methods of using disclosed constructs for the treatment of hearing loss and/or deafness.

### CROSS-REFERENCE TO RELATED APPLICATION

This application claims priority to U.S. Provisional Patent Application Ser. No. 63/251,017, filed Sep. 30, 2021, the entire contents of which are herein incorporated by reference.

### REFERENCE TO SEQUENCE LISTING SUBMITTED ELECTRONICALLY

The content of the electronically submitted sequence listing (Name: 4833_013PC01_SeqListing_ST26.xml; Size: 164,451 bytes; and Date of Creation: Sep. 29, 2022) is herein incorporated by reference in its entirety.

### BACKGROUND

Hearing loss can be conductive (arising from the ear canal or middle ear), sensorineural (arising from the inner ear or auditory nerve), or mixed. Most forms of nonsyndromic deafness are associated with permanent hearing loss caused by damage to structures in the inner ear (sensorineural deafness), although some forms may involve changes in the middle ear (conductive hearing loss). The great majority of human sensorineural hearing loss is caused by abnormalities in the hair cells of the organ of Corti in the cochlea (poor hair cell function). The hair cells may be abnormal at birth, or may be damaged during the lifetime of an individual (e.g., as a result of noise trauma or infection).

Treatments for hearing loss currently include hearing amplification for mild to severe losses and cochlear implantation for severe to profound losses (Kral and O'Donoghue, 2010, N. Engl. J. Med. 363:1438-1450). There is a need for improved treatment options for nonsyndromic deafness and other forms of hearing loss.

### SUMMARY

Certain aspects of the disclosure are directed to a construct comprising a polynucleotide encoding a polypeptide operably linked to a promoter which expresses the polynucleotide in an outer hair cell, wherein the promoter is selected from one or more of an oncomodulin (OCM) promoter, prestin promoter, cholinergic receptor nicotinic alpha 10 (CHRNA10) promoter, dynamin 3 (DNM3) promoter, mucin 14 (MUC15) promoter, phospholipase D (PLDB1) promoter, RAR related orphan receptor B (RORB) promoter, striatin interacting protein 2 (STRIP2) promoter, aquaporin 11 (AQP11) promoter, potassium voltage-gated channel subfamily Q member 4 (KCNQ4) promoter, LBH promoter, stereocilin (STRC) promoter, tubulin alpha 8 (TUBA8) promoter, or combinations thereof, an oncomodulin (OCM) promoter.

Certain aspects of the disclosure are directed to a construct comprising a polynucleotide encoding a polypeptide operably linked to a promoter which expresses the polynucleotide in an outer hair cell, wherein the promoter is heterologous to the polynucleotide.

In some aspects, promoter comprises a nucleic acid sequence having at least 85%, at least 90%, at least 95%, at least 96%, at least 97%, at least 98%, at least 99%, or 100% identity to any one of SEQ ID NOs: 1-15.

Certain aspects of the disclosure are directed to a construct comprising a polynucleotide encoding a polypeptide operably linked to a promoter which expresses the polynucleotide in an outer hair cell, wherein the promoter comprises a nucleic acid sequence having at least 85%, at least 90%, at least 95%, at least 96%, at least 97%, at least 98%, at least 99%, or 100% identity to any one of SEQ ID NOs: 1-15.

In some aspects, the prestin promoter comprises a nucleic acid sequence having at least 85%, at least 90%, at least 95%, at least 96%, at least 97%, at least 98%, at least 99%, or 100% identity to SEQ ID NO: 3 or SEQ ID NO: 15.

In some aspects, the oncomodulin (OCM) promoter comprises a nucleic acid sequence having at least 85%, at least 90%, at least 95%, at least 96%, at least 97%, at least 98%, at least 99%, or 100% identity to SEQ ID NO: 1 or SEQ ID NO: 2.

In some aspects, the promoter is heterologous to the polynucleotide.

In some aspects, polypeptide is an outer hair cell polypeptide, therapeutic polypeptide, or a reporter polypeptide.

In some aspects, the polynucleotide encoding a outer hair cell polypeptide comprises a gene selected from actin gamma 1 (ACTG1), adenylate cyclase type 1 (ADCY1), calcium binding protein 2 (CABP2), coiled-coil domain-containing 50 (CCDC50), cadherin-related 23 (CDH23), carcinoembryonic antigen-related cell adhesion molecule 16 (CEACAM16), chromodomain helicase DNA-binding protein 7 (CHD7), calcium- and integrin-binding family member 2 (CIB2), claudin 14 (CLDN14), chloride intracellular channel 5 (CLIC5), caseinolytic mitochondrial matrix peptidase proteolytic subunit (CLPP), clarin 1 (CLRN1), pejvakin (DFNB59), endothelin 3 (EDN3), ELMO domain-containing protein 3 (ELMOD3), epidermal growth factor receptor kinase substrate 8 (EPS8), espin (ESPN), estrogen-related receptor beta (ESRRB), eyes absent homolog 1 (EYA1), GIPC PDZ domain-containing family, member 3 (GIPC3), G protein-coupled receptor 98 (GPR98), G-protein signaling modulator 2 (GPSM2), glutaredoxin, cysteine-rich 1 (GRXCR1), glutaredoxin, cysteine-rich 2 (GRXCR2), immunoglobulin-like domain-containing receptor 1 (ILDR1), lysyl-tRNA synthetase (KARS), Potassium voltage-gated channel, KQT-like subfamily, member 4 (KCNQ4), lipoma HMGIC fusion partner-like 5 (LHFPL5), leucine-rich transmembrane and O-methyltransferase domain-containing (LRTOMT1 COMT2), tricellulin (MARVELD2), micro-ma 96 (MIR96), methionine sulfoxide reductase B3 (MSRB3), myosin, heavy chain 9, non-muscle (MYH9), myosin, heavy chain 14, non-muscle (MYH14), unconventional myosin IIIA (MYO3A), unconventional myosin VI (MYO6), unconventional myosin VIIA (MYO7A), unconventional myosin XVA (MYOl5A), otoferlin (OTOF), otogelin-like protein (OTOGL), purinergic recetpro P2X, ligand-gated ion channel, 2 (P2RX2), protocadherin 15 (PCDH15), PDZ domain-containing 7 (PDZD7), polyribonucleotide nucleotidyltransferase 1, mitochondrial (PNPT1), POU domain, class 4, transcription factor 3 (POU4F3), phosphoribosyl pyrophosphate synthetase 1 (PRPS1), protein tyrosine phosphatase, receptor type Q (PTPRQ), radixin (RDX), scaffold-containing ankyrin repeats and SAM domain (SANS), serpin peptidase inhibitor, clade B, member 6 (SERPINB6), SIX homeobox 1 (SIX1), SIX homeobox 5 (SIX5), prestin (SLC26A5), second mitochondrial-derived activator of caspase (SMAC/DIABLO), small muscle protein, x-linked (SMPX), stereocilin (STRC), nesprin-4 (SYNE4), TBC1 domain family, member 24 (TBC/D24), tight junction protein XO 2 (TJP2), transmembrane channel-like protein 1 (TMC1), transmembrane inner ear-expressed protein (TMIE), transmembrane protease, serine 3 (TMPRSS3), taperin (TPRN), TRIO and F-actin-binding protein (TRIOBP), Thrombospondin-type laminin G domain and EAR repeats (TSPEAR), harmonin (USH1C), usherin (USH2A), wolframin (WFS1), and whirlin (WHRN), or any combination thereof.

Certain aspects of the disclosure are directed to methods of using the constructs, vectors, viral particles (e.g., AAV), cells, compositions, and pharmaceutical compositions disclosed herein for expressing the polypeptide in an outer hair cell.

Certain aspects of the disclosure are directed to methods of using the constructs, vectors, viral particles (e.g., AAV), cells, compositions, and pharmaceutical compositions disclosed herein for increasing expression of the polypeptide in an outer hair cell. In some aspects, the increased expression is relative to the endogenous polypeptide expression in the outer hair cell.

Certain aspects of the disclosure are directed to methods of using the constructs, vectors, viral particles (e.g., AAV), cells, compositions, and pharmaceutical compositions disclosed herein for treating hearing loss.

### BRIEF DESCRIPTION OF THE DRAWINGS

FIGS. 1A-1C depicts in vitro expression of KCNQ4 protein from constructs including outer hair cell promoters. FIG. 1A shows KCNQ4-FLAG protein levels (“KCNQ4-FLAG”) in HEK293 cells transfected with 500 ng of exemplary plasmids comprising constructs driven by prestin, oncomodulin, CMV, or CAG promoters (red bands, white box). GAPDH is shown as a loading control (green). FIG. 1B shows KCNQ4-FLAG protein levels in HEK293 cells transfected with 400 ng of exemplary plasmids comprising constructs driven by DNM3, STRIP2, MUC15, LBD1, RORB, CHRNA10, prestin, oncomodulin, or CMV promoters (red bands, white box). GAPDH is shown as a loading control (green). FIG. 1C shows KCNQ4-FLAG protein levels in HEK293 cells transfected with 400 ng of exemplary plasmids comprising constructs driven by AQP11, KCNQ4, LBH, TUBA8, STRC, prestin, oncomodulin, or CMV promoters (red bands, white box). GAPDH is shown as a loading control (green).

FIG. 2 illustrates a perspective of a device for delivering fluid to an inner ear, according to aspects of the present disclosure.

FIG. 3 illustrates a sideview of a bent needle sub-assembly, according to aspects of the present disclosure.

FIG. 4 illustrates a perspective view of a device for delivering fluid to an inner ear, according to aspects of the present disclosure.

FIG. 5 illustrates a perspective view of a bent needle sub-assembly coupled to the distal end of a device, according to aspects of the present disclosure.

FIGS. 6A-6C depict in vivo expression of an rAAV construct encoding KCNQ4 protein under the control of a prestin promoter. FIG. 6A shows phalloidin staining of F-actin in the cochlea 28 days following administration of rAAV particles, comprising a construct of SEQ ID NO: 26, to the inner ear of postnatal day 2Kcnq4^{dn/+} KI mice. FIG. 6B shows expression of heterologous KCNQ4 in outer hair cells 28 days following administration of rAAV particles, comprising a construct of SEQ ID NO: 26, to the inner ear of postnatal day 2Kcnq4^{dn/+} KI mice. FIG. 6C shows a magnified view of heterologous KCNQ4 expression in outer hair cells in the 16 kHz frequency position of the cochlea 28 days following administration of rAAV particles, comprising a construct of SEQ ID NO: 26, to the inner ear of postnatal day 2Kcnq4^{dn/+} KI mice.

### DEFINITIONS

The scope of the present disclosure is defined by the claims appended hereto and is not limited by certain aspects described herein. Those skilled in the art, reading the present specification, will be aware of various modifications that may be equivalent to such described aspects, or otherwise within the scope of the claims. In general, terms used herein are in accordance with their understood meaning in the art, unless clearly indicated otherwise. Explicit definitions of certain terms are provided below; meanings of these and other terms in particular instances throughout this specification will be clear to those skilled in the art from context.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The articles “a” and “an,” as used herein, should be understood to include the plural referents unless clearly indicated to the contrary. Claims or descriptions that include “or” between one or more members of a group are considered satisfied if one, more than one, or all of the group members are present in, employed in, or otherwise relevant to a given product or process unless indicated to the contrary or otherwise evident from the context. In some aspects, exactly one member of a group is present in, employed in, or otherwise relevant to a given product or process. In some aspects, more than one, or all group members are present in, employed in, or otherwise relevant to a given product or process. It is to be understood that the present disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists (e.g., in Markush group or similar format), it is to be understood that each subgroup of the elements is also disclosed, and any element(s) can be removed from the group. It should be understood that, in general, where aspects or aspects are referred to as “comprising” particular elements, features, etc., certain aspects or aspects “consist,” or “consist essentially of,” such elements, features, etc. For purposes of simplicity, those aspects have not in every case been specifically set forth in so many words herein. It should also be understood that any embodiment or aspect can be explicitly excluded from the claims, regardless of whether the specific exclusion is recited in the specification.

Throughout the specification, whenever a polynucleotide or polypeptide is represented by a sequence of letters (e.g., A, C, G, and T, which denote adenosine, cytidine, guanosine, and thymidine, respectively in the case of a polynucleotide), such polynucleotides or polypeptides are presented in 5′ to 3′ or N-terminus to C-terminus order, from left to right.

Administration: As used herein, the term “administration” typically refers to administration of a construct or composition to a subject or system to achieve delivery of an agent to a subject or system. In some aspects, an agent is, or is included in, a composition; in some aspects, an agent is generated through metabolism of a composition or one or more components thereof. Those of ordinary skill in the art will be aware of a variety of routes that may, in appropriate circumstances, be utilized for administration to a subject, for example a human. For example, in some aspects, administration may be systematic or local. In some aspects, a systematic administration can be intravenous. In some aspects, administration can be local. Local administration can involve delivery to cochlear perilymph via, e.g., injection through a round-window membrane or into scala-tympani, a scala-media injection through endolymph, perilymph and/or endolymph following canalostomy. In some aspects, administration may involve only a single dose. In some aspects, administration may involve application of a fixed number of doses. In some aspects, administration may involve dosing that is intermittent (e.g., a plurality of doses separated in time) and/or periodic (e.g., individual doses separated by a common period of time) dosing. In some aspects, administration may involve continuous dosing (e.g., perfusion) for at least a selected period of time.

Allele: As used herein, the term “allele” refers to one of two or more existing genetic variants of a specific polymorphic genomic locus.

Amelioration: As used herein, the term “amelioration” refers to prevention, reduction or palliation of a state, or improvement of a state of a subject. Amelioration may include, but does not require, complete recovery or complete prevention of a disease, disorder or condition.

Amino acid: In its broadest sense, as used herein, the term “amino acid” refers to any compound and/or substance that can be incorporated into a polypeptide chain, e.g., through formation of one or more peptide bonds. In some aspects, an amino acid has a general structure, e.g., H₂N—C(H)(R)—COOH. In some aspects, an amino acid is a naturally-occurring amino acid. In some aspects, an amino acid is a non-natural amino acid; in some aspects, an amino acid is a D-amino acid; in some aspects, an amino acid is an L-amino acid. “Standard amino acid” refers to any of the twenty standard L-amino acids commonly found in naturally occurring peptides. “Nonstandard amino acid” refers to any amino acid, other than standard amino acids, regardless of whether it is prepared synthetically or obtained from a natural source. In some aspects, an amino acid, including a carboxy- and/or amino-terminal amino acid in a polypeptide, can contain a structural modification as compared with general structure as shown above. For example, in some aspects, an amino acid may be modified by methylation, amidation, acetylation, pegylation, glycosylation, phosphorylation, and/or substitution (e.g., of an amino group, a carboxylic acid group, one or more protons, and/or a hydroxyl group) as compared with a general structure. In some aspects, such modification may, for example, alter circulating half-life of a polypeptide containing a modified amino acid as compared with one containing an otherwise identical unmodified amino acid. In some aspects, such modification does not significantly alter a relevant activity of a polypeptide containing a modified amino acid, as compared with one containing an otherwise identical unmodified amino acid.

Approximately or About: As used herein, the terms “approximately” or “about” may be applied to one or more values of interest, including a value that is similar to a stated reference value. In some aspects, the term “approximately” or “about” refers to a range of values that fall within ±10% (greater than or less than) of a stated reference value unless otherwise stated or otherwise evident from context (except where such number would exceed 100% of a possible value). For example, in some aspects, the term “approximately” or “about” may encompass a range of values that within 10%, 9%, 8%, 7%, 6%, 5%, 4%, 3%, 2%, 1%, or less of a reference value.

Associated: As used herein, the term “associated” describes two events or entities as “associated” with one another, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some aspects, two or more entities are physically “associated” with one another if they interact, directly or indirectly, so that they are and/or remain in physical proximity with one another. In some aspects, two or more entities that are physically associated with one another are covalently linked to one another; in some aspects, two or more entities that are physically associated with one another are not covalently linked to one another but are non-covalently associated, for example by means of hydrogen bonds, van der Waals interaction, hydrophobic interactions, magnetism, and combinations thereof.

Biologically active: As used herein, the term “biologically active” refers to an observable biological effect or result achieved by an agent or entity of interest. For example, in some aspects, a specific binding interaction is a biological activity. In some aspects, modulation (e.g., induction, enhancement, or inhibition) of a biological pathway or event is a biological activity. In some aspects, presence or extent of a biological activity is assessed through detection of a direct or indirect product produced by a biological pathway or event of interest.

Cell Selective Promoter: As used herein, the term “cell selective promoter” refers to a promoter that is predominately active in certain cell types (e.g., transcription of a specific gene occurs only within cells expressing transcription regulatory and/or control proteins that bind to the tissue-specific promoter). In some aspects, an inner ear outer hair cell selective promoter is a promoter that is predominately active in one or more outer hair cells of the inner ear.

Characteristic portion: As used herein, the term “characteristic portion,” in the broadest sense, refers to a portion of a substance whose presence (or absence) correlates with presence (or absence) of a particular feature, attribute, or activity of the substance. In some aspects, a characteristic portion of a substance is a portion that is found in a given substance and in related substances that share a particular feature, attribute or activity, but not in those that do not share the particular feature, attribute or activity. In some aspects, a characteristic portion shares at least one functional characteristic with the intact substance. For example, in some aspects, a “characteristic portion” of a protein or polypeptide is one that contains a continuous stretch of amino acids, or a collection of continuous stretches of amino acids, that together are characteristic of a protein or polypeptide. In some aspects, each such continuous stretch generally contains at least 2, 5, 10, 15, 20, 50, or more amino acids. In general, a characteristic portion of a substance (e.g., of a protein, antibody, etc.) is one that, in addition to a sequence and/or structural identity specified above, shares at least one functional characteristic with the relevant intact substance. In some aspects, a characteristic portion may be biologically active.

Characteristic sequence: As used herein, the term “characteristic sequence” is a sequence that is found in all members of a family of polypeptides or nucleic acids, and therefore can be used by those of ordinary skill in the art to define members of the family.

Characteristic sequence element: As used herein, the phrase “characteristic sequence element” refers to a sequence element found in a polymer (e.g., in a polypeptide or nucleic acid) that represents a characteristic portion of that polymer. In some aspects, presence of a characteristic sequence element correlates with presence or level of a particular activity or property of a polymer. In some aspects, presence (or absence) of a characteristic sequence element defines a particular polymer as a member (or not a member) of a particular family or group of such polymers. A characteristic sequence element typically comprises at least two monomers (e.g., amino acids or nucleotides). In some aspects, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some aspects, a characteristic sequence element includes at least first and second stretches of contiguous monomers spaced apart by one or more spacer regions whose length may or may not vary across polymers that share a sequence element.

Combination therapy: As used herein, the term “combination therapy” refers to those situations in which a subject is simultaneously exposed to two or more therapeutic regimens (e.g., two or more therapeutic agents). In some aspects, two or more agents may be administered simultaneously. In some aspects, two or more agents may be administered sequentially. In some aspects, two or more agents may be administered in overlapping dosing regimens.

Comparable: As used herein, the term “comparable” refers to two or more agents, entities, situations, sets of conditions, subjects, populations, etc., that may not be identical to one another but that are sufficiently similar to permit comparison therebetween so that one skilled in the art will appreciate that conclusions may reasonably be drawn based on differences or similarities observed. In some aspects, comparable sets of agents, entities, situations, sets of conditions, subjects, populations, etc. are characterized by a plurality of substantially identical features and one or a small number of varied features. Those of ordinary skill in the art will understand, in context, what degree of identity is required in any given circumstance for two or more such agents, entities, situations, sets of conditions, subjects, populations, etc. to be considered comparable. For example, those of ordinary skill in the art will appreciate that sets of agents, entities, situations, sets of conditions, subjects, populations, etc. are comparable to one another when characterized by a sufficient number and type of substantially identical features to warrant a reasonable conclusion that differences in results obtained or phenomena observed under or with different sets of circumstances, stimuli, agents, entities, situations, sets of conditions, subjects, populations, etc. are caused by or indicative of the variation in those features that are varied.

Construct: As used herein, the term “construct” refers to a composition including a polynucleotide capable of carrying at least one heterologous polynucleotide. In some aspects, a construct can be a plasmid, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral vector, capsid, viral particle and any Gateway® plasmids. A construct can, e.g., include sufficient cis-acting elements for expression; other elements for expression can be supplied by the host primate cell or in an in vitro expression system. A construct may include any genetic element (e.g., a plasmid, a transposon, a cosmid, an artificial chromosome, or a viral vector, capsid, viral particle etc.) that is capable of replicating when associated with proper control elements. Thus, in some aspects, “construct” may include a cloning and/or expression construct and/or a viral construct (e.g., an adeno-associated virus (AAV) construct, an adenovirus construct, a lentivirus construct, or a retrovirus construct).

Conservative: As used herein, the term “conservative” refers to instances describing a conservative amino acid substitution, including a substitution of an amino acid residue by another amino acid residue having a side chain R group with similar chemical properties (e.g., charge or hydrophobicity). In general, a conservative amino acid substitution will not substantially change functional properties of interest of a protein, for example, ability of a receptor to bind to a ligand. Examples of groups of amino acids that have side chains with similar chemical properties include: aliphatic side chains such as glycine (Gly, G), alanine (Ala, A), valine (Val, V), leucine (Leu, L), and isoleucine (Ile, I); aliphatic-hydroxyl side chains such as serine (Ser, S) and threonine (Thr, T); amide-containing side chains such as asparagine (Asn, N) and glutamine (Gln, Q); aromatic side chains such as phenylalanine (Phe, F), tyrosine (Tyr, Y), and tryptophan (Trp, W); basic side chains such as lysine (Lys, K), arginine (Arg, R), and histidine (His, H); acidic side chains such as aspartic acid (Asp, D) and glutamic acid (Glu, E); and sulfur-containing side chains such as cysteine (Cys, C) and methionine (Met, M). Conservative amino acids substitution groups include, for example, valine/leucine/isoleucine (Val/Leu/Ile, V/L/I), phenylalanine/tyrosine (Phe/Tyr, F/Y), lysine/arginine (Lys/Arg, K/R), alanine/valine (Ala/Val, A/V), glutamate/aspartate (Glu/Asp, E/D), and asparagine/glutamine (Asn/Gln, N/Q). In some aspects, a conservative amino acid substitution can be a substitution of any native residue in a protein with alanine, as used in, for example, alanine scanning mutagenesis. In some aspects, a conservative substitution is made that has a positive value in the PAM250 log-likelihood matrix disclosed in Gonnet et al., 1992, Science 256:1443-1445, which is incorporated herein by reference in its entirety. In some aspects, a substitution is a moderately conservative substitution wherein the substitution has a nonnegative value in the PAM250 log-likelihood matrix. One skilled in the art would appreciate that a change (e.g., substitution, addition, deletion, etc.) of amino acids that are not conserved between the same protein from different species is less likely to have an effect on the function of a protein and therefore, these amino acids should be selected for mutation. Amino acids that are conserved between the same protein from different species should not be changed (e.g., deleted, added, substituted, etc.), as these mutations are more likely to result in a change in function of a protein. Exemplary conservative amino acid substitutions are shown in Table 1.

TABLE 1
Conservative Amino Acid Substitutions
CONSERVATIVE AMINO ACID SUBSTITUTIONS

| For Amino Acid | Code | Replace With |
|---|---|---|
| Alanine | A | D-ala, Gly, Aib, β-Ala, Acp, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, Aib, β-Ala, Acp |
| Isoleucine | I | D-Ile, Val, D-Val, AdaA, AdaG, Leu, D-Leu, |
| | | Met, D-Met |
| Leucine | L | D-Leu, Val, D-Val, AdaA, AdaG, Leu, D-Leu, |
| | | Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4 or 5-phenylproline, AdaA, |
| | | AdaG, cis-3,4 or 5-phenylproline, Bpa, D-Bpa |
| Proline | P | D-Pro, L-I-thioazolidine-4-carboxylic acid, |
| | | D-or-L-1-oxazolidine-4-carboxylic acid (Kauer, |
| | | U.S. Pat. No. 4,511,390) |
| Serine | S | D-Ser, Thr, D-Thr, allo-Thr, Met, D-Met, Met |
| | | (O), D-Met (O), L-Cys, D-Cys |
| Threonine | T | D-Thr, Ser, D-Ser, allo-Thr, Met, D-Met, Met |
| | | (O), D-Met (O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met, |
| | | AdaA, AdaG |

Control: As used herein, the term “control” refers to the art-understood meaning of a “control” being a standard against which results are compared. Typically, controls are used to augment integrity in experiments by isolating variables in order to make a conclusion about such variables. In some aspects, a control is a reaction or assay that is performed simultaneously with a test reaction or assay to provide a comparator. For example, in one experiment, a “test” (i.e., a variable being tested) is applied. In a second experiment, a “control,” the variable being tested is not applied. In some aspects, a control is a historical control (e.g., of a test or assay performed previously, or an amount or result that is previously known). In some aspects, a control is or comprises a printed or otherwise saved record. In some aspects, a control is a positive control. In some aspects, a control is a negative control.

Determining, measuring, evaluating, assessing, assaying and analyzing: As used herein, the terms “determining,” “measuring,” “evaluating,” “assessing,” “assaying,” and “analyzing” may be used interchangeably to refer to any form of measurement, and include determining if an element is present or not. These terms include both quantitative and/or qualitative determinations. Assaying may be relative or absolute. For example, in some aspects, “Assaying for the presence of” can be determining an amount of something present and/or determining whether or not it is present or absent.

Endogenous: As used herein in reference to a substances or process refers to a naturally occurring substances or processes that originates from within a system such as an organism, tissue, or cell.

Engineered: In general, as used herein, the term “engineered” refers to an aspect of having been manipulated by the hand of man. For example, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, the term “excipient” refers to an inactive (e.g., non-therapeutic) agent that may be included in a pharmaceutical composition, for example to provide or contribute to a desired consistency or stabilizing effect. In some aspects, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, the term “expression” of a nucleic acid sequence refers to generation of any gene product (e.g., transcript, e.g., mRNA, e.g., polypeptide, etc.) from a nucleic acid sequence. In some aspects, a gene product can be a transcript. In some aspects, a gene product can be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Flanked: As used herein, the term “flanked” refers to a position relative to ends of a reference item. For example, in referring to reference nucleic acid sequence(s), “flanked” refers to having a sequences upstream and downstream of the reference nucleic acid sequence(s). In some aspects, a flanked referenced nucleic acid sequence has a first sequence or series of nucleotide residues positioned adjacent to the 5′ end of the referenced nucleic acid and a second sequence or series of nucleotide residues positioned adjacent to the 3′ end of the referenced nucleic acid. In some aspects, the upstream and/or downstream flanking sequences are immediately adjacent to the referenced nucleic acid sequence. In some aspects, there are intervening nucleic acids between the upstream and/or downstream flanking sequences and the referenced nucleic acid sequence.

Functional: As used herein, the term “functional” describes something that exists in a form in which it exhibits a property and/or activity by which it is characterized. For example, in some aspects, a “functional” biological molecule is a biological molecule in a form in which it exhibits a property and/or activity by which it is characterized. In some such aspects, a functional biological molecule is characterized relative to another biological molecule which is non-functional in that the “non-functional” version does not exhibit the same or equivalent property and/or activity as the “functional” molecule. A biological molecule may have one function, two functions (i.e., bifunctional) or many functions (i.e., multifunctional).

Gene: As used herein, the term “gene” refers to a DNA sequence in a chromosome that codes for a gene product (e.g., an RNA product, e.g., a polypeptide product). In some aspects, a gene includes coding sequence (i.e., sequence that encodes a particular product). In some aspects, a gene includes non-coding sequence. In some particular aspects, a gene may include both coding (e.g., exonic) and non-coding (e.g., intronic) sequence. In some aspects, a gene may include one or more regulatory sequences (e.g., promoters, enhancers, etc.) and/or intron sequences that, for example, may control or impact one or more aspects of gene expression (e.g., cell-type-specific expression, inducible expression, etc.). As used herein, the term “gene” generally refers to a portion of a nucleic acid that encodes a polypeptide or fragment thereof, the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a polypeptide-coding nucleic acid. In some aspects, a gene may encode a polypeptide, but that polypeptide may not be functional, e.g., a gene variant may encode a polypeptide that does not function in the same way, or at all, relative to the wild-type gene. In some aspects, a gene may encode a transcript which, in some aspects, may be toxic beyond a threshold level. In some aspects, a gene may encode a polypeptide, but that polypeptide may not be functional and/or may be toxic beyond a threshold level.

Hair cell: As used herein, the term “hair cell” or “inner ear hair cell” refers to the sensory receptors of both the auditory system and the vestibular system in the ears of all vertebrates. The terms “hair cell” or “inner ear hair cell” refer to an inner hair cell and/or outer hair cell. The term “inner hair cell” or “inner ear inner hair cell” refers to cells of the inner ear that convert sound vibrations from the fluid in the cochlea into electrical signals that are then transmitted via the auditory nerve to the brain. The term “outer hair cell” or “inner ear outer hair cell” refers to cells of the inner ear that that amplify low-level sounds that enter into the fluids of the cochlea mechanically.

Hearing loss: As used herein, the term “hearing loss” may be used to a partial or total inability of a living organism to hear. In some aspects, hearing loss may be acquired. In some aspects, hearing loss may be hereditary. In some aspects, hearing loss may be genetic. In some aspects, hearing loss may be as a result of disease or trauma (e.g., physical trauma, treatment with one or more agents resulting in hearing loss, etc.). In some aspects, hearing loss may be due to one or more known genetic causes and/or syndromes. In some aspects, hearing loss may be of unknown etiology. In some aspects, hearing loss may or may not be mitigated by use of hearing aids or other treatments.

Heterologous: As used herein, the term “heterologous” the relationship between two or more nucleic acid or protein sequences that are derived from different sources. In some aspects, the promoter operably linked to the nucleic acid encoding the protein may be derived from a different gene other than the gene encoding the protein.

Identity: As used herein, the term “identity” refers to overall relatedness between polymeric molecules, e.g., between nucleic acid molecules (e.g., DNA molecules and/or RNA molecules) and/or between polypeptide molecules. In some aspects, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some aspects, a length of a sequence aligned for comparison purposes is at least 30%, at least 40%, at least 50%, at least 60%, at least 70%, at least 80%, at least 90%, at least 95%, or substantially 100% of length of a reference sequence; nucleotides at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4: 11-17, which is herein incorporated by reference in its entirety), which has been incorporated into the ALIGN program (version 2.0). In some aspects, nucleic acid sequence comparisons made with the ALIGN program use a PAM120 weight residue table, a gap length penalty of 12 and a gap penalty of 4.

Improve, increase, enhance, inhibit or reduce: As used herein, the terms “improve,” “increase,” “enhance,” “inhibit,” “reduce,” or grammatical equivalents thereof, indicate values that are relative to a baseline or other reference measurement. In some aspects, a value is statistically significantly difference that a baseline or other reference measurement. In some aspects, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some aspects, an appropriate reference measurement may be or comprise a measurement in comparable system known or expected to respond in a particular way, in presence of the relevant agent or treatment. In some aspects, an appropriate reference is a negative reference; in some aspects, an appropriate reference is a positive reference.

Knockdown: As used herein, the term “knockdown” refers to a decrease in expression of one or more gene products. In some aspects, an inhibitory nucleic acid achieve knockdown. In some aspects, a genome editing system described herein achieves knockdown.

Knockout: As used herein, the term “knockout” refers to ablation of expression of one or more gene products. In some aspects, a genome editing system described herein achieve knockout.

Nucleic acid: As used herein, the term “nucleic acid”, in its broadest sense, refers to any compound and/or substance that is or can be incorporated into an oligonucleotide chain. In some aspects, a nucleic acid is a compound and/or substance that is or can be incorporated into an oligonucleotide chain via a phosphodiester linkage. As will be clear from context, in some aspects, “nucleic acid” refers to an individual nucleic acid residue (e.g., a nucleotide and/or nucleoside); in some aspects, “nucleic acid” refers to an oligonucleotide chain comprising individual nucleic acid residues. In some aspects, a “nucleic acid” is or comprises RNA; in some aspects, a “nucleic acid” is or comprises DNA. In some aspects, a nucleic acid is, comprises, or consists of one or more natural nucleic acid residues. In some aspects, a nucleic acid is, comprises, or consists of one or more nucleic acid analogs. In some aspects, a nucleic acid analog differs from a nucleic acid in that it does not utilize a phosphodiester backbone. Alternatively or additionally, in some aspects, a nucleic acid has one or more phosphorothioate and/or 5′-N-phosphoramidite linkages rather than phosphodiester bonds. In some aspects, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some aspects, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some aspects, a nucleic acid comprises one or more modified sugars (e.g., 2′-fluororibose, ribose, 2′-deoxyribose, arabinose, and hexose) as compared with those in natural nucleic acids. In some aspects, a nucleic acid has a nucleotide sequence that encodes a functional gene product such as an RNA or protein. In some aspects, a nucleic acid includes one or more introns. In some aspects, nucleic acids are prepared by one or more of isolation from a natural source, enzymatic synthesis by polymerization based on a complementary template (in vivo or in vitro), reproduction in a recombinant cell or system, and chemical synthesis. In some aspects, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some aspects, a nucleic acid is partly or wholly single stranded; in some aspects, a nucleic acid is partly or wholly double stranded. In some aspects, a nucleic acid has a nucleotide sequence comprising at least one element that encodes, or is complementary to a sequence that encodes, a polypeptide. In some aspects, a nucleic acid has enzymatic activity.

Operably linked: As used herein, refers to a juxtaposition wherein the components described are in a relationship permitting them to function in their intended manner. A control element “operably linked” to a functional element is associated in such a way that expression and/or activity of the functional element is achieved under conditions compatible with the control element. In some aspects, “operably linked” control elements are contiguous (e.g., covalently linked) with coding elements of interest; in some aspects, control elements act in trans to or otherwise at a from the functional element of interest. In some aspects, “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. In some aspects, for example, a functional linkage may include transcriptional control. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Pharmaceutical composition: As used herein, the term “pharmaceutical composition” refers to a composition in which an active agent is formulated together with one or more pharmaceutically acceptable carriers. In some aspects, an active agent is present in unit dose amount appropriate for administration in a therapeutic regimen that shows a statistically significant probability of achieving a predetermined therapeutic effect when administered to a relevant population. In some aspects, a pharmaceutical composition may be specially formulated for administration in solid or liquid form, including those adapted for, e.g., administration, for example, an injectable formulation that is, e.g., an aqueous or non-aqueous solution or suspension or a liquid drop designed to be administered into an ear canal. In some aspects, a pharmaceutical composition may be formulated for administration via injection either in a particular organ or compartment, e.g., directly into an ear, or systemic, e.g., intravenously. In some aspects, a formulation may be or comprise drenches (aqueous or non-aqueous solutions or suspensions), tablets, boluses, powders, granules, pastes, capsules, powders, etc. In some aspects, an active agent may be or comprise an isolated, purified, or pure compound.

Pharmaceutically acceptable: As used herein, the term “pharmaceutically acceptable” which, for example, may be used in reference to a carrier, diluent, or excipient used to formulate a pharmaceutical composition as disclosed herein, means that a carrier, diluent, or excipient is compatible with other ingredients of a composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting a subject compound from one organ, or portion of a body, to another organ, or portion of a body. Each carrier must be is “acceptable” in the sense of being compatible with other ingredients of a formulation and not injurious to a patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polyadenylation: As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. In some aspects, a 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, a poly(A) tail can be added onto transcripts that contain a specific sequence, the polyadenylation signal or “poly(A) sequence.” A poly(A) tail and proteins bound to it aid in protecting mRNA from degradation by exonucleases. Polyadenylation can be affect transcription termination, export of the mRNA from the nucleus, and translation. Typically, polyadenylation occurs in the nucleus immediately after transcription of DNA into RNA, but additionally can also occur later in the cytoplasm. After transcription has been terminated, the mRNA chain can be cleaved through the action of an endonuclease complex associated with RNA polymerase. The cleavage site can be characterized by the presence of the base sequence AAUAAA near the cleavage site. After mRNA has been cleaved, adenosine residues can be added to the free 3′ end at the cleavage site. As used herein, a “poly(A) sequence” is a sequence that triggers the endonuclease cleavage of an mRNA and the additional of a series of adenosines to the 3′ end of the cleaved mRNA.

Polypeptide: As used herein, the term “polypeptide” refers to any polymeric chain of residues (e.g., amino acids) that are typically linked by peptide bonds.

In some aspects, a polypeptide has an amino acid sequence that occurs in nature. In some aspects, a polypeptide has an amino acid sequence that does not occur in nature. In some aspects, a polypeptide has an amino acid sequence that is engineered in that it is designed and/or produced through action of the hand of man. In some aspects, a polypeptide may comprise or consist of natural amino acids, non-natural amino acids, or both. In some aspects, a polypeptide may include one or more pendant groups or other modifications, e.g., modifying or attached to one or more amino acid side chains, at a polypeptide's N-terminus, at a polypeptide's C-terminus, or any combination thereof. In some aspects, such pendant groups or modifications may be acetylation, amidation, lipidation, methylation, pegylation, etc., including combinations thereof. In some aspects, polypeptides may contain L-amino acids, D-amino acids, or both and may contain any of a variety of amino acid modifications or analogs known in the art. In some aspects, useful modifications may be or include, e.g., terminal acetylation, amidation, methylation, etc. In some aspects, a protein may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

Polynucleotide: As used herein, the term “polynucleotide” refers to any polymeric chain of nucleic acids. In some aspects, a polynucleotide is or comprises RNA; in some aspects, a polynucleotide is or comprises DNA. In some aspects, a polynucleotide is, comprises, or consists of one or more natural nucleic acid residues. In some aspects, a polynucleotide is, comprises, or consists of one or more nucleic acid analogs. In some aspects, a polynucleotide analog differs from a nucleic acid in that it does not utilize a phosphodiester backbone. Alternatively or additionally, in some aspects, a polynucleotide has one or more phosphorothioate and/or 5′-N-phosphoramidite linkages rather than phosphodiester bonds. In some aspects, a polynucleotide is, comprises, or consists of one or more natural nucleosides (e.g., adenosine, thymidine, guanosine, cytidine, uridine, deoxyadenosine, deoxythymidine, deoxy guanosine, and deoxycytidine). In some aspects, a polynucleotide is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some aspects, a polynucleotide comprises one or more modified sugars (e.g., 2′-fluororibose, ribose, 2′-deoxyribose, arabinose, and hexose) as compared with those in natural nucleic acids. In some aspects, a polynucleotide has a nucleotide sequence that encodes a functional gene product such as an RNA or protein. In some aspects, a polynucleotide includes one or more introns. In some aspects, a polynucleotide is prepared by one or more of isolation from a natural source, enzymatic synthesis by polymerization based on a complementary template (in vivo or in vitro), reproduction in a recombinant cell or system, and chemical synthesis. In some aspects, a polynucleotide is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some aspects, a polynucleotide is partly or wholly single stranded; in some aspects, a polynucleotide is partly or wholly double stranded. In some aspects, a polynucleotide has a nucleotide sequence comprising at least one element that encodes, or is the complement of a sequence that encodes, a polypeptide. In some aspects, a polynucleotide has enzymatic activity.

Promoter: As used herein, the term “promoter” refers to a nucleic acid sequence that functions to control the transcription of one or more coding sequences (e.g., a gene or transgene, e.g., encoding a polypeptide), located upstream with respect to the direction of transcription of the transcription initiation site of the coding sequence. In some aspects, the promoter is structurally identified by the presence of a binding site for DNA-dependent RNA polymerase, transcription initiation sites or other DNA sequence (e.g., a transcription factor binding site, a repressor and/or activator protein binding site, or other sequences of nucleotides that act directly or indirectly to regulate the amount of transcription from the promoter).

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means.

Recombinant: As used herein, the term “recombinant” is intended to refer to polypeptides that are designed, engineered, prepared, expressed, created, manufactured, and/or or isolated by recombinant means, such as polypeptides expressed using a recombinant expression construct transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof, and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of a polypeptide or one or more component(s), portion(s), element(s), or domain(s) thereof. In some aspects, one or more of such selected sequence elements is found in nature. In some aspects, one or more of such selected sequence elements is designed in silico. In some aspects, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc).

Reference: As used herein, the term “reference” describes a standard or control relative to which a comparison is performed. For example, in some aspects, an agent, animal, individual, population, sample, sequence or value of interest is compared with a reference or control agent, animal, individual, population, sample, sequence or value. In some aspects, a reference or control is tested and/or determined substantially simultaneously with the testing or determination of interest. In some aspects, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control. In some aspects, a reference is a negative control reference; in some aspects, a reference is a positive control reference. In some aspects, the reference can be a compound, a protein, a polypeptide, or a polynucleotide disclosed in the present disclosure.

Regulatory Element: As used herein, the term “regulatory element” or “regulatory sequence” refers to non-coding regions of DNA that regulate, in some way, expression of one or more particular genes. In some aspects, such genes are apposed or “in the neighborhood” of a given regulatory element. In some aspects, such genes are located quite far from a given regulatory element. In some aspects, a regulatory element impairs or enhances transcription of one or more genes. In some aspects, a regulatory element may be located in cis to a gene being regulated. In some aspects, a regulatory element may be located in trans to a gene being regulated. For example, in some aspects, a regulatory sequence refers to a nucleic acid sequence which is regulates expression of a gene product operably linked to a regulatory sequence. In some such aspects, this sequence may be an enhancer sequence and other regulatory elements which regulate expression of a gene product.

Sample: As used herein, the term “sample” typically refers to an aliquot of material obtained or derived from a source of interest. In some aspects, a source of interest is a biological or environmental source. In some aspects, a source of interest may be or comprise a cell or an organism, such as a microbe (e.g., virus), a plant, or an animal (e.g., a human). In some aspects, a source of interest is or comprises biological tissue or fluid. In some aspects, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secretions, vitreous humour, vomit, and/or combinations or component(s) thereof. In some aspects, a biological fluid may be or comprise an intracellular fluid, an extracellular fluid, an intravascular fluid (blood plasma), an interstitial fluid, a lymphatic fluid, and/or a transcellular fluid. In some aspects, a biological fluid may be or comprise a plant exudate. In some aspects, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchioalveolar, ductal, nasal, ocular, oral, uterine, vaginal, or other washing or lavage). In some aspects, a biological sample is or comprises cells obtained from an individual. In some aspects, a sample is a “primary sample” obtained directly from a source of interest by any appropriate means. In some aspects, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Selective expression: As used herein, the term “selective expression” or “selectively expresses” refers to expression of a gene or polypeptide of interest predominately in certain specific cell types (e.g., inner ear cells, e.g., inner ear outer hair cells).

Subject: As used herein, the term “subject” refers to an organism, typically a mammal (e.g., a human, in some aspects including prenatal human forms). In some aspects, a subject is suffering from a relevant disease, disorder or condition. In some aspects, a subject is susceptible to a disease, disorder, or condition. In some aspects, a subject displays one or more symptoms or characteristics of a disease, disorder or condition. In some aspects, a subject does not display any symptom or characteristic of a disease, disorder, or condition. In some aspects, a subject is someone with one or more features characteristic of susceptibility to or risk of a disease, disorder, or condition. In some aspects, a subject is a patient. In some aspects, a subject is an individual to whom diagnosis and/or therapy is and/or has been administered.

Substantially: As used herein, the term “substantially” refers to a qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result. The term “substantially” is therefore used herein to capture a potential lack of completeness inherent in many biological and chemical phenomena.

Treatment: As used herein, the term “treatment” (also “treat” or “treating”) refers to any administration of a therapy that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some aspects, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some aspects, treatment may be of a subject who has been diagnosed as suffering from the relevant disease, disorder, and/or condition. In some aspects, treatment may be of a subject known to have one or more susceptibility factors that are statistically correlated with increased risk of development of a given disease, disorder, and/or condition.

Variant: As used herein, the term “variant” refers to a version of something, e.g., a gene sequence, that is different, in some way, from another version. To determine if something is a variant, a reference version is typically chosen and a variant is different relative to that reference version. In some aspects, a variant can have the same or a different (e.g., increased or decreased) level of activity or functionality than a wild type sequence. For example, in some aspects, a variant can have improved functionality as compared to a wild-type sequence if it is, e.g., codon-optimized to resist degradation, e.g., by an inhibitory nucleic acid, e.g., miRNA. Such a variant is referred to herein as a gain-of-function variant. In some aspects, a variant has a reduction or elimination in activity or functionality or a change in activity that results in a negative outcome (e.g., increased electrical activity resulting in chronic depolarization that leads to cell death). Such a variant is referred to herein as a loss-of-function variant. In some aspects, a gain-of-function variant is a codon-optimized sequence which encodes a transcript or polypeptide that may have improved properties (e.g., less susceptibility to degradation, e.g., less susceptibility to miRNA mediated degradation) than its corresponding wild type (e.g., non-codon optimized) version. In some aspects, a loss-of-function variant has one or more changes that result in a transcript or polypeptide that is defective in some way (e.g., decreased function, non-functioning) relative to the wild type transcript and/or polypeptide.

### DETAILED DESCRIPTION

The present disclosure is directed to constructs comprising a polynucleotide encoding a polypeptide and compositions comprising the same which are designed for selective transgene expression e.g., preferential expression in outer hair cells.

### Hearing Loss

Generally, an ear can be described as including: an outer ear, middle ear, inner ear, hearing (acoustic) nerve, and auditory system (which processes sound as it travels from the ear to the brain). In addition to detecting sound, ears also help to maintain balance. Thus, in some aspects, disorders of the inner ear can cause hearing loss, tinnitus, vertigo, imbalance, or combinations thereof.

Hearing loss can be the result of genetic factors, environmental factors, or a combination of genetic and environmental factors. About half of all people who have tinnitus—phantom noises in their auditory system (ringing, buzzing, chirping, humming, or beating)—also have an over-sensitivity to/reduced tolerance for certain sound frequency and volume ranges, known as hyperacusis (also spelled hyperacousis). A variety of nonsyndromic and syndromic-related hearing losses will be known to those of skill in the art (e.g., DFNB1 and DFNA3, or Bart-Pumphrey syndrome, hystrix-like ichthyosis with deafness (HID), palmoplantar keratoderma with deafness, keratitis-ichthyosis-deafness (KID) syndrome and Vohwinkel syndrome, respectively). Environmental causes of hearing impairment or loss may include, e.g., certain medications, specific infections before or after birth, and/or exposure to loud noise over an extended period. In some aspects, hearing loss can result from noise, ototoxic agents, presbycusis, disease, infection or cancers that affect specific parts of the ear. In some aspects, ischemic damage can cause hearing loss via pathophysiological mechanisms. In some aspects, intrinsic abnormalities, like congenital mutations to genes that play an important role in cochlear anatomy or physiology, or genetic or anatomical changes in supporting and/or hair cells can be responsible for or contribute to hearing loss.

Hearing loss and/or deafness is one of the most common human sensory deficits, and can occur for many reasons. In some aspects, a subject may be born with hearing loss or without hearing, while others may lose hearing slowly over time. Approximately 36 million American adults report some degree of hearing loss, and one in three people older than 60 and half of those older than 85 experience hearing loss. Approximately 1.5 in 1,000 children are born with profound hearing loss, and another two to three per 1,000 children are born with partial hearing loss (Smith et al., 2005, Lancet 365:879-890, which is incorporated in its entirety herein by reference). More than half of these cases are attributed to a genetic basis (Di Domenico, et al., 2011, J. Cell. Physiol. 226:2494-2499, which is incorporated in its entirety herein by reference).

Treatments for hearing loss currently consist of hearing amplification for mild to severe losses and cochlear implantation for severe to profound losses (Kral and O'Donoghue, 2010, N. Engl. J. Med. 363:1438-1450, which is incorporated in its entirety herein by reference). Recent research in this arena has focused on cochlear hair cell regeneration, applicable to the most common forms of hearing loss, including presbycusis, noise damage, infection, and ototoxicity. There remains a need for effective treatments, such as gene therapy, which can repair and/or mitigate a source of a hearing problem (see e.g., WO 2018/039375, WO 2019/165292, and PCT filing application US2019/060328, each of which is incorporated in its entirety herein by reference).

In some aspects, deafness and/or hearing loss can be conductive (arising from the ear canal or middle ear), sensorineural (arising from the inner ear or auditory nerve), or mixed. In some aspects, nonsyndromic deafness and/or hearing loss is associated with permanent hearing loss caused by damage to structures in the inner ear (sensorineural deafness). In some aspects, sensorineural hearing loss can be due to poor hair cell function. In some aspects, sensorineural hearing impairments involve the eighth cranial nerve (the vestibulocochlear nerve) or the auditory portions of the brain. In some such aspects, only the auditory centers of the brain are affected. In such a situation, cortical deafness may occur, where sounds may be heard at normal thresholds, but quality of sound perceived is so poor that speech cannot be understood. Hearing loss that results from changes in the middle ear is called conductive hearing loss. Some forms of nonsyndromic deafness and/or hearing loss involve changes in both the inner ear and the middle ear, called mixed hearing loss. Hearing loss and/or deafness that is present before a child learns to speak can be classified as prelingual or congenital. Hearing loss and/or deafness that occurs after the development of speech can be classified as postlingual. Most autosomal recessive loci related to syndromic or nonsyndromic hearing loss cause prelingual severe-to-profound hearing loss.

In some aspects, deafness or hearing loss may be nonsyndromic. In some aspects, deafness or hearing loss may syndromic. Nonsyndromic deafness or hearing loss is hearing loss that is not associated with other signs and symptoms. In contrast, syndromic deafness involves hearing loss that occurs with abnormalities in other parts of the body. Most cases of genetic deafness (70 percent to 80 percent) are nonsyndromic; the remaining cases are caused by specific genetic syndromes.

Nonsyndromic deafness can have different patterns of inheritance, and can occur at any age. Types of nonsyndromic deafness are named according to their inheritance patterns. Autosomal dominant forms are designated DFNA, autosomal recessive forms are DFNB, and X-linked forms are DFNX. Each type is also numbered in the order in which it was described. For example, DFNA1 was the first described autosomal dominant type of nonsyndromic deafness. In some aspects, nonsyndromic deafness or hearing loss can have autosomal dominant, autosomal rececessive, or X-linked inheritance patterns.

Between 75 percent and 80 percent of nonsyndromic deafness cases are inherited in an autosomal recessive pattern, which means both copies of the gene in each cell have mutations. Usually, each parent of an individual with autosomal recessive deafness is a carrier of one copy of the mutated gene, but is not affected by this form of hearing loss. In some aspects, nonsydromic deafness or hearing loss can be inherited in an autosomal recessive inheritance pattern (Venkatesh, et al., 2015, Med. J. Armed Forces India, 71(4) 363-368).

Another 20 percent to 25 percent of nonsyndromic deafness cases are autosomal dominant, which means one copy of the altered gene in each cell is sufficient to result in hearing loss. People with autosomal dominant deafness most often inherit an altered copy of the gene from a parent who has hearing loss. In some aspects, nonsydromic deafness or hearing loss can be inherited in an autosomal dominant inheritance pattern (e.g., DFNA2) (Venkatesh, et al., 2015, Med. J. Armed Forces India, 71(4) 363-368).

Between 1 percent and 2 percent of deafness and hearing loss cases show an X-linked pattern of inheritance, which means the mutated gene responsible for the condition is located on the X chromosome. In some aspects, nonsyndromic deafness or hearing loss can be inherited in an X-linked inheritance pattern (Venkatesh, et al., 2015, Med. J. Armed Forces India, 71(4) 363-368).

The causes of nonsyndromic deafness are complex. Researchers have identified more than 30 genes that, when altered, are associated with nonsyndromic deafness; however, some of these genes have not been fully characterized. Different mutations in the same gene can be associated with different types of hearing loss, and some genes are associated with both syndromic and nonsyndromic deafness (Venkatesh, et al., 2015, Med. J. Armed Forces India, 71(4) 363-368).

For example, genes associated with nonsyndromic deafness include, but are not limited to, ATP2B2, ACTG1, CDH23, CLDN14, COCH, COL11A2, DFNA5, DFNB31 (WHRN), DFNB59, ESPN, EYA4, GJB3, KCNQ4, LHFPL5, MYO15A, MYO6, MYO7A, OTOF, PCDH15, SLC26A4, STRC, TECTA, TMC1, TMIE, TMPRSS3, TRIOBP, USH1C, and WFS1 (Athena Diagnostics, 2017, “Hearing Loss Advanced Sequencing and CNV Evaluation”, 1-3). In some aspects the nonsyndromic deafness or hearing loss is associated with a gene selected from ATP2B2, ACTG1, CDH23, CLDN14, COCH, COL11A2, DFNA5, DFNB31, DFNB59, ESPN, EYA4, GJB3, KCNQ4, LHFPL5, MYO15A, MYO6, MYO7A, OTOF, PCDH15, SLC26A4, STRC, TECTA, TMC1, TMIE, TMPRSS3, TRIOBP, USH1C, and WFS1

OTOF-related deafness (DFNB9 nonsyndromic hearing loss) is characterized by two phenotypes: prelingual nonsyndromic hearing loss and, less frequently, temperature-sensitive nonsyndromic auditory neuropathy (TS-NSAN) (Azaiez, et al., 2008, “OTOF-Related Deafness”, GeneReviews, 1-16). Another form of progressive hearing impairment is associated with a mutation in the otoferlin gene (e.g., a E1700Q mutation), or is not temperature sensitive (Iwasa, et al. 2019, PLoS ONE 14(5):e0215932). In some aspects, hearing loss or deafness is otoferlin-related. In some aspects, the hearing loss or deafness is DFNB9 nonsyndromic hearing loss. In some aspects, hearing loss or deafness is associated with a mutation in the otoferlin gene.

DFNB59 (deafness, autosomal recessive 59), also known as Pejvakin or PJVK, is a 352 amino acid protein belonging to the gasdermin family in vertebrates. DFNB59 is encoded by a gene that maps to human chromosome 2q31.2, essential for the proper function of auditory pathway neurons and outer hair cell function. Mutations in DFNB59 are believed to cause non-syndromic sensorineural deafness autosomal recessive type 59, a form of sensorineural hearing impairment characterized by absent or severely abnormal auditory brainstem response but normal otoacoustic emissions (auditory neuropathy or auditory dys-synchrony). DFNB59 shares significant similarity with DFNA5, indicating that these genes share a common origin (Delmaghani, et al., 2006, Nat. Genet. 38:770-778). In some aspects, hearing loss or deafness is pejvakin-related. In some aspects, the hearing loss or deafness is DFNB59 nonsyndromic hearing loss. In some aspects, hearing loss or deafness is DFNA5 nonsydromic hearing loss.

Defects in ion channels are associated with deafness: DFNA2 nonsyndromic hearing loss is inherited as an autosomal dominant mutation in the KCNQ4 gene, which encodes the potassium voltage-gated channel subfamily KQT member 4 also known as voltage-gated potassium channel subunit Kv7.4. DFNA2 nonsyndromic hearing loss is characterized by symmetric, predominantly high-frequency sensorineural hearing loss (SNHL) that is progressive across all frequencies (Jung, et al., 2019, Exp. Mol. Med. 51:1-12). At younger ages, hearing loss tends to be mild in the low frequencies and moderate in the high frequencies; in older persons, the hearing loss is moderate in the low frequencies and severe to profound in the high frequencies. Although the hearing impairment is often detected during routine hearing assessment of a school-age child, it is likely that hearing is impaired from birth, especially at high frequencies. Most affected persons initially require hearing aids to assist with sound amplification between ages ten and 40 years. By age 70 years, all persons with DFNA2 hearing loss have severe-to-profound hearing impairment (Smith and Hildebrand, 2008, DFNA2 Nonsydrmoic Hearing Loss, GeneReviews, 1-14).

Usher syndrome (also known as Hallgren syndrome, Usher-Hallgren syndrome, retinitis pigmentosa-dysacusis syndrome, and dystrophia retinae dysacusis syndrome) is a rare disorder caused by a mutation in any one of at least ten genes, resulting in a combination of hearing loss and a gradual visual impairment, and is a leading cause of deaf blindness. The hearing loss is caused by a defective inner ear, whereas the vision loss results from retinitis pigmentosa (RP), a degeneration of the retinal cells. Usher syndrome has three clinical subtypes, denoted as I, II, and III. Subjects with Usher I are born profoundly deaf and begin to lose their vision in the first decade of life, learn to walk slowly as children due to problems in their vestibular system, and exhibit balance difficulties. Subjects with Usher II are not born deaf, but do have hearing loss, but do not seem to have noticeable problems with balance; they also begin to lose their vision later (in the second decade of life) and may preserve some vision even into middle age. Subjects with Usher syndrome III are not born deaf, but experience a gradual loss of their hearing and vision; they may or may not have balance difficulties (Toms, et al., 2020, Ther. Adv. Ophthalmol., 12:2515841420952194). In some aspects, hearing loss is syndromic. In some aspects, hearing loss is associated with Usher syndrome. In some aspects the deafness or hearing loss is associated with Usher syndrome I, Usher syndrome IL, or Usher syndrome III.

Mutations in the WFS1 gene cause more than 90 percent of Wolfram syndrome type 1 cases; Wolfram syndrome is a condition that affects many of the body's systems, most often characterized by high blood sugar levels resulting from a shortage of the hormone insulin (diabetes mellitus) and progressive vision loss due to degeneration of the nerves that carry information from the eyes to the brain (optic atrophy). However, people with Wolfram syndrome often also have pituitary gland dysfunction that results in the excretion of excessive amounts of urine (diabetes insipidus), hearing loss caused by changes in the inner ear (sensorineural deafness), urinary tract problems, reduced amounts of the sex hormone testosterone in males (hypogonadism), or neurological or psychiatric disorders. About 65 percent of people with Wolfram syndrome have sensorineural deafness that can range in severity from deafness beginning at birth to mild hearing loss beginning in adolescence that worsens over time. Furthermore, about 60 percent of people with Wolfram syndrome develop a neurological or psychiatric disorder, most commonly problems with balance and coordination (ataxia), typically beginning in early adulthood (Medlej, et al., 2004, J. Clin. Endocrinol. Metab., 89(4):1656-1661).

The WFS1 gene encodes a protein called wolframin thought to regulate the amount of calcium in cells. When Wolfram syndrome is caused by mutations in the WFS1 gene, it is inherited in an autosomal recessive pattern, and the wolframin protein has reduced or absent function. As a result, calcium levels within cells are not regulated and the endoplasmic reticulum does not work correctly. When the endoplasmic reticulum does not have enough functional wolframin, the cell triggers its own cell death (apoptosis) (Zmyslowska, et al., 2021, Cell Commun Signal, 19:116). The death of cells in the pancreas, specifically cells that make insulin (beta cells), causes diabetes mellitus in people with Wolfram syndrome. The gradual loss of cells along the optic nerve eventually leads to blindness in affected individuals. The death of cells in other body systems likely causes the various signs and symptoms of Wolfram syndrome type 1 (Urano, 2016, Curr. Diab. Rep., 16:6). In some aspects, hearing loss or deafness is syndromic hearing loss or deafness. In some aspects, the syndromic hearing loss or deafness is WFS1-related. In some aspects, the syndromic hearing loss or deafness is associated with Wolfram syndrome.

As is known to those of skill in the art, hair cells are sensory receptors for both auditory and vestibular systems of vertebrate ears. Hair cells detect movement in the environment and, in mammals, hair cells are located within the cochlea of the ear, in the organ of Corti. Mammalian ears are known to have two types of hair cells—inner hair cells and outer hair cells. Outer hair cells can amplify low level sound frequencies, either through mechanical movement of hair cell bundles or electrically-driven movement of hair cell soma. Inner hair cells transform vibrations in cochlear fluid into electrical signals that the auditory nerve transmits to the brain. In some aspects, hair cells may be abnormal at birth, or damaged during the lifetime of an individual.

### Polypeptides

Certain aspects of the disclosure are directed to polynucleotides encoding a polypeptide. In some aspects, the polynucleotide can encode a polypeptide that is capable of being expressed in a cell (e.g., an inner ear cell). In some aspects, the polynucleotide can encode a polypeptide that is capable of being expressed in a hair cell. In some aspects, the polynucleotide can encode a polypeptide that is capable of being expressed in an outer hair cell. In some aspects, the polynucleotide can encode a full length polypeptide or a functional fragment thereof.

Exemplary polypeptides encoded by the polynucleotide include, but are not limited to, transmembrane proteins, enzymes, growth factors, cytokines, receptors, receptor ligands, hormones, membrane proteins, membrane-associated proteins, antigens, and antibodies.

Exemplary polynucleotides encoding polypeptides include, but are not limited to, actin gamma 1 (ACTG1), adenylate cyclase type 1 (ADCY1), calcium binding protein 2 (CABP2), coiled-coil domain-containing 50 (CCDC50), cadherin-related 23 (CDH23), carcinoembryonic antigen-related cell adhesion molecule 16 (CEACAM16), chromodomain helicase DNA-binding protein 7 (CHD7), calcium- and integrin-binding family member 2 (CIB2), claudin 14 (CLDN14), chloride intracellular channel 5 (CLIC5), caseinolytic mitochondrial matrix peptidase proteolytic subunit (CLPP), clarin 1 (CLRN1), pejvakin (DFNB59), endothelin 3 (EDN3), ELMO domain-containing protein 3 (ELMOD3), epidermal growth factor receptor kinase substrate 8 (EPS8), espin (ESPN), estrogen-related receptor beta (ESRRB), eyes absent homolog 1 (EYA1), GIPC PDZ domain-containing family, member 3 (GIPC3), G protein-coupled receptor 98 (GPR98), G-protein signaling modulator 2 (GPSM2), glutaredoxin, cysteine-rich 1 (GRXCR1), glutaredoxin, cysteine-rich 2 (GRXCR2), immunoglobulin-like domain-containing receptor 1 (ILDR1), lysyl-tRNA synthetase (KARS), Potassium voltage-gated channel, KQT-like subfamily, member 4 (KCNQ4), lipoma HMGIC fusion partner-like 5 (LHFPL5), leucine-rich transmembrane and O-methyltransferase domain-containing (LRTOMT1 COMT2), tricellulin (MARVELD2), micro-ma 96 (MIR96), methionine sulfoxide reductase B3 (MSRB3), myosin, heavy chain 9, non-muscle (MYH9), myosin, heavy chain 14, non-muscle (MYH14), unconventional myosin IIIA (MYO3A), unconventional myosin VI (MYO6), unconventional myosin VIIA (MYO7A), unconventional myosin XVA (MYO15A), otoferlin (OTOF), otogelin-like protein (OTOGL), purinergic recetpro P2X, ligand-gated ion channel, 2 (P2RX2), protocadherin 15 (PCDH15), PDZ domain-containing 7 (PDZD7), polyribonucleotide nucleotidyltransferase 1, mitochondrial (PNPT1), POU domain, class 4, transcription factor 3 (POU4F3), phosphoribosyl pyrophosphate synthetase 1 (PRPS1), protein tyrosine phosphatase, receptor type Q (PTPRQ), radixin (RDX), scaffold-containing ankyrin repeats and SAM domain (SANS), serpin peptidase inhibitor, clade B, member 6 (SERPINB6), SIX homeobox 1 (SIX1), SIX homeobox 5 (SIX5), prestin (SLC26A5), second mitochondrial-derived activator of caspase (SMAC/DIABLO), small muscle protein, x-linked (SMPX), stereocilin (STRC), nesprin-4 (SYNE4), TBC1 domain family, member 24 (TBC/D24), tight junction protein XO 2 (TJP2), transmembrane channel-like protein 1 (TMC1), transmembrane inner ear-expressed protein (TMIE), transmembrane protease, serine 3 (TMPRSS3), taperin (TPRN), TRIO and F-actin-binding protein (TRIOBP), Thrombospondin-type laminin G domain and EAR repeats (TSPEAR), harmonin (USH1C), usherin (USH2A), wolframin (WFS1), and whirlin (WHRN).

In some aspects, the polynucleotide encoding an outer hair cell polynucleotide comprises a gene selected from cadherin-related 23 (CDH23), clarin 1 (CLRN1), pejvakin (DFNB59), Potassium voltage-gated channel, KQT-like subfamily, member 4 (KCNQ4), otoferlin (OTOF), protocadherin 15 (PCDH15), POU domain, class 4, transcription factor 3 (POU4F3), prestin (SLC26A5), stereocilin (STRC), transmembrane channel-like protein 1 (TMC1), TRIO and F-actin-binding protein (TRIOBP), harmonin (USH1C), usherin (USH2A), wolframin (WFS1), and whirlin (WHRN). In some aspects, the polynucleotide encoding an outer hair cell polynucleotide comprises KQT-like subfamily, member 4 (KCNQ4).

In some aspects, the encoded polypeptide is a human polypeptide. In some aspects, the encoded polypeptide is a functional fragment of a human polypeptide disclosed herein.

Exemplary polypeptides are disclosed in Li, Y. et al. Transcriptomes of cochlear inner and outer hair cells from adult mice. Sci. Data. 5:180199 doi: 10.1038/sdata.2018.199 (2018) and Nishio, S. et al. Gene Expression Profiles of the Cochlea and Vestibular Endorgans: Localization and Function of Genes Causing Deafness. Annals Otology, Rhinology & Laryngology 124(55) (2015), which are herein incorporated by reference in their entirety.

In some aspects, the polypeptides are outer hair cell polypeptides. In some aspects, the polypeptides are therapeutic polypeptides. In some aspects, the polypeptides are reporter polypeptides.

### Outer Hair Cell Polypeptides

Certain aspects of the disclosure are directed to polynucleotides encoding an outer hair cell polypeptide. The polynucleotide can encode a full length polypeptide or a functional fragment thereof.

Exemplary polypeptides encoded by the polynucleotide include, but are not limited to, transmembrane proteins, enzymes, growth factors, cytokines, receptors, receptor ligands, hormones, membrane proteins, membrane-associated proteins, antigens, and antibodies.

Exemplary polynucleotides encoding polypeptides include, but are not limited to, actin gamma 1 (ACTG1), adenylate cyclase type 1 (ADCY1), calcium binding protein 2 (CABP2), coiled-coil domain-containing 50 (CCDC50), cadherin-related 23 (CDH23), carcinoembryonic antigen-related cell adhesion molecule 16 (CEACAM16), chromodomain helicase DNA-binding protein 7 (CHD7), calcium- and integrin-binding family member 2 (CIB2), claudin 14 (CLDN14), chloride intracellular channel 5 (CLIC5), caseinolytic mitochondrial matrix peptidase proteolytic subunit (CLPP), clarin 1 (CLRN1), pejvakin (DFNB59), endothelin 3 (EDN3), ELMO domain-containing protein 3 (ELMOD3), epidermal growth factor receptor kinase substrate 8 (EPS8), espin (ESPN), estrogen-related receptor beta (ESRRB), eyes absent homolog 1 (EYA1), GIPC PDZ domain-containing family, member 3 (GIPC3), G protein-coupled receptor 98 (GPR98), G-protein signaling modulator 2 (GPSM2), glutaredoxin, cysteine-rich 1 (GRXCR1), glutaredoxin, cysteine-rich 2 (GRXCR2), immunoglobulin-like domain-containing receptor 1 (ILDR1), lysyl-tRNA synthetase (KARS), Potassium voltage-gated channel, KQT-like subfamily, member 4 (KCNQ4), lipoma HMGIC fusion partner-like 5 (LHFPL5), leucine-rich transmembrane and O-methyltransferase domain-containing (LRTOMT1 COMT2), tricellulin (MARVELD2), micro-ma 96 (MIR96), methionine sulfoxide reductase B3 (MSRB3), myosin, heavy chain 9, non-muscle (MYH9), myosin, heavy chain 14, non-muscle (MYH14), unconventional myosin IIIA (MYO3A), unconventional myosin VI (MYO6), unconventional myosin VIIA (MYO7A), unconventional myosin XVA (MYO15A), otoferlin (OTOF), otogelin-like protein (OTOGL), purinergic recetpro P2X, ligand-gated ion channel, 2 (P2RX2), protocadherin 15 (PCDH15), PDZ domain-containing 7 (PDZD7), polyribonucleotide nucleotidyltransferase 1, mitochondrial (PNPT1), POU domain, class 4, transcription factor 3 (POU4F3), phosphoribosyl pyrophosphate synthetase 1 (PRPS1), protein tyrosine phosphatase, receptor type Q (PTPRQ), radixin (RDX), scaffold-containing ankyrin repeats and SAM domain (SANS), serpin peptidase inhibitor, clade B, member 6 (SERPINB6), SIX homeobox 1 (SIX1), SIX homeobox 5 (SIX5), prestin (SLC26A5), second mitochondrial-derived activator of caspase (SMAC/DIABLO), small muscle protein, x-linked (SMPX), stereocilin (STRC), nesprin-4 (SYNE4), TBC1 domain family, member 24 (TBC/D24), tight junction protein XO 2 (TJP2), transmembrane channel-like protein 1 (TMC1), transmembrane inner ear-expressed protein (TMIE), transmembrane protease, serine 3 (TMPRSS3), taperin (TPRN), TRIO and F-actin-binding protein (TRIOBP), Thrombospondin-type laminin G domain and EAR repeats (TSPEAR), harmonin (USH1C), usherin (USH2A), wolframin (WFS1), and whirlin (WHRN).

In some aspects, the polynucleotide encoding an outer hair cell polynucleotide comprises a gene selected from cadherin-related 23 (CDH23), clarin 1 (CLRN1), pejvakin (DFNB59), Potassium voltage-gated channel, KQT-like subfamily, member 4 (KCNQ4), otoferlin (OTOF), protocadherin 15 (PCDH15), POU domain, class 4, transcription factor 3 (POU4F3), prestin (SLC26A5), stereocilin (STRC), transmembrane channel-like protein 1 (TMC1), TRIO and F-actin-binding protein (TRIOBP), harmonin (USH1C), usherin (USH2A), wolframin (WFS1), and whirlin (WHRN). In some aspects, the polynucleotide encoding an outer hair cell polynucleotide comprises KQT-like subfamily, member 4 (KCNQ4).

In some aspects, the encoded polypeptide is a human polypeptide. In some aspects, the encoded polypeptide is a functional fragment of a human polypeptide disclosed herein.

Exemplary polypeptides are disclosed in Li, Y. et al. Transcriptomes of cochlear inner and outer hair cells from adult mice. Sci. Data. 5:180199 doi: 10.1038/sdata.2018.199 (2018) and Nishio, S. et al. Gene Expression Profiles of the Cochlea and Vestibular Endorgans: Localization and Function of Genes Causing Deafness. Annals Otology, Rhinology & Laryngology 124(55) (2015), which are herein incorporated by reference in their entirety.

Certain aspects of the disclosure are directed to polynucleotides encoding a therapeutic polypeptide. The polynucleotide can encode a polypeptide that is capable of being expressed in a cell (e.g., an inner ear cell). The polynucleotide can encode a full length polypeptide or a functional fragment thereof.

Exemplary polypeptides encoded by the polynucleotide include, but are not limited to, transmembrane proteins, enzymes, growth factors, cytokines, receptors, receptor ligands, hormones, membrane proteins, membrane-associated proteins, antigens, and antibodies.

Exemplary polynucleotides encoding therapeutic polypeptides include, but are not limited to, actin gamma 1 (ACTG1), adenylate cyclase type 1 (ADCY1), calcium binding protein 2 (CABP2), coiled-coil domain-containing 50 (CCDC50), cadherin-related 23 (CDH23), carcinoembryonic antigen-related cell adhesion molecule 16 (CEACAM16), chromodomain helicase DNA-binding protein 7 (CHD7), calcium- and integrin-binding family member 2 (CIB2), claudin 14 (CLDN14), chloride intracellular channel 5 (CLIC5), caseinolytic mitochondrial matrix peptidase proteolytic subunit (CLPP), clarin 1 (CLRN1), pejvakin (DFNB59), endothelin 3 (EDN3), ELMO domain-containing protein 3 (ELMOD3), epidermal growth factor receptor kinase substrate 8 (EPS8), espin (ESPN), estrogen-related receptor beta (ESRRB), eyes absent homolog 1 (EYA1), GIPC PDZ domain-containing family, member 3 (GIPC3), G protein-coupled receptor 98 (GPR98), G-protein signaling modulator 2 (GPSM2), glutaredoxin, cysteine-rich 1 (GRXCR1), glutaredoxin, cysteine-rich 2 (GRXCR2), immunoglobulin-like domain-containing receptor 1 (ILDR1), lysyl-tRNA synthetase (KARS), Potassium voltage-gated channel, KQT-like subfamily, member 4 (KCNQ4), lipoma HMGIC fusion partner-like 5 (LHFPL5), leucine-rich transmembrane and O-methyltransferase domain-containing (LRTOMT1 COMT2), tricellulin (MARVELD2), micro-ma 96 (MIR96), methionine sulfoxide reductase B3 (MSRB3), myosin, heavy chain 9, non-muscle (MYH9), myosin, heavy chain 14, non-muscle (MYH14), unconventional myosin IIIA (MYO3A), unconventional myosin VI (MYO6), unconventional myosin VIIA (MYO7A), unconventional myosin XVA (MYO15A), otoferlin (OTOF), otogelin-like protein (OTOGL), purinergic recetpro P2X, ligand-gated ion channel, 2 (P2RX2), protocadherin 15 (PCDH15), PDZ domain-containing 7 (PDZD7), polyribonucleotide nucleotidyltransferase 1, mitochondrial (PNPT1), POU domain, class 4, transcription factor 3 (POU4F3), phosphoribosyl pyrophosphate synthetase 1 (PRPS1), protein tyrosine phosphatase, receptor type Q (PTPRQ), radixin (RDX), scaffold-containing ankyrin repeats and SAM domain (SANS), serpin peptidase inhibitor, clade B, member 6 (SERPINB6), SIX homeobox 1 (SIX1), SIX homeobox 5 (SIX5), prestin (SLC26A5), second mitochondrial-derived activator of caspase (SMAC/DIABLO), small muscle protein, x-linked (SMPX), stereocilin (STRC), nesprin-4 (SYNE4), TBC1 domain family, member 24 (TBC/D24), tight junction protein XO 2 (TJP2), transmembrane channel-like protein 1 (TMC1), transmembrane inner ear-expressed protein (TMIE), transmembrane protease, serine 3 (TMPRSS3), taperin (TPRN), TRIO and F-actin-binding protein (TRIOBP), Thrombospondin-type laminin G domain and EAR repeats (TSPEAR), harmonin (USH1C), usherin (USH2A), wolframin (WFS1), and whirlin (WHRN).

Exemplary polypeptides are disclosed in Li, Y. et al. Transcriptomes of cochlear inner and outer hair cells from adult mice. Sci. Data. 5:180199 doi: 10.1038/sdata.2018.199 (2018) and Nishio, S. et al. Gene Expression Profiles of the Cochlea and Vestibular Endorgans: Localization and Function of Genes Causing Deafness. Annals Otology, Rhinology & Laryngology 124(55) (2015), which are herein incorporated by reference in their entirety.

### Constructs

Among other things, the present disclosure provides that some polynucleotides as described herein are polynucleotide constructs. Polynucleotide constructs according to the present disclosure include all those known in the art, including cosmids, plasmids (e.g., naked or contained in liposomes) and viral constructs (e.g., lentiviral, retroviral, adenoviral, and adeno-associated viral constructs) that incorporate a polynucleotide encoding a polypeptide or characteristic portion thereof. Those of skill in the art will be capable of selecting suitable constructs, as well as cells, for making any of the polynucleotides described herein. In some aspects, a construct is a plasmid (i.e., a circular DNA molecule that can autonomously replicate inside a cell). In some aspects, a construct can be a cosmid (e.g., pWE or sCos series). In some aspects, the construct is a mammalian or a viral vector.

In some aspects, a construct is a viral construct. In some aspects, a viral construct is a lentivirus, retrovirus, adenovirus, or adeno-associated virus construct. In some aspects, a construct is an adeno-associated virus (AAV) construct (see, e.g., Asokan et al., Mol. Ther. 20: 699-7080, 2012, which is incorporated in its entirety herein by reference). In some aspects, the construct is a viral vector. In some aspects, the construct is a lentivirus, retrovirus, adenovirus, or adeno-associated virus vector. In some aspects, the construct is an AAV vector. In some aspects, a viral construct is an adenovirus construct. In some aspects, a viral construct may also be based on or derived from an alphavirus. Alphaviruses include Sindbis (and VEEV) virus, Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus, Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, Semliki Forest virus (SFV), and Venezuelan equine encephalitis virus (VEEV) have all been used to develop viral constructs for coding sequence delivery. Pseudotyped viruses may be formed by combining alphaviral envelope glycoproteins and retroviral capsids. Examples of alphaviral constructs can be found in U.S. Publication Nos. 20150050243, 20090305344, and 20060177819; constructs and methods of their making are incorporated herein by reference to each of the publications in its entirety.

Constructs provided herein can be of different sizes. In some aspects, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some aspects, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some aspects, a construct is a viral construct and can have a total number of nucleotides of up to 10 kb. In some aspects, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some aspects, a construct is an adeno-associated virus construct and can have a total number of nucleotides of up to 8 kb. In some aspects, an adeno-associated virus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

In some aspects, a construct is an adenovirus construct and can have a total number of nucleotides of up to 8 kb. In some aspects, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some aspects, a promoter can be a native promoter, a constitutive promoter, an inducible promoter, and/or a tissue-specific promoter. Non-limiting examples of control sequences are described herein.

In some aspects, the construct comprises a polynucleotide encoding a polypeptide operably linked to a promoter which selectively expresses the polynucleotide in an inner ear outer hair cell.

In some aspects, the construct comprises a 5′ ITR, a promoter which selectively expresses the polynucleotide in an inner ear outer hair, a polynucleotide encoding a polypeptide, a polyA, and a 3′ ITR. In some aspects, the construct comprises a 5′ ITR, a promoter which selectively expresses the polynucleotide in an inner ear outer hair, a 5′ UTR, a polynucleotide encoding a polypeptide, a polyA, and a 3′ ITR.

In some aspects, the construct comprises a 5′ ITR, a promoter which selectively expresses the polynucleotide in an inner ear outer hair, a 5′ UTR, a polynucleotide encoding a polypeptide, a 3′ UTR, a polyA, and a 3′ ITR. In some aspects, the construct comprises a 5′ ITR, a promoter which selectively expresses the polynucleotide in an inner ear outer hair, a 5′ UTR, a polynucleotide encoding a polypeptide, a polyA, and a 3′ ITR.

In some aspects, the construct comprise a 5′ ITR, a promoter which selectively expresses the polynucleotide in an inner ear outer hair cell, a 5′ UTR, a polynucleotide encoding a polypeptide, a tag, a 3′ UTR, a polyA, and a 3′ ITR. In some aspects, the construct comprise a 5′ ITR, a promoter which selectively expresses the polynucleotide in an inner ear outer hair cell, a 5′ UTR, a polynucleotide encoding a polypeptide, a tag, a polyA, and a 3′ ITR.

In some aspects, the construct comprises a 5′ ITR, an enhancer, a promoter which selectively expresses the polynucleotide in an inner ear outer hair, a polynucleotide encoding a polypeptide, a 3′ UTR, and a 3′ ITR. In some aspects, the construct comprises a 5′ ITR, an enhancer, a promoter which selectively expresses the polynucleotide in an inner ear outer hair, a 5′ UTR, a polynucleotide encoding a polypeptide, a 3′ UTR, a polyA, and a 3′ ITR. In some aspects, the construct comprises a 5′ ITR, an enhancer, a promoter which selectively expresses the polynucleotide in an inner ear outer hair, a 5′ UTR, a polynucleotide encoding a polypeptide, a polyA, and a 3′ ITR.

In some aspects, the construct comprise a 5′ ITR, an enhancer, a promoter which selectively expresses the polynucleotide in an inner ear outer hair cell, a 5′ UTR, a polynucleotide encoding a polypeptide, a tag, a 3′ UTR, a polyA, and a 3′ ITR. In some aspects, the construct comprise a 5′ ITR, an enhancer, a promoter which selectively expresses the polynucleotide in an inner ear outer hair cell, a 5′ UTR, a polynucleotide encoding a polypeptide, a tag, a polyA, and a 3′ ITR.

In some aspects, the construct comprises a polynucleotide encoding a polypeptide operably linked to a promoter which expresses the polynucleotide in an inner ear outer hair cell.

In some aspects, the construct comprises a 5′ ITR, a promoter which expresses the polynucleotide in an inner ear outer hair, a polynucleotide encoding a polypeptide, a polyA, and a 3′ ITR. In some aspects, the construct comprises a 5′ ITR, a promoter which expresses the polynucleotide in an inner ear outer hair, a 5′ UTR, a polynucleotide encoding a polypeptide, a polyA, and a 3′ ITR.

In some aspects, the construct comprises a 5′ ITR, a promoter which expresses the polynucleotide in an inner ear outer hair, a 5′ UTR, a polynucleotide encoding a polypeptide, a 3′ UTR, a polyA, and a 3′ ITR. In some aspects, the construct comprises a 5′ ITR, a promoter which expresses the polynucleotide in an inner ear outer hair, a 5′ UTR, a polynucleotide encoding a polypeptide, a polyA, and a 3′ ITR.

In some aspects, the construct comprise a 5′ ITR, a promoter which expresses the polynucleotide in an inner ear outer hair cell, a 5′ UTR, a polynucleotide encoding a polypeptide, a tag, a 3′ UTR, a polyA, and a 3′ ITR. In some aspects, the construct comprise a 5′ ITR, a promoter which expresses the polynucleotide in an inner ear outer hair cell, a 5′ UTR, a polynucleotide encoding a polypeptide, a tag, a polyA, and a 3′ ITR.

In some aspects, the construct comprises a 5′ ITR, an enhancer, a promoter which expresses the polynucleotide in an inner ear outer hair, a polynucleotide encoding a polypeptide, a 3′ UTR, and a 3′ ITR. In some aspects, the construct comprises a 5′ ITR, an enhancer, a promoter which expresses the polynucleotide in an inner ear outer hair, a 5′ UTR, a polynucleotide encoding a polypeptide, a 3′ UTR, a polyA, and a 3′ ITR. In some aspects, the construct comprises a 5′ ITR, an enhancer, a promoter which expresses the polynucleotide in an inner ear outer hair, a 5′ UTR, a polynucleotide encoding a polypeptide, a polyA, and a 3′ ITR.

In some aspects, the construct comprise a 5′ ITR, an enhancer, a promoter which expresses the polynucleotide in an inner ear outer hair cell, a 5′ UTR, a polynucleotide encoding a polypeptide, a tag, a 3′ UTR, a polyA, and a 3′ ITR. In some aspects, the construct comprise a 5′ ITR, an enhancer, a promoter which expresses the polynucleotide in an inner ear outer hair cell, a 5′ UTR, a polynucleotide encoding a polypeptide, a tag, a polyA, and a 3′ITR.

### AAV Particles

Among other things, the present disclosure provides AAV particles that comprise a construct encoding a polypeptide, and a capsid described herein. In some aspects, AAV particles can be described as having a serotype, which is a description of the construct strain and the capsid strain. In some aspects, the AAV particle has an AAV1, AAV2, AAV3 (e.g., AAV3B), AAV4, AAV5, AAV6, AAV7, AAV8, AAV9, AAV10, AAV11, or an AAV Anc80 serotype. In some aspects, the AAV particle has an AAVAnc80 serotype. In some aspects an AAV particle may be described as AAV2, wherein the particle has an AAV2 capsid and a construct that comprises characteristic AAV2 Inverted Terminal Repeats (ITRs). In some aspects, an AAV particle may be described as a pseudotype, wherein the capsid and construct are derived from different AAV strains, for example, AAV2/9 would refer to an AAV particle that comprises a construct utilizing the AAV2 ITRs and an AAV9 capsid.

### AAV Construct

The present disclosure provides polynucleotide constructs that comprise a polypeptide or characteristic portion thereof. In some aspects described herein, a polynucleotide comprising a polypeptide or characteristic portion thereof can be included in an AAV particle.

In some aspects, a polynucleotide construct comprises one or more components derived from or modified from naturally occurring AAV genomic construct. In some aspects, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV9 construct, an AAV2.7m8 construct, an AAV8BP2 construct, an AAV293 construct, or AAV Anc80 construct. In some aspects, the construct is derived from an AAV Anc80 construct. Additional exemplary AAV constructs that can be used herein are known in the art. See, e.g., Kanaan et al., Mol. Ther. Nucleic Acids 8:184-197, 2017; Li et al., Mol. Ther. 16(7): 1252-1260, 2008; Adachi et al., Nat. Commun. 5: 3075, 2014; Isgrig et al., Nat. Commun. 10(1): 427, 2019; and Gao et al., J. Virol. 78(12): 6381-6388, 2004; each of which is incorporated in its entirety herein by reference.

In some aspects, provided constructs comprise coding sequence, e.g., a nucleic acid encoding a polypeptide, one or more regulatory and/or control sequences, and optionally 5′ and 3′ AAV derived inverted terminal repeats (ITRs). In some aspects wherein a 5′ and 3′ AAV derived ITR is utilized, the polynucleotide construct may be referred to as a recombinant AAV (rAAV) construct. In some aspects, provided rAAV constructs are packaged into an AAV capsid to form an AAV particle. In some aspects, an AAV capsid is an Anc80 capsid (e.g., an Anc80L65 capsid).

In some aspects, AAV derived sequences (which are comprised in a polynucleotide construct) typically include the cis-acting 5′ and 3′ ITR sequences (see, e.g., B. J. Carter, in “Handbook of Parvoviruses,” ed., P. Tijsser, CRC Press, pp. 155 168, 1990, which is incorporated herein by reference in its entirety). Typical AAV2-derived ITR sequences are about 145 nucleotides in length. In some aspects, at least 75% of a typical ITR sequence (e.g., at least 80%, at least 85%, at least 90%, or at least 95%) is incorporated into a construct provided herein. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996, each of which is incorporated in its entirety by reference). In some aspects, any of the coding sequences and/or constructs described herein are flanked by 5′ and 3′ AAV ITR sequences. The AAV ITR sequences may be obtained from any known AAV, including presently identified AAV types.

In some aspects, polynucleotide constructs described in accordance with this disclosure and in a pattern known to the art (see, e.g., Asokan et al., Mol. Ther. 20: 699-7080, 2012, which is incorporated herein by reference in its entirety) are typically comprised of, a coding sequence or a portion thereof, at least one and/or control sequence, and optionally 5′ and 3′ AAV inverted terminal repeats (ITRs). In some aspects, provided constructs can be packaged into a capsid to create an AAV particle. An AAV particle may be delivered to a selected target cell. In some aspects, a nucleic acid coding sequence is operatively linked to and/or control components in a manner that permits coding sequence transcription, translation, and/or expression in a cell of a target tissue.

In some aspects, a construct is an rAAV construct. In some aspects, an rAAV construct can include at least 500 bp, at least 1 kb, at least 1.5 kb, at least 2 kb, at least 2.5 kb, at least 3 kb, at least 3.5 kb, at least 4 kb, or at least 4.5 kb. In some aspects, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some aspects, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some aspects, a promoter can be a native promoter, a constitutive promoter, an inducible promoter, and/or a tissue-specific promoter. Non-limiting examples of control sequences are described herein.

### Exemplary Construct Components

### Inverted Terminal Repeat Sequences (ITRs)

AAV derived sequences of a construct typically comprises the cis-acting 5′ and 3′ ITRs (See, e.g., B. J. Carter, in “Handbook of Parvoviruses”, ed., P. Tijsser, CRC Press, pp. 155 168 (1990), which is incorporated in its entirety herein by reference). Generally, ITRs are able to form a hairpin. The ability to form a hairpin can contribute to an ITRs ability to self-prime, allowing primase-independent synthesis of a second DNA strand. ITRs also play a role in integration of AAV construct (e.g., a coding sequence, e.g., a polynucleotide encoding a polypeptide into a genome of a subject's cell. ITRs can also aid in efficient encapsidation of an AAV construct in an AAV particle.

An rAAV particle (e.g., an AAV2/Anc80 particle) of the present disclosure can comprise a rAAV construct comprising a coding sequence (e.g., a polynucleotide encoding a polypeptide) and associated elements flanked by a 5′ and a 3′ AAV ITR sequences. In some aspects, an ITR is or comprises about 145 nucleic acids. In some aspects, an ITR is or comprises about 119 nucleic acids. In some aspects, an ITR is or comprises about 130 nucleic acids. In some aspects, all or substantially all of a sequence encoding an ITR is used. An AAV ITR sequence may be obtained from any known AAV, including presently identified mammalian AAV types. In some aspects an ITR is an AAV2 ITR.

An example of a construct molecule employed in the present disclosure is a “cis-acting” construct containing a transgene, in which the selected transgene sequence and associated regulatory elements are flanked by 5′ or “left” and 3′ or “right” AAV ITR sequences. 5′ and left designations refer to a position of an ITR sequence relative to an entire construct, read left to right, in a sense direction. For example, in some aspects, a 5′ or left ITR is an ITR that is closest to a promoter (as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some aspects, a 3′ or right ITR is an ITR that is closest to a polyadenylation sequence (as opposed to a promoter sequence) for a given construct, when a construct is depicted in a sense orientation, linearly. ITRs as provided herein are depicted in 5′ to 3′ order in accordance with a sense strand. Accordingly, one of skill in the art will appreciate that a 5′ or“left” orientation ITR can also be depicted as a 3′ or “right” ITR when converting from sense to antisense direction. Further, it is well within the ability of one of skill in the art to transform a given sense ITR sequence (e.g., a 5′/left AAV ITR) into an antisense sequence (e.g., 3′/right ITR sequence). One of ordinary skill in the art would understand how to modify a given ITR sequence for use as either a 5′/left or 3′/right ITR, or an antisense version thereof.

For example, in some aspects an ITR (e.g., a 5′ ITR) can have a sequence according to SEQ ID NO: 16. In some aspects, an ITR (e.g., a 3′ ITR) can have a sequence according to SEQ ID NO: 17. In some aspects, an ITR includes one or more modifications, e.g., truncations, deletions, substitutions or insertions, as is known in the art. In some aspects, an ITR comprises fewer than 145 nucleotides, e.g., 119, 127, 130, 134 or 141 nucleotides. For example, in some aspects, an ITR comprises 110, 111, 112, 113, 114, 115, 116, 117, 118, 119, 120, 121, 122, 123,124, 125, 126, 127, 128, 129, 130, 131, 132, 133, 134, 135, 136, 137, 138, 139, 140, 141, 142, 143 144, or 145 nucleotides. In some aspects, the ITR comprises about 119 nucleotides. In some aspects, the ITR comprises about 130 nucleotides. In some aspects an ITR (e.g., a 5′ ITR) can have a sequence according to SEQ ID NO: 16. In some aspects, an ITR (e.g., a 3′ ITR) can have a sequence according to SEQ ID NO: 17.

A non-limiting example of 5′ AAV ITR sequences includes SEQ ID NO: 16 or 46. A non-limiting example of 3′ AAV ITR sequences includes SEQ ID NO: 17 or 47. In some aspects, the 5′ and a 3′ AAV ITRs (e.g., SEQ ID NOs: 16 and 17, or SEQ ID NOs: 46 and 47) flank a portion of a coding sequence, e.g., all or a portion of a polynucleotide encoding a polypeptide. The ability to modify these ITR sequences is within the skill of the art. (See, e.g., texts such as Sambrook et al. “Molecular Cloning. A Laboratory Manual”, 2d ed., Cold Spring Harbor Laboratory, New York (1989); and K. Fisher et al., J Virol., 70:520 532 (1996), each of which is incorporated in its entirety herein by reference). In some aspects, a 5′ ITR comprises a nucleic acid sequence having at least 80%, at least 85%, at least 90%, at least 95%, at least 96%, at least 97%, at least 98% or at least 99%, or 100% identity to SEQ ID NO: 16. In some aspects, the 5′ ITR sequence has the nucleic acid sequence of SEQ ID NO: 16. In some aspects, the 5′ ITR comprises a nucleic acid sequence having at least 80%, at least 85%, at least 90%, at least 95%, at least 96%, at least 97%, at least 98% or at least 99%, or 100% identity to SEQ ID NO: 46. In some aspects, the 5′ ITR comprises the nucleic acid sequence of SEQ ID NO: 46.

In some aspects, the 3′ ITR comprises a nucleic acid sequence having at least 80%, at least 85%, at least 90%, at least 95%, at least 96%, at least 97%, at least 98% or at least 99%, or 100% identity to SEQ ID NO: 17. In some aspects, the 3′ ITR comprises the nucleic acid sequence of SEQ ID NO: 17. In some aspects, the 3′ ITR comprises a nucleic acid sequence having at least 80%, at least 85%, at least 90%, at least 95%, at least 96%, at least 97%, at least 98% or at least 99%, or 100% identity to SEQ ID NO: 47. In some aspects, the 3′ ITR comprises the nucleic acid sequence of SEQ ID NO: 47.

In some aspects, the 3′ ITR comprises a nucleic acid sequence having at least 80%, at least 85%, at least 90%, at least 95%, at least 96%, at least 97%, at least 98% or at least 99%, or 100% identity to SEQ ID NO: 51. In some aspects, the 3′ ITR comprises the nucleic acid sequence of SEQ ID NO: 51.

### Exemplary 5′ AAV ITR (SEQ ID NO: 46)

TTGGCCACTCCCTCTCTGCGCGCTCGCTCGCTCACTGAGGCCGCCCGGGCAAAGCCC GGGCGTCGGGCGACCTTTGGTCGCCCGGCCTCAGTGAGCGAGCGAGCGCGCAGAGA GGGAGTGGCCAACTCCATCACTAGGGGTTCCT

### Exemplary 3′ AAV ITR (SEQ ID NO: 47)

AGGAACCCCTAGTGATGGAGTTGGCCACTCCCTCTCTGCGCGCTCGCTCGCTCACTG AGGCCGGGCGACCAAAGGTCGCCCGACGCCCGGGCTTTGCCCGGGCGGCCTCAGTG AGCGAGCGAGCGCGCAGAGAGGGAGTGGCCAA

Exemplary 5′ AAV ITR
(SEQ ID NO: 16)
CTGCGCGCTCGCTCGCTCACTGAGGCCGCCCGGGCGTCGGGCGACCTTT
GGTCGCCCGGCCTCAGTGAGCGAGCGAGCGCGCAGAGAGGGAGTGGCCA
ACTCCATCACTAGGGGTTCCT
Exemplary 3′ AAV ITR
(SEQ ID NO: 17)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAG
Exemplary 5′ ITR
(SEQ ID NO: 51)
AGGAACCCCTAGTGATGGAGTTGGCCACTCCCTCTCTGCGCGCTCGCTC
GCTCACTGAGGCCGCCCGGGCAAAGCCCGGGCGTCGGGCGACCTTTGGT
CGCCCGGCCTCAGTGAGCGAGCGAGCGCGCAGAGAGGGAGTGGCCAA

### Promoters

In some aspects, the disclosure is directed to constructs comprising a cell selective promoter which can be used to regulate (e.g., increase) expression of a polynucleotide encoding a polypeptide in a cell (e.g., an inner ear cell, e.g., an outer hair cell). In some aspects, the increased expression is relative to the endogenous polynucleotide expression in the cell.

In some aspects, a construct (e.g., an rAAV construct) comprises a promoter. The term “promoter” refers to a DNA sequence recognized by enzymes/proteins that can promote and/or initiate transcription of an operably linked gene (e.g., a polynucleotide encoding a polypeptide). For example, a promoter typically refers to, e.g., a nucleotide sequence to which an RNA polymerase and/or any associated factor binds and from which it can initiate transcription. Thus, in some aspects, a construct (e.g., an rAAV construct) comprises a polynucleotide operably linked to one of the non-limiting example promoters described herein.

In some aspects, a promoter is an inducible promoter, a constitutive promoter, a mammalian cell promoter, a viral promoter, a chimeric promoter, an engineered promoter, a tissue-specific promoter, a cell-selective promoter or any other type of promoter known in the art. In some aspects, a promoter is a RNA polymerase II promoter, such as a mammalian RNA polymerase II promoter. In some aspects, a promoter is a RNA polymerase III promoter, including, but not limited to, a HI promoter, a human U6 promoter, a mouse U6 promoter, or a swine U6 promoter. A promoter will generally be one that is able to promote transcription in an inner ear cell. In some aspects, a promoter is a cochlea-selective promoter or a cochlea-oriented promoter. In some aspects, a promoter is a hair cell selective promoter, or a outer hair cell selective promoter. In some aspects, a promoter is an inner ear outer hair cell selective promoter.

The term “constitutive” promoter refers to a nucleotide sequence that, when operably linked with a nucleic acid encoding a protein (e.g., a polypeptide), causes RNA to be transcribed from the nucleic acid in a cell under most or all physiological conditions.

Examples of constitutive promoters include, without limitation, the retroviral Rous sarcoma virus (RSV) LTR promoter, the cytomegalovirus (CMV) promoter (see, e.g., Boshart et al., Cell 41:521-530, 1985, which is incorporated in its entirety herein by reference), the SV40 promoter, the dihydrofolate reductase promoter, the beta-actin promoter, the phosphoglycerol kinase (PGK) promoter, and the EF1-alpha promoter (Invitrogen). In some aspects, the promoter is a constitutive promoter. In some aspects, the constitutive promoter is a CAG promoter, a CBA promoter, a CMV promoter, a CMV/CBA enhancer/promoter, or a CB7 promoter.

In some aspects, regulatory and/or control sequences impart cell selective gene expression capabilities. In some cases, cell selective regulatory and/or control sequences bind cell selective transcription factors that induce transcription in a cell selective manner.

In some aspects, a cell selective promoter is an ear cell selective promoter. In some aspects, a cell selective promoter is an inner ear cell selective promoter. In some aspects, the promoter is an inner ear outer hair cell selective promoter.

In some aspects, inner ear outer hair cell selective promoters are selected from one or more of oncomodulin, prestin, CHRNA10, DNM3, MUC15, PLBD1, RORB, STRIP2, AQP11, KCNQ4, LBH, STRC, TUBA8, or any combination thereof.

In some aspects, the inner ear outer hair cell selective promoter is an oncomodulin promoter. In some aspects, the oncomodulin promoter has at least 80%, at least 85%, at least 90%, at least 95%, at least 96%, at least 97%, at least 98% at least 99%, or 100% identity to any one of SEQ ID NOs: 1-2. In some aspects, the oncomodulin promoter has the nucleic acid sequence of any one of SEQ ID NOs: 1-2.

In some aspects, the oncomodulin promoter comprises a nucleic acid sequence at least 80%, at least 85%, at least 90%, at least 95%, at least 96%, at least 97%, at least 98% at least 99%, or 100% identity to any one of SEQ ID NO: 1. In some aspects, the oncomodulin promoter is the nucleic acid sequence of SEQ ID NO: 1.

In some aspects, the oncomodulin promoter comprises a nucleic acid sequence at least 80%, at least 85%, at least 90%, at least 95%, at least 96%, at least 97%, at least 98% at least 99%, or 100% identity to any one of SEQ ID NO: 2. In some aspects, the oncomodulin promoter is the nucleic acid sequence of SEQ ID NO: 2.

In some aspects, the oncomodulin promoter is 100-2000, 200-1800, 300-1700, 400-1600, 500-1500, 600-1400, 700-1300, 800-1200, 900-1100, 950-1050, or 1000-1050 nucleotides in length. In some aspects, the oncomodulin promoter is 1000-1050 nucleotides in length.

In some aspects, the oncomodulin promoter is 500-2500, 600-2400, 700-2300, 800-2200, 900-2100, 1000-2000, 1100-1900, 1200-1800, 1300-1700, 1400-1600, or 1450-1500 nucleotides in length. In some aspects, the oncomodulin promoter is 1450-1500 nucleotides in length.

Exemplary oncomodulin promoter
(SEQ ID NO: 1)
GTGCAATTTATGGTATAGCTGGGAAACGTCAAAGTCAAGAGTTTTGTAGGAAAGTCA
CGTCACTTAGCCCTGTCTCCTGTGCCGGGTGAGACCTGTGTGTGCACTTGGTGACAAT
GGCTTTGAGTCTGTCAACTCCAGACTGAGGTCAGCCTTACACACCCATAGTTCCCAA
AGCTGAAAACAGGCCTGCCTCCAACGGTACCTGCTAATATCAGGGGAGCCTTTTCAG
CTTACAGAGCACCCTGTATGTGTTTGTCTTAGTTCAGGCCACCATCTCCACCTTACCA
GGCATCTAGAACCTTCTCCACACTTTGCCAACAGGGTTCGTTTGCAGAATTGAAATC
TTAGTTAAGGTTTGTTGAAGTTTGTTGTTGTTTTTTTTTTTTTTTTACAATTGGCTGTTC
CCACCCACATTCCCTTGAGACATAAATAGAAAAAAAAAAAAAAAGAGGTTTCATGA
GTAAGACAAGACATTTGAGCTGCATCCACTTGATCCTTGAAAAGTGCAATTTATGGT
ATAGCTGGGAAACGTCAAAGTCAAGAGTTTTGTAGGAAAGTCACGTCACTTAGCCCT
GTCTCCTGTGCCGGGTGAGACCTGTGTGTGCACTTGGTGACAATGGCTTTGAGTCTGT
CAACTCCAGACTGAGGTCAGCCTTACACACCCATAGTTCCCAAAGCTGAAAACAGGC
CTGCCTCCAACGGTACCTGCTAATATCAGGGGAGCCTTTTCAGCTTACAGAGCACCC
TGTATGTGTTTGTCTTAGTTCAGGCCACCATCTCCACCTTACCAGGCATCTAGAACCT
TCTCCACACTTTGCCAACAGGGTTCGTTTGCAGAATTGAAATCTTAGTTAAGGTTTGT
TGAAGTTTGTTGTTGTTTTTTTTTTTTTTTTACAATTGGCTGTTCCCACCCACATTCCCT
TGAGACATAAATAGAAAAAAAAAAAAAAAGAGGTTTCATGAGTAAGACAAGACATT
TGAGCTGCATCCACTTGATCCTTGAAAA
Exemplary oncomodulin promoter
(SEQ ID NO: 2)
GTGCAATTTATGGTATAGCTGGGAAACGTCAAAGTCAAGAGTTTTGTAGGAAAGTCA
CGTCACTTAGCCCTGTCTCCTGTGCCGGGTGAGACCTGTGTGTGCACTTGGTGACAAT
GGCTTTGAGTCTGTCAACTCCAGACTGAGGTCAGCCTTACACACCCATAGTTCCCAA
AGCTGAAAACAGGCCTGCCTCCAACGGTACCTGCTAATATCAGGGGAGCCTTTTCAG
CTTACAGAGCACCCTGTATGTGTTTGTCTTAGTTCAGGCCACCATCTCCACCTTACCA
GGCATCTAGAACCTTCTCCACACTTTGCCAACAGGGTTCGTTTGCAGAATTGAAATC
TTAGTTAAGGTTTGTTGAAGTTTGTTGTTGTTTTTTTTTTTTTTTTACAATTGGCTGTTC
CCACCCACATTCCCTTGAGACATAAATAGAAAAAAAAAAAAAAAGAGGTTTCATGA
GTAAGACAAGACATTTGAGCTGCATCCACTTGATCCTTGAAAAGGAAATCTAAGAG
GTTGTAACTATCACTTTTTCTAGCCTATATAAGGTAGGTCAGTAAGGTAGCAAAAAC
ACATCTGTTGTTTTGCTCCTTCAACTCTTTTTCCTGATTCTTCCTGGGGGGAAACCGA
AAACGGTGAGTAACTGGTGGACACATCAGACCCCAGACTCTTTTCTTCACTGCATGC
ATTCATATTAGGCTCAGGTGCTTAGACTCCTGTTTTCCGGTGGCTCTGACACCTGGAA
GGATTTTAATCTCTGGGAGATGGGCTTTTCATCCATCTGCTTCCCACCTTTCAGGACA
GGTGCATGCCTTCTTCCACAGAATGTCTGCAAGCAGCCCAAACTGTATCCTTTCCCAC
GTGGAATTTGCAACATTGCATCTCTCGGGCTGCTGTAGGAAAATGCCAGTGCATGTG
TAACATGGTTTACGGCTGCCTATGCAAATGACTGATTATGTCAGTATAATTTTTATAA
GAAAACAATTGAATCCTTCTTTGGGTCATTTTTTTTTTCCATTTTTGGCATGTATTCAA
AAGAAGGCTCTGAGACAAAAAAGGCTGGGGTGTTTTCCGTATCTGGTTTTAATTTGG
ATATTCTGTCCCGTCACTTAATACAAAACCATGCTTATCACATTTTAAAAATTCTAGA
CAGGCCTGGCTCGGTGGCTTGCATCTGTCATCCCAGCACTTTGTGAGGCCAAGGCAG
GCAGATCACCTGAGGTCAGGAGCTCAAGACCAGCCTGGCCAACATGGCAAAACCCC
GTCTCTACTAAAAACACAAAAATTAGCCAGGCATGGTAGTGCGCACCTGTAATCCCA
GCTACTGGGAAGGCTTAGGCAGGAGAATCACTTGAGCCCAGGAGGCGGAGGTTGCG
GTGAGCCGAGATCACGCTCTTGCACTCCAGCCTGGGTGACAGAGTGAGACTCCGTCT
TAATTTAAAAAAAAAAATAA

In some aspects, the inner ear outer hair cell selective promoter is a prestin promoter. In some aspects, the prestin promoter has at least 80%, at least 85%, at least 90%, at least 95%, at least 96%, at least 97%, at least 98% at least 99%, or 100% identity to any one of SEQ ID NOs: 3 or 15. In some aspects, the prestin promoter has the nucleic acid sequence of any one of SEQ ID NOs: 3 or 15.

In some aspects, the inner ear outer hair cell selective promoter is a prestin promoter. In some aspects, the prestin promoter comprises a nucleic acid sequence at least 80%, at least 85%, at least 90%, at least 95%, at least 96%, at least 97%, at least 98% at least 99%, or 100% identity to any one of SEQ ID NO: 3. In some aspects, the prestin promoter is the nucleic acid sequence of SEQ ID NO: 3.

In some aspects, the inner ear outer hair cell selective promoter is a prestin promoter. In some aspects, the prestin promoter comprises a nucleic acid sequence at least 80%, at least 85%, at least 90%, at least 95%, at least 96%, at least 97%, at least 98% at least 99%, or 100% identity to any one of SEQ ID NO: 15. In some aspects, the prestin promoter is the nucleic acid sequence of SEQ ID NO: 15.

In some aspects, the prestin promoter is 500-2500, 600-2400, 700-2300, 800-2200, 900-2100, 1000-2000, 1100-1900, 1200-1800, 1300-1700, 1400-1600, 1450-1550, or 1500-1550 nucleotides in length. In some aspects, the prestin promoter is 1500-1550 nucleotides in length.

In some aspects, the prestin promoter is 1000-3000, 1100-2900, 1200-2800, 1300-2700, 1400-2600, 1500-2500, 1600-2400, 1700-2300, 1800-2200, 1850-1950, 1900-1950 nucleotides in length. In some aspects, the prestin promoter is 1900-1950 nucleotides in length.

Exemplary prestin promoter
(SEQ ID NO: 3)
AAAGCAAACTCATCTCTAAACCAGAAATAATAGCAATATCTATACAAGTAAATACAT
GTACTCAGAACAGTGCCTACTACATGTAAACACTGAACAGGTGTTAGCAACATTGCC
ATTATTGTGTTAGTATATTAGGTACCTGGTGCTACCGGCAAAACCAGTTTATCATCCA
ACTGTCTCCAGTGTTGCTACTCAAAGTTTGGTCCTCCAGTAGCCTATCAGGATCACCC
AGGGGCCTGTTAGAAAGGCACATCTCAGACCCCACCCCAGACCTACTGAATCAGAA
TCTGCGTTTTTAACGGGATCCGCAGGTGATTCCTATGCACATTAAAGTGTAAGAAGT
ACTGGGCTACAGACAGGTATGTGACAAAATAATTTCATAGGATGGCAAAGGCCAAG
TGGCAAATGAAGGACACCAGAAATGCACGTCCCAGGAGCCCAACTCCTCCTTAGTA
AATTACCCTATTAAGATTTGTTTAGAGATGTTCAAAAGCGTGGAGAAAAGCAAATTT
GGTTTCCCTGGTGCCCTTGAAGAGATCGCCCTCGTGTGGAGTAGGGAGGGAATCTCT
AGCCTTTCCTCTCGGATGAAGAACAGCACCAGCGCTCCCAGCCAAAGGCCTGGCCCA
GGTTCTGGAGGTGGGGTCTCCTTGGCAGAAGCCTCTGGTGTCTGCAGGCGTGCATTT
ACAGCTTTAAGACCAAACAGCTAGTCCGCCACGTGTCACTACAGTGTGCACGCGCAG
AAATGCACAAAGCAAAAAAAAAAAAAAAGATGCTCTTAATGAACCAACTATAATCC
TTGCTAAGGCATAAAGCCAGAGGGAAGTATGTATCTGAAATCATTTTCTACCCCTCA
CCCTCTTGGAGCCCGGCACTCTGGCTGCGGTGCTCTCTTGTATCCCAGTTGCTAGATG
CAAAACAAGCTATTTCCTATCTAATTTTTTTTTTGTTTTATAAATTCTAACTTAAATGC
CCAGAAAATAACTACTCATACTCACATTGTCCTCTAATTGAAAAGATAAGTCAGGTT
TTTTGTGTTTTTTTTTCATTTTAAAATCATAATACGCAATGTTTTCCACTTGAACGCTA
TACCTTGTGTATTGTGCTTGCTTCAGCCTCGAGCCTCTACTGATGTTCCACCTCAAGG
CGACAGGAATGCCACCTGGAGAAACTCCTGGGCGGTATGGGAAGAAAGCCGGTCTC
ATCAGAGTATATTTGCGGGGATCGACGACCAAGGTGTTAAATTCCAAGCACGCTTTG
GAAAGTTCTAGGTGCTTGGGAAGAGATCCGTAGGCGGCAGGGATGCCCGCGCCCCG
GCGTCCCAGCGCGGAGGGTGGCGGCGGGGCCTGGCCCTAGCGGGGCGGGGGGGGCT
CGGGTTACCGGGAGTCGCGGGGCGCGGCCGGCACTGCCCGCGGCGCCTCCTCCTAG
AGCCGCACCTGGAGGCAGCGCGCGCGTCGAAGAGGCAGCGGCTGTGGAGCGCGGCG
GGGCGGCTCCGCCCAGGGCAGCCCGGGCTG
Exemplary prestin promoter
(SEQ ID NO: 15)
TAAACACTGAACAGGTGTTAGCAACATTGCCATTATTGTGTTAGTATATTAGGTACC
TGGTGCTACCGGCAAAACCAGTTTATCATCCAACTGTCTCCAGTGTTGCTACTCAAA
GTTTGGTCCTCCAGTAGCCTATCAGGATCACCCAGGGGCCTGTTAGAAAGGCACATC
TCAGACCCCACCCCAGACCTACTGAATCAGAATCTGCGTTTTTAACGGGATCCGCAG
GTGATTCCTATGCACATTAAAGTGTAAGAAGTACTGGGCTACAGACAGGTATGTGAC
AAAATAATTTCATAGGATGGCAAAGGCCAAGTGGCAAATGAAGGACACCAGAAATG
CACGTCCCAGGAGCCCAACTCCTCCTTAGTAAATTACCCTATTAAGATTTGTTTAGAG
ATGTTCAAAAGCGTGGAGAAAAGCAAATTTGGTTTCCTCAGCTAGGGACGCGGAGA
GTGGTCTGGTGCCCTTGAAGAGATCGCCCTCGTGTGGAGTAGGGAGGGAATCTCTAG
CCTTTCCTCTCGGATGAAGAACAGCACCAGCGCTCCCAGCCAAAGGCCTGGCCCAGG
TTCTGGAGGTGGGGTCTCCTTGGCAGAAGCCTCTGGTGTCTGCAGGCGTGCATTTAC
AGCTTTAAGACCAAACAGCTAGTCCGCCACGTGTCACTACAGTGTGCACGCGCAGAA
ATGCACAAAGCAAAAAAAAAAAAAAAGATGCTCTTAATGAACCAACTATAATCCTT
GCTAAGGCATAAAGCCAGAGGGAAGTATGTATCTGAAATCATTTTCTACCCCTCACC
CTCTTGGAGCCCGGCACTCTGGCTGCGGTGCTCTCTTGTATCCCAGTTGCTAGATGCA
AAACAAGCTATTTCCTATCTAATTTTTTTTTTTAAGAGACGGAGTCTCGCTTTGTTGC
CCAGGCTGGTCTCAAACTCCTGGACTCAAGCAATTCTCCCAGCTTGGGGTAACGTGT
TACATTATTCTACTTAATAAAAAGCAAAAGTTGTTTTATAAATTCTAACTTAAATGCC
CAGAAAATAACTTATCATGCATTGCCTTGTCGTGCAATAGTCAATATTTGCAAACCA
AGTGTTAACCAAAGGCAGTTCATCAAAGATTTTTGAAAATTAAAAAAAAAAAAAAA
CTCATACTCACATTGTCCTCAGGATTTCCTGTTTTCGAAATGTTCCTGTACGAATCGG
AGTCTCTATAATGATTGTAATTGAAAAGATAAGTCAGGTTTTTTGTGTTTTTTTTTCA
TTTTAAAATCATAATACGCAATGTTTTCCACTTGAACGCTATACCTTGTGTATTGTGC
TTGCTTCAGCCTCGAGCCTCTACTGATGTTCCACCTCAAGGCGACAGGAATGCCACC
TGGAGAAACTCCTGGGCGGTATGGGAAGAAAGCCGGTCTCATCAGAGTATATTTGC
GGGGATCGACGACCAAGGTGTTAAATTCCAAGCACGCTTTGGAAAGTTCTAGGTGCT
TGGGAAGAGATCCGTAGGCGGCAGGGATGCCCGCGCCCCGGCGTCCCAGCGCGGAG
GGTGGCGGCGGGGCCTGGCCCTAGCGGGGCGGGGGGGGCTCGGGTTACCGGGAGTC
GCGGGGCGCGGCCGGCACTGCCCGCGGCGCCTCCTCCTAGAGCCGCACCTGGAGGC
AGCGCGCGCGTCGAAGAGGCAGCGGCTGTGGAGCGCGGCGGGGCGGCTCCGCCCAG
GGCAGCCCGGGCTGGGCCAAGGAGCGAGCTCTCCCTTCTCCTGCTCTCAGCCTCAGT
GATCAAGGCTTCAGTGAACTGCACTGGAGCTCCCAGCGGGGGATCTTGTCCCCTGTC
CCGACTTTTGTGCTGCACATTGGATCTGGTGACACTCAGGAAATTGCTTGTCTCCGGC
TGTTAAGGAATAATTTCAGAGTACT

In some aspects, the inner ear outer hair cell selective promoter is a CHRNA10 promoter. In some aspects, the CHRNA10 promoter has at least 80%, at least 85%, at least 90%, at least 95%, at least 96%, at least 97%, at least 98% at least 99%, or 100% identity to SEQ ID NO: 4. In some aspects, the CHRNA10 promoter has the nucleic acid sequence of SEQ ID NO: 4.

In some aspects, the CHRNA10 promoter is 100-1200, 200-1100, 300-1000, 400-950, 500-900, 600-850, 700-800, or 700-750 nucleotides in length. In some aspects, the CHRNA10 promoter is 740 nucleotides in length.

Exemplary CHRNA10 promoter
(SEQ ID NO: 4)
TTCAGATGCCATCATTAATGAGAACTATGACTACCTGAAGGGGTTCTTG
GAAGACCTGGCAAGGAACTCCCCTTGGATTAATTGGCTTCTCTGCTTCT
TTGTAGGTGGATTGCTCAGGTAATGACCTGGAGCAGTTACACATCAAAG
TGACTTCACTGTGCAGTCGGATAGAGCAGATTCAGTGTCTGGTATTGGC
TTTCCCTTTGTATTTTTGAATAGAATATACCATTCAAAGCCTCCTCGCT
CTTCTACTATAGTGGTTTTGTTTTTAAACCCTGAGTGACGCTTCACCTT
TCTAAATCAGATTCCCTTTTGTAAAGGGGATAATGATTGCTGATGTTAC
TTCACACAGGGCTATTTTCAAGAGGAATCAATTGAGTAGCATGAGTACT
ATTCCAGATCTTATTTTGATCTGTCAAGCTGAAGATGTGAGCAAATTCC
AATTAAGATTAGACCAAAGACTTCTGAGACTTTCAGGAATTCAGGGATG
AGAAAGCAGAGTGGGTCAGCTCTGTTGTCTGGAACTTCCATTTAACTTA
GATGCCTCAGGATAGGGGTTACTCAGCTGGAATCCCCTCCACTACTGAC
TCACTATGTGAACCTGAGTGAGTCACAAAACATAGTTGGACTTCCAGCA
AAGAACACCTGACCTGGTTTCCTTACCAGAGGAATGTTTCAGAAAGTGA
GTATGCTATAGAAATGGTTAGCTCTTAGCAGTGTTCGGAATTGTGGGCC
AGGAG

In some aspects, the inner ear outer hair cell selective promoter is a DNM3 promoter. In some aspects, the DNM3 promoter has at least 80%, at least 85%, at least 90%, at least 95%, at least 96%, at least 97%, at least 98% at least 99%, or 100% identity to SEQ ID NO: 5. In some aspects, the DNM3 promoter has the nucleic acid sequence of SEQ ID NO: 5.

In some aspects, the DNM3 promoter is 100-2000, 200-1800, 300-1700, 400-1600, 500-1500, 600-1400, 700-1300, 800-1200, 850-1000, or 900-950 nucleotides in length. In some aspects, the DNM3 promoter is 900-950 nucleotides in length.

Exemplary DNM3 promoter
(SEQ ID NO: 5)
CCTTGATTCAGAGTTAAAGCTATGGGAAAGTCCTCAGGCAGAGGACAAA
CATTAGACAAGAAAATGCCCATATATGAAACCCTGCGAAGCATCAGTAT
TTGAGGAGCAGACTAAAAAGGAACCGTCTGTGGAGGCTAAGAGAAGCAT
GGCCATTTATCTTTGTGTCCCGATCATCAGGCACAGGACCCCACACACA
GTCACTTCTCAATGTGCTAAATTTCACAGAATGCGTCCAGGGTACCTGG
TTCTGGATAGATCCGGTAGAAGGAGATAGACCGGGAGGGCAAATGGCAT
GAGGAGTCTCACAGGCCAGAGTGATTAAAGGGGTGTATCGGGGCGGTAA
ACCCTACAGACTCTACCTGTGCTTATGCGGGGCTGGGGAGGACGAGTCA
TTACAGATGAAGAATTAAGTAAGGTCAGACCACTCAGGGCCTTAGATGG
ATGTCACATTGAAGAATTTAGACTCCAACAGGCCTGCCACCCTGGGAGG
AGTCATCGCGGATTCTGGAGAAGGGCGTGACAGAGGAGATTTCCTTTCG
GGAAGTGTAGTCTGGCAGCGGTGCCCCGGTGGTGGCGGCGGCGGTGCTG
CTGTTGCTGGTGATCGTGTGGTGGTGTTAGCGGCGATAGTGCTTTCCAC
TGGGCTTTGGCTTGGTAGCCGCTGAAAGAGAACAACGCTGCCGCTGCTG
CTGATTTCATGCCATTTCCTGACCCGGCGCTGTAACTTGGCCTCTGAGC
CTTGGCCACAGAACGCAGAGGCCGTGGCATCTGGCCGCAGCTGGGCTGC
AGTGCGTGCGCGCCTGGCCTGGTGGTCCGATGGGAAGCCCGGGGCGGGG
CAGCCGCGGGGGGGGGGCGGGGCGTCGCGGAGATAGGCCACGCCCCTGC
CCGCCCGCGCAGGCGCGCTGCGGGTCGTTAGCTGTC

In some aspects, the inner ear outer hair cell selective promoter is a MUC15 promoter. In some aspects, the MUC15 promoter has at least 80%, at least 85%, at least 90%, at least 95%, at least 96%, at least 97%, at least 98% at least 99%, or 100% identity to SEQ ID NO: 6. In some aspects, the MUC15 promoter has the nucleic acid sequence of SEQ ID NO: 6.

In some aspects, the MUC15 promoter is 500-2500, 600-2400, 700-2300, 800-2200, 900-2100, 1000-2000, 1100-1900, 1200-1800, 1400-1700, 1500-1650, or 1600-nucleotides in length. In some aspects, the MUC15 promoter is 1600-1650 nucleotides in length.

Exemplary MUC15 promoter
(SEQ ID NO: 6)
TTTCTCCTAATTCAGCACAAAAATTGAGTTCCTTTTCTGTAGCTAAAGAGCTTGTATG
AACTGTCAGCTTAGCTAACCATATGTTTTCAATGTTCCCTGCAAATTGTTTAAGGTAT
GTATAGTCCTTTCAATGGATGAGTAAGTCTTTTGTCATTGTTATTTGCTGCCTGTGGA
CTTGATTTCAAAATCTTCTTCAGGTCATGAATAAATTTCCTTTTCCTTCTGTCCCTACT
TTTGAGCCAAGGAACAAATCAAGATTCTTCCTCAGAGTGTACACACCTTCCCAGGCA
TCTCACTCTCTCCTCACTCTATCTGCTTCAAGTTATGGCTCGTTGGTGAGAACACTCT
GCTGCTGAGGTTATTATTTAGCTATAATAACTTTTTCTAACTAGACAGAAACAAATTA
GATATGCCAGGATTTTCTAATTACCTGCCTTAAGTGCTTTTTTAGAAAGCATTAATAA
ATCATGTGGATCTTTTCCTAGCAGTGGTAAGATAAGTTATAATATTATCAAACTGTCA
GTTTTGCCACTTCAATATATGTATGCCTGGTTGTAACCTCACTTAATAAGTTAAGTCC
ATGTAAAAATAGTTGATAGTTAATAAATTGGGCAAGAGTTGCTTAAACAGATTAGAC
TATATAACAAAATTAGGGTTTTAAAAGAATAAAGCTGCTATAACAGTACGCTTCATC
TCACAGGAATTAATCAGTTATGGTATCTCCACAAAACAGAATATCACGTATTGTTGA
AGAGAGCCGTCTCATTTCCCCGGGGTTGGTTTAATTTCTAATCAAACTCTGAAGGGG
CCTTTGGGCTTCAGAAAATTTAAAACTATAGAATTACCTTGTTCTTTCCTCGGGCCAA
TTAACTGGGCAGATTCTTTGCATTCCATTTGAAGCTTACTAGCTCCTGCATTTTAGCT
AAAGTTTCGTTTCTCGCTCAGCAGTTGAAAACCTATCTCCTTGTGCAGCAGAAACCA
AGTATGAACCTCAGGCATATTGAGCTGAACGGCCCTTGGCGCCATCCCCAAACGCTG
ATGTGCGGAAGATCCCAGTTTCACTCTTCTCCCTTTCATAAGCTCTGAAAGGAAGTGT
AGGAAGTATGCCAAGTTGTTATTCAACTCTAGTATTTAATCAAGCATTACCTGGGCA
CTTCTGAAATTCTCCAGCTTCTAAAGTGAGAGTAAACCAGAGAGAACACAGGGTGG
AAACTACTTAATCGAGAAGGCTCCTAGGATAAGTGAGGATCACATGGCCATTCTCAG
GCCCCAGTTCCTCTCCAAACTCCTGAAAGTCAGCAAGAAACCGAATCTCAGTCATGA
TGATTATTTTTCATGTAACACCTCACAGCGTTCTCAGGGATCCCAATATATGCTACTA
ATTCACTTTGTGTTAAGTAGGAGTTTCTTAAAAAAACAATTTCAGTGGAGAAATTCC
TGCTATACCAGATGACTTTGCCAAAATCTTTGTCCTTTTTTTCACTTAGGGTGAAAAA
AAAAATTGATGACCCGTGTTTTGCTACCACTGACGAGAGTAATACCTTGTCCCAAAG
CTAAAACGATCAACCTATGAAAACTGGAGGGTTGGGCTTTTGTTGTTGTTGTTAAAG
GCCTGAATGAGGTGATATCTT

In some aspects, the inner ear outer hair cell selective promoter is a PLBD1 promoter. In some aspects, the PLBD1 promoter has at least 80%, at least 85%, at least 90%, at least 95%, at least 96%, at least 97%, at least 98% at least 99%, or 100% identity to SEQ ID NO: 7. In some aspects, the PLBD1 promoter has the nucleic acid sequence of SEQ ID NO: 7.

In some aspects, the PLBD1 promoter is 100-2000, 200-1800, 300-1700, 400-1600, 500-1500, 600-1400, 700-1300, 800-1200, 850-1100, 900-1050, or 950-1000 nucleotides in length. In some aspects, the PLBD1 promoter is 950-1000 nucleotides in length.

Exemplary PLBD1 promoter
(SEQ ID NO: 7)
GACCCATTATTCAATGGGAGTTGTCAGGATGTCAGCAATGTACAAAATC
ATTGCTTAATTTGTTTGACAATGGAAATGGCCATTATGGTTTTTATGTA
ACTTTGCTTCTGTTACATAATTCTTGCTGACACGGTGTTTCAACCAAGG
TACTTGGTAGCAAGTGTTGTACAGAAAAGGATCTGTAAGTGGTTTATGT
GGTCATCAACCACAGCAAAGATTTCATCTGAGCTGTGCTATGAAGAATG
TAGCTTGAGAAACACAAAATGTATCACTGGGCAAAAAGGAAGCAGAAGA
AAAATACAGTTCTGCTAATGAGAGCTCTGACTGGTATCTGGAGTATAAG
ATGGGCCCAGCCAATGCTGAGTGAATGAATGAAATGCCTTTTGCCTACT
TCACAATGTCACCTAGGGCACCCGGTGCCAACTTCACAATATCACCCAA
GGCATAACTTTTGACTACTTCACAATGTCACTTTTAACTGACCCCACAC
AGAAATGGGGACTCCACAGAAACGTAGGAGTGTGTCTAGTGTCAGCCCC
GTCTGAATCACTCTCCTGTGGTGGCTCCAGCCAACGAAGAGGAAGCAAA
AAGGATAAAAAATCTGAGCTACAGCGCATGGATTTAGGTTAAACAGCCT
GGGAATGAGGGGTACGCTAATCGCTGAGGAAAACGCACCTGTGGAGGCC
TCTCCAGAAACAGCAGAGGATCCGAGCTGCGTGTAGGCAGGGCGCGCAT
GTCACCCTGGCCCGGGCGCCTGGTCCGCTGCTGGAGATAAATGGTCGAC
CCCGGAGGGAGAGGCTAGTAGGGGTGTTGATGTGAACTGATTCGCCCAA
GCCTTGGGCCGCAAAACTGCGAAAGAAAGCGGCAGGCAGCCTCTGCATT
TCCCAGAAGTGCAGCTGGGGAACTTTCCCAGACCGGCCCAGGGGTTGCT
AGAGGGTCAGACGTAAAGGATCCGCCTTTCCTAGGCGGGGTGGGCCCCA
GGCC

In some aspects, the inner ear outer hair cell selective promoter is a RORB promoter. In some aspects, the RORB promoter has at least 80%, at least 85%, at least 90%, at least 95%, at least 96%, at least 97%, at least 98% at least 99%, or 100% identity to SEQ ID NO: 8. In some aspects, the RORB promoter has the nucleic acid sequence of SEQ ID NO: 8.

In some aspects, the RORB promoter is 500-2500, 600-2400, 700-2300, 800-2200, 900-2100, 1000-2000, 1100-1900, 1200-1800, 1250-1700, 1300-1600, 1350-1550, 1400-1500, or 1400-1450 nucleotides in length. In some aspects, the RORB promoter is 1400-1450 nucleotides in length.

Exemplary RORB promoter
(SEQ ID NO: 8)
CCAATAAATGTTGGCTCTTGTTTTTTCTGACCTGTATGTTTTGTCTTTGTTCCAAAGCT
AGCCTTACCTCTCCCACATACTGGGGTTAATTCATGCTTTGGCCCTTATCACCTTTTC
CAATTTATTTCAAAATTACATGCTCTATTTTAATATTTGCTTTCTTTTTTTTATTTTGA
AAACTTATTGAACTTGCATCTACACTTTAAAATGAAGCAGAACTTAAAGAACTCAAA
GATTATGAAGAAGACTCAGTACCTGGGAATAAAATTGAGAATAGGTTCCTTTTATGA
CTATATAACCAATCTCAACCATTATTTTTTGCTTCCCCAAATTAGGAGAGTTTAAAAT
GCAGATTCTCCCCACTCTCCTCTTCCCATTCAATAGAAACTGAGAAAGAAGGATCTT
ATTCAGGTCTTCACTCCATTTGTGATTCATATTCAGTGGCTGAAAGGTTAGAAAGCAT
TCACTCCACCAATAATGATCAAGCACCCATAAAGTACCAGGAGCTCTTACAAACTCT
AGGGAAATCCTGGCTCCTGTTGTCATGAATTTTGCATTCTCAGGTAGGAAATGTGGC
TCTGATGCCTGCTGGGGCAGTGTACACTTAGAGCTACAGAGGATCTTGGAGGTAATC
TAAAACCTTTCTAAAGAGCACCCTGCAATCACACCTTCTAGCAACAGCCATTTCTCTT
GAATTAGTAAGGTGGCTACACCGCCAATTTGAGCTGTTCTCCTTCAGTCCTGTAGTCC
ATCGCCAGGGGAGTCTCCAAATGCTAATAAAAATCAATTTCCCAGACAAAAGAACA
TAGAGGGTCAGGGAGCATCTGACGGACGTTTTTAAAGGAAGGGGACAGCTACTTCC
ATGGGACTGCATTTTAGTTGTGCTAAAAGTGATGAAAGTGGGTTTGCATTATTCTAC
CACCAACACCCAAACCACCTGCCCACGGAAACCCCCGCCGGAGACCGAAGTTTACC
CAAATAGCGCTCGGCAAAGCGCTGCCATAAATTCAAAACTAACTCTGCCGGGCCCGC
GGGGGTTGCGAGACAGGGACCGAACGTGAAACCCGGGGAGCCCCGCGTCTCTTGCC
TCCGAAGGTTTTCCGTGATCAGTGTCCCCTTCTCTGCTGGAGTCGGAAGTGCCTGTCA
CCTGCGGATCTGCCCGACTCTCCCGGTCGGCCCTTCTTCTCTGCCCAGTTCGGACAGT
CTCGAATTCCCCGTCGCAGCCCCGGCCACCTCGGACTCCCTGGTCCCCAGCCCCCGC
CCCACCCCCCGCCTCCACCACGTCCCCTCCCCGCGGTCCCAGCCTCTCCAGGCGCTGC
TGGGCTCTGATTGGCGGCTGCGCTGACAGCAGGCGGGGCCTGGAAGTCGCGGCCAA
GCCCGCCCTCGCGTATAAGCCCCTCTCAGCGCTCTCTCTCC

In some aspects, the inner ear outer hair cell selective promoter is a STRIP2 promoter. In some aspects, the STRIP2 promoter has at least 80%, at least 85%, at least 90%, at least 95%, at least 96%, at least 97%, at least 98% at least 99%, or 100% identity to SEQ ID NO: 9. In some aspects, the STRIP2 promoter has the nucleic acid sequence of SEQ ID NO: 9.

In some aspects, the STRIP2 promoter is 500-2000, 600-1900, 700-1800, 800-1700, 900-1600, 1000-1500, 1100-1400, 1200-1300, or 1250-1300 nucleotides in length. In some aspects, the STRIP2 promoter is 1250-1300 nucleotides in length.

Exemplary STRIP2 promoter
(SEQ ID NO: 9)
CCTTGAATATTTATGTCCATTTTAACACTTCCTGGTTGCAAGAGGGATGTGCCTCCAT
TATTTCCTCCACAGTTTTGGTATTTGTCAGACATTTGTTCTGCTGTCTTTCTAATCCAG
CCAACGTCTGCTCAGGAAGTGGGGCCAGCTCCACTGGGACCCATAGTTTTACTTCCT
TGTCATTTGATTGGATAGTTTCCAAGGAAGCCCCTCCAGATTGGCACTATCTCAGAA
AAGGAGAGCTTGTTGTGAAACACTGCTTCCTGAAACTTCCTGCTATTGCCTAAAGCT
ACGTCTGAAACTGAGTAGGGAAAGGCATACTTTTCCAGGGACTTAGGGGGATAGGC
TTTGAGGTCTCTCCTCGTGTTGACTCTATGCAATCTTCATAGCACCAGTTTTACACAT
TCCTTCTCTGAAATTAAAGCCAGATGGAGCCTCTAGGCTTAGCAAGTGGCTTTAGAT
AGCCACCAGAGGGGACTTGCAAGCTGTCCTCTATCCTACTCCCAAGATCAGTCTGCC
CTTTCCCCTAGGAATAGGCAGGAAAAGAATAAAGGAAAGAAAGGACTGGCGAGCA
GGTGAGGGTGGGGGCTTGCTCTACCCTCAACATTTACACACCATGAGGAAGAGGCCC
CCTACAGCAGAGAAGGGCAGATGACAGGAGCAGCCCTCGAGGGCACCACCAATTTC
AGTGATGGAAAAACTCCCCCATCCCACCCTTAGACCTCCAGTCTCCCAGCCAAGCCC
TAGCTCCGGGCGAGATGCGTTCTCTTCAGAAAAACGCTGAGAATTCTCAGCTTCCAG
AGACAGCGAGTCCCTCGTTTCGGGCGATGTCCCTGGCCACCTGGCGGTGCCATCCCT
CCCCTGAGACTAAGCGGGATATGGGACGTGTGCAGGAGCCGGGATATGGGGGGCCG
GGTCGGTGGTAACAGGGAAACGGAGACTGCTGTGGAGCAGTAGGCGGAGACTAGAG
CTCCGGAAAAGGTCGCTACAGGGACGGGGGTGAGAGCTGAGAGACACCGAGTGAG
GAGCACAGAGATAACCCGCCTGATCTCAAGGCCCAGCTTTCGCGAGGTGTGGAGCCT
GTAGCTAACCTAGGAGTCTCCGTCCGCCAGCAATGCCGCAGGACTAAAAAGATCCCC
TCAAAAATCTCTTCATTGAGCCCCCACCTCCTCGAGTCCCGCTCCGGCCGGTCGAGC
AGCCAATCGCCTCGCGGGGCGGGGTTGCGGCGAGCTGCCGTAACCAATAGAGGTGG
AGGGGGCGGGGCCTGGCTCCCGGCGCGCGGCGGTAGGGTCGCCTCCGG

In some aspects, the inner ear outer hair cell selective promoter is a AQP11 promoter. In some aspects, the AQP11 promoter has at least 80%, at least 85%, at least 90%, at least 95%, at least 96%, at least 97%, at least 98% at least 99%, or 100% identity to SEQ ID NO: 10. In some aspects, the AQP11 promoter has the nucleic acid sequence of SEQ ID NO: 10.

In some aspects, the AQP11 promoter is 500-2000, 600-1900, 700-1800, 800-1700, 900-1600, 1000-1500, 1100-1400, 1200-1300, or 1250-1300 nucleotides in length. In some aspects, the AQP11 promoter is 1250-1300 nucleotides in length.

Exemplary AQP11 promoter
(SEQ ID NO: 10)
AGGCATGAGCCACTATGCCCAAATGAGAAATAATTTTGTATGAAAAATAATCTTGTA
TGGTAAATTTAGACCAAGAATAAAATGAGTGGTTGTATAAGAAAGAAAGATGTTCA
GAACAAACCAAAAAGTCCAAGCATGTCACGAATGGTCTGTGTAAGTCATAATAAAA
GGATTTATCTAAAAAAACCAAAAACTTTTATATGATCAAGTCGTCTATAATTAAAGG
AAAATTATAATGGGTTTTTCTAGACATTGGGTGTGATGTAATGAAACGTACACACTA
AAGAATTCATTACAAGGCTTTCATGTTTTGTTTTTTGTTTGTTTGACTGGTTTGTTGTT
GTTGTTGTTGTTGTTGTTGTTGTTTTTGAGACGGAGTTTCGCTCTTGTTGCCCAGGCTG
GAGTACAATGGCGCGATCTAGGCTCACCACAACCTCTGCCTCCCGGGTTCAAGCGAT
TCTCCTGCATCATCCTCCCGAGTAGCTGGGATGACAGGCATGCGCCACCATGCCCGG
CTAATTTTGTATTTTTAGTAGAGACGGGGGTTTCTCATGTTGGTCAGGCTAGTCTCGA
ACTCCCGACCTCCGGTGATCCGCCCGCCTCGGCCTCCCAAAGTGCTGGGATTATAGG
CGTGAGCCACCGCGCCCAGCCGCGCCCGGTTTTTGTTGTTGTTTTTGTTTCTAAAAAC
AGCGTCTCGCTCTGTGGCCCAGGCAGGGGTGCAGTGGCGCGATCTCAGCTCACTGCA
GCCTGGAACTCCTGGGGTCAAGCGGTCTTCCCACCTAAGCCTCTCCGTGCTGGGACT
CCGGACGCGCTCCACCTCACGCAGCCGTATTCCTGCTTTCAAAGCAGATGGAAGAGG
TGCGCCAGGACCCCCAGTTCTTGGAAACAGACCTCTCCAGTTACCTGTTGTTTCCTCT
TCACGAAGAGTGCATGTAACAGTAAGACACAACTGTTTCATATTATACGTAAAGAGT
TCATGCCAAAGGTTATAGACAGTCACATGCTAAAACTAGGCTACACTTTGAAGAATC
ACCGCTCAAGTTCTGGAAAAAAGAGGTGACTGTTGAACAACACTGTGAGGGTAATC
GATGCCACTGAAATATACACTTAAATTGATTAAAGTGGCGAATTTTATCTGGCATAT
ATTACCACCATTTTTAGAAATGTTTTTTGGCAGGTGAAGAAAAGCAAGGCTCCAGGA
GGCCCTGCGCACCGGTCTACGCCCACTAACTCACCCGCCCCCTGCGCCGCGTCTCCC
CTCTCAATTTCAGTCGCCCATTGAT

In some aspects, the inner ear outer hair cell selective promoter is a KCNQ4 promoter. In some aspects, the KCNQ4 promoter has at least 80%, at least 85%, at least 90%, at least 95%, at least 96%, at least 97%, at least 98% at least 99%, or 100% identity to SEQ ID NO: 11. In some aspects, the KCNQ4 promoter has the nucleic acid sequence of SEQ ID NO: 11.

In some aspects, the KCNQ4 promoter is 500-2500, 600-2400, 700-2300, 800-2200, 900-2100, 1000-2000, 1100-1900, 1200-1800, 1300-1750, 1400-1700, 1450-1650, or 1550-1600 nucleotides in length. In some aspects, the KCNQ4 promoter is 1550-1600 nucleotides in length.

Exemplary KCNQ4 promoter
(SEQ ID NO: 11)
AGGGCCCATCCTGGTGTAAACAAACCCTTTGGCGCAGCCCAAGAGAGCCC
CTATCTAATACCCAGCACGATCCCCTTACATCCGGAGCACTCTTTAAACA
TTTTTCCTAGCTGATCTTCACAGTGACCCTGCAGGGGAGACAGGAAGAGG
TATCATGATCCCTGTGTTAGCGTGGGAGGGCTAGTGAAGTGCAGTGACTT
GCCCAAGGTCACTCCATGAATTGAGGGTGGAATTGAAACCAAAACACTGA
TCTCCTGACCCCCTGTGCATACACAGTTGCTCTTGGAGATTGGAGACCCC
TGGAATCTGGAGCAGACAATCTGGCTGGCTTCCTTGCAGCTCAGGTCTGC
GGAGGCCACAAGGGGGCAGCATGCAGCCCTCACCTGTGTCTCTGGGACCT
TTGAAGGGAGGGTCCTCCCTAGGATAACAGTGAGAGCTGGAAACTCTACC
CTCTCCAGAGTATTGCCTCAAGATCCCTGAACTTAGCTCCATGTTTTCAG
AATGTGCTAGCTACAATTCCTGAAATGCCCTTTTACTTCCCTTTTCACTT
ATTGAGCTCCTATACATCCATCAAGGCCCAATTTAAATGGCCCTTTCAGC
AGCTATTTCTTTGGCACCTTCTGTGTGTCAGACGTTGTTTTAAACATTGT
GAATACAGCTTAAAACAAGTCTGACGGGTGGAAAGGAAACTGCTGAGGGT
GGGGTCAGGGGAACAGGTGGGAGAGGGACCAGTCCCCTCCAGCAGAGGGG
CCAATTGAGGGAGCCTGAGACAGCTGTTTGCTCAGAAAAGTGTCTTAGTC
ACTAAAGGTTGTGGTGGGGAAAGTCCGTCCTCCCAGTCATGTCCTGGGAA
TCCGGATGGCGCAGGAAGGCCACCCGGTGACCCTAAGAGTGGCCACCTGT
CCTCTCTGAACTGGACTTTCTCTTCTGGCCCTTCCCCTCCCTCCCTCCCT
CACTGGCGCTCAGCAGATCAATGCTGCCTTTGCTGACAGCTGAGAATCGA
GCTCGCCTTCCCGCCCCTTCCCCCGCCCCTCCCGCTCGGCTTCGTCCCTC
GAGATCCTCCCGGAGGAACCGGGAAGAGTTTGCTGCGGAAGGCTCACCCT
GGGGCAGGGCCTGCGGAGGGAGCGGCTGGTGTGGCCGCAGCTTTCCGTGG
AGGAAGAGGGAAAGAGGATCGGGAAACCCAAGTTACCAACCCTGTGCAGG
GGAGATGGAGGTCGGGGACTAAGAAAAACTGCTGCCCACCCAGCCACACA
CAGCACTGGGCACACTTTAAGCACCCGCACCAGGCACACAGTGCTCGACC
CCAACGGACACACCTCATCCTGCCGCCCGCGGCCACAACTCCACATTCAC
TTGCACGCGTCCGGCTTCCCGGCCCCGCGCGCTGCCCCCGCCACGCGGTT
CGGCCCAGGCACCAACTCGGCCGCCCGTGCGCCCTGCCCCGCCGCCTGCT
CCGCGCGTTCCCTCCCTCCGCCTCGCCTCGCTTGCTCGCTCGCTCCCTCC
CGATTTGGGAAGGCGGCCGCGGGGGGGGCGGGGGAGGGGCGGGGCGGGGG
AGGGT

In some aspects, the inner ear outer hair cell selective promoter is a LBH promoter. In some aspects, the LBH promoter has at least 80%, at least 85%, at least 90%, at least 95%, at least 96%, at least 97%, at least 98% at least 99%, or 100% identity to SEQ ID NO: 12. In some aspects, the LBH promoter has the nucleic acid sequence of SEQ ID NO: 12.

In some aspects, the LBH promoter is 500-2000, 600-1900, 700-1800, 800-1700, 900-1600, 1000-1500, 1100-1400, 1150-1300, or 1200-1250 nucleotides in length. In some aspects, the LBH promoter is 1200-1250 nucleotides in length.

Exemplary LBH promoter
(SEQ ID NO: 12)
GTGAATTCGATGATGTGCTTGTGTGGACATGTGGAGGTCTCAAGAACAAA
AGAAGAGCTGGGCCTGGCACACAGTGGGTGCTAATGCCTGTTAGAATTGT
TGTTGAGAGGGCAGGAGGGTGTAACATGGACCCAGCTATCTGATCCTGAG
GCTGGGCGCCATGTGGGTGTGAAGTACACCAGGGGCTCCAACCAGCAAGT
GCTAGCTCAGGTTACAGTCAGCTGCCCCTGGAGGAAGCTAGCAGACATCC
TGTGTACTTGAAAAGAAAACTGAAAGTGCTATCTGCATCCTGGTGATAGT
AACCTCTCTTTTCTGGCTGTTGAAGTGCATTCCTGTGCGGATGTGGAAAG
AGAGAAAGCAAGATACAGCCAGGGCTAGGACAGGAATGTGAGTATTTCCT
TAATTGGACATGAGAGCCTTGAACTGATTCCAGTTGGAGTGTTTTCTTTT
AGGGCCTGGACCCTAAAGATTTCATACAGTTTTCTTTGTCAGAAAAATCC
CTTTGGTTCAAAGGCCCCTCGATAGAAATAAAGAAAAAGCCAGGGCTGAA
TTTCTTTGATATGTGGGAAGGCAAGAGTTTATGAGCTGCCAGATCTCAGG
CTTCTTTTGGGGTGGAGGATTTTGTCTGGTGGGTTCGGGTTGCTTTGTGT
TGTTGACTGCTAATTCACTGATGACCAAGTTTCTCAAATACCTTAAAAAC
AAGCCCTACGTCTGCTCAGTGCTTTCCAATTTACCAAGTGTTTTCATAAC
ATTTCTTATGTACGCAAATGAGTTTCACCGAAAAATTGGCTAGAAACTTC
CCTTCTCCTACTCACGTTCATAGTGTAGCTGTGAAAACAAACAAAACCAC
AGAGGCATGGTAAGTGTGGTATGGTGGGGAAAACAAAGCCATTTTACAGG
CGTGATTGAAGCGGAGGCCACAGAGCGGCAGCGCTGGGTCCCGAGTGAGA
CTCCCATCATGTGGCTCAATGGAAAAATCCTACCCAGGACGACACCACAT
CCTTGCTCCCACAAATAAAACCTTCCACGGAACTCAGGGCTGCAGACGCA
GAGCCGAGCGCGCCCCCGAGCCGCCGCCCGCCGGAGCTGCGAGCGCTGAA
GCCATTCATGATTTTGGTGACGTTATTCCAGGAGTGGGCGAGGGAGGGCG
GGGCCTCTCGGGGCCAAGCCCCGCCCCCGCCCCTATAAATACGGCTTCCC
GGGCTCTTTGTGGG

In some aspects, the inner ear outer hair cell selective promoter is a STRC promoter. In some aspects, the STRC promoter has at least 80%, at least 85%, at least 90%, at least 95%, at least 96%, at least 97%, at least 98% at least 99%, or 100% identity to SEQ ID NO: 13. In some aspects, the STRC promoter has the nucleic acid sequence of SEQ ID NO: 13.

In some aspects, the STRC promoter is 100-2100, 200-2000, 300-1900, 400-1800, 500-1700, 600-1600, 700-1500, 800-1400, 900-1300, 1100-1200, or 1100-1150 nucleotides in length. In some aspects, the STRC promoter is 1100-1150 nucleotides in length.

Exemplary STRC promoter
(SEQ ID NO: 13)
CACTGCCATCTCAACATGTGGTTTCTAGGTTGCCTCAACAGGGAAGAGAT
TGTTGGAGGCTCATTTGCTAGCTCTTAAATTCTTTGGTCAACCACAGTCC
ATTGAACAGAACTAATCACCTGACTGCAAGAGATCTGGGAAATGTGAGAA
ACACCTAGATATCTAGTAAGCAATAAATATTTCAGTTACCAAAGCCAAAC
CAAAAAAAGAGAAAAATAATTGTACTTTACAAAGGGAGGCATCTGGGTCC
TGTGGGAGTTTTGGGGAGTGAGGATGTTTCAGAGTTCTCAACTCCTGTGG
CTATCCATTTCATTTTAGCAGGACATATGATTAATTTCTTGTTCTGGACC
TTTGTAATTTAAAGTCTGAATCCTTAGCGGCAAGAGAATTGCTTAATCAA
TGGCTTACAACAGCAGAACGTGGACTGCCAGGAAAATTTCCATCCTGAGT
TAAGAAAGAAGGATAATTTATTATAAGAGGGTTGTTACAGAATGAAGGGC
AGAAATTCAGAAGGATTACAGGATGGGCTGGAACCACAAAGCACTGTCTG
CTTTTTAGACTAGGTGTGGTATCCTTGATGGGCAAAGGGAATATTGGTAA
AATTATTTGTGACCTGGGTTAAGTCATTCCATTTCTCTGGGCTTCAATTC
CCCTGTCTATAAAATGTTTGAGGGAGAGAATGGGGAAGGGTTCTAGGGAA
AGAAGGACAGAATAAAAGTTTGGGTATATGAATTACTATTTAGAGTTGGT
ATAAAGTGAAGGCCTTTGGGGAGATATACCCTGACCAGACCAGATTATTT
TGAATGAAATCTCTTTCTCTGTTCCATGAGCAGTTCTGTGTGTAGGGAGA
ACATTTGAATGGCCTAATGAGCAAATCACATTTCTCTGGGTCTGTTTCCT
TATCCATAAGTTCTGCATCACTGGCTCCTAACTCAAGCAATCTCCTTGGG
TTTCTCTGAGGGGCCCCTGGGATCCCCTATCATTAGTCCCTCTCACAGAA
GCATACCCTTCTCCAGAGCTAAAGGATCAGATATTCAGCGGCTCAGGTAA
CAAACCTGCTGTCAGGTTACACATATTGTTTCCTGAAAGACCACACTACA
GTGTCAGTGGAGCCTCAGGTTGCCTGCAGT

In some aspects, the inner ear outer hair cell selective promoter is a TUBA8 promoter. In some aspects, the TUBA8 promoter has at least 80%, at least 85%, at least 90%, at least 95%, at least 96%, at least 97%, at least 98% at least 99%, or 100% identity to SEQ ID NO: 14. In some aspects, the TUBA8 promoter has the nucleic acid sequence of SEQ ID NO: 14.

In some aspects, the TUBA8 promoter is 1000-2600, 1100-2500, 1200-2400, 1300-2300, 1400-2200, 1500-2100, 1600-2000, 1700-1900, 1800-1900, or 1800-1850 nucleotides in length. In some aspects, the TUBA8 promoter is 1800-1850 nucleotides in length.

Exemplary TUBA8 promoter
(SEQ ID NO: 14)
GAAGACATAGTTCCAGTCTGAGTCTGAAGCCTGGGACCCATGAGAGCTGA
AGACGTGGTCCCAGTCTGAGTCTGAAGCCTGAGACCCAGGAGAGCTGAAG
ACGTGGTTCCAGTCTGAGTCTGAAGCCTGAGATCCAGGAGAGCTAAGGAC
ATGGTTCTAGTCTGAGTCTGAAGCCTGAGACCCAGGAGAGCTGATGGTGT
GGTTCCAGTCTGAGTCTGAAGCCTGAGACCCAGGAGAGCTGAATACGTAG
TTCCAGTCTGAGTCTGAAGCCTGAGTTCCAGGAGAGCTGAGGACATGGTT
CCAGTCTGAATCTGAAGCCTGAGACCCAGGAGAGCTGATGGTGTGGTCTG
AAGACGTAGTTCCAGTCTGAGTCTGAAGCCTGAGACCCAGGAGAGCTGAA
GATGTGGTTTCAGTCTGTCTGAAGCCTGAGACCCAGGAGAGCTGATGGTG
TGGTTCCAGTCTGAGTCTGAAGTCTGAGACCCAGGAGAGCTGAAGATGTG
GTTCCAGTCTGAGTCTGAAGCCTGAGACCCAGCAGAGCTGAAGACATGGT
TCCAGTCTGAGTCTGAAGCCTGAGACCCAGGAGAGCTGAAGATGTGGTTT
CAGTCTGTCTGAAGCCTGAGACCCGGGAGAGCTGAAGACGTAGTTCCAGT
CTGAGTCTGAAGCCTGAGAGACCCAGGAGAGCTGATGGTGTGGTTCCAGT
CTGAGTCTGAAGCCTGAGACCCAGGAGAGCTGAAGATGTGGTTTCAGTCT
GTCTGAAGCTTGAGACCCAGGGGAGCTGAAGATGTAGTTCCAGTCTGAGT
CTGAAGCCTGAGACCCAGGAGAGGTGAAGACGTGGTTTCAGTCTGAGTCA
AGGCCTGAGAACCAGGAGAGCTGCTGGTGAAAGTTCTAGTGCAAGGGCAG
AAGACCAATGTCCTACCTAGCTCAACAGTCAGGCAGGCAGAAGTTCCCTG
TTTCTCAGCCTTTTTGTTCTATTCTGTTCTTCAGTTGGTTGGATGAGGCC
CCTGCACATTAAGGATAGACAAAAATTCAACGCATGCTTTACTAAGTACC
GTTTGTATCAGTGGGTAAAGCACTGTGTTTGGTACTCTCTCAAATGCAAA
GATGATTACGACACATGTACTATCGTTTATGAATGGGTGGCCAACAGAAC
AGATTGCCGCATAGGTAAGCAGAAATCTGCTCTCATTCTCTATTGGCCAC
AAGCAGGCATGTCTTAGGAGCAGAAGGGTAGGAAGATCTCTAACTGTGCT
TGGAAACTTGGGGAGTTACCACGTCTGGCTAAAGTGGTATTGTCTTAAGG
AAAACCTCTTACTACTGGGCAGAGGCAGGGGAACCCTGGTATGAGTTCTG
GATTACATAGGAGATGTGACTTGGACACGTTTGGGGCTTAAAAGTAGGAA
GGGATCAAGGGGGGAGATTTGAAAATCCCGGTGGAGGTGCGAGGTATCCG
GGGAGAGGTGGGAGCAGAGGCCCTGCAGCTTGCCAAGCACACACGGCCCT
AGGGCGCCCAGCTGAGACGGCACCTTGGCACCCGGGCCCGCTGCAGCCCG
CTCCGGTCAGCTGCACCCCAGTCAGGAGCCTTTCCAGCGGGTCGGAGGAG
AACGGAAGTTTGGGGAGACCCGCGCGATTCGCCTGGCTGCATTTTACATT
TCTTTCTCCGGCAGCTGGGGTCACGAAGGCTGCTCTCGCCGGCGGTGTTG
GAACGTGGACACGTGCGCTTTGGTAATAGGGCAGCCTCCCCCGCGGGCGC
AGTCCCCGCTGCGAGCGCCCCCGGCTGCTGAGGCGGGACCGAGGACCCGG
AGATTT

TABLE 2
Exemplary Promoters

| Promoter | SEQ ID NO(s) |
|---|---|
| Oncomodulin | 1, 2 |
| Prestin | 3, 15 |
| CHRNA10 | 4 |
| DNM3 | 5 |
| MUC15 | 6 |
| PLBD1 | 7 |
| RORB | 8 |
| STRIP2 | 9 |
| AQP11 | 10 |
| KCNQ4 | 11 |
| LBH | 12 |
| STRC | 13 |
| TUBA8 | 14 |

### Enhancers

In some aspects, a construct can include an enhancer sequence. In some aspects, the construct does not comprise an enhancer sequence. The term “enhancer” refers to a nucleotide sequence that can increase the level of transcription of a nucleic acid encoding a protein of interest (e.g., a polypeptide). Enhancer sequences (generally 50-1500 bp in length) generally increase the level of transcription by providing additional binding sites for transcription-associated proteins (e.g., transcription factors). In some aspects, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and/or a SV40 enhancer. In some aspects, a construct comprises a CMV enhancer with at least 80%, at least 85%, at least 90%, at least 95%, at least 96%, at least 97%, at least 98% at least 99%, or 100% identity to SEQ ID NO: 18. In some aspects, the CMV enhancer comprises the nucleic acid sequence of SEQ ID NO: 18.

Exemplary CMV enhancer
(SEQ ID NO: 18)
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGACTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATG

### Flanking Untranslated Regions, 5′ UTRs and 3′ UTRs

In some aspects, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, kits, or methods as described herein to enhance or otherwise modulate the expression of a polypeptide.

Natural 5′ UTRs include a sequence that plays a role in translation initiation, in some aspects, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. The 5′ UTRs have also been known to form secondary structures that are involved in elongation factor binding.

In some aspects, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

In some aspects, a 5′ UTR from an mRNA that is transcribed by a cell in the cochlea can be included in any of the constructs, compositions, kits, and methods described herein. In some aspects, the 5′ UTR is derived from the 5′ UTR of the polynucleotide encoding the polypeptide. In some aspects, the 5′ UTR is derived from the endogenous KCNQ4 5′ UTR. In some aspects, the 5′ UTR comprises at least 80%, at least 85%, at least 90%, at least 95%, at least 96%, at least 97%, at least 98% at least 99%, or 100% identity to SEQ ID NO: 19. In some aspects, the 5′ UTR comprises the nucleic acid sequence of SEQ ID NO: 19.

3′ UTRs are found immediately 3′ to the stop codon of the gene of interest. In some aspects, a 3′ UTR from an mRNA that is transcribed by a cell in the cochlea can be included in any of the constructs, compositions, kits, and methods described herein. In some aspects, the

In some aspects, the 3′ UTR is derived from the 3′ UTR of the polynucleotide encoding the polypeptide. In some aspects, the 3′ UTR is derived from the endogenous KCNQ4 3′ UTR. In some aspects, the KCNQ4 3′ UTR comprises at least 80%, at least 85%, at least 90%, at least 95%, at least 96%, at least 97%, at least 98% at least 99%, or 100% identity to SEQ ID NO: 45. In some aspects, the KCNQ4 3′ UTR comprises the nucleic acid sequence of SEQ ID NO: 45.

In some aspects, the 3′ UTR comprises at least 80%, at least 85%, at least 90%, at least 95%, at least 96%, at least 97%, at least 98% at least 99%, or 100% identity to SEQ ID NO: 45. In some aspects, the 3′ UTR comprises the nucleic acid sequence of SEQ ID NO: 45.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995, each of which is incorporated herein by reference in its entirety): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some aspects, the introduction, removal, or modification of 3′ UTR AREs can be used to modulate the stability of an mRNA encoding a polypeptide. In other aspects, AREs can be removed or mutated to increase the intracellular stability and thus increase translation and production of a polypeptide.

In other aspects, non-ARE sequences may be incorporated into the 5′ or 3′ UTRs. In some aspects, introns or portions of intron sequences may be incorporated into the flanking regions of the polynucleotides in any of the constructs, compositions, kits, and methods provided herein. Incorporation of intronic sequences may increase protein production as well as mRNA levels.

Exemplary 5′ UTR Sequence
(SEQ ID NO: 19)
CGCCGGTGGCAGGTGGAAAGGCGAGCGGCATGGAGCGCGTAATAAGAGAG
TTGGAGTCGGAAAGAGCAGCCCCAGTCGCCGGGGAAGCGGGAGGTCAGTG
CGGGCTCCGGCGGCCCCCAGGCTCCGAGCGCCCGCCCGCGGCCCCGGCCC
GGCCCCTAGCCCCCGCCGCCCGCGCCCGCCCCGGGTCGCCCCTCTGGCCC
CGGGTCCGAGCCATGCGTCTCTGAGCGCCCCGAGCGCGCCCCCGCCCCGG
ACCGTGCCCGGGCCCCGGCGCCCCCAGCCCGGCGCCGCCC
Exemplary 3′ UTR Sequence
(SEQ ID NO: 45)
GGGACTTCTCAGAGGCAGGGCAGCACACGGCCAGCCCCGCGGCCTGGCGC
TCCGACTGCCCTCTGAGGCCTCCGGACTCCTCTCGTACTTGAACTCACTC
CCTCACGGGGAGAGAGACCACACGCAGTATTGAGCTGCCTGAGTGGGCGT
GGTACCTGCTGTGGGTGCCAGCGCCCCTTCCCCACCTCAGGAGCGTGAGA
TGCCAGGTCGCACAGAGGGCAGCAGCAGCGGCCGTCCCGCGGCCTCTGGG
CCCCCCAGTGCCCTGCCCACTCCATCAAGGCCCTATGTGGCCCACCTGGC
AGGGGCACAGCCCCGGGAGTGGGAGCGGGCGCTGGGGCCCTGGGCCCTGA
CCCAGCTTCCAGCTATGCAAGGTGAGGTCTCTGGCCCACCCTTCGGACAC
AGCAGGGAAGCCCTCCCGCCAAGTCCCCGCCCCACTTGGGGGTGGGCCAA
GGTGCCCCCACAGGTACCCACAAAGCACAGGACCCTGCCACAAGGCAGGT
GGACACCATATATGCAAACCATGTTAAATATGCAACTTTGGGGACCCCCA
TGGGGTCTCTCTGTCCCTCCCCCATTGGGAGCTGGGCCCCCAGCAGTAGC
TGGTCTCAGGCTGCTTGGCCACCACCCTGTCCCTATTCTTTGGCTTATCA
CTCCTTCCCCTCCCAGCATGGGGCCTGTTTCTCCCCTGCCCTCTCCTAAG
GGCAATGCCTGGGCCTTTCTTCCCATTTGCAAGTGTCAGCTCCCAGGGGC
TCCCTCCTCCTGCTGGGTGGCCACTCCCCTCCTTGGCCCTCCAGACACCA
CTCATAGTCAGCACAGGTTTCTGTATCCTCCCCAAAACTCCCAGACAGTG
CTTCGTGGACGATCGCACAAACATAGCCTTTTAGTTTCTCCAGACAGGAA
GAAAGCCTCTCACACTTAAACATGCAATGACGTGACACACTTGGAGACAT
GAGTGCAGAGCCACTCAGCCGCTCCTGGGCCTCTGCAGCAGATGCCAGTG
GACTGGCCTTGCAGGGTGACGACCACTAAGAGGAAGACCCCCAACTCCAT
CTGAGCAGGAGAAGGAGCTTTGAAGTAACCCGAGAGCTCTCCAGGCCCCA
CCCAGACCTTTACCCGCTCCCCTTCTTCAAGAAGATCTCCTCCTCTCTGG
TCCAGGAGCCCTAACCCACTGCCTCTGCCTGTCCCCAAGGGCCCGCCTCC
GTGTCTCCACAGCACAACTCGGGCCCAGGCCTGACACCACTGGAGAGACC
CCAGGCCCACTTCTAGCCAGGCCTGTGCCTTCCTAGTCACTCTAACTCCC
AGAGAGAATAAGAATGCATGTAATAGCTATACCAACCGCGCATCCGGCTT
TCACATGCACTGTCTCCCCTCCCTCCACACCCCACTTCTTCACTTCAATT
GGCAGCGCCACATCCAGGCGTCAGCCCCCATTCACTCCAGGAACACTTTC
TTATCCCCACCCCTTTGCTCCTCTTCTGCAAAGCCAATGCAGGTGGCAGG
AAGGTGAGGGGTAGTGGACCAATGGCAACCCTCTGTGGGAACAAGGGGCC
GAGGCCACGCTGCCTGCATCTCGTGCTGGGGACCTGCATGCGCCAGCACC
AGGGCTTGGACTGGATCTTACTCAGTCCATGGTGCCCAGCCTCTGCCCCA
ACATGCCCTCTGCATGTGACCGTCATGCCCTGGATGGAGCCACTCCTGGC
TCACCCCACCTGCACTGCACTGTCCCCAGAGAGCCACCCCTCCACCCACT
CAGAGACAGCTGTGGAGAGGGCCAGGAGAATGGGATTACCCTATGACCAA
GGAGACATGGGAAGAAGCCCTCCTTCCTTCCACGATCGAGGTTCCGCCAT
CAACTCGGTTCTCGGATATGCAAGTACCTCACTTTGTTAACTTATTAACT
TATTGGTTTCATTAAAGTTTTCAAGAGGA

### Polyadenylation Sequences

In some aspects, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002, which is incorporated herein by reference in its entirety). A poly(A) tail confers mRNA stability and transferability (Molecular Biology of the Cell, Third Edition by B. Alberts et al., Garland Publishing, 1994, which is incorporated herein by reference in its entirety). In some aspects, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some aspects, a poly(A) tail is added onto transcripts that contain a specific sequence, e.g., a polyadenylation (or poly(A)) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH) (Woychik et al., Proc. Natl. Acad Sci. US.A. 81(13):3944-3948, 1984; U.S. Pat. No. 5,122,458, each of which is incorporated herein by reference in its entirety), mouse-β-globin, mouse-α-globin (Orkin et al., EMBO J 4(2):453-456, 1985; Thein et al., Blood7l(2):313-319, 1988, each of which is incorporated herein by reference in its entirety), human collagen, polyoma virus (Batt et al., Mol. Cell Biol. 15(9):4783-4790, 1995, which is incorporated herein by reference in its entirety), the Herpes simplex virus thymidine kinase gene (HSV TK), IgG heavy-chain gene polyadenylation signal (US 2006/0040354, which is incorporated herein by reference in its entirety), human growth hormone (hGH) (Szymanski et al., Mol. Therapy 15(7):1340-1347, 2007, which is incorporated herein by reference in its entirety), the group comprising a SV40 poly(A) site, such as the SV40 late and early poly(A) site (Schek et al., Mol. Cell Biol. 12(12):5386-5393, 1992, which is incorporated herein by reference in its entirety).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414, which is incorporated herein by reference in its entirety).

In some aspects, a poly(A) signal sequence can be a synthetic polyadenylation site (see, e.g., the pCl-neo expression construct of Promega that is based on Levitt et al., Genes Dev. 3(7):1019-1025, 1989, which is incorporated herein by reference in its entirety). In some aspects, a poly(A) signal sequence comprises or consists of the SV40 poly(A) site. In some aspects, a poly(A) signal sequence comprises or consists of bGHpA. In some aspects, the poly(A) signal sequence comprises at least 80%, at least 85%, at least 90%, at least 95%, at least 96%, at least 97%, at least 98% at least 99%, or 100% identity to SEQ ID NO: 22. In some aspects, the poly(A) signal sequence comprises the nucleic acid sequence of SEQ ID NO: 22. In some aspects, the poly(A) signal sequence comprises at least 80%, at least 85%, at least 90%, at least 95%, at least 96%, at least 97%, at least 98% at least 99%, or 100% identity to SEQ ID NO: 48. In some aspects, the poly(A) signal sequence comprises the nucleic acid sequence of SEQ ID NO: 48.

Exemplary bGH poly(A) signal sequence
(SEQ ID NO: 22)
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGATGCGGTGGGCTCTATG
Exemplary SV40 poly(A) signal sequence
(SEQ ID NO: 48)
AACTTGTTTATTGCAGCTTATAATGGTTACAAATAAAGCAATAGCATCAC
AAATTTCACAAATAAAGCATTTTTTTCACTGCATTCTAGTTGTGGTTTGT
CCAAACTCATCAATGTATCTTA

### Additional Sequences

In some aspects, constructs of the present disclosure may include one or more filler sequences. In some aspects, filler sequences may function as regulatory elements, altering construct expression. In some such aspects, filler sequences may not be fully removed prior to manufacturing for administration to a subject. In some aspects, filler sequences may have functional roles including as linker sequences, as regulatory regions, or as stabilizing regions. As will be appreciated by those skilled in the art, filler sequences may vary significantly in primary sequence while retaining their desired function.

In some aspects, constructs of the present disclosure may include one or more cloning sites. In some such aspects, cloning sites may not be fully removed prior to manufacturing for administration to a subject. In some aspects, cloning sites may have functional roles including as linker sequences, portions of a Kozak site, or as sites encoding a stop codon. As will be appreciated by those skilled in the art, cloning sites may vary significantly in primary sequence while retaining their desired function. In some aspects, constructs may contain additional cloning sites less than five nucleotides in length.

### Reporter Polypeptides, Sequences, or Elements

In some aspects, constructs provided herein can optionally include a sequence encoding a reporter polypeptide and/or protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. Non-limiting examples of reporter polypeptides include a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art.

When associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some aspects, a reporter sequence is the LacZ gene, and the presence of a construct carrying the LacZ gene in a mammalian cell (e.g., a cochlear hair cell) is detected by assays for beta-galactosidase activity. When the reporter is a fluorescent protein (e.g., green fluorescent protein) or luciferase, the presence of a construct carrying the fluorescent protein or luciferase in a mammalian cell (e.g., a cochlear hair cell) may be measured by fluorescent techniques (e.g., fluorescent microscopy or FACS) or light production in a luminometer (e.g., a spectrophotometer or an IVIS imaging instrument). In some aspects, a reporter sequence can be used to verify the tissue-specific targeting capabilities and tissue-specific promoter regulatory and/or control activity of any of the constructs described herein.

In some aspects, a reporter polypeptide is a FLAG tag (e.g., a 3×FLAG tag), and the presence of a construct carrying the FLAG tag in a mammalian cell (e.g., an inner ear cell, e.g., a outer hair cell) is detected by protein binding or detection assays (e.g., Western blots, immunohistochemistry, radioimmunoassay (RIA), mass spectrometry). Exemplary 3×FLAG tag sequences are provided as SEQ ID NOs: 21 and 39.

Exemplary 3xFLAG tag sequence
(SEQ ID NO: 21)
GACTACAAAGACCATGACGGTGATTATAAAGATCATGACATCGACTACAA
GGATGACGATGACAAG
Exemplary 3xFLAG tag sequence with stop codon
(SEQ ID NO: 39)
GACTACAAAGACCATGACGGTGATTATAAAGATCATGACATCGACTACAA
GGATGACGATGACAAGTAA

### AAV Capsids

The present disclosure provides one or more polynucleotide constructs packaged into an AAV capsid. In some aspects, an AAV capsid is from or derived from an AAV capsid of an AAV2, 3, 4, 5, 6, 7, 8, 9, 10, rh8, rh10, rh39, rh43 or Anc80 serotype, or one or more hybrids thereof. In some aspects, an AAV capsid is from an AAV ancestral serotype. In some aspects, an AAV capsid is an ancestral (Anc) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. For example, in some aspects, an AAV capsid is an Anc80 capsid (e.g., an Anc80L65 capsid). In some aspects, an AAV capsid is created using a template nucleotide coding sequence comprising SEQ ID NO: 40. In some aspects, the capsid comprises a polypeptide represented by SEQ ID NO: 41. In some aspects, the capsid comprises a polypeptide with at least 85%, at least 90%, at least 95%, at least 96%, at least 97%, at least 98% at least 99%, or 100% identical to the polypeptide represented by SEQ ID NO: 41.

As provided herein, any combination of AAV capsids and AAV constructs (e.g., comprising AAV ITRs) may be used in recombinant AAV (rAAV) particles of the present disclosure. For example, wild-type or variant AAV2 ITRs and Anc80 capsid (e.g., an Anc80L65 capsid), wild-type or variant AAV2 ITRs and AAV6 capsid, etc. In some aspects of the present disclosure, an AAV particle is wholly comprised of AAV2 components (e.g., capsid and ITRs are AAV2 serotype). In some aspects, an AAV particle is an AAV2/6, AAV2/8 or AAV2/9 particle (e.g., an AAV6, AAV8 or AAV9 capsid with an AAV construct having AAV2 ITRs). In some aspects of the present disclosure, an AAV particle is an AAV2/Anc80 particle that comprises an Anc80 capsid (e.g., comprising a polypeptide of SEQ ID NO: 41) that encapsidates an AAV construct with AAV2 ITRs (e.g., SEQ ID NOs: 16 and 17) flanking a portion of a coding sequence, for example, a nucleic acid encoding a polypeptide. Other AAV particles are known in the art and are described in, e.g., Sharma et al., Brain Res Bull. 2010 Feb. 15; 81(2-3): 273, which is incorporated in its entirety herein by reference. In some aspects, a capsid sequence is at least 85%, at least 90%, at least 95%, at least 96%, at least 97%, at least 98% at least 99%, or 100% identical to a capsid nucleotide or amino acid sequence represented by SEQ ID NO: 40 or 41, respectively.

In some aspects, the composition comprises a construct comprising: (i) a 5′ ITR (e.g., an AAV 5′ ITR), (ii) a promoter comprising the nucleic acid sequence having at least 85%, at least 90%, at least 95%, at least 96%, at least 97%, at least 98% at least 99%, or 100% identity to any of one of SEQ ID NOs: 1-15, (iii) a polynucleotide encoding a polypeptide (e.g., a therapeutic polypeptide), (v) optionally, a 3× FLAG tag (e.g., comprising the nucleic acid sequence of SEQ ID NO: 39), (vi) a polyA sequence, and (vii) a 3′ ITR (e.g., an AAV 3′ ITR).

In some aspects, the composition comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a polynucleotide encoding a polypeptide, (iv) optionally, a 3× FLAG tag comprising the nucleic acid sequence of SEQ ID NO: 39, (v) a polyA sequence comprising the nucleic acid sequence of SEQ ID NO: 22, and (vi) a 3′ ITR comprising the nucleic acid sequence of SEQ ID NO: 17.

In some aspects, the composition comprises a construct comprising (i) a 5′ ITR; (ii) a promoter comprising the nucleic acid sequence of any of one of SEQ ID NOs: 1-15; (iii) a polynucleotide encoding an outer hair cell polypeptide comprising a gene selected from actin gamma 1 (ACTG1), adenylate cyclase type 1 (ADCY1), calcium binding protein 2 (CABP2), coiled-coil domain-containing 50 (CCDC50), cadherin-related 23 (CDH23), carcinoembryonic antigen-related cell adhesion molecule 16 (CEACAM16), chromodomain helicase DNA-binding protein 7 (CHD7), calcium- and integrin-binding family member 2 (CIB2), claudin 14 (CLDN14), chloride intracellular channel 5 (CLIC5), caseinolytic mitochondrial matrix peptidase proteolytic subunit (CLPP), clarin 1 (CLRN1), pejvakin (DFNB59), endothelin 3 (EDN3), ELMO domain-containing protein 3 (ELMOD3), epidermal growth factor receptor kinase substrate 8 (EPS8), espin (ESPN), estrogen-related receptor beta (ESRRB), eyes absent homolog 1 (EYA1), GIPC PDZ domain-containing family, member 3 (GIPC3), G protein-coupled receptor 98 (GPR98), G-protein signaling modulator 2 (GPSM2), glutaredoxin, cysteine-rich 1 (GRXCR1), glutaredoxin, cysteine-rich 2 (GRXCR2), immunoglobulin-like domain-containing receptor 1 (ILDR1), lysyl-tRNA synthetase (KARS), Potassium voltage-gated channel, KQT-like subfamily, member 4 (KCNQ4), lipoma HMGIC fusion partner-like 5 (LHFPL5), leucine-rich transmembrane and O-methyltransferase domain-containing (LRTOMT1 COMT2), tricellulin (MARVELD2), micro-ma 96 (MIR96), methionine sulfoxide reductase B3 (MSRB3), myosin, heavy chain 9, non-muscle (MYH9), myosin, heavy chain 14, non-muscle (MYH14), unconventional myosin IIIA (MYO3A), unconventional myosin VI (MYO6), unconventional myosin VIIA (MYO7A), unconventional myosin XVA (MYO15A), otoferlin (OTOF), otogelin-like protein (OTOGL), purinergic receptor P2X, ligand-gated ion channel, 2 (P2RX2), protocadherin 15 (PCDH15), PDZ domain-containing 7 (PDZD7), polyribonucleotide nucleotidyltransferase 1, mitochondrial (PNPT1), POU domain, class 4, transcription factor 3 (POU4F3), phosphoribosyl pyrophosphate synthetase 1 (PRPS1), protein tyrosine phosphatase, receptor type Q (PTPRQ), radixin (RDX), scaffold-containing ankyrin repeats and SAM domain (SANS), serpin peptidase inhibitor, clade B, member 6 (SERPINB6), SIX homeobox 1 (SIX1), SIX homeobox 5 (SIX5), prestin (SLC26A5), second mitochondrial-derived activator of caspase (SMAC/DIABLO), small muscle protein, x-linked (SMPX), stereocilin (STRC), nesprin-4 (SYNE4), TBC1 domain family, member 24 (TBC/D24), tight junction protein XO 2 (TJP2), transmembrane channel-like protein 1 (TMC1), transmembrane inner ear-expressed protein (TMIE), transmembrane protease, serine 3 (TMPRSS3), taperin (TPRN), TRIO and F-actin-binding protein (TRIOBP), Thrombospondin-type laminin G domain and EAR repeats (TSPEAR), harmonin (USH1C), usherin (USH2A), wolframin (WFS1), and whirlin (WHRN), or any combination thereof; (v) optionally, a 3× FLAG tag comprising the nucleic acid sequence of SEQ ID NO: 39; (vi) a polyA sequence; and (vii) a 3′ ITR.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a polynucleotide encoding a polypeptide, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid sequence of SEQ ID NO: 19, (iv) a polynucleotide encoding a polypeptide, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid sequence of SEQ ID NO: 19, (v) a polynucleotide encoding a polypeptide, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid sequence of SEQ ID NO: 19, (iv) a polynucleotide encoding a polypeptide, (v) optionally, a 3× FLAG tag comprising the nucleic acid sequence of SEQ ID NO: 39, (vi) a 3′ UTR comprising the nucleic acid sequence of SEQ ID NO: 45, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid of SEQ ID NO: 19, (v) a polynucleotide encoding a polypeptide, (vi) optionally, a 3× FLAG tag comprising the nucleic acid sequence of SEQ ID NO: 39, (vii) a 3′ UTR comprising the nucleic acid sequence of SEQ ID NO: 45, (viii) a polyA sequence comprising the nucleic acid sequence of SEQ ID NO: 22, and (ix) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16; (ii) a promoter comprising the nucleic acid sequence of any one of SEQ ID NOs: 1-15; (iii) a polynucleotide encoding a polypeptide encoding an outer hair cell polypeptide comprising a gene selected from actin gamma 1 (ACTG1), adenylate cyclase type 1 (ADCY1), calcium binding protein 2 (CABP2), coiled-coil domain-containing 50 (CCDC50), cadherin-related 23 (CDH23), carcinoembryonic antigen-related cell adhesion molecule 16 (CEACAM16), chromodomain helicase DNA-binding protein 7 (CHD7), calcium- and integrin-binding family member 2 (CIB2), claudin 14 (CLDN14), chloride intracellular channel 5 (CLIC5), caseinolytic mitochondrial matrix peptidase proteolytic subunit (CLPP), clarin 1 (CLRN1), pejvakin (DFNB59), endothelin 3 (EDN3), ELMO domain-containing protein 3 (ELMOD3), epidermal growth factor receptor kinase substrate 8 (EPS8), espin (ESPN), estrogen-related receptor beta (ESRRB), eyes absent homolog 1 (EYA1), GIPC PDZ domain-containing family, member 3 (GIPC3), G protein-coupled receptor 98 (GPR98), G-protein signaling modulator 2 (GPSM2), glutaredoxin, cysteine-rich 1 (GRXCR1), glutaredoxin, cysteine-rich 2 (GRXCR2), immunoglobulin-like domain-containing receptor 1 (ILDR1), lysyl-tRNA synthetase (KARS), Potassium voltage-gated channel, KQT-like subfamily, member 4 (KCNQ4), lipoma HMGIC fusion partner-like 5 (LHFPL5), leucine-rich transmembrane and O-methyltransferase domain-containing (LRTOMT1 COMT2), tricellulin (MARVELD2), micro-ma 96 (MIR96), methionine sulfoxide reductase B3 (MSRB3), myosin, heavy chain 9, non-muscle (MYH9), myosin, heavy chain 14, non-muscle (MYH14), unconventional myosin IIIA (MYO3A), unconventional myosin VI (MYO6), unconventional myosin VIIA (MYO7A), unconventional myosin XVA (MYOl5A), otoferlin (OTOF), otogelin-like protein (OTOGL), purinergic receptor P2X, ligand-gated ion channel, 2 (P2RX2), protocadherin 15 (PCDH15), PDZ domain-containing 7 (PDZD7), polyribonucleotide nucleotidyltransferase 1, mitochondrial (PNPT1), POU domain, class 4, transcription factor 3 (POU4F3), phosphoribosyl pyrophosphate synthetase 1 (PRPS1), protein tyrosine phosphatase, receptor type Q (PTPRQ), radixin (RDX), scaffold-containing ankyrin repeats and SAM domain (SANS), serpin peptidase inhibitor, clade B, member 6 (SERPINB6), SIX homeobox 1 (SIX1), SIX homeobox 5 (SIX5), prestin (SLC26A5), second mitochondrial-derived activator of caspase (SMAC/DIABLO), small muscle protein, x-linked (SMPX), stereocilin (STRC), nesprin-4 (SYNE4), TBC1 domain family, member 24 (TBC/D24), tight junction protein XO 2 (TJP2), transmembrane channel-like protein 1 (TMC1), transmembrane inner ear-expressed protein (TMIE), transmembrane protease, serine 3 (TMPRSS3), taperin (TPRN), TRIO and F-actin-binding protein (TRIOBP), Thrombospondin-type laminin G domain and EAR repeats (TSPEAR), harmonin (USH1C), usherin (USH2A), wolframin (WFS1), and whirlin (WHRN), or any combination thereof, (iv) optionally, a 3× FLAG tag comprising the nucleic acid sequence of SEQ ID NO: 39; (v) a polyA sequence comprising the nucleic acid sequence of SEQ ID NO: 22; and (vi) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16; (ii) a promoter comprising the nucleic acid sequence of any one of SEQ ID NOs: 1-15; (iii) a polynucleotide encoding a polypeptide encoding an outer hair cell polypeptide comprising a gene selected from cadherin-related 23 (CDH23), clarin 1 (CLRN1), pejvakin (DFNB59), Potassium voltage-gated channel, KQT-like subfamily, member 4 (KCNQ4), otoferlin (OTOF), protocadherin 15 (PCDH15), POU domain, class 4, transcription factor 3 (POU4F3), prestin (SLC26A5), stereocilin (STRC), transmembrane channel-like protein 1 (TMC1), TRIO and F-actin-binding protein (TRIOBP), harmonin (USH1C), usherin (USH2A), wolframin (WFS1), and whirlin (WHRN), or any combination thereof; (iv) optionally, a 3× FLAG tag comprising the nucleic acid sequence of SEQ ID NO: 39; (v) a polyA sequence comprising the nucleic acid sequence of SEQ ID NO: 22; and (vi) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16; (ii) a promoter comprising the nucleic acid sequence of any one of SEQ ID NOs: 1-15; (iii) a polynucleotide encoding a polypeptide encoding Potassium voltage-gated channel, KQT-like subfamily, member 4 (KCNQ4); (iv) optionally, a 3× FLAG tag comprising the nucleic acid sequence of SEQ ID NO: 39; (v) a polyA sequence comprising the nucleic acid sequence of SEQ ID NO: 22; and (vi) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a KCNQ4 coding region comprising the nucleic acid sequence of SEQ ID NO: 20, (iv) optionally, a 3× FLAG tag comprising the nucleic acid sequence of SEQ ID NO: 39, (v) a polyA sequence comprising the nucleic acid sequence of SEQ ID NO: 22, and (vi) a 3′ ITR comprising the nucleic acid sequence of SEQ ID NO: 17.In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a 3′ UTR, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid of SEQ ID NO: 19, (v a KCNQ4 coding region comprising the nucleic acid sequence of SEQ ID NO: 20, (vi) optionally, a 3× FLAG tag comprising the nucleic acid sequence of SEQ ID NO: 39, (vii) a 3′ UTR comprising the nucleic acid sequence of SEQ ID NO: 45, (viii) a polyA sequence comprising the nucleic acid sequence of SEQ ID NO: 22, and (ix) a 3′ ITR comprising the nucleic acid sequence of SEQ ID NO: 17.

In some aspects, the rAAVAnc80 particle comprises a construct at least 80%, at least 85%, at least 90%, at least 95%, at least 96%, at least 97%, at least 98% at least 99%, or 100% identity to any one of SEQ ID NOs: 23-38, and 49-50. In some aspects, the rAAVAnc80 particle comprises a construct comprising the nucleic acid sequence of any of SEQ ID NOs: 23-38 and 49-50. In some aspects, the construct has at least 80%, at least 85%, at least 90%, at least 95%, at least 96%, at least 97%, at least 98% at least 99%, or 100% identity to any one of nucleotides 12-4396 of SEQ ID NO: 23, 12-4464 of SEQ ID NO: 24, nucleotides 12-4016 of SEQ ID NO: 25, nucleotides 12-4521 of SEQ ID NO: 26, nucleotides 12-3750 of SEQ ID NO: 27, nucleotides 12-3928 of SEQ ID NO: 28, nucleotides 12-4641 of SEQ ID NO: 29, nucleotides 12-3994 of SEQ ID NO: 30, nucleotides 12-4426 of SEQ ID NO: 31, nucleotides 12-4307 of SEQ ID NO: 32, nucleotides 12-4293 of SEQ ID NO: 33, nucleotides 12-4565 of SEQ ID NO: 34, nucleotides 12-4224 of SEQ ID NO: 35, nucleotides 12-4140 of SEQ ID NO: 36, nucleotides 12-4816 of SEQ ID NO: 37, or nucleotides 12-4915 of SEQ ID NO: 38. In some aspects, the rAAVAnc80 particle comprises a construct comprising a nucleic acid sequence comprising any one of nucleotides 12-4396 of SEQ ID NO: 23, 12-4464 of SEQ ID NO: 24, nucleotides 12-4016 of SEQ ID NO: 25, nucleotides 12-4521 of SEQ ID NO: 26, nucleotides 12-3750 of SEQ ID NO: 27, nucleotides 12-3928 of SEQ ID NO: 28, nucleotides 12-4641 of SEQ ID NO: 29, nucleotides 12-3994 of SEQ ID NO: 30, nucleotides 12-4426 of SEQ ID NO: 31, nucleotides 12-4307 of SEQ ID NO: 32, nucleotides 12-4293 of SEQ ID NO: 33, nucleotides 12-4565 of SEQ ID NO: 34, nucleotides 12-4224 of SEQ ID NO: 35, nucleotides 12-4140 of SEQ ID NO: 36, nucleotides 12-4816 of SEQ ID NO: 37, or nucleotides 12-4915 of SEQ ID NO: 38.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 23. In some aspects, the rAAVAnc80 particle comprises a construct comprising the nucleic acid sequence of SEQ ID NO: 23.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4396 of SEQ ID NO: 23. In some aspects, the composition comprises a construct comprising nucleotides 12-4396 of SEQ ID NO: 23.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) an oncomodulin promoter comprising the nucleic acid sequence of SEQ ID NO: 1, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 24. In some aspects, the rAAVAnc80 particle comprises a construct comprising the nucleic acid sequence of SEQ ID NO: 24.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4464 of SEQ ID NO: 24. In some aspects, the rAAVAnc80 particle comprises a construct comprising nucleotides 12-4464 of SEQ ID NO: 24.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) an oncomodulin promoter comprising the nucleic acid sequence of SEQ ID NO: 2, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises (i) a 5′ ITR comprising the nucleic acid sequence of SEQ ID NO: 16, (ii) an oncomodulin promoter comprising the nucleic acid sequence of SEQ ID NO: 2, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 25. In some aspects, the rAAVAnc80 particle comprises the nucleic acid sequence of SEQ ID NO: 25.

In some aspects, the rAAVAnc80 particle comprises a nucleic acid sequence having at least 80%, at least 85%, at least 90%, at least 95%, at least 96%, at least 97%, at least 98% at least 99%, or 100% identity to nucleotides 12-4016 of SEQ ID NO: 25. In some aspects, the rAAVAnc80 particle comprises a construct comprising nucleotides 12-4016 of SEQ ID NO: 25.

In some aspects, the rAAVAnc80 particle comprises a construct comprising (i) a 5′ ITR comprising the nucleic acid sequence of SEQ ID NO: 16, (ii) an oncomodulin promoter comprising the nucleic acid sequence of SEQ ID NO: 1, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 26. In some aspects, the rAAVAnc80 particle comprises a construct comprising the nucleic acid sequence of SEQ ID NO: 26.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4521 of SEQ ID NO: 26. In some aspects, the rAAVAnc80 particle comprises a construct comprising nucleotides 12-4521 of SEQ ID NO: 26.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a prestin promoter comprising the nucleic acid sequence of SEQ ID NO: 3, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a prestin promoter comprising the nucleic acid sequence of SEQ ID NO: 3, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 27. In some aspects, the rAAVAnc80 particle comprises a construct comprising the nucleic acid sequence of SEQ ID NO: 27.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-3750 of SEQ ID NO: 27. In some aspects, the rAAVAnc80 particle comprises a construct comprising nucleotides 12-3750 of SEQ ID NO: 27.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a CHRNA10 promoter comprising the nucleic acid sequence of SEQ ID NO: 4, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a CHRNA10 promoter comprising the nucleic acid sequence of SEQ ID NO: 4, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 28. In some aspects, the rAAVAnc80 particle comprises a construct comprising the nucleic acid sequence of SEQ ID NO: 28.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-3928 of SEQ ID NO: 28. In some aspects, the rAAVAnc80 particle comprises a construct comprising nucleotides 12-3928 of SEQ ID NO: 28.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a DNM3 promoter comprising the nucleic acid sequence of SEQ ID NO: 5, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a DNM3 promoter comprising the nucleic acid sequence of SEQ ID NO: 5, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a nucleic acid having at least 80%, at least 85%, at least 90%, at least 95%, at least 96%, at least 97%, at least 98% at least 99%, or 100% identity to SEQ ID NO: 29. In some aspects, the rAAVAnc80 particle comprises the nucleic acid sequence of SEQ ID NO: 29.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4641 of SEQ ID NO: 29. In some aspects, the rAAVAnc80 particle comprises a construct comprising nucleotides 12-4641 of SEQ ID NO: 29.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a MUC15 promoter comprising the nucleic acid sequence of SEQ ID NO: 6, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a MUC15 promoter comprising the nucleic acid sequence of SEQ ID NO: 6, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 30. In some aspects, the rAAVAnc80 particle comprises a construct comprising the nucleic acid sequence of SEQ ID NO: 30.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-3994 of SEQ ID NO: 30. In some aspects, the rAAVAnc80 particle comprises a construct comprising nucleotides 12-3994 of SEQ ID NO: 30.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a PLBD1 promoter comprising the nucleic acid sequence of SEQ ID NO: 7, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a PLBD1 promoter comprising the nucleic acid sequence of SEQ ID NO: 7, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 31. In some aspects, the rAAVAnc80 particle comprises a construct comprising the nucleic acid sequence of SEQ ID NO: 31.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4426 of SEQ ID NO: 31. In some aspects, the rAAVAnc80 particle comprises a construct comprising nucleotides 12-4426 of SEQ ID NO: 31.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a RORB promoter comprising the nucleic acid sequence of SEQ ID NO: 8, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a RORB promoter comprising the nucleic acid sequence of SEQ ID NO: 8, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 32. In some aspects, the rAAVAnc80 particle comprises a construct comprising the nucleic acid sequence of SEQ ID NO: 32.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4307 of SEQ ID NO: 32. In some aspects, the rAAVAnc80 particle comprises a construct comprising nucleotides 12-4307 of SEQ ID NO: 32.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a STRIP2 promoter comprising the nucleic acid sequence of SEQ ID NO: 9, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a STRIP2 promoter comprising the nucleic acid sequence of SEQ ID NO: 9, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 33. In some aspects, the rAAVAnc80 particle comprises a construct comprising the nucleic acid sequence of SEQ ID NO: 33.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4293 of SEQ ID NO: 33. In some aspects, the rAAVAnc80 particle comprises a construct comprising nucleotides 12-4293 of SEQ ID NO: 33.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a AQP11 promoter comprising the nucleic acid sequence of SEQ ID NO: 10, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a AQP11 promoter comprising the nucleic acid sequence of SEQ ID NO: 10, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 34. In some aspects, the rAAVAnc80 particle comprises a construct comprising the nucleic acid sequence of SEQ ID NO: 34.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4565 of SEQ ID NO: 34. In some aspects, the rAAVAnc80 particle comprises a construct comprising nucleotides 12-4565 of SEQ ID NO: 34.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a KCNQ4 promoter comprising the nucleic acid sequence of SEQ ID NO: 11, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a KCNQ4 promoter comprising the nucleic acid sequence of SEQ ID NO: 11, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 35. In some aspects, the rAAVAnc80 particle comprises a construct comprising the nucleic acid sequence of SEQ ID NO: 35.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4224 of SEQ ID NO: 35. In some aspects, the rAAVAnc80 particle comprises a construct comprising nucleotides 12-4224 of SEQ ID NO: 35.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a LBH promoter comprising the nucleic acid sequence of SEQ ID NO: 12, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a LBH promoter comprising the nucleic acid sequence of SEQ ID NO: 12, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 36. In some aspects, the rAAVAnc80 particle comprises a construct comprising the nucleic acid sequence of SEQ ID NO: 36.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4140 of SEQ ID NO: 36. In some aspects, the rAAVAnc80 particle comprises a construct comprising nucleotides 12-4140 of SEQ ID NO: 36.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a STRC promoter comprising the nucleic acid sequence of SEQ ID NO: 13, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a STRC promoter comprising the nucleic acid sequence of SEQ ID NO: 13, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises s a construct comprising a nucleic acid sequence having at least 80%, at least 85%, at least 90%, at least 95%, at least 96%, at least 97%, at least 98% at least 99%, or 100% identity to SEQ ID NO: 37. In some aspects, the rAAVAnc80 particle comprises a construct comprising the nucleic acid sequence of SEQ ID NO: 37.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4816 of SEQ ID NO: 37. In some aspects, the rAAVAnc80 particle comprises a construct comprising nucleotides 12-4816 of SEQ ID NO: 37.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a TUBA8 promoter comprising the nucleic acid sequence of SEQ ID NO: 14, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a TUBA8 promoter comprising the nucleic acid sequence of SEQ ID NO: 14, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 38. In some aspects, the rAAVAnc80 particle comprises a construct comprising the nucleic acid sequence of SEQ ID NO: 38.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4915 of SEQ ID NO: 38. In some aspects, the rAAVAnc80 particle comprises a construct comprising nucleotides 12-4915 of SEQ ID NO: 38.

In some aspects, the rAAVAnc80 particle comprises a construct comprising (i) a 5′ ITR comprising the nucleic acid sequence of SEQ ID NO: 16, (ii) a CMV enhancer comprising the nucleic acid sequence of SEQ ID NO: 18, (iii) a prestin promoter comprising the nucleic acid sequence of SEQ ID NO: 15, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the rAAVAnc80 particle comprises a construct comprising (i) a 5′ ITR comprising the nucleic acid sequence of SEQ ID NO: 16, (ii) a prestin promoter comprising the nucleic acid sequence of SEQ ID NO: 15, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 49. In some aspects, the rAAVAnc80 particle comprises a construct comprising the nucleic acid sequence of SEQ ID NO: 49.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to nucleotides 12-4070 of SEQ ID NO: 49. In some aspects, the rAAVAnc80 particle comprises a construct comprising nucleotides 12-4070 of SEQ ID NO: 49.

In some aspects, the rAAVAnc80 particle comprises a construct comprising a nucleic acid sequence having at least 80%, at least 85%, at least 90%, at least 95%, at least 96%, at least 97%, at least 98% at least 99%, or 100% identity to SEQ ID NO: 50. In some aspects, the rAAVAnc80 particle comprises a construct comprising the nucleic acid sequence of SEQ ID NO: 50.

Exemplary AAV Anc80 Capsid DNA Sequence
(SEQ ID NO: 40)
ATGGCTGCCGATGGTTATCTTCCAGATTGGCTCGAGGACAACCTCTCTGA
GGGCATTCGCGAGTGGTGGGACTTGAAACCTGGAGCCCCGAAACCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCA
AAGCGGGTGACAATCCGTACCTGCGGTATAACCACGCCGACGCCGAGTTT
CAGGAGCGTCTGCAAGAAGATACGTCTTTTGGGGGCAACCTCGGGCGAGC
AGTCTTCCAGGCCAAGAAGCGGGTTCTCGAACCTCTCGGTCTGGTTGAGG
AAGGCGCTAAGACGGCTCCTGGAAAGAAGAGACCGGTAGAGCAATCACCC
CAGGAACCAGACTCCTCTTCGGGCATCGGCAAGAAAGGCCAGCAGCCCGC
GAAGAAGAGACTCAACTTTGGGCAGACAGGCGACTCAGAGTCAGTGCCCG
ACCCTCAACCACTCGGAGAACCCCCCGCAGCCCCCTCTGGTGTGGGATCT
AATACAATGGCAGCAGGCGGTGGCGCTCCAATGGCAGACAATAACGAAGG
CGCCGACGGAGTGGGTAACGCCTCAGGAAATTGGCATTGCGATTCCACAT
GGCTGGGCGACAGAGTCATCACCACCAGCACCCGAACCTGGGCCCTCCCC
ACCTACAACAACCACCTCTACAAGCAAATCTCCAGCCAATCGGGAGCAAG
CACCAACGACAACACCTACTTCGGCTACAGCACCCCCTGGGGGTATTTTG
ACTTTAACAGATTCCACTGCCACTTCTCACCACGTGACTGGCAGCGACTC
ATCAACAACAACTGGGGATTCCGGCCCAAGAGACTCAACTTCAAGCTCTT
CAACATCCAGGTCAAGGAGGTCACGACGAATGATGGCACCACGACCATCG
CCAATAACCTTACCAGCACGGTTCAGGTCTTTACGGACTCGGAATACCAG
CTCCCGTACGTCCTCGGCTCTGCGCACCAGGGCTGCCTGCCTCCGTTCCC
GGCGGACGTCTTCATGATTCCTCAGTACGGGTACCTGACTCTGAACAATG
GCAGTCAGGCCGTGGGCCGTTCCTCCTTCTACTGCCTGGAGTACTTTCCT
TCTCAAATGCTGAGAACGGGCAACAACTTTGAGTTCAGCTACACGTTTGA
GGACGTGCCTTTTCACAGCAGCTACGCGCACAGCCAAAGCCTGGACCGGC
TGATGAACCCCCTCATCGACCAGTACCTGTACTACCTGTCTCGGACTCAG
ACCACGAGTGGTACCGCAGGAAATCGGACGTTGCAATTTTCTCAGGCCGG
GCCTAGTAGCATGGCGAATCAGGCCAAAAACTGGCTACCCGGGCCCTGCT
ACCGGCAGCAACGCGTCTCCAAGACAGCGAATCAAAATAACAACAGCAAC
TTTGCCTGGACCGGTGCCACCAAGTATCATCTGAATGGCAGAGACTCTCT
GGTAAATCCCGGTCCCGCTATGGCAACCCACAAGGACGACGAAGACAAAT
TTTTTCCGATGAGCGGAGTCTTAATATTTGGGAAACAGGGAGCTGGAAAT
AGCAACGTGGACCTTGACAACGTTATGATAACCAGTGAGGAAGAAATTAA
AACCACCAACCCAGTGGCCACAGAACAGTACGGCACGGTGGCCACTAACC
TGCAATCGTCAAACACCGCTCCTGCTACAGGGACCGTCAACAGTCAAGGA
GCCTTACCTGGCATGGTCTGGCAGAACCGGGACGTGTACCTGCAGGGTCC
TATCTGGGCCAAGATTCCTCACACGGACGGACACTTTCATCCCTCGCCGC
TGATGGGAGGCTTTGGACTGAAACACCCGCCTCCTCAGATCCTGATTAAG
AATACACCTGTTCCCGCGAATCCTCCAACTACCTTCAGTCCAGCTAAGTT
TGCGTCGTTCATCACGCAGTACAGCACCGGACAGGTCAGCGTGGAAATTG
AATGGGAGCTGCAGAAAGAAAACAGCAAACGCTGGAACCCAGAGATTCAA
TACACTTCCAACTACAACAAATCTACAAATGTGGACTTTGCTGTTGACAC
AAATGGCGTTTATTCTGAGCCTCGCCCCATCGGCACCCGTTACCTCACCC
GTAATCTG
Exemplary AAV Anc80 Capsid Amino Acid Sequence
(SEQ ID NO: 41)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAKTAPGKKRPVEQSP
QEPDSSSGIGKKGQQPAKKRLNFGQTGDSESVPDPQPLGEPPAAPSGVGS
NTMAAGGGAPMADNNEGADGVGNASGNWHCDSTWLGDRVITTSTRTWALP
TYNNHLYKQISSQSGASTNDNTYFGYSTPWGYFDFNRFHCHFSPRDWQRL
INNNWGFRPKRLNFKLFNIQVKEVTTNDGTTTIANNLTSTVQVFTDSEYQ
LPYVLGSAHQGCLPPFPADVFMIPQYGYLTLNNGSQAVGRSSFYCLEYFP
SQMLRTGNNFEFSYTFEDVPFHSSYAHSQSLDRLMNPLIDQYLYYLSRTQ
TTSGTAGNRTLQFSQAGPSSMANQAKNWLPGPCYRQQRVSKTANQNNNSN
FAWTGATKYHLNGRDSLVNPGPAMATHKDDEDKFFPMSGVLIFGKQGAGN
SNVDLDNVMITSEEEIKTTNPVATEQYGTVATNLQSSNTAPATGTVNSQG
ALPGMVWQNRDVYLQGPIWAKIPHTDGHFHPSPLMGGFGLKHPPPQILIK
NTPVPANPPTTFSPAKFASFITQYSTGQVSVEIEWELQKENSKRWNPEIQ
YTSNYNKSTNVDFAVDTNGVYSEPRPIGTRYLTRNL

### Compositions

Among other things, the present disclosure provides compositions. In some aspects, a composition comprises a construct as described herein. In some aspects, a composition comprises one or more constructs as described herein. In some aspects, a composition comprises a plurality of constructs as described herein. In some aspects, when more than one construct is included in the composition, the constructs are each different.

In some aspects, a composition comprises an AAV particle as described herein. In some aspects, a composition comprises one or more AAV particles as described herein. In some aspects, a composition comprises a plurality of AAV particles. In come aspects, when more than one AAV particle is included in the composition, the AAV particles are each different.

In some aspects, a composition comprises a vector as described herein. In some aspects, a composition comprises one or more vectors as described herein. In some aspects, a composition comprises a plurality of vectors as described herein. In some aspects, when more than one vector is included in the composition, the vectors are each different.

In some aspects, a composition comprises a cell as described herein. In some aspects, a composition comprise one or more cells as described herein.

In some aspects, a composition is or comprises a pharmaceutical composition. In some aspects, the pharmaceutic composition comprises a pharmaceutically acceptable carrier. In some aspects, a composition is or comprises a synthetic perilymph solution. In some aspects, a synthetic perilymph solution comprises 20-200 mM NaCl; 1-5 mM KCl; 0.1-10 mM CaCl₂); 1-10 mM glucose; and 2-50 mM HEPES, with a pH between about 6 and about 9.

In some aspects, the composition comprises a construct comprising (i) a 5′ ITR (e.g., an AAV 5′ ITR), (ii) a promoter comprising the nucleic acid sequence having at least 85%, at least 90%, at least 95%, at least 96%, at least 97%, at least 98%, at least 99%, or 100% identity to of any of one of SEQ ID NOs: 1-15, (iii) a polynucleotide encoding a polypeptide (e.g., a therapeutic polypeptide), (v) optionally, a 3× FLAG tag (e.g., comprising the nucleic acid sequence of SEQ ID NO: 39), (vi) a polyA sequence, and (vii) a 3′ ITR (e.g., an AAV 3′ ITR).

In some aspects, the composition comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a polynucleotide encoding a polypeptide, (iv) optionally, a 3× FLAG tag comprising the nucleic acid sequence of SEQ ID NO: 39, (v) a polyA sequence comprising the nucleic acid sequence of SEQ ID NO: 22, and (vi) a 3′ ITR comprising the nucleic acid sequence of SEQ ID NO: 17.

In some aspects, the composition comprises a construct comprising (i) a 5′ ITR; (ii) a promoter comprising the nucleic acid sequence of any of one of SEQ ID NOs: 1-15; (iii) a polynucleotide encoding an outer hair cell polypeptide comprising a gene selected from actin gamma 1 (ACTG1), adenylate cyclase type 1 (ADCY1), calcium binding protein 2 (CABP2), coiled-coil domain-containing 50 (CCDC50), cadherin-related 23 (CDH23), carcinoembryonic antigen-related cell adhesion molecule 16 (CEACAM16), chromodomain helicase DNA-binding protein 7 (CHD7), calcium- and integrin-binding family member 2 (CIB2), claudin 14 (CLDN14), chloride intracellular channel 5 (CLIC5), caseinolytic mitochondrial matrix peptidase proteolytic subunit (CLPP), clarin 1 (CLRN1), pejvakin (DFNB59), endothelin 3 (EDN3), ELMO domain-containing protein 3 (ELMOD3), epidermal growth factor receptor kinase substrate 8 (EPS8), espin (ESPN), estrogen-related receptor beta (ESRRB), eyes absent homolog 1 (EYA1), GIPC PDZ domain-containing family, member 3 (GIPC3), G protein-coupled receptor 98 (GPR98), G-protein signaling modulator 2 (GPSM2), glutaredoxin, cysteine-rich 1 (GRXCR1), glutaredoxin, cysteine-rich 2 (GRXCR2), immunoglobulin-like domain-containing receptor 1 (ILDR1), lysyl-tRNA synthetase (KARS), Potassium voltage-gated channel, KQT-like subfamily, member 4 (KCNQ4), lipoma HMGIC fusion partner-like 5 (LHFPL5), leucine-rich transmembrane and O-methyltransferase domain-containing (LRTOMT1 COMT2), tricellulin (MARVELD2), micro-ma 96 (MIR96), methionine sulfoxide reductase B3 (MSRB3), myosin, heavy chain 9, non-muscle (MYH9), myosin, heavy chain 14, non-muscle (MYH14), unconventional myosin IIIA (MYO3A), unconventional myosin VI (MYO6), unconventional myosin VIIA (MYO7A), unconventional myosin XVA (MYO15A), otoferlin (OTOF), otogelin-like protein (OTOGL), purinergic receptor P2X, ligand-gated ion channel, 2 (P2RX2), protocadherin 15 (PCDH15), PDZ domain-containing 7 (PDZD7), polyribonucleotide nucleotidyltransferase 1, mitochondrial (PNPT1), POU domain, class 4, transcription factor 3 (POU4F3), phosphoribosyl pyrophosphate synthetase 1 (PRPS1), protein tyrosine phosphatase, receptor type Q (PTPRQ), radixin (RDX), scaffold-containing ankyrin repeats and SAM domain (SANS), serpin peptidase inhibitor, clade B, member 6 (SERPINB6), SIX homeobox 1 (SIX1), SIX homeobox 5 (SIX5), prestin (SLC26A5), second mitochondrial-derived activator of caspase (SMAC/DIABLO), small muscle protein, x-linked (SMPX), stereocilin (STRC), nesprin-4 (SYNE4), TBC1 domain family, member 24 (TBC/D24), tight junction protein XO 2 (TJP2), transmembrane channel-like protein 1 (TMC1), transmembrane inner ear-expressed protein (TMIE), transmembrane protease, serine 3 (TMPRSS3), taperin (TPRN), TRIO and F-actin-binding protein (TRIOBP), Thrombospondin-type laminin G domain and EAR repeats (TSPEAR), harmonin (USH1C), usherin (USH2A), wolframin (WFS1), and whirlin (WHRN), or any combination thereof; (v) optionally, a 3× FLAG tag comprising the nucleic acid sequence of SEQ ID NO: 39; (vi) a polyA sequence; and (vii) a 3′ ITR.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a polynucleotide encoding a polypeptide, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid sequence of SEQ ID NO: 19, (iv) a polynucleotide encoding a polypeptide, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid sequence of SEQ ID NO: 19, (v) a polynucleotide encoding a polypeptide, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid sequence of SEQ ID NO: 19, (iv) a polynucleotide encoding a polypeptide, (v) optionally, a 3× FLAG tag comprising the nucleic acid sequence of SEQ ID NO: 39, (vi) a 3′ UTR comprising the nucleic acid sequence of SEQ ID NO: 45, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid of SEQ ID NO: 19, (v) a polynucleotide encoding a polypeptide, (vi) optionally, a 3× FLAG tag comprising the nucleic acid sequence of SEQ ID NO: 39, (vii) a 3′ UTR comprising the nucleic acid sequence of SEQ ID NO: 45, (viii) a polyA sequence comprising the nucleic acid sequence of SEQ ID NO: 22, and (ix) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16; (ii) a promoter comprising the nucleic acid sequence of any one of SEQ ID NOs: 1-15; (iii) a polynucleotide encoding a polypeptide encoding an outer hair cell polypeptide comprising a gene selected from actin gamma 1 (ACTG1), adenylate cyclase type 1 (ADCY1), calcium binding protein 2 (CABP2), coiled-coil domain-containing 50 (CCDC50), cadherin-related 23 (CDH23), carcinoembryonic antigen-related cell adhesion molecule 16 (CEACAM16), chromodomain helicase DNA-binding protein 7 (CHD7), calcium- and integrin-binding family member 2 (CIB2), claudin 14 (CLDN14), chloride intracellular channel 5 (CLIC5), caseinolytic mitochondrial matrix peptidase proteolytic subunit (CLPP), clarin 1 (CLRN1), pejvakin (DFNB59), endothelin 3 (EDN3), ELMO domain-containing protein 3 (ELMOD3), epidermal growth factor receptor kinase substrate 8 (EPS8), espin (ESPN), estrogen-related receptor beta (ESRRB), eyes absent homolog 1 (EYA1), GIPC PDZ domain-containing family, member 3 (GIPC3), G protein-coupled receptor 98 (GPR98), G-protein signaling modulator 2 (GPSM2), glutaredoxin, cysteine-rich 1 (GRXCR1), glutaredoxin, cysteine-rich 2 (GRXCR2), immunoglobulin-like domain-containing receptor 1 (ILDR1), lysyl-tRNA synthetase (KARS), Potassium voltage-gated channel, KQT-like subfamily, member 4 (KCNQ4), lipoma HMGIC fusion partner-like 5 (LHFPL5), leucine-rich transmembrane and O-methyltransferase domain-containing (LRTOMT1 COMT2), tricellulin (MARVELD2), micro-ma 96 (MIR96), methionine sulfoxide reductase B3 (MSRB3), myosin, heavy chain 9, non-muscle (MYH9), myosin, heavy chain 14, non-muscle (MYH14), unconventional myosin IIIA (MYO3A), unconventional myosin VI (MYO6), unconventional myosin VIIA (MYO7A), unconventional myosin XVA (MYO15A), otoferlin (OTOF), otogelin-like protein (OTOGL), purinergic receptor P2X, ligand-gated ion channel, 2 (P2RX2), protocadherin 15 (PCDH15), PDZ domain-containing 7 (PDZD7), polyribonucleotide nucleotidyltransferase 1, mitochondrial (PNPT1), POU domain, class 4, transcription factor 3 (POU4F3), phosphoribosyl pyrophosphate synthetase 1 (PRPS1), protein tyrosine phosphatase, receptor type Q (PTPRQ), radixin (RDX), scaffold-containing ankyrin repeats and SAM domain (SANS), serpin peptidase inhibitor, clade B, member 6 (SERPINB6), SIX homeobox 1 (SIX1), SIX homeobox 5 (SIX5), prestin (SLC26A5), second mitochondrial-derived activator of caspase (SMAC/DIABLO), small muscle protein, x-linked (SMPX), stereocilin (STRC), nesprin-4 (SYNE4), TBC1 domain family, member 24 (TBC/D24), tight junction protein XO 2 (TJP2), transmembrane channel-like protein 1 (TMC1), transmembrane inner ear-expressed protein (TMIE), transmembrane protease, serine 3 (TMPRSS3), taperin (TPRN), TRIO and F-actin-binding protein (TRIOBP), Thrombospondin-type laminin G domain and EAR repeats (TSPEAR), harmonin (USH1C), usherin (USH2A), wolframin (WFS1), and whirlin (WHRN), or any combination thereof; (iv) optionally, a 3× FLAG tag comprising the nucleic acid sequence of SEQ ID NO: 39; (v) a polyA sequence comprising the nucleic acid sequence of SEQ ID NO: 22; and (vi) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16; (ii) a promoter comprising the nucleic acid sequence of any one of SEQ ID NOs: 1-15; (iii) a polynucleotide encoding a polypeptide encoding an outer hair cell polypeptide comprising a gene selected from cadherin-related 23 (CDH23), clarin 1 (CLRN1), pejvakin (DFNB59), Potassium voltage-gated channel, KQT-like subfamily, member 4 (KCNQ4), otoferlin (OTOF), protocadherin 15 (PCDH15), POU domain, class 4, transcription factor 3 (POU4F3), prestin (SLC26A5), stereocilin (STRC), transmembrane channel-like protein 1 (TMC1), TRIO and F-actin-binding protein (TRIOBP), harmonin (USH1C), usherin (USH2A), wolframin (WFS1), and whirlin (WHRN), or any combination thereof; (iv) optionally, a 3× FLAG tag comprising the nucleic acid sequence of SEQ ID NO: 39; (v) a polyA sequence comprising the nucleic acid sequence of SEQ ID NO: 22; and (vi) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16; (ii) a promoter comprising the nucleic acid sequence of any one of SEQ ID NOs: 1-15; (iii) a polynucleotide encoding a polypeptide encoding Potassium voltage-gated channel, KQT-like subfamily, member 4 (KCNQ4); (iv) optionally, a 3× FLAG tag comprising the nucleic acid sequence of SEQ ID NO: 39; (v) a polyA sequence comprising the nucleic acid sequence of SEQ ID NO: 22; and (vi) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a KCNQ4 coding region comprising the nucleic acid sequence of SEQ ID NO: 20, (iv) optionally, a 3× FLAG tag comprising the nucleic acid sequence of SEQ ID NO: 39, (v) a polyA sequence comprising the nucleic acid sequence of SEQ ID NO: 22, and (vi) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a 3′ UTR, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid of SEQ ID NO: 19, (v a KCNQ4 coding region comprising the nucleic acid sequence of SEQ ID NO: 20, (vi) optionally, a 3× FLAG tag comprising the nucleic acid sequence of SEQ ID NO: 39, (vii) a 3′ UTR comprising the nucleic acid sequence of SEQ ID NO: 45, (viii) a polyA sequence comprising the nucleic acid sequence of SEQ ID NO: 22, and (ix) a 3′ ITR comprising the nucleic acid sequence of SEQ ID NO: 17.

In some aspects, the composition comprises a construct having at least 80%, at least 85%, at least 90%, at least 95%, at least 96%, at least 97%, at least 98% at least 99%, or 100% identity to any one of SEQ ID NOs: 23-38, and 49-50. In some aspects, the composition comprises a construct comprising the nucleic acid sequence of any of SEQ ID NOs: 23-38 and 49-50. In some aspects, the construct has at least 80%, at least 85%, at least 90%, at least 95%, at least 96%, at least 97%, at least 98% at least 99%, or 100% identity to any one of nucleotides 12-4396 of SEQ ID NO: 23, 12-4464 of SEQ ID NO: 24, nucleotides 12-4016 of SEQ ID NO: 25, nucleotides 12-4521 of SEQ ID NO: 26, nucleotides 12-3750 of SEQ ID NO: 27, nucleotides 12-3928 of SEQ ID NO: 28, nucleotides 12-4641 of SEQ ID NO: 29, nucleotides 12-3994 of SEQ ID NO: 30, nucleotides 12-4426 of SEQ ID NO: 31, nucleotides 12-4307 of SEQ ID NO: 32, nucleotides 12-4293 of SEQ ID NO: 33, nucleotides 12-4565 of SEQ ID NO: 34, nucleotides 12-4224 of SEQ ID NO: 35, nucleotides 12-4140 of SEQ ID NO: 36, nucleotides 12-4816 of SEQ ID NO: 37, or nucleotides 12-4915 of SEQ ID NO: 38. In some aspects, the composition comprises a construct comprising a nucleic acid sequence comprising any one of nucleotides 12-4396 of SEQ ID NO: 23, 12-4464 of SEQ ID NO: 24, nucleotides 12-4016 of SEQ ID NO: 25, nucleotides 12-4521 of SEQ ID NO: 26, nucleotides 12-3750 of SEQ ID NO: 27, nucleotides 12-3928 of SEQ ID NO: 28, nucleotides 12-4641 of SEQ ID NO: 29, nucleotides 12-3994 of SEQ ID NO: 30, nucleotides 12-4426 of SEQ ID NO: 31, nucleotides 12-4307 of SEQ ID NO: 32, nucleotides 12-4293 of SEQ ID NO: 33, nucleotides 12-4565 of SEQ ID NO: 34, nucleotides 12-4224 of SEQ ID NO: 35, nucleotides 12-4140 of SEQ ID NO: 36, nucleotides 12-4816 of SEQ ID NO: 37, or nucleotides 12-4915 of SEQ ID NO: 38.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 23. In some aspects, the composition comprises a construct comprising the nucleic acid sequence of SEQ ID NO: 23.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4396 of SEQ ID NO: 23. In some aspects, the composition comprises a construct comprising nucleotides 12-4396 of SEQ ID NO: 23.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) an oncomodulin promoter comprising the nucleic acid sequence of SEQ ID NO: 1, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 24. In some aspects, the composition comprises a construct comprising the nucleic acid sequence of SEQ ID NO: 24.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4464 of SEQ ID NO: 24. In some aspects, the composition comprises a construct comprising nucleotides 12-4464 of SEQ ID NO: 24.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 24. In some aspects, the rAAVAnc80 particle comprises the nucleic acid sequence of SEQ ID NO: 24.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) an oncomodulin promoter comprising the nucleic acid sequence of SEQ ID NO: 2, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) an oncomodulin promoter comprising the nucleic acid sequence of SEQ ID NO: 2, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the construct comprises a nucleic acid sequence having at least 80%, at least 85%, at least 90%, at least 95%, at least 96%, at least 97%, at least 98% at least 99%, or 100% identity to SEQ ID NO: 25. In some aspects, the composition comprises a construct comprising the nucleic acid sequence of SEQ ID NO: 25.

In some aspects, the construct comprises a nucleic acid sequence having at least 80%, at least 85%, at least 90%, at least 95%, at least 96%, at least 97%, at least 98% at least 99%, or 100% identity to nucleotides 12-4016 of SEQ ID NO: 25. In some aspects, the composition comprises a construct comprising nucleotides 12-4016 of SEQ ID NO: 25.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 25. In some aspects, the rAAVAnc80 particle comprises the nucleic acid sequence of SEQ ID NO: 25.

In some aspects, the composition comprises a construct comprising (i) a 5′ ITR comprising the nucleic acid sequence of SEQ ID NO: 16, (ii) an oncomodulin promoter comprising the nucleic acid sequence of SEQ ID NO: 1, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 26. In some aspects, the composition comprises a construct comprising the nucleic acid sequence of SEQ ID NO: 26.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4521 of SEQ ID NO: 26. In some aspects, the composition comprises a construct comprising nucleotides 12-4521 of SEQ ID NO: 26.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 26. In some aspects, the rAAVAnc80 particle comprises the nucleic acid sequence of SEQ ID NO: 26.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a prestin promoter comprising the nucleic acid sequence of SEQ ID NO: 3, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a prestin promoter comprising the nucleic acid sequence of SEQ ID NO: 3, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 27. In some aspects, the composition comprises a construct comprising the nucleic acid sequence of SEQ ID NO: 27.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-3750 of SEQ ID NO: 27. In some aspects, the composition comprises a construct comprising nucleotides 12-3750 of SEQ ID NO: 27.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 27. In some aspects, the rAAVAnc80 particle comprises the nucleic acid sequence of SEQ ID NO: 27.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a CHRNA10 promoter comprising the nucleic acid sequence of SEQ ID NO: 4, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a CHRNA10 promoter comprising the nucleic acid sequence of SEQ ID NO: 4, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 28. In some aspects, the composition comprises a construct comprising the nucleic acid sequence of SEQ ID NO: 28.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-3928 of SEQ ID NO: 28. In some aspects, the composition comprises a construct comprising nucleotides 12-3928 of SEQ ID NO: 28.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 28. In some aspects, the rAAVAnc80 particle comprises the nucleic acid sequence of SEQ ID NO: 28.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a DNM3 promoter comprising the nucleic acid sequence of SEQ ID NO: 5, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a DNM3 promoter comprising the nucleic acid sequence of SEQ ID NO: 5, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 29. In some aspects, the composition comprises a construct comprising the nucleic acid sequence of SEQ ID NO: 29.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4641 of SEQ ID NO: 29. In some aspects, the composition comprises a construct comprising nucleotides 12-4641 of SEQ ID NO: 29.

In some aspects, the rAAVAnc80 particle comprises a nucleic acid having at least 80%, at least 85%, at least 90%, at least 95%, at least 96%, at least 97%, at least 98% at least 99%, or 100% identity to SEQ ID NO: 29. In some aspects, the rAAVAnc80 particle comprises the nucleic acid sequence of SEQ ID NO: 29.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a MUC15 promoter comprising the nucleic acid sequence of SEQ ID NO: 6, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a MUC15 promoter comprising the nucleic acid sequence of SEQ ID NO: 6, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 30. In some aspects, the composition comprises a construct comprising the nucleic acid sequence of SEQ ID NO: 30.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-3994 of SEQ ID NO: 30. In some aspects, the composition comprises a construct comprising nucleotides 12-3994 of SEQ ID NO: 30.

In some aspects, the rAAVAnc80 particle comprises a nucleic acid having at least 80%, at least 85%, at least 90%, at least 95%, at least 96%, at least 97%, at least 98% at least 99%, or 100% identity to SEQ ID NO: 30. In some aspects, the rAAVAnc80 particle comprises the nucleic acid sequence of SEQ ID NO: 30.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a PLBD1 promoter comprising the nucleic acid sequence of SEQ ID NO: 7, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a PLBD1 promoter comprising the nucleic acid sequence of SEQ ID NO: 7, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 31. In some aspects, the composition comprises a construct comprising the nucleic acid sequence of SEQ ID NO: 31.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4426 of SEQ ID NO: 31. In some aspects, the composition comprises a construct comprising nucleotides 12-4426 of SEQ ID NO: 31.

In some aspects, the rAAVAnc80 particle comprises a nucleic acid having at least 80%, at least 85%, at least 90%, at least 95%, at least 96%, at least 97%, at least 98% at least 99%, or 100% identity to SEQ ID NO: 31. In some aspects, the construct rAAVAnc80 particle the nucleic acid sequence of SEQ ID NO: 31.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a RORB promoter comprising the nucleic acid sequence of SEQ ID NO: 8, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a RORB promoter comprising the nucleic acid sequence of SEQ ID NO: 8, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 32. In some aspects, the composition comprises a construct comprising the nucleic acid sequence of SEQ ID NO: 32.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4307 of SEQ ID NO: 32. In some aspects, the composition comprises a construct comprising nucleotides 12-4307 of SEQ ID NO: 32.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 32. In some aspects, the rAAVAnc80 particle comprises the nucleic acid sequence of SEQ ID NO: 32.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a STRIP2 promoter comprising the nucleic acid sequence of SEQ ID NO: 9, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a STRIP2 promoter comprising the nucleic acid sequence of SEQ ID NO: 9, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 33. In some aspects, the composition comprises a construct comprising the nucleic acid sequence of SEQ ID NO: 33.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4293 of SEQ ID NO: 33. In some aspects, the composition comprises a construct comprising nucleotides 12-4293 of SEQ ID NO: 33.

In some aspects, the rAAVAnc80 particle comprises a nucleic acid having at least 80%, at least 85%, at least 90%, at least 95%, at least 96%, at least 97%, at least 98% at least 99%, or 100% identity to SEQ ID NO: 33. In some aspects, the rAAVAnc80 particle comprises the nucleic acid sequence of SEQ ID NO: 33.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a AQP11 promoter comprising the nucleic acid sequence of SEQ ID NO: 10, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a AQP11 promoter comprising the nucleic acid sequence of SEQ ID NO: 10, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 34. In some aspects, the composition comprises a construct comprising the nucleic acid sequence of SEQ ID NO: 34.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4565 of SEQ ID NO: 34. In some aspects, the composition comprises a construct comprising nucleotides 12-4565 of SEQ ID NO: 34.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 34. In some aspects, the rAAVAnc80 particle comprises the nucleic acid sequence of SEQ ID NO: 34.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a KCNQ4 promoter comprising the nucleic acid sequence of SEQ ID NO: 11, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a KCNQ4 promoter comprising the nucleic acid sequence of SEQ ID NO: 11, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 35. In some aspects, the composition comprises a construct comprising the nucleic acid sequence of SEQ ID NO: 35.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4224 of SEQ ID NO: 35. In some aspects, the composition comprises a construct comprising nucleotides 12-4224 of SEQ ID NO: 35.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 35. In some aspects, the construct rAAVAnc80 particle the nucleic acid sequence of SEQ ID NO: 35.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a LBH promoter comprising the nucleic acid sequence of SEQ ID NO: 12, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a LBH promoter comprising the nucleic acid sequence of SEQ ID NO: 12, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 36. In some aspects, the composition comprises a construct comprising the nucleic acid sequence of SEQ ID NO: 36.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4140 of SEQ ID NO: 36. In some aspects, the composition comprises a construct comprising nucleotides 12-4140 of SEQ ID NO: 36.

In some aspects, the rAAVAnc80 particle comprises a nucleic acid having at least 80%, at least 85%, at least 90%, at least 95%, at least 96%, at least 97%, at least 98% at least 99%, or 100% identity to SEQ ID NO: 36. In some aspects, the rAAVAnc80 particle comprises the nucleic acid sequence of SEQ ID NO: 36.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a STRC promoter comprising the nucleic acid sequence of SEQ ID NO: 13, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a STRC promoter comprising the nucleic acid sequence of SEQ ID NO: 13, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 37. In some aspects, the composition comprises a construct comprising the nucleic acid sequence of SEQ ID NO: 37.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4816 of SEQ ID NO: 37. In some aspects, the composition comprises a construct comprising nucleotides 12-4816 of SEQ ID NO: 37.

In some aspects, the rAAVAnc80 particle comprises a nucleic acid having at least 80%, at least 85%, at least 90%, at least 95%, at least 96%, at least 97%, at least 98% at least 99%, or 100% identity to SEQ ID NO: 37. In some aspects, the rAAVAnc80 particle comprises the nucleic acid sequence of SEQ ID NO: 37.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a TUBA8 promoter comprising the nucleic acid sequence of SEQ ID NO: 14, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a TUBA8 promoter comprising the nucleic acid sequence of SEQ ID NO: 14, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 38. In some aspects, the composition comprises a construct comprising the nucleic acid sequence of SEQ ID NO: 38.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4915 of SEQ ID NO: 38. In some aspects, the composition comprises a construct comprising nucleotides 12-4915 of SEQ ID NO: 38.

In some aspects, the rAAVAnc80 particle comprises a nucleic acid having at least 80%, at least 85%, at least 90%, at least 95%, at least 96%, at least 97%, at least 98% at least 99%, or 100% identity to SEQ ID NO: 38. In some aspects, the rAAVAnc80 particle comprises the nucleic acid sequence of SEQ ID NO: 38.

In some aspects, the composition comprises a construct comprising (i) a 5′ ITR comprising the nucleic acid sequence of SEQ ID NO: 16, (ii) a CMV enhancer comprising the nucleic acid sequence of SEQ ID NO: 18, (iii) a prestin promoter comprising the nucleic acid sequence of SEQ ID NO: 15, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the composition comprises a construct comprising (i) a 5′ ITR comprising the nucleic acid sequence of SEQ ID NO: 16, (ii) a prestin promoter comprising the nucleic acid sequence of SEQ ID NO: 15, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 49. In some aspects, the composition comprises a construct comprising the nucleic acid sequence of SEQ ID NO: 49.

In some aspects, the composition comprises a construct comprising a nucleic acid sequence having at least 80%, at least 85%, at least 90%, at least 95%, at least 96%, at least 97%, at least 98% at least 99%, or 100% identity to nucleotides 12-4070 of SEQ ID NO: 49. In some aspects, the composition comprises a construct comprising nucleotides 12-4070 of SEQ ID NO: 49.

In some aspects, the rAAVAnc80 particle comprises a nucleic acid sequence having at least 80%, at least 85%, at least 90%, at least 95%, at least 96%, at least 97%, at least 98% at least 99%, or 100% identity to SEQ ID NO: 49. In some aspects, the rAAVAnc80 particle comprises the nucleic acid sequence of SEQ ID NO: 49.

In some aspects, the composition comprises a construct comprising a nucleic acid sequence having at least 80%, at least 85%, at least 90%, at least 95%, at least 96%, at least 97%, at least 98% at least 99%, or 100% identity to SEQ ID NO: 50. In some aspects, the composition comprises a construct comprising the nucleic acid sequence of SEQ ID NO: 50.

In some aspects, the rAAVAnc80 particle comprises a nucleic acid having at least 80%, at least 85%, at least 90%, at least 95%, at least 96%, at least 97%, at least 98% at least 99%, or 100% identity to SEQ ID NO: 50. In some aspects, the rAAVAnc80 particle comprises the nucleic acid sequence of SEQ ID NO: 50.

### Dosing and Volume of Administration

In some aspects, a composition disclosed herein, e.g., one or a plurality of AAV vectors disclosed herein, is administered as a single dose or as a plurality of doses.

In some aspects, a composition disclosed herein is administered as a single dose. In some aspects, a composition disclosed herein is administered as a plurality of doses, e.g., 2, 3, 4, 5, 6, 7, 8, 9 or 10 doses.

In some aspects, a composition disclosed herein (e.g., a composition comprising one or a plurality of rAAV constructs disclosed herein) is administered at a volume of between about 0.01 mL to about 2.00 mL, between about 0.05 mL to about 1.5 mL, between about 0.08 mL to about 1.10 mL, or between about 0.09 mL to about 1.0 mL. In some aspects, a composition disclosed herein (e.g., a composition comprising one or a plurality of rAAV constructs disclosed herein) is administered at a volume of about 0.01 mL, about 0.02 mL, about 0.03 mL, about 0.04 mL, about 0.05 mL, about 0.06 mL, about 0.07 mL, about 0.08 mL, about 0.09 mL, about 1.00 mL, about 1.10 mL, about 1.20 mL, about 1.30 mL, about 1.40 mL, about 1.50 mL, about 1.60 mL, about 1.70 mL, about 1.80 mL, about 1.90 mL, or about 2.00 mL. In some aspects, a composition disclosed herein is administered at a volume of about 0.01 mL. In some aspects, a composition disclosed herein is administered at a volume of about 0.02 mL. In some aspects, a composition disclosed herein is administered at a volume of about 0.03 mL. In some aspects, a composition disclosed herein is administered at a volume of about 0.04 mL. In some aspects, a composition disclosed herein is administered at a volume of about 0.05 mL. In some aspects, a composition disclosed herein is administered at a volume of about 0.06 mL. In some aspects, a composition disclosed herein is administered at a volume of about 0.07 mL. In some aspects, a composition disclosed herein is administered at a volume of about 0.08 mL. In some aspects, a composition disclosed herein is administered at a volume of about 0.09 mL. In some aspects, a composition disclosed herein is administered at a volume of about 0.10 mL. In some aspects, a composition disclosed herein is administered at a volume of about 0.20 mL. In some aspects, a composition disclosed herein is administered at a volume of about 0.3 mL. In some aspects, a composition disclosed herein is administered at a volume of about 0.4 mL. In some aspects, a composition disclosed herein is administered at a volume of about 0.5 mL. In some aspects, a composition disclosed herein is administered at a volume of about 0.6 mL. In some aspects, a composition disclosed herein is administered at a volume of about 0.7 mL. In some aspects, a composition disclosed herein is administered at a volume of about 0.8 mL. In some aspects, a composition disclosed herein is administered at a volume of about 0.9 mL. In some aspects, a composition disclosed herein is administered at a volume of about 1.00 mL. In some aspects, a composition disclosed herein is administered at a volume of about 1.10 mL. In some aspects, a composition disclosed herein is administered at a volume of about 1.20 mL. In some aspects, a composition disclosed herein is administered at a volume of about 1.30 mL. In some aspects, a composition disclosed herein is administered at a volume of about 1.40 mL. In some aspects, a composition disclosed herein is administered at a volume of about 1.50 mL. In some aspects, a composition disclosed herein is administered at a volume of about 1.60 mL. In some aspects, a composition disclosed herein is administered at a volume of about 1.70 mL. In some aspects, a composition disclosed herein is administered at a volume of about 1.80 mL. In some aspects, a composition disclosed herein is administered at a volume of about 1.90 mL. In some aspects, a composition disclosed herein is administered at a volume of about 2.00 mL.

In some aspects, a composition disclosed herein (e.g., a composition comprising one or a plurality of rAAV constructs disclosed herein) is administered at a volume of about 0.01 to 2.00 mL, about 0.02 to 1.90 mL, about 0.03 to 1.8 mL, about 0.04 to 1.70 mL, about 0.05 to 1.60 mL, about 0.06 to 1.50 mL, about 0.06 to 1.40 mL, about 0.07 to 1.30 mL, about 0.08 to 1.20 mL, or about 0.09 to 1.10 mL. In some aspects a composition disclosed herein (e.g., a composition comprising one or a plurality of rAAV constructs disclosed herein) is administered at a volume of about 0.01 to 2.00 mL, about 0.02 to 2.00 mL, about 0.03 to 2.00 mL, about 0.04 to 2.00 mL, about 0.05 to 2.00 mL, about 0.06 to 2.00 mL, about 0.07 to 2.00 mL, about 0.08 to 2.00 mL, about 0.09 to 2.00 mL, about 0.01 to 1.90 mL, about 0.01 to 1.80 mL, about 0.01 to 1.70 mL, about 0.01 to 1.60 mL, about 0.01 to 1.50 mL, about 0.01 to 1.40 mL, about 0.01 to 1.30 mL, about 0.01 to 1.20 mL, about 0.01 to 1.10 mL, about 0.01 to 1.00 mL, about 0.01 to 0.09 mL.

In some aspects, a dosing regimen comprises delivery in a volume of at least 0.01 mL, at least 0.02 mL, at least 0.03 mL, at least 0.04 mL, at least 0.05 mL, at least 0.06 mL, at least 0.07 mL, at least 0.08 mL, at least 0.09 mL, at least 0.10 mL, at least 0.11 mL, at least 0.12 mL, at least 0.13 mL, at least 0.14 mL, at least 0.15 mL, at least 0.16 mL, at least 0.17 mL, at least 0.18 mL, at least 0.19 mL, or at least 0.20 mL per cochlea. In some aspects, a dosing regimen comprises delivery in a volume of at most 0.30 mL, at most 0.25 mL, at most 0.20 mL, at most 0.15 mL, at most 0.14 mL, at most 0.13 mL, at most 0.12 mL, at most 0.11 mL, at most 0.10 mL, at most 0.09 mL, at most 0.08 mL, at most 0.07 mL, at most 0.06 mL, or at most 0.05 mL per cochlea. In some aspects, the dosing regimen comprises delivery in a volume of about 0.05 mL, about 0.06 mL, about 0.07 mL, about 0.08 mL, about 0.09 mL, about 0.10 mL, about 0.11 mL, about 0.12 mL, about 0.13 mL, about 0.14 mL, or about 0.15 mL per cochlea, depending on the population.

### Single AAV Construct Compositions

In some aspects, the present disclosure provides compositions or systems comprising AAV particles comprised of a single construct. In some such aspects, a single construct may deliver a polynucleotide that encodes a functional (e.g., wild-type or otherwise functional, e.g., codon optimized) polypeptide. In some aspects, a construct is or comprises an rAAV construct. In some aspects described herein, a single rAAV construct is capable of expressing a polypeptide thereof in a target cell (e.g., an inner ear outer hair cell).

In some aspects, a single construct composition or system may comprise any or all of the exemplary construct components described herein.

In some aspects, the construct comprises (i) a 5′ ITR comprising the nucleic acid sequence of SEQ ID NO: 16, (ii) a promoter comprising the nucleic acid sequence of any one of SEQ ID NOs: 1-15, (iii) a polynucleotide encoding a polypeptide, (iv) optionally, a 3× FLAG tag comprising the nucleic acid sequence of SEQ ID NO: 39, (v) a polyA sequence comprising the nucleic acid sequence of SEQ ID NO: 22, and (vi) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a polynucleotide encoding a polypeptide, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid sequence of SEQ ID NO: 19, (iv) a polynucleotide encoding a polypeptide, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid sequence of SEQ ID NO: 19, (v) a polynucleotide encoding a polypeptide, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid sequence of SEQ ID NO: 19, (iv) a polynucleotide encoding a polypeptide, (v) optionally, a 3× FLAG tag comprising the nucleic acid sequence of SEQ ID NO: 39, (vi) a 3′ UTR comprising the nucleic acid sequence of SEQ ID NO: 45, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid of SEQ ID NO: 19, (v) a polynucleotide encoding a polypeptide, (vi) optionally, a 3× FLAG tag comprising the nucleic acid sequence of SEQ ID NO: 39, (vii) a 3′ UTR comprising the nucleic acid sequence of SEQ ID NO: 45, (viii) a polyA sequence comprising the nucleic acid sequence of SEQ ID NO: 22, and (ix) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a promoter comprising the nucleic acid sequence of any one of SEQ ID NOs: 1-15, (iii) a KCNQ4 coding region comprising the nucleic acid sequence of SEQ ID NO: 20, (iv) optionally, a 3× FLAG tag comprising the nucleic acid sequence of SEQ ID NO: 39, (v) a polyA sequence comprising the nucleic acid sequence of SEQ ID NO: 22, and (vi) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a promoter comprising the nucleic acid sequence of any one of SEQ ID NOs: 1-15, (iii) a 5′ UTR comprising the nucleic acid of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a 3′ UTR, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a promoter comprising the nucleic acid sequence of any one of SEQ ID NOs: 1-15, (iv) a 5′ UTR comprising the nucleic acid of SEQ ID NO: 19, (v a KCNQ4 coding region comprising the nucleic acid sequence of SEQ ID NO: 20, (vi) optionally, a 3× FLAG tag comprising the nucleic acid sequence of SEQ ID NO: 39, (vii) a 3′ UTR comprising the nucleic acid sequence of SEQ ID NO: 45, (viii) a polyA sequence comprising the nucleic acid sequence of SEQ ID NO: 22, and (ix) a 3′ ITR comprising the nucleic acid sequence of SEQ ID NO: 17.

In some aspects, the construct comprises at least 80%, at least 85%, at least 90%, at least 95%, at least 96%, at least 97%, at least 98% at least 99%, or 100% identity to any one of SEQ ID NOs: 23-38, and 49-50. In some aspects, the construct comprises the nucleic acid sequence of any of SEQ ID NOs: 23-38 and 49-50. In some aspects, the construct comprises at least 80%, at least 85%, at least 90%, at least 95%, at least 96%, at least 97%, at least 98% at least 99%, or 100% identity to any one of nucleotides 12-4396 of SEQ ID NO: 23, 12-4464 of SEQ ID NO: 24, nucleotides 12-4016 of SEQ ID NO: 25, nucleotides 12-4521 of SEQ ID NO: 26, nucleotides 12-3750 of SEQ ID NO: 27, nucleotides 12-3928 of SEQ ID NO: 28, nucleotides 12-4641 of SEQ ID NO: 29, nucleotides 12-3994 of SEQ ID NO: 30, nucleotides 12-4426 of SEQ ID NO: 31, nucleotides 12-4307 of SEQ ID NO: 32, nucleotides 12-4293 of SEQ ID NO: 33, nucleotides 12-4565 of SEQ ID NO: 34, nucleotides 12-4224 of SEQ ID NO: 35, nucleotides 12-4140 of SEQ ID NO: 36, nucleotides 12-4816 of SEQ ID NO: 37, or nucleotides 12-4915 of SEQ ID NO: 38. In some aspects, the construct comprises a nucleic acid sequence comprising any one of nucleotides 12-4396 of SEQ ID NO: 23, 12-4464 of SEQ ID NO: 24, nucleotides 12-4016 of SEQ ID NO: 25, nucleotides 12-4521 of SEQ ID NO: 26, nucleotides 12-3750 of SEQ ID NO: 27, nucleotides 12-3928 of SEQ ID NO: 28, nucleotides 12-4641 of SEQ ID NO: 29, nucleotides 12-3994 of SEQ ID NO: 30, nucleotides 12-4426 of SEQ ID NO: 31, nucleotides 12-4307 of SEQ ID NO: 32, nucleotides 12-4293 of SEQ ID NO: 33, nucleotides 12-4565 of SEQ ID NO: 34, nucleotides 12-4224 of SEQ ID NO: 35, nucleotides 12-4140 of SEQ ID NO: 36, nucleotides 12-4816 of SEQ ID NO: 37, or nucleotides 12-4915 of SEQ ID NO: 38.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 23. In some aspects, the construct comprises the nucleic acid sequence of SEQ ID NO: 23.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4396 of SEQ ID NO: 23. In some aspects, the construct comprises nucleotides 12-4396 of SEQ ID NO: 23.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 23. In some aspects, the rAAVAnc80 particle comprises the nucleic acid sequence of SEQ ID NO: 23.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) an oncomodulin promoter comprising the nucleic acid sequence of SEQ ID NO: 1, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

Exemplary Construct
(SEQ ID NO: 23)
CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCGCACGCGTGACATT
GATTATTGACTAGTTATTAATAGTAATCAATTACGGGGTCATTAGTTCAT
AGCCCATATATGGAGTTCCGCGTTACATAACTTACGGTAAATGGCCCGCC
TGGCTGACCGCCCAACGACCCCCGCCCATTGACGTCAATAATGACGTATG
TTCCCATAGTAACGCCAATAGGGACTTTCCATTGACGTCAATGGGTGGAC
TATTTACGGTAAACTGCCCACTTGGCAGTACATCAAGTGTATCATATGCC
AAGTACGCCCCCTATTGACGTCAATGACGGTAAATGGCCCGCCTGGCATT
ATGCCCAGTACATGACCTTATGGGACTTTCCTACTTGGCAGTACATCTAC
GTATTAGTCATCGCTATTACCATGGTGCAATTTATGGTATAGCTGGGAAA
CGTCAAAGTCAAGAGTTTTGTAGGAAAGTCACGTCACTTAGCCCTGTCTC
CTGTGCCGGGTGAGACCTGTGTGTGCACTTGGTGACAATGGCTTTGAGTC
TGTCAACTCCAGACTGAGGTCAGCCTTACACACCCATAGTTCCCAAAGCT
GAAAACAGGCCTGCCTCCAACGGTACCTGCTAATATCAGGGGAGCCTTTT
CAGCTTACAGAGCACCCTGTATGTGTTTGTCTTAGTTCAGGCCACCATCT
CCACCTTACCAGGCATCTAGAACCTTCTCCACACTTTGCCAACAGGGTTC
GTTTGCAGAATTGAAATCTTAGTTAAGGTTTGTTGAAGTTTGTTGTTGTT
TTTTTTTTTTTTTTACAATTGGCTGTTCCCACCCACATTCCCTTGAGACA
TAAATAGAAAAAAAAAAAAAAAGAGGTTTCATGAGTAAGACAAGACATTT
GAGCTGCATCCACTTGATCCTTGAAAAGTGCAATTTATGGTATAGCTGGG
AAACGTCAAAGTCAAGAGTTTTGTAGGAAAGTCACGTCACTTAGCCCTGT
CTCCTGTGCCGGGTGAGACCTGTGTGTGCACTTGGTGACAATGGCTTTGA
GTCTGTCAACTCCAGACTGAGGTCAGCCTTACACACCCATAGTTCCCAAA
GCTGAAAACAGGCCTGCCTCCAACGGTACCTGCTAATATCAGGGGAGCCT
TTTCAGCTTACAGAGCACCCTGTATGTGTTTGTCTTAGTTCAGGCCACCA
TCTCCACCTTACCAGGCATCTAGAACCTTCTCCACACTTTGCCAACAGGG
TTCGTTTGCAGAATTGAAATCTTAGTTAAGGTTTGTTGAAGTTTGTTGTT
GTTTTTTTTTTTTTTTTACAATTGGCTGTTCCCACCCACATTCCCTTGAG
ACATAAATAGAAAAAAAAAAAAAAAGAGGTTTCATGAGTAAGACAAGACA
TTTGAGCTGCATCCACTTGATCCTTGAAAACGCCGGTGGCAGGTGGAAAG
GCGAGCGGCATGGAGCGCGTAATAAGAGAGTTGGAGTCGGAAAGAGCAGC
CCCAGTCGCCGGGGAAGCGGGAGGTCAGTGCGGGCTCCGGCGGCCCCCAG
GCTCCGAGCGCCCGCCCGCGGCCCCGGCCCGGCCCCTAGCCCCCGCCGCC
CGCGCCCGCCCCGGGTCGCCCCTCTGGCCCCGGGTCCGAGCCATGCGTCT
CTGAGCGCCCCGAGCGCGCCCCCGCCCCGGACCGTGCCCGGGCCCCGGCG
CCCCCAGCCCGGCGCCGCCCACCGGTCGCTAGCCACCATGGCTGAAGCCC
CTCCTAGAAGGCTTGGACTGGGACCTCCTCCTGGGGATGCTCCTAGAGCT
GAACTGGTGGCTCTGACAGCCGTGCAGTCTGAACAAGGCGAAGCTGGTGG
CGGCGGATCTCCACGTAGACTTGGACTGCTGGGAAGCCCTCTTCCTCCTG
GTGCTCCACTTCCTGGACCTGGCAGTGGATCTGGATCTGCCTGTGGCCAG
AGAAGCTCTGCCGCTCACAAGAGATACCGGCGGCTGCAGAACTGGGTGTA
CAACGTGCTGGAAAGACCCAGAGGCTGGGCCTTCGTGTACCACGTGTTCA
TCTTTCTGCTGGTGTTCAGCTGCCTGGTGCTGTCCGTGCTGAGCACCATC
CAAGAACATCAAGAGCTGGCTAACGAGTGCCTGTTAATACTGGAGTTTGT
GATGATTGTGGTGTTCGGCCTCGAGTACATCGTCCGCGTTTGGAGCGCCG
GCTGCTGCTGCAGATATAGAGGTTGGCAAGGCAGATTCCGCTTCGCCAGA
AAGCCCTTCTGCGTGATCGACTTCATCGTGTTCGTGGCCAGCGTGGCCGT
GATTGCTGCTGGCACACAGGGCAACATCTTCGCCACAAGCGCCCTGCGGA
GCATGCGGTTTCTGCAGATCCTGAGAATGGTCCGAATGGACAGAAGAGGC
GGCACCTGGAAGCTGCTGGGCTCTGTGGTGTACGCCCACAGCAAAGAGCT
GATCACCGCCTGGTACATCGGATTTCTGGTGCTGATCTTCGCCTCCTTCC
TGGTGTACCTGGCCGAGAAGGACGCCAACAGCGACTTTAGCAGCTACGCC
GACTCTCTTTGGTGGGGCACCATCACACTGACCACCATCGGCTACGGCGA
CAAGACCCCTCACACATGGCTGGGAAGAGTGCTGGCCGCTGGATTTGCTC
TGCTGGGCATCAGCTTTTTCGCCCTGCCTGCCGGAATCCTCGGATCTGGC
TTTGCCCTGAAGGTGCAAGAGCAGCACCGGCAGAAGCACTTCGAGAAGAG
AAGAATGCCTGCCGCCAACCTGATTCAGGCCGCTTGGAGACTGTACAGCA
CCGACATGAGCAGAGCCTACCTGACCGCCACGTGGTATTATTACGACTCG
ATCCTGCCTAGCTTCCGCGAACTGGCCCTGCTGTTTGAGCATGTGCAGAG
AGCCAGAAACGGCGGCCTCAGACCTCTGGAAGTTCGGAGAGCACCTGTGC
CTGATGGCGCCCCTTCTAGATATCCTCCAGTGGCCACCTGTCACAGACCC
GGCAGCACATCTTTTTGCCCTGGCGAGTCTAGCCGGATGGGCATCAAGGA
CAGAATCAGAATGGGCAGCAGCCAGCGGAGAACAGGCCCTTCTAAACAGC
ATCTGGCCCCTCCAACCATGCCTACAAGCCCTAGCTCTGAGCAAGTGGGC
GAAGCCACCTCTCCTACCAAGGTGCAGAAGTCCTGGTCCTTCAACGACCG
GACCAGATTCAGAGCCAGCCTGAGACTGAAGCCCAGAACCTCTGCCGAGG
ATGCCCCTTCTGAAGAGGTGGCCGAAGAGAAGTCCTACCAGTGCGAGCTG
ACCGTGGACGACATCATGCCAGCCGTGAAAACCGTGATACGGTCTATCCG
GATCCTGAAGTTCCTGGTGGCCAAGCGGAAGTTCAAAGAGACACTGCGGC
CCTACGACGTGAAGGACGTGATCGAGCAGTATTCTGCCGGCCACCTGGAC
ATGCTGGGCAGAATCAAGAGCCTGCAGACCAGAGTGGACCAGATCGTTGG
AAGAGGCCCAGGCGACAGAAAGGCCAGAGAGAAGGGCGATAAGGGCCCAT
CTGATGCCGAGGTTGTCGACGAGATATCAATGATGGGCAGAGTGGTCAAG
GTGGAAAAACAGGTGCAGAGCATCGAGCACAAGCTGGACCTGCTGCTGGG
ATTCTACAGCCGGTGTCTGAGAAGCGGCACATCTGCATCTCTGGGCGCTG
TGCAGGTCCCACTGTTCGACCCTGATATCACCAGCGACTATCACAGCCCC
GTGGACCACGAGGACATCTCCGTTTCTGCTCAGACCCTGAGCATCAGCAG
ATCCGTGTCCACCAACATGGACGGATCCCGGGCTGACTACAAAGACCATG
ACGGTGATTATAAAGATCATGACATCGACTACAAGGATGACGATGACAAG
TAATAAGAGCTCGCTGATCAGCCTCGACTGTGCCTTCTAGTTGCCAGCCA
TCTGTTGTTTGCCCCTCCCCCGTGCCTTCCTTGACCCTGGAAGGTGCCAC
TCCCACTGTCCTTTCCTAATAAAATGAGGAAATTGCATCGCATTGTCTGA
GTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTAT
GttaattCGGACCGCTAGGAACCCCTAGTGATGGAGTTGGCCACTCCCTC
TCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGCTGC
CTGCAGG

TABLE 3
Construct Components (SEQ ID NO: 23)

| Component (5′ to | Position |
|---|---|
| 3′ orientation) | (nucleotides) |
| 5′ ITR | 12-130 |
| CMV Enhancer | 145-524 |
| Oncomodulin | 525-1530 |
| promoter | |
| 5′ UTR | 1531-1820 |
| KCNQ4 coding | 1838-3922 |
| region | |
| 3x Flag | 3935-4000 |
| polyA | 4028-4251 |
| 3′ ITR | 4267-4396 |

In some aspects, the oncomodulin promoter comprises a nucleic acid sequence that comprises at least 80%, at least 85%, at least 90%, at least 95%, at least 96%, at least 97%, at least 98% at least 99%, or 100% identity to nucleotides 525-1530 of SEQ ID NO: 23.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 24. In some aspects, the construct comprises the nucleic acid sequence of SEQ ID NO: 24.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4464 of SEQ ID NO: 24. In some aspects, the construct comprises nucleotides 12-4464 of SEQ ID NO: 24.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 24. In some aspects, the rAAVAnc80 particle comprises the nucleic acid sequence of SEQ ID NO: 24.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) an oncomodulin promoter comprising the nucleic acid sequence of SEQ ID NO: 2, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) an oncomodulin promoter comprising the nucleic acid sequence of SEQ ID NO: 2, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 24)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGTGCAATTTATGGTATAGCTGGGAAA
CGTCAAAGTCAAGAGTTTTGTAGGAAAGTCACGTCACTTAGCCCTGTCTCCTGTGCC
GGGTGAGACCTGTGTGTGCACTTGGTGACAATGGCTTTGAGTCTGTCAACTCCAGAC
TGAGGTCAGCCTTACACACCCATAGTTCCCAAAGCTGAAAACAGGCCTGCCTCCAAC
GGTACCTGCTAATATCAGGGGAGCCTTTTCAGCTTACAGAGCACCCTGTATGTGTTT
GTCTTAGTTCAGGCCACCATCTCCACCTTACCAGGCATCTAGAACCTTCTCCACACTT
TGCCAACAGGGTTCGTTTGCAGAATTGAAATCTTAGTTAAGGTTTGTTGAAGTTTGTT
GTTGTTTTTTTTTTTTTTTTACAATTGGCTGTTCCCACCCACATTCCCTTGAGACATAA
ATAGAAAAAAAAAAAAAAAGAGGTTTCATGAGTAAGACAAGACATTTGAGCTGCAT
CCACTTGATCCTTGAAAAGGAAATCTAAGAGGTTGTAACTATCACTTTTTCTAGCCTA
TATAAGGTAGGTCAGTAAGGTAGCAAAAACACATCTGTTGTTTTGCTCCTTCAACTC
TTTTTCCTGATTCTTCCTGGGGGGAAACCGAAAACGGTGAGTAACTGGTGGACACAT
CAGACCCCAGACTCTTTTCTTCACTGCATGCATTCATATTAGGCTCAGGTGCTTAGAC
TCCTGTTTTCCGGTGGCTCTGACACCTGGAAGGATTTTAATCTCTGGGAGATGGGCTT
TTCATCCATCTGCTTCCCACCTTTCAGGACAGGTGCATGCCTTCTTCCACAGAATGTC
TGCAAGCAGCCCAAACTGTATCCTTTCCCACGTGGAATTTGCAACATTGCATCTCTCG
GGCTGCTGTAGGAAAATGCCAGTGCATGTGTAACATGGTTTACGGCTGCCTATGCAA
ATGACTGATTATGTCAGTATAATTTTTATAAGAAAACAATTGAATCCTTCTTTGGGTC
ATTTTTTTTTTCCATTTTTGGCATGTATTCAAAAGAAGGCTCTGAGACAAAAAAGGCT
GGGGTGTTTTCCGTATCTGGTTTTAATTTGGATATTCTGTCCCGTCACTTAATACAAA
ACCATGCTTATCACATTTTAAAAATTCTAGACAGGCCTGGCTCGGTGGCTTGCATCTG
TCATCCCAGCACTTTGTGAGGCCAAGGCAGGCAGATCACCTGAGGTCAGGAGCTCA
AGACCAGCCTGGCCAACATGGCAAAACCCCGTCTCTACTAAAAACACAAAAATTAG
CCAGGCATGGTAGTGCGCACCTGTAATCCCAGCTACTGGGAAGGCTTAGGCAGGAG
AATCACTTGAGCCCAGGAGGCGGAGGTTGCGGTGAGCCGAGATCACGCTCTTGCACT
CCAGCCTGGGTGACAGAGTGAGACTCCGTCTTAATTTAAAAAAAAAAATAACGCCG
GTGGCAGGTGGAAAGGCGAGCGGCATGGAGCGCGTAATAAGAGAGTTGGAGTCGG
AAAGAGCAGCCCCAGTCGCCGGGGAAGCGGGAGGTCAGTGCGGGCTCCGGCGGCCC
CCAGGCTCCGAGCGCCCGCCCGCGGCCCCGGCCCGGCCCCTAGCCCCCGCCGCCCGC
GCCCGCCCCGGGTCGCCCCTCTGGCCCCGGGTCCGAGCCATGCGTCTCTGAGCGCCC
CGAGCGCGCCCCCGCCCCGGACCGTGCCCGGGCCCCGGCGCCCCCAGCCCGGCGCC
GCCCACCGGTCGCTAGCCACCATGGCTGAAGCCCCTCCTAGAAGGCTTGGACTGGGA
CCTCCTCCTGGGGATGCTCCTAGAGCTGAACTGGTGGCTCTGACAGCCGTGCAGTCT
GAACAAGGCGAAGCTGGTGGCGGCGGATCTCCACGTAGACTTGGACTGCTGGGAAG
CCCTCTTCCTCCTGGTGCTCCACTTCCTGGACCTGGCAGTGGATCTGGATCTGCCTGT
GGCCAGAGAAGCTCTGCCGCTCACAAGAGATACCGGCGGCTGCAGAACTGGGTGTA
CAACGTGCTGGAAAGACCCAGAGGCTGGGCCTTCGTGTACCACGTGTTCATCTTTCT
GCTGGTGTTCAGCTGCCTGGTGCTGTCCGTGCTGAGCACCATCCAAGAACATCAAGA
GCTGGCTAACGAGTGCCTGTTAATACTGGAGTTTGTGATGATTGTGGTGTTCGGCCTC
GAGTACATCGTCCGCGTTTGGAGCGCCGGCTGCTGCTGCAGATATAGAGGTTGGCAA
GGCAGATTCCGCTTCGCCAGAAAGCCCTTCTGCGTGATCGACTTCATCGTGTTCGTG
GCCAGCGTGGCCGTGATTGCTGCTGGCACACAGGGCAACATCTTCGCCACAAGCGCC
CTGCGGAGCATGCGGTTTCTGCAGATCCTGAGAATGGTCCGAATGGACAGAAGAGG
CGGCACCTGGAAGCTGCTGGGCTCTGTGGTGTACGCCCACAGCAAAGAGCTGATCAC
CGCCTGGTACATCGGATTTCTGGTGCTGATCTTCGCCTCCTTCCTGGTGTACCTGGCC
GAGAAGGACGCCAACAGCGACTTTAGCAGCTACGCCGACTCTCTTTGGTGGGGCACC
ATCACACTGACCACCATCGGCTACGGCGACAAGACCCCTCACACATGGCTGGGAAG
AGTGCTGGCCGCTGGATTTGCTCTGCTGGGCATCAGCTTTTTCGCCCTGCCTGCCGGA
ATCCTCGGATCTGGCTTTGCCCTGAAGGTGCAAGAGCAGCACCGGCAGAAGCACTTC
GAGAAGAGAAGAATGCCTGCCGCCAACCTGATTCAGGCCGCTTGGAGACTGTACAG
CACCGACATGAGCAGAGCCTACCTGACCGCCACGTGGTATTATTACGACTCGATCCT
GCCTAGCTTCCGCGAACTGGCCCTGCTGTTTGAGCATGTGCAGAGAGCCAGAAACGG
CGGCCTCAGACCTCTGGAAGTTCGGAGAGCACCTGTGCCTGATGGCGCCCCTTCTAG
ATATCCTCCAGTGGCCACCTGTCACAGACCCGGCAGCACATCTTTTTGCCCTGGCGA
GTCTAGCCGGATGGGCATCAAGGACAGAATCAGAATGGGCAGCAGCCAGCGGAGAA
CAGGCCCTTCTAAACAGCATCTGGCCCCTCCAACCATGCCTACAAGCCCTAGCTCTG
AGCAAGTGGGCGAAGCCACCTCTCCTACCAAGGTGCAGAAGTCCTGGTCCTTCAACG
ACCGGACCAGATTCAGAGCCAGCCTGAGACTGAAGCCCAGAACCTCTGCCGAGGAT
GCCCCTTCTGAAGAGGTGGCCGAAGAGAAGTCCTACCAGTGCGAGCTGACCGTGGA
CGACATCATGCCAGCCGTGAAAACCGTGATACGGTCTATCCGGATCCTGAAGTTCCT
GGTGGCCAAGCGGAAGTTCAAAGAGACACTGCGGCCCTACGACGTGAAGGACGTGA
TCGAGCAGTATTCTGCCGGCCACCTGGACATGCTGGGCAGAATCAAGAGCCTGCAG
ACCAGAGTGGACCAGATCGTTGGAAGAGGCCCAGGCGACAGAAAGGCCAGAGAGA
AGGGCGATAAGGGCCCATCTGATGCCGAGGTTGTCGACGAGATATCAATGATGGGC
AGAGTGGTCAAGGTGGAAAAACAGGTGCAGAGCATCGAGCACAAGCTGGACCTGCT
GCTGGGATTCTACAGCCGGTGTCTGAGAAGCGGCACATCTGCATCTCTGGGCGCTGT
GCAGGTCCCACTGTTCGACCCTGATATCACCAGCGACTATCACAGCCCCGTGGACCA
CGAGGACATCTCCGTTTCTGCTCAGACCCTGAGCATCAGCAGATCCGTGTCCACCAA
CATGGACGGATCCCGGGCTGACTACAAAGACCATGACGGTGATTATAAAGATCATG
ACATCGACTACAAGGATGACGATGACAAGTAATAAGAGCTCGCTGATCAGCCTCGA
CTGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCAT
TGTCTGAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTATGttaattC
GGACCGCTAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGCGG
CCTCAGTGAGCGAGCGAGCGCGCAGCTGCCTGCAGG

TABLE 4
Construct Components (SEQ ID NO: 24)

| Component (5′ to | Position |
|---|---|
| 3′ orientation) | (nucleotides) |
| 5′ ITR | 12-130 |
| Oncomodulin | 145-1598 |
| promoter | |
| 5′ UTR | 1599-1888 |
| KCNQ4 coding | 1906-3990 |
| region | |
| 3x Flag | 4003-4071 |
| polyA | 4096-4319 |
| 3′ ITR | 4335-4464 |

In some aspects, the oncomodulin promoter comprises a nucleic acid sequence that comprises at least 80%, at least 85%, at least 90%, at least 95%, at least 96%, at least 97%, at least 98% at least 99%, or 100% identity to nucleotides 145-1598 of SEQ ID NO: 24.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 25. In some aspects, the construct comprises the nucleic acid sequence of SEQ ID NO: 25.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4016 of SEQ ID NO: 25. In some aspects, the construct comprises nucleotides 12-4016 of SEQ ID NO: 25.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 25. In some aspects, the rAAVAnc80 particle comprises the nucleic acid sequence of SEQ ID NO: 25.

In some aspects, the construct comprises (i) a 5′ ITR comprising the nucleic acid sequence of SEQ ID NO: 16, (ii) an oncomodulin promoter comprising the nucleic acid sequence of SEQ ID NO: 1, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 25)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGTGCAATTTATGGTATAGCTGGGAAA
CGTCAAAGTCAAGAGTTTTGTAGGAAAGTCACGTCACTTAGCCCTGTCTCCTGTGCC
GGGTGAGACCTGTGTGTGCACTTGGTGACAATGGCTTTGAGTCTGTCAACTCCAGAC
TGAGGTCAGCCTTACACACCCATAGTTCCCAAAGCTGAAAACAGGCCTGCCTCCAAC
GGTACCTGCTAATATCAGGGGAGCCTTTTCAGCTTACAGAGCACCCTGTATGTGTTT
GTCTTAGTTCAGGCCACCATCTCCACCTTACCAGGCATCTAGAACCTTCTCCACACTT
TGCCAACAGGGTTCGTTTGCAGAATTGAAATCTTAGTTAAGGTTTGTTGAAGTTTGTT
GTTGTTTTTTTTTTTTTTTTACAATTGGCTGTTCCCACCCACATTCCCTTGAGACATAA
ATAGAAAAAAAAAAAAAAAGAGGTTTCATGAGTAAGACAAGACATTTGAGCTGCAT
CCACTTGATCCTTGAAAAGTGCAATTTATGGTATAGCTGGGAAACGTCAAAGTCAAG
AGTTTTGTAGGAAAGTCACGTCACTTAGCCCTGTCTCCTGTGCCGGGTGAGACCTGT
GTGTGCACTTGGTGACAATGGCTTTGAGTCTGTCAACTCCAGACTGAGGTCAGCCTT
ACACACCCATAGTTCCCAAAGCTGAAAACAGGCCTGCCTCCAACGGTACCTGCTAAT
ATCAGGGGAGCCTTTTCAGCTTACAGAGCACCCTGTATGTGTTTGTCTTAGTTCAGGC
CACCATCTCCACCTTACCAGGCATCTAGAACCTTCTCCACACTTTGCCAACAGGGTTC
GTTTGCAGAATTGAAATCTTAGTTAAGGTTTGTTGAAGTTTGTTGTTGTTTTTTTTTTT
TTTTTACAATTGGCTGTTCCCACCCACATTCCCTTGAGACATAAATAGAAAAAAAAA
AAAAAAGAGGTTTCATGAGTAAGACAAGACATTTGAGCTGCATCCACTTGATCCTTG
AAAACGCCGGTGGCAGGTGGAAAGGCGAGCGGCATGGAGCGCGTAATAAGAGAGT
TGGAGTCGGAAAGAGCAGCCCCAGTCGCCGGGGAAGCGGGAGGTCAGTGCGGGCTC
CGGCGGCCCCCAGGCTCCGAGCGCCCGCCCGCGGCCCCGGCCCGGCCCCTAGCCCCC
GCCGCCCGCGCCCGCCCCGGGTCGCCCCTCTGGCCCCGGGTCCGAGCCATGCGTCTC
TGAGCGCCCCGAGCGCGCCCCCGCCCCGGACCGTGCCCGGGCCCCGGCGCCCCCAG
CCCGGCGCCGCCCACCGGTCGCTAGCCACCATGGCTGAAGCCCCTCCTAGAAGGCTT
GGACTGGGACCTCCTCCTGGGGATGCTCCTAGAGCTGAACTGGTGGCTCTGACAGCC
GTGCAGTCTGAACAAGGCGAAGCTGGTGGCGGCGGATCTCCACGTAGACTTGGACT
GCTGGGAAGCCCTCTTCCTCCTGGTGCTCCACTTCCTGGACCTGGCAGTGGATCTGG
ATCTGCCTGTGGCCAGAGAAGCTCTGCCGCTCACAAGAGATACCGGCGGCTGCAGA
ACTGGGTGTACAACGTGCTGGAAAGACCCAGAGGCTGGGCCTTCGTGTACCACGTGT
TCATCTTTCTGCTGGTGTTCAGCTGCCTGGTGCTGTCCGTGCTGAGCACCATCCAAGA
ACATCAAGAGCTGGCTAACGAGTGCCTGTTAATACTGGAGTTTGTGATGATTGTGGT
GTTCGGCCTCGAGTACATCGTCCGCGTTTGGAGCGCCGGCTGCTGCTGCAGATATAG
AGGTTGGCAAGGCAGATTCCGCTTCGCCAGAAAGCCCTTCTGCGTGATCGACTTCAT
CGTGTTCGTGGCCAGCGTGGCCGTGATTGCTGCTGGCACACAGGGCAACATCTTCGC
CACAAGCGCCCTGCGGAGCATGCGGTTTCTGCAGATCCTGAGAATGGTCCGAATGGA
CAGAAGAGGCGGCACCTGGAAGCTGCTGGGCTCTGTGGTGTACGCCCACAGCAAAG
AGCTGATCACCGCCTGGTACATCGGATTTCTGGTGCTGATCTTCGCCTCCTTCCTGGT
GTACCTGGCCGAGAAGGACGCCAACAGCGACTTTAGCAGCTACGCCGACTCTCTTTG
GTGGGGCACCATCACACTGACCACCATCGGCTACGGCGACAAGACCCCTCACACAT
GGCTGGGAAGAGTGCTGGCCGCTGGATTTGCTCTGCTGGGCATCAGCTTTTTCGCCC
TGCCTGCCGGAATCCTCGGATCTGGCTTTGCCCTGAAGGTGCAAGAGCAGCACCGGC
AGAAGCACTTCGAGAAGAGAAGAATGCCTGCCGCCAACCTGATTCAGGCCGCTTGG
AGACTGTACAGCACCGACATGAGCAGAGCCTACCTGACCGCCACGTGGTATTATTAC
GACTCGATCCTGCCTAGCTTCCGCGAACTGGCCCTGCTGTTTGAGCATGTGCAGAGA
GCCAGAAACGGCGGCCTCAGACCTCTGGAAGTTCGGAGAGCACCTGTGCCTGATGG
CGCCCCTTCTAGATATCCTCCAGTGGCCACCTGTCACAGACCCGGCAGCACATCTTTT
TGCCCTGGCGAGTCTAGCCGGATGGGCATCAAGGACAGAATCAGAATGGGCAGCAG
CCAGCGGAGAACAGGCCCTTCTAAACAGCATCTGGCCCCTCCAACCATGCCTACAAG
CCCTAGCTCTGAGCAAGTGGGCGAAGCCACCTCTCCTACCAAGGTGCAGAAGTCCTG
GTCCTTCAACGACCGGACCAGATTCAGAGCCAGCCTGAGACTGAAGCCCAGAACCT
CTGCCGAGGATGCCCCTTCTGAAGAGGTGGCCGAAGAGAAGTCCTACCAGTGCGAG
CTGACCGTGGACGACATCATGCCAGCCGTGAAAACCGTGATACGGTCTATCCGGATC
CTGAAGTTCCTGGTGGCCAAGCGGAAGTTCAAAGAGACACTGCGGCCCTACGACGT
GAAGGACGTGATCGAGCAGTATTCTGCCGGCCACCTGGACATGCTGGGCAGAATCA
AGAGCCTGCAGACCAGAGTGGACCAGATCGTTGGAAGAGGCCCAGGCGACAGAAA
GGCCAGAGAGAAGGGCGATAAGGGCCCATCTGATGCCGAGGTTGTCGACGAGATAT
CAATGATGGGCAGAGTGGTCAAGGTGGAAAAACAGGTGCAGAGCATCGAGCACAA
GCTGGACCTGCTGCTGGGATTCTACAGCCGGTGTCTGAGAAGCGGCACATCTGCATC
TCTGGGCGCTGTGCAGGTCCCACTGTTCGACCCTGATATCACCAGCGACTATCACAG
CCCCGTGGACCACGAGGACATCTCCGTTTCTGCTCAGACCCTGAGCATCAGCAGATC
CGTGTCCACCAACATGGACGGATCCCGGGCTGACTACAAAGACCATGACGGTGATT
ATAAAGATCATGACATCGACTACAAGGATGACGATGACAAGTAATAAGAGCTCGCT
GATCAGCCTCGACTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGGGGGTGGGGCA
GGACAGCAAGGGGGAGGATTGGGAAGACAATAGCAGGCATGCTGGGGATGCGGTG
GGCTCTATGttaattCGGACCGCTAGGAACCCCTAGTGATGGAGTTGGCCACTCCCTCTC
TGCGCGCTCGCTCGCTCACTGAGGCCGGGCGACCAAAGGTCGCCCGACGCCCGGGCT
TTGCCCGGGCGGCCTCAGTGAGCGAGCGAGCGCGCAGCTGCCTGCAGG

TABLE 5
Construct Components (SEQ ID NO: 25)

| Component (5′ to | Position |
|---|---|
| 3′ orientation) | (nucleotides) |
| 5′ ITR | 12-130 |
| Oncomodulin | 145-1150 |
| promoter | |
| 5′ UTR | 1151-1440 |
| KCNQ4 coding | 1458-3542 |
| region | |
| 3x Flag | 3555-3623 |
| polyA | 3648-3871 |
| 3′ ITR | 3887-4016 |

In some aspects, the oncomodulin promoter comprises a nucleic acid sequence that comprises at least 80%, at least 85%, at least 90%, at least 95%, at least 96%, at least 97%, at least 98% at least 99%, or 100% identity to nucleotides 145-1150 of SEQ ID NO: 25.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 26. In some aspects, the construct comprises the nucleic acid sequence of SEQ ID NO: 26.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4521 of SEQ ID NO: 26. In some aspects, the construct comprises nucleotides 12-4521 of SEQ ID NO: 26.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 26. In some aspects, the rAAVAnc80 particle comprises the nucleic acid sequence of SEQ ID NO: 26.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a prestin promoter comprising the nucleic acid sequence of SEQ ID NO: 3, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a prestin promoter comprising the nucleic acid sequence of SEQ ID NO: 3, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 26)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTAAAGCAAACTCATCTCTAAACCAGAA
ATAATAGCAATATCTATACAAGTAAATACATGTACTCAGAACAGTGCCTACTACATG
TAAACACTGAACAGGTGTTAGCAACATTGCCATTATTGTGTTAGTATATTAGGTACC
TGGTGCTACCGGCAAAACCAGTTTATCATCCAACTGTCTCCAGTGTTGCTACTCAAA
GTTTGGTCCTCCAGTAGCCTATCAGGATCACCCAGGGGCCTGTTAGAAAGGCACATC
TCAGACCCCACCCCAGACCTACTGAATCAGAATCTGCGTTTTTAACGGGATCCGCAG
GTGATTCCTATGCACATTAAAGTGTAAGAAGTACTGGGCTACAGACAGGTATGTGAC
AAAATAATTTCATAGGATGGCAAAGGCCAAGTGGCAAATGAAGGACACCAGAAATG
CACGTCCCAGGAGCCCAACTCCTCCTTAGTAAATTACCCTATTAAGATTTGTTTAGAG
ATGTTCAAAAGCGTGGAGAAAAGCAAATTTGGTTTCCCTGGTGCCCTTGAAGAGATC
GCCCTCGTGTGGAGTAGGGAGGGAATCTCTAGCCTTTCCTCTCGGATGAAGAACAGC
ACCAGCGCTCCCAGCCAAAGGCCTGGCCCAGGTTCTGGAGGTGGGGTCTCCTTGGCA
GAAGCCTCTGGTGTCTGCAGGCGTGCATTTACAGCTTTAAGACCAAACAGCTAGTCC
GCCACGTGTCACTACAGTGTGCACGCGCAGAAATGCACAAAGCAAAAAAAAAAAAA
AAGATGCTCTTAATGAACCAACTATAATCCTTGCTAAGGCATAAAGCCAGAGGGAA
GTATGTATCTGAAATCATTTTCTACCCCTCACCCTCTTGGAGCCCGGCACTCTGGCTG
CGGTGCTCTCTTGTATCCCAGTTGCTAGATGCAAAACAAGCTATTTCCTATCTAATTT
TTTTTTTGTTTTATAAATTCTAACTTAAATGCCCAGAAAATAACTACTCATACTCACA
TTGTCCTCTAATTGAAAAGATAAGTCAGGTTTTTTGTGTTTTTTTTTCATTTTAAAATC
ATAATACGCAATGTTTTCCACTTGAACGCTATACCTTGTGTATTGTGCTTGCTTCAGC
CTCGAGCCTCTACTGATGTTCCACCTCAAGGCGACAGGAATGCCACCTGGAGAAACT
CCTGGGCGGTATGGGAAGAAAGCCGGTCTCATCAGAGTATATTTGCGGGGATCGAC
GACCAAGGTGTTAAATTCCAAGCACGCTTTGGAAAGTTCTAGGTGCTTGGGAAGAGA
TCCGTAGGCGGCAGGGATGCCCGCGCCCCGGCGTCCCAGCGCGGAGGGTGGCGGCG
GGGCCTGGCCCTAGCGGGGCGGGGGGGGCTCGGGTTACCGGGAGTCGCGGGGCGCG
GCCGGCACTGCCCGCGGCGCCTCCTCCTAGAGCCGCACCTGGAGGCAGCGCGCGCGT
CGAAGAGGCAGCGGCTGTGGAGCGCGGCGGGGCGGCTCCGCCCAGGGCAGCCCGGG
CTGCGCCGGTGGCAGGTGGAAAGGCGAGCGGCATGGAGCGCGTAATAAGAGAGTTG
GAGTCGGAAAGAGCAGCCCCAGTCGCCGGGGAAGCGGGAGGTCAGTGCGGGCTCCG
GCGGCCCCCAGGCTCCGAGCGCCCGCCCGCGGCCCCGGCCCGGCCCCTAGCCCCCGC
CGCCCGCGCCCGCCCCGGGTCGCCCCTCTGGCCCCGGGTCCGAGCCATGCGTCTCTG
AGCGCCCCGAGCGCGCCCCCGCCCCGGACCGTGCCCGGGCCCCGGCGCCCCCAGCC
CGGCGCCGCCCACCGGTCGCTAGCCACCATGGCTGAAGCCCCTCCTAGAAGGCTTGG
ACTGGGACCTCCTCCTGGGGATGCTCCTAGAGCTGAACTGGTGGCTCTGACAGCCGT
GCAGTCTGAACAAGGCGAAGCTGGTGGCGGCGGATCTCCACGTAGACTTGGACTGC
TGGGAAGCCCTCTTCCTCCTGGTGCTCCACTTCCTGGACCTGGCAGTGGATCTGGATC
TGCCTGTGGCCAGAGAAGCTCTGCCGCTCACAAGAGATACCGGCGGCTGCAGAACT
GGGTGTACAACGTGCTGGAAAGACCCAGAGGCTGGGCCTTCGTGTACCACGTGTTCA
TCTTTCTGCTGGTGTTCAGCTGCCTGGTGCTGTCCGTGCTGAGCACCATCCAAGAACA
TCAAGAGCTGGCTAACGAGTGCCTGTTAATACTGGAGTTTGTGATGATTGTGGTGTT
CGGCCTCGAGTACATCGTCCGCGTTTGGAGCGCCGGCTGCTGCTGCAGATATAGAGG
TTGGCAAGGCAGATTCCGCTTCGCCAGAAAGCCCTTCTGCGTGATCGACTTCATCGT
GTTCGTGGCCAGCGTGGCCGTGATTGCTGCTGGCACACAGGGCAACATCTTCGCCAC
AAGCGCCCTGCGGAGCATGCGGTTTCTGCAGATCCTGAGAATGGTCCGAATGGACA
GAAGAGGCGGCACCTGGAAGCTGCTGGGCTCTGTGGTGTACGCCCACAGCAAAGAG
CTGATCACCGCCTGGTACATCGGATTTCTGGTGCTGATCTTCGCCTCCTTCCTGGTGT
ACCTGGCCGAGAAGGACGCCAACAGCGACTTTAGCAGCTACGCCGACTCTCTTTGGT
GGGGCACCATCACACTGACCACCATCGGCTACGGCGACAAGACCCCTCACACATGG
CTGGGAAGAGTGCTGGCCGCTGGATTTGCTCTGCTGGGCATCAGCTTTTTCGCCCTGC
CTGCCGGAATCCTCGGATCTGGCTTTGCCCTGAAGGTGCAAGAGCAGCACCGGCAGA
AGCACTTCGAGAAGAGAAGAATGCCTGCCGCCAACCTGATTCAGGCCGCTTGGAGA
CTGTACAGCACCGACATGAGCAGAGCCTACCTGACCGCCACGTGGTATTATTACGAC
TCGATCCTGCCTAGCTTCCGCGAACTGGCCCTGCTGTTTGAGCATGTGCAGAGAGCC
AGAAACGGCGGCCTCAGACCTCTGGAAGTTCGGAGAGCACCTGTGCCTGATGGCGC
CCCTTCTAGATATCCTCCAGTGGCCACCTGTCACAGACCCGGCAGCACATCTTTTTGC
CCTGGCGAGTCTAGCCGGATGGGCATCAAGGACAGAATCAGAATGGGCAGCAGCCA
GCGGAGAACAGGCCCTTCTAAACAGCATCTGGCCCCTCCAACCATGCCTACAAGCCC
TAGCTCTGAGCAAGTGGGCGAAGCCACCTCTCCTACCAAGGTGCAGAAGTCCTGGTC
CTTCAACGACCGGACCAGATTCAGAGCCAGCCTGAGACTGAAGCCCAGAACCTCTG
CCGAGGATGCCCCTTCTGAAGAGGTGGCCGAAGAGAAGTCCTACCAGTGCGAGCTG
ACCGTGGACGACATCATGCCAGCCGTGAAAACCGTGATACGGTCTATCCGGATCCTG
AAGTTCCTGGTGGCCAAGCGGAAGTTCAAAGAGACACTGCGGCCCTACGACGTGAA
GGACGTGATCGAGCAGTATTCTGCCGGCCACCTGGACATGCTGGGCAGAATCAAGA
GCCTGCAGACCAGAGTGGACCAGATCGTTGGAAGAGGCCCAGGCGACAGAAAGGCC
AGAGAGAAGGGCGATAAGGGCCCATCTGATGCCGAGGTTGTCGACGAGATATCAAT
GATGGGCAGAGTGGTCAAGGTGGAAAAACAGGTGCAGAGCATCGAGCACAAGCTG
GACCTGCTGCTGGGATTCTACAGCCGGTGTCTGAGAAGCGGCACATCTGCATCTCTG
GGCGCTGTGCAGGTCCCACTGTTCGACCCTGATATCACCAGCGACTATCACAGCCCC
GTGGACCACGAGGACATCTCCGTTTCTGCTCAGACCCTGAGCATCAGCAGATCCGTG
TCCACCAACATGGACGGATCCCGGGCTGACTACAAAGACCATGACGGTGATTATAA
AGATCATGACATCGACTACAAGGATGACGATGACAAGTAATAAGAGCTCGCTGATC
AGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGCAGGAC
AGCAAGGGGGAGGATTGGGAAGACAATAGCAGGCATGCTGGGGATGCGGTGGGCTC
TATGttaattCGGACCGCTAGGAACCCCTAGTGATGGAGTTGGCCACTCCCTCTCTGCGC
GCTCGCTCGCTCACTGAGGCCGGGCGACCAAAGGTCGCCCGACGCCCGGGCTTTGCC
CGGGCGGCCTCAGTGAGCGAGCGAGCGCGCAGCTGCCTGCAGG

TABLE 6
Construct Components (SEQ ID NO: 26)

| Component (5′ to | Position |
|---|---|
| 3′ orientation) | (nucleotides) |
| 5′ ITR | 12-130 |
| Prestin promoter | 145-1655 |
| 5′ UTR | 1656-1945 |
| KCNQ4 | 1963-4047 |
| coding region | |
| 3x Flag | 4060-4128 |
| polyA | 4153-4376 |
| 3′ ITR | 4392-4521 |

In some aspects, the prestin promoter comprises a nucleic acid sequence that comprises at least 80%, at least 85%, at least 90%, at least 95%, at least 96%, at least 97%, at least 98% at least 99%, or 100% identity to nucleotides 145-1655 of SEQ ID NO: 26.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 27. In some aspects, the construct comprises the nucleic acid sequence of SEQ ID NO: 27.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-3750 of SEQ ID NO: 27. In some aspects, the construct comprises nucleotides 12-3750 of SEQ ID NO: 27.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 27. In some aspects, the rAAVAnc80 particle comprises the nucleic acid sequence of SEQ ID NO: 27.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a CHRNA10 promoter comprising the nucleic acid sequence of SEQ ID NO: 4, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a CHRNA10 promoter comprising the nucleic acid sequence of SEQ ID NO: 4, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 27)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTTTCAGATGCCATCATTAATGAGAACT
ATGACTACCTGAAGGGGTTCTTGGAAGACCTGGCAAGGAACTCCCCTTGGATTAATT
GGCTTCTCTGCTTCTTTGTAGGTGGATTGCTCAGGTAATGACCTGGAGCAGTTACACA
TCAAAGTGACTTCACTGTGCAGTCGGATAGAGCAGATTCAGTGTCTGGTATTGGCTT
TCCCTTTGTATTTTTGAATAGAATATACCATTCAAAGCCTCCTCGCTCTTCTACTATA
GTGGTTTTGTTTTTAAACCCTGAGTGACGCTTCACCTTTCTAAATCAGATTCCCTTTTG
TAAAGGGGATAATGATTGCTGATGTTACTTCACACAGGGCTATTTTCAAGAGGAATC
AATTGAGTAGCATGAGTACTATTCCAGATCTTATTTTGATCTGTCAAGCTGAAGATGT
GAGCAAATTCCAATTAAGATTAGACCAAAGACTTCTGAGACTTTCAGGAATTCAGGG
ATGAGAAAGCAGAGTGGGTCAGCTCTGTTGTCTGGAACTTCCATTTAACTTAGATGC
CTCAGGATAGGGGTTACTCAGCTGGAATCCCCTCCACTACTGACTCACTATGTGAAC
CTGAGTGAGTCACAAAACATAGTTGGACTTCCAGCAAAGAACACCTGACCTGGTTTC
CTTACCAGAGGAATGTTTCAGAAAGTGAGTATGCTATAGAAATGGTTAGCTCTTAGC
AGTGTTCGGAATTGTGGGCCAGGAGCGCCGGTGGCAGGTGGAAAGGCGAGCGGCAT
GGAGCGCGTAATAAGAGAGTTGGAGTCGGAAAGAGCAGCCCCAGTCGCCGGGGAA
GCGGGAGGTCAGTGCGGGCTCCGGCGGCCCCCAGGCTCCGAGCGCCCGCCCGCGGC
CCCGGCCCGGCCCCTAGCCCCCGCCGCCCGCGCCCGCCCCGGGTCGCCCCTCTGGCC
CCGGGTCCGAGCCATGCGTCTCTGAGCGCCCCGAGCGCGCCCCCGCCCCGGACCGTG
CCCGGGCCCCGGCGCCCCCAGCCCGGCGCCGCCCACCGGTCGCTAGCCACCATGGCT
GAAGCCCCTCCTAGAAGGCTTGGACTGGGACCTCCTCCTGGGGATGCTCCTAGAGCT
GAACTGGTGGCTCTGACAGCCGTGCAGTCTGAACAAGGCGAAGCTGGTGGCGGCGG
ATCTCCACGTAGACTTGGACTGCTGGGAAGCCCTCTTCCTCCTGGTGCTCCACTTCCT
GGACCTGGCAGTGGATCTGGATCTGCCTGTGGCCAGAGAAGCTCTGCCGCTCACAAG
AGATACCGGCGGCTGCAGAACTGGGTGTACAACGTGCTGGAAAGACCCAGAGGCTG
GGCCTTCGTGTACCACGTGTTCATCTTTCTGCTGGTGTTCAGCTGCCTGGTGCTGTCC
GTGCTGAGCACCATCCAAGAACATCAAGAGCTGGCTAACGAGTGCCTGTTAATACTG
GAGTTTGTGATGATTGTGGTGTTCGGCCTCGAGTACATCGTCCGCGTTTGGAGCGCC
GGCTGCTGCTGCAGATATAGAGGTTGGCAAGGCAGATTCCGCTTCGCCAGAAAGCCC
TTCTGCGTGATCGACTTCATCGTGTTCGTGGCCAGCGTGGCCGTGATTGCTGCTGGCA
CACAGGGCAACATCTTCGCCACAAGCGCCCTGCGGAGCATGCGGTTTCTGCAGATCC
TGAGAATGGTCCGAATGGACAGAAGAGGCGGCACCTGGAAGCTGCTGGGCTCTGTG
GTGTACGCCCACAGCAAAGAGCTGATCACCGCCTGGTACATCGGATTTCTGGTGCTG
ATCTTCGCCTCCTTCCTGGTGTACCTGGCCGAGAAGGACGCCAACAGCGACTTTAGC
AGCTACGCCGACTCTCTTTGGTGGGGCACCATCACACTGACCACCATCGGCTACGGC
GACAAGACCCCTCACACATGGCTGGGAAGAGTGCTGGCCGCTGGATTTGCTCTGCTG
GGCATCAGCTTTTTCGCCCTGCCTGCCGGAATCCTCGGATCTGGCTTTGCCCTGAAGG
TGCAAGAGCAGCACCGGCAGAAGCACTTCGAGAAGAGAAGAATGCCTGCCGCCAAC
CTGATTCAGGCCGCTTGGAGACTGTACAGCACCGACATGAGCAGAGCCTACCTGACC
GCCACGTGGTATTATTACGACTCGATCCTGCCTAGCTTCCGCGAACTGGCCCTGCTGT
TTGAGCATGTGCAGAGAGCCAGAAACGGCGGCCTCAGACCTCTGGAAGTTCGGAGA
GCACCTGTGCCTGATGGCGCCCCTTCTAGATATCCTCCAGTGGCCACCTGTCACAGA
CCCGGCAGCACATCTTTTTGCCCTGGCGAGTCTAGCCGGATGGGCATCAAGGACAGA
ATCAGAATGGGCAGCAGCCAGCGGAGAACAGGCCCTTCTAAACAGCATCTGGCCCC
TCCAACCATGCCTACAAGCCCTAGCTCTGAGCAAGTGGGCGAAGCCACCTCTCCTAC
CAAGGTGCAGAAGTCCTGGTCCTTCAACGACCGGACCAGATTCAGAGCCAGCCTGA
GACTGAAGCCCAGAACCTCTGCCGAGGATGCCCCTTCTGAAGAGGTGGCCGAAGAG
AAGTCCTACCAGTGCGAGCTGACCGTGGACGACATCATGCCAGCCGTGAAAACCGT
GATACGGTCTATCCGGATCCTGAAGTTCCTGGTGGCCAAGCGGAAGTTCAAAGAGAC
ACTGCGGCCCTACGACGTGAAGGACGTGATCGAGCAGTATTCTGCCGGCCACCTGGA
CATGCTGGGCAGAATCAAGAGCCTGCAGACCAGAGTGGACCAGATCGTTGGAAGAG
GCCCAGGCGACAGAAAGGCCAGAGAGAAGGGCGATAAGGGCCCATCTGATGCCGA
GGTTGTCGACGAGATATCAATGATGGGCAGAGTGGTCAAGGTGGAAAAACAGGTGC
AGAGCATCGAGCACAAGCTGGACCTGCTGCTGGGATTCTACAGCCGGTGTCTGAGA
AGCGGCACATCTGCATCTCTGGGCGCTGTGCAGGTCCCACTGTTCGACCCTGATATC
ACCAGCGACTATCACAGCCCCGTGGACCACGAGGACATCTCCGTTTCTGCTCAGACC
CTGAGCATCAGCAGATCCGTGTCCACCAACATGGACGGATCCCGGGCTGACTACAA
AGACCATGACGGTGATTATAAAGATCATGACATCGACTACAAGGATGACGATGACA
AGTAATAAGAGCTCGCTGATCAGCCTCGACTGTGCCTTCTAGTTGCCAGCCATCTGTT
GTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGG
GGGGTGGGGTGGGGCAGGACAGCAAGGGGGAGGATTGGGAAGACAATAGCAGGCA
TGCTGGGGATGCGGTGGGCTCTATGttaattCGGACCGCTAGGAACCCCTAGTGATGGAG
TTGGCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGCTG
CCTGCAGG

TABLE 7
Construct Components (SEQ ID NO: 27)

| Component (5′ to | Position |
|---|---|
| 3′ orientation) | (nucleotides) |
| 5′ ITR | 12-130 |
| CHRNA10 | 145-884 |
| promoter | |
| 5′ UTR | 885-1174 |
| KCNQ4 | 1192-3276 |
| coding region | |
| 3x Flag | 3289-3357 |
| polyA | 3382-3605 |
| 3′ ITR | 3621-3750 |

In some aspects, the CHRNA10 promoter comprises a nucleic acid sequence that comprises at least 80%, at least 85%, at least 90%, at least 95%, at least 96%, at least 97%, at least 98% at least 99%, or 100% identity to nucleotides 145-884 of SEQ ID NO: 27.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 28. In some aspects, the construct comprises the nucleic acid sequence of SEQ ID NO: 28.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-3928 of SEQ ID NO: 28. In some aspects, the construct comprises nucleotides 12-3928 of SEQ ID NO: 28.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 28. In some aspects, the rAAVAnc80 particle comprises the nucleic acid sequence of SEQ ID NO: 28.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a DNM3 promoter comprising the nucleic acid sequence of SEQ ID NO: 5, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a DNM3 promoter comprising the nucleic acid sequence of SEQ ID NO: 5, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 28)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTCCTTGATTCAGAGTTAAAGCTATGGG
AAAGTCCTCAGGCAGAGGACAAACATTAGACAAGAAAATGCCCATATATGAAACCC
TGCGAAGCATCAGTATTTGAGGAGCAGACTAAAAAGGAACCGTCTGTGGAGGCTAA
GAGAAGCATGGCCATTTATCTTTGTGTCCCGATCATCAGGCACAGGACCCCACACAC
AGTCACTTCTCAATGTGCTAAATTTCACAGAATGCGTCCAGGGTACCTGGTTCTGGA
TAGATCCGGTAGAAGGAGATAGACCGGGAGGGCAAATGGCATGAGGAGTCTCACAG
GCCAGAGTGATTAAAGGGGTGTATCGGGGCGGTAAACCCTACAGACTCTACCTGTGC
TTATGCGGGGCTGGGGAGGACGAGTCATTACAGATGAAGAATTAAGTAAGGTCAGA
CCACTCAGGGCCTTAGATGGATGTCACATTGAAGAATTTAGACTCCAACAGGCCTGC
CACCCTGGGAGGAGTCATCGCGGATTCTGGAGAAGGGCGTGACAGAGGAGATTTCC
TTTCGGGAAGTGTAGTCTGGCAGCGGTGCCCCGGTGGTGGCGGCGGCGGTGCTGCTG
TTGCTGGTGATCGTGTGGTGGTGTTAGCGGCGATAGTGCTTTCCACTGGGCTTTGGCT
TGGTAGCCGCTGAAAGAGAACAACGCTGCCGCTGCTGCTGATTTCATGCCATTTCCT
GACCCGGCGCTGTAACTTGGCCTCTGAGCCTTGGCCACAGAACGCAGAGGCCGTGGC
ATCTGGCCGCAGCTGGGCTGCAGTGCGTGCGCGCCTGGCCTGGTGGTCCGATGGGAA
GCCCGGGGCGGGGCAGCCGCGGGGCGGGGGCGGGGCGTCGCGGAGATAGGCCACG
CCCCTGCCCGCCCGCGCAGGCGCGCTGCGGGTCGTTAGCTGTCCGCCGGTGGCAGGT
GGAAAGGCGAGCGGCATGGAGCGCGTAATAAGAGAGTTGGAGTCGGAAAGAGCAG
CCCCAGTCGCCGGGGAAGCGGGAGGTCAGTGCGGGCTCCGGCGGCCCCCAGGCTCC
GAGCGCCCGCCCGCGGCCCCGGCCCGGCCCCTAGCCCCCGCCGCCCGCGCCCGCCCC
GGGTCGCCCCTCTGGCCCCGGGTCCGAGCCATGCGTCTCTGAGCGCCCCGAGCGCGC
CCCCGCCCCGGACCGTGCCCGGGCCCCGGCGCCCCCAGCCCGGCGCCGCCCACCGGT
CGCTAGCCACCATGGCTGAAGCCCCTCCTAGAAGGCTTGGACTGGGACCTCCTCCTG
GGGATGCTCCTAGAGCTGAACTGGTGGCTCTGACAGCCGTGCAGTCTGAACAAGGC
GAAGCTGGTGGCGGCGGATCTCCACGTAGACTTGGACTGCTGGGAAGCCCTCTTCCT
CCTGGTGCTCCACTTCCTGGACCTGGCAGTGGATCTGGATCTGCCTGTGGCCAGAGA
AGCTCTGCCGCTCACAAGAGATACCGGCGGCTGCAGAACTGGGTGTACAACGTGCT
GGAAAGACCCAGAGGCTGGGCCTTCGTGTACCACGTGTTCATCTTTCTGCTGGTGTT
CAGCTGCCTGGTGCTGTCCGTGCTGAGCACCATCCAAGAACATCAAGAGCTGGCTAA
CGAGTGCCTGTTAATACTGGAGTTTGTGATGATTGTGGTGTTCGGCCTCGAGTACATC
GTCCGCGTTTGGAGCGCCGGCTGCTGCTGCAGATATAGAGGTTGGCAAGGCAGATTC
CGCTTCGCCAGAAAGCCCTTCTGCGTGATCGACTTCATCGTGTTCGTGGCCAGCGTG
GCCGTGATTGCTGCTGGCACACAGGGCAACATCTTCGCCACAAGCGCCCTGCGGAGC
ATGCGGTTTCTGCAGATCCTGAGAATGGTCCGAATGGACAGAAGAGGCGGCACCTG
GAAGCTGCTGGGCTCTGTGGTGTACGCCCACAGCAAAGAGCTGATCACCGCCTGGTA
CATCGGATTTCTGGTGCTGATCTTCGCCTCCTTCCTGGTGTACCTGGCCGAGAAGGAC
GCCAACAGCGACTTTAGCAGCTACGCCGACTCTCTTTGGTGGGGCACCATCACACTG
ACCACCATCGGCTACGGCGACAAGACCCCTCACACATGGCTGGGAAGAGTGCTGGC
CGCTGGATTTGCTCTGCTGGGCATCAGCTTTTTCGCCCTGCCTGCCGGAATCCTCGGA
TCTGGCTTTGCCCTGAAGGTGCAAGAGCAGCACCGGCAGAAGCACTTCGAGAAGAG
AAGAATGCCTGCCGCCAACCTGATTCAGGCCGCTTGGAGACTGTACAGCACCGACAT
GAGCAGAGCCTACCTGACCGCCACGTGGTATTATTACGACTCGATCCTGCCTAGCTT
CCGCGAACTGGCCCTGCTGTTTGAGCATGTGCAGAGAGCCAGAAACGGCGGCCTCA
GACCTCTGGAAGTTCGGAGAGCACCTGTGCCTGATGGCGCCCCTTCTAGATATCCTC
CAGTGGCCACCTGTCACAGACCCGGCAGCACATCTTTTTGCCCTGGCGAGTCTAGCC
GGATGGGCATCAAGGACAGAATCAGAATGGGCAGCAGCCAGCGGAGAACAGGCCC
TTCTAAACAGCATCTGGCCCCTCCAACCATGCCTACAAGCCCTAGCTCTGAGCAAGT
GGGCGAAGCCACCTCTCCTACCAAGGTGCAGAAGTCCTGGTCCTTCAACGACCGGAC
CAGATTCAGAGCCAGCCTGAGACTGAAGCCCAGAACCTCTGCCGAGGATGCCCCTTC
TGAAGAGGTGGCCGAAGAGAAGTCCTACCAGTGCGAGCTGACCGTGGACGACATCA
TGCCAGCCGTGAAAACCGTGATACGGTCTATCCGGATCCTGAAGTTCCTGGTGGCCA
AGCGGAAGTTCAAAGAGACACTGCGGCCCTACGACGTGAAGGACGTGATCGAGCAG
TATTCTGCCGGCCACCTGGACATGCTGGGCAGAATCAAGAGCCTGCAGACCAGAGT
GGACCAGATCGTTGGAAGAGGCCCAGGCGACAGAAAGGCCAGAGAGAAGGGCGAT
AAGGGCCCATCTGATGCCGAGGTTGTCGACGAGATATCAATGATGGGCAGAGTGGT
CAAGGTGGAAAAACAGGTGCAGAGCATCGAGCACAAGCTGGACCTGCTGCTGGGAT
TCTACAGCCGGTGTCTGAGAAGCGGCACATCTGCATCTCTGGGCGCTGTGCAGGTCC
CACTGTTCGACCCTGATATCACCAGCGACTATCACAGCCCCGTGGACCACGAGGACA
TCTCCGTTTCTGCTCAGACCCTGAGCATCAGCAGATCCGTGTCCACCAACATGGACG
GATCCCGGGCTGACTACAAAGACCATGACGGTGATTATAAAGATCATGACATCGACT
ACAAGGATGACGATGACAAGTAATAAGAGCTCGCTGATCAGCCTCGACTGTGCCTTC
TAGTTGCCAGCCATCTGTTGTTTGCCCCTCCCCCGTGCCTTCCTTGACCCTGGAAGGT
GCCACTCCCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGAGGATTG
GGAAGACAATAGCAGGCATGCTGGGGATGCGGTGGGCTCTATGttaattCGGACCGCTA
GGAACCCCTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCTCAGTGA
GCGAGCGAGCGCGCAGCTGCCTGCAGG

TABLE 8
Construct Components (SEQ ID NO: 28)

| Component (5′ to | Position |
|---|---|
| 3′ orientation) | (nucleotides) |
| 5′ ITR | 12-130 |
| DNM3 promoter | 145-1062 |
| 5′ UTR | 1063-1352 |
| KCNQ4 | 1370-3454 |
| coding region | |
| 3x Flag | 3467-3535 |
| polyA | 3560-3783 |
| 3′ ITR | 3799-3928 |

In some aspects, the DNM3 promoter comprises a nucleic acid sequence that comprises at least 80%, at least 85%, at least 90%, at least 95%, at least 96%, at least 97%, at least 98% at least 99%, or 100% identity to nucleotides 145-1062 of SEQ ID NO: 28.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 29. In some aspects, the construct comprises the nucleic acid sequence of SEQ ID NO: 29.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4641 of SEQ ID NO: 29. In some aspects, the construct comprises nucleotides 12-4641 of SEQ ID NO: 29.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 29. In some aspects, the rAAVAnc80 particle comprises the nucleic acid sequence of SEQ ID NO: 29.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a MUC15 promoter comprising the nucleic acid sequence of SEQ ID NO: 6, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a MUC15 promoter comprising the nucleic acid sequence of SEQ ID NO: 6, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 29)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTTTTCTCCTAATTCAGCACAAAAATTG
AGTTCCTTTTCTGTAGCTAAAGAGCTTGTATGAACTGTCAGCTTAGCTAACCATATGT
TTTCAATGTTCCCTGCAAATTGTTTAAGGTATGTATAGTCCTTTCAATGGATGAGTAA
GTCTTTTGTCATTGTTATTTGCTGCCTGTGGACTTGATTTCAAAATCTTCTTCAGGTCA
TGAATAAATTTCCTTTTCCTTCTGTCCCTACTTTTGAGCCAAGGAACAAATCAAGATT
CTTCCTCAGAGTGTACACACCTTCCCAGGCATCTCACTCTCTCCTCACTCTATCTGCT
TCAAGTTATGGCTCGTTGGTGAGAACACTCTGCTGCTGAGGTTATTATTTAGCTATAA
TAACTTTTTCTAACTAGACAGAAACAAATTAGATATGCCAGGATTTTCTAATTACCTG
CCTTAAGTGCTTTTTTAGAAAGCATTAATAAATCATGTGGATCTTTTCCTAGCAGTGG
TAAGATAAGTTATAATATTATCAAACTGTCAGTTTTGCCACTTCAATATATGTATGCC
TGGTTGTAACCTCACTTAATAAGTTAAGTCCATGTAAAAATAGTTGATAGTTAATAA
ATTGGGCAAGAGTTGCTTAAACAGATTAGACTATATAACAAAATTAGGGTTTTAAAA
GAATAAAGCTGCTATAACAGTACGCTTCATCTCACAGGAATTAATCAGTTATGGTAT
CTCCACAAAACAGAATATCACGTATTGTTGAAGAGAGCCGTCTCATTTCCCCGGGGT
TGGTTTAATTTCTAATCAAACTCTGAAGGGGCCTTTGGGCTTCAGAAAATTTAAAAC
TATAGAATTACCTTGTTCTTTCCTCGGGCCAATTAACTGGGCAGATTCTTTGCATTCC
ATTTGAAGCTTACTAGCTCCTGCATTTTAGCTAAAGTTTCGTTTCTCGCTCAGCAGTT
GAAAACCTATCTCCTTGTGCAGCAGAAACCAAGTATGAACCTCAGGCATATTGAGCT
GAACGGCCCTTGGCGCCATCCCCAAACGCTGATGTGCGGAAGATCCCAGTTTCACTC
TTCTCCCTTTCATAAGCTCTGAAAGGAAGTGTAGGAAGTATGCCAAGTTGTTATTCA
ACTCTAGTATTTAATCAAGCATTACCTGGGCACTTCTGAAATTCTCCAGCTTCTAAAG
TGAGAGTAAACCAGAGAGAACACAGGGTGGAAACTACTTAATCGAGAAGGCTCCTA
GGATAAGTGAGGATCACATGGCCATTCTCAGGCCCCAGTTCCTCTCCAAACTCCTGA
AAGTCAGCAAGAAACCGAATCTCAGTCATGATGATTATTTTTCATGTAACACCTCAC
AGCGTTCTCAGGGATCCCAATATATGCTACTAATTCACTTTGTGTTAAGTAGGAGTTT
CTTAAAAAAACAATTTCAGTGGAGAAATTCCTGCTATACCAGATGACTTTGCCAAAA
TCTTTGTCCTTTTTTTCACTTAGGGTGAAAAAAAAAATTGATGACCCGTGTTTTGCTA
CCACTGACGAGAGTAATACCTTGTCCCAAAGCTAAAACGATCAACCTATGAAAACTG
GAGGGTTGGGCTTTTGTTGTTGTTGTTAAAGGCCTGAATGAGGTGATATCTTCGCCG
GTGGCAGGTGGAAAGGCGAGCGGCATGGAGCGCGTAATAAGAGAGTTGGAGTCGG
AAAGAGCAGCCCCAGTCGCCGGGGAAGCGGGAGGTCAGTGCGGGCTCCGGCGGCCC
CCAGGCTCCGAGCGCCCGCCCGCGGCCCCGGCCCGGCCCCTAGCCCCCGCCGCCCGC
GCCCGCCCCGGGTCGCCCCTCTGGCCCCGGGTCCGAGCCATGCGTCTCTGAGCGCCC
CGAGCGCGCCCCCGCCCCGGACCGTGCCCGGGCCCCGGCGCCCCCAGCCCGGCGCC
GCCCACCGGTCGCTAGCCACCATGGCTGAAGCCCCTCCTAGAAGGCTTGGACTGGGA
CCTCCTCCTGGGGATGCTCCTAGAGCTGAACTGGTGGCTCTGACAGCCGTGCAGTCT
GAACAAGGCGAAGCTGGTGGCGGCGGATCTCCACGTAGACTTGGACTGCTGGGAAG
CCCTCTTCCTCCTGGTGCTCCACTTCCTGGACCTGGCAGTGGATCTGGATCTGCCTGT
GGCCAGAGAAGCTCTGCCGCTCACAAGAGATACCGGCGGCTGCAGAACTGGGTGTA
CAACGTGCTGGAAAGACCCAGAGGCTGGGCCTTCGTGTACCACGTGTTCATCTTTCT
GCTGGTGTTCAGCTGCCTGGTGCTGTCCGTGCTGAGCACCATCCAAGAACATCAAGA
GCTGGCTAACGAGTGCCTGTTAATACTGGAGTTTGTGATGATTGTGGTGTTCGGCCTC
GAGTACATCGTCCGCGTTTGGAGCGCCGGCTGCTGCTGCAGATATAGAGGTTGGCAA
GGCAGATTCCGCTTCGCCAGAAAGCCCTTCTGCGTGATCGACTTCATCGTGTTCGTG
GCCAGCGTGGCCGTGATTGCTGCTGGCACACAGGGCAACATCTTCGCCACAAGCGCC
CTGCGGAGCATGCGGTTTCTGCAGATCCTGAGAATGGTCCGAATGGACAGAAGAGG
CGGCACCTGGAAGCTGCTGGGCTCTGTGGTGTACGCCCACAGCAAAGAGCTGATCAC
CGCCTGGTACATCGGATTTCTGGTGCTGATCTTCGCCTCCTTCCTGGTGTACCTGGCC
GAGAAGGACGCCAACAGCGACTTTAGCAGCTACGCCGACTCTCTTTGGTGGGGCACC
ATCACACTGACCACCATCGGCTACGGCGACAAGACCCCTCACACATGGCTGGGAAG
AGTGCTGGCCGCTGGATTTGCTCTGCTGGGCATCAGCTTTTTCGCCCTGCCTGCCGGA
ATCCTCGGATCTGGCTTTGCCCTGAAGGTGCAAGAGCAGCACCGGCAGAAGCACTTC
GAGAAGAGAAGAATGCCTGCCGCCAACCTGATTCAGGCCGCTTGGAGACTGTACAG
CACCGACATGAGCAGAGCCTACCTGACCGCCACGTGGTATTATTACGACTCGATCCT
GCCTAGCTTCCGCGAACTGGCCCTGCTGTTTGAGCATGTGCAGAGAGCCAGAAACGG
CGGCCTCAGACCTCTGGAAGTTCGGAGAGCACCTGTGCCTGATGGCGCCCCTTCTAG
ATATCCTCCAGTGGCCACCTGTCACAGACCCGGCAGCACATCTTTTTGCCCTGGCGA
GTCTAGCCGGATGGGCATCAAGGACAGAATCAGAATGGGCAGCAGCCAGCGGAGAA
CAGGCCCTTCTAAACAGCATCTGGCCCCTCCAACCATGCCTACAAGCCCTAGCTCTG
AGCAAGTGGGCGAAGCCACCTCTCCTACCAAGGTGCAGAAGTCCTGGTCCTTCAACG
ACCGGACCAGATTCAGAGCCAGCCTGAGACTGAAGCCCAGAACCTCTGCCGAGGAT
GCCCCTTCTGAAGAGGTGGCCGAAGAGAAGTCCTACCAGTGCGAGCTGACCGTGGA
CGACATCATGCCAGCCGTGAAAACCGTGATACGGTCTATCCGGATCCTGAAGTTCCT
GGTGGCCAAGCGGAAGTTCAAAGAGACACTGCGGCCCTACGACGTGAAGGACGTGA
TCGAGCAGTATTCTGCCGGCCACCTGGACATGCTGGGCAGAATCAAGAGCCTGCAG
ACCAGAGTGGACCAGATCGTTGGAAGAGGCCCAGGCGACAGAAAGGCCAGAGAGA
AGGGCGATAAGGGCCCATCTGATGCCGAGGTTGTCGACGAGATATCAATGATGGGC
AGAGTGGTCAAGGTGGAAAAACAGGTGCAGAGCATCGAGCACAAGCTGGACCTGCT
GCTGGGATTCTACAGCCGGTGTCTGAGAAGCGGCACATCTGCATCTCTGGGCGCTGT
GCAGGTCCCACTGTTCGACCCTGATATCACCAGCGACTATCACAGCCCCGTGGACCA
CGAGGACATCTCCGTTTCTGCTCAGACCCTGAGCATCAGCAGATCCGTGTCCACCAA
CATGGACGGATCCCGGGCTGACTACAAAGACCATGACGGTGATTATAAAGATCATG
ACATCGACTACAAGGATGACGATGACAAGTAATAAGAGCTCGCTGATCAGCCTCGA
CTGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCAT
TGTCTGAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTATGttaattC
GGACCGCTAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGCGG
CCTCAGTGAGCGAGCGAGCGCGCAGCTGCCTGCAGG

TABLE 9
Construct Components (SEQ ID NO: 29)

| Component (5′ to | Position |
|---|---|
| 3′ orientation) | (nucleotides) |
| 5′ ITR | 12-130 |
| MUC15 promoter | 145-1775 |
| 5′ UTR | 1776-2065 |
| KCNQ4 | 2083-4167 |
| coding region | |
| 3x Flag | 4180-4248 |
| polyA | 4273-4496 |
| 3′ ITR | 4512-4641 |

In some aspects, the MUC15 promoter comprises a nucleic acid sequence that comprises at least 80%, at least 85%, at least 90%, at least 95%, at least 96%, at least 97%, at least 98% at least 99%, or 100% identity to nucleotides 145-1775 of SEQ ID NO: 29.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 30. In some aspects, the construct comprises the nucleic acid sequence of SEQ ID NO: 30.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-3994 of SEQ ID NO: 30. In some aspects, the construct comprises nucleotides 12-3994 of SEQ ID NO: 30.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 30. In some aspects, the rAAVAnc80 particle comprises the nucleic acid sequence of SEQ ID NO: 30.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a PLBD1 promoter comprising the nucleic acid sequence of SEQ ID NO: 7, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a PLBD1 promoter comprising the nucleic acid sequence of SEQ ID NO: 7, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 30)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGACCCATTATTCAATGGGAGTTGTCA
GGATGTCAGCAATGTACAAAATCATTGCTTAATTTGTTTGACAATGGAAATGGCCAT
TATGGTTTTTATGTAACTTTGCTTCTGTTACATAATTCTTGCTGACACGGTGTTTCAAC
CAAGGTACTTGGTAGCAAGTGTTGTACAGAAAAGGATCTGTAAGTGGTTTATGTGGT
CATCAACCACAGCAAAGATTTCATCTGAGCTGTGCTATGAAGAATGTAGCTTGAGAA
ACACAAAATGTATCACTGGGCAAAAAGGAAGCAGAAGAAAAATACAGTTCTGCTAA
TGAGAGCTCTGACTGGTATCTGGAGTATAAGATGGGCCCAGCCAATGCTGAGTGAAT
GAATGAAATGCCTTTTGCCTACTTCACAATGTCACCTAGGGCACCCGGTGCCAACTT
CACAATATCACCCAAGGCATAACTTTTGACTACTTCACAATGTCACTTTTAACTGACC
CCACACAGAAATGGGGACTCCACAGAAACGTAGGAGTGTGTCTAGTGTCAGCCCCG
TCTGAATCACTCTCCTGTGGTGGCTCCAGCCAACGAAGAGGAAGCAAAAAGGATAA
AAAATCTGAGCTACAGCGCATGGATTTAGGTTAAACAGCCTGGGAATGAGGGGTAC
GCTAATCGCTGAGGAAAACGCACCTGTGGAGGCCTCTCCAGAAACAGCAGAGGATC
CGAGCTGCGTGTAGGCAGGGCGCGCATGTCACCCTGGCCCGGGCGCCTGGTCCGCTG
CTGGAGATAAATGGTCGACCCCGGAGGGAGAGGCTAGTAGGGGTGTTGATGTGAAC
TGATTCGCCCAAGCCTTGGGCCGCAAAACTGCGAAAGAAAGCGGCAGGCAGCCTCT
GCATTTCCCAGAAGTGCAGCTGGGGAACTTTCCCAGACCGGCCCAGGGGTTGCTAGA
GGGTCAGACGTAAAGGATCCGCCTTTCCTAGGCGGGGTGGGCCCCAGGCCCGCCGG
TGGCAGGTGGAAAGGCGAGCGGCATGGAGCGCGTAATAAGAGAGTTGGAGTCGGA
AAGAGCAGCCCCAGTCGCCGGGGAAGCGGGAGGTCAGTGCGGGCTCCGGCGGCCCC
CAGGCTCCGAGCGCCCGCCCGCGGCCCCGGCCCGGCCCCTAGCCCCCGCCGCCCGCG
CCCGCCCCGGGTCGCCCCTCTGGCCCCGGGTCCGAGCCATGCGTCTCTGAGCGCCCC
GAGCGCGCCCCCGCCCCGGACCGTGCCCGGGCCCCGGCGCCCCCAGCCCGGCGCCG
CCCACCGGTCGCTAGCCACCATGGCTGAAGCCCCTCCTAGAAGGCTTGGACTGGGAC
CTCCTCCTGGGGATGCTCCTAGAGCTGAACTGGTGGCTCTGACAGCCGTGCAGTCTG
AACAAGGCGAAGCTGGTGGCGGCGGATCTCCACGTAGACTTGGACTGCTGGGAAGC
CCTCTTCCTCCTGGTGCTCCACTTCCTGGACCTGGCAGTGGATCTGGATCTGCCTGTG
GCCAGAGAAGCTCTGCCGCTCACAAGAGATACCGGCGGCTGCAGAACTGGGTGTAC
AACGTGCTGGAAAGACCCAGAGGCTGGGCCTTCGTGTACCACGTGTTCATCTTTCTG
CTGGTGTTCAGCTGCCTGGTGCTGTCCGTGCTGAGCACCATCCAAGAACATCAAGAG
CTGGCTAACGAGTGCCTGTTAATACTGGAGTTTGTGATGATTGTGGTGTTCGGCCTCG
AGTACATCGTCCGCGTTTGGAGCGCCGGCTGCTGCTGCAGATATAGAGGTTGGCAAG
GCAGATTCCGCTTCGCCAGAAAGCCCTTCTGCGTGATCGACTTCATCGTGTTCGTGGC
CAGCGTGGCCGTGATTGCTGCTGGCACACAGGGCAACATCTTCGCCACAAGCGCCCT
GCGGAGCATGCGGTTTCTGCAGATCCTGAGAATGGTCCGAATGGACAGAAGAGGCG
GCACCTGGAAGCTGCTGGGCTCTGTGGTGTACGCCCACAGCAAAGAGCTGATCACCG
CCTGGTACATCGGATTTCTGGTGCTGATCTTCGCCTCCTTCCTGGTGTACCTGGCCGA
GAAGGACGCCAACAGCGACTTTAGCAGCTACGCCGACTCTCTTTGGTGGGGCACCAT
CACACTGACCACCATCGGCTACGGCGACAAGACCCCTCACACATGGCTGGGAAGAG
TGCTGGCCGCTGGATTTGCTCTGCTGGGCATCAGCTTTTTCGCCCTGCCTGCCGGAAT
CCTCGGATCTGGCTTTGCCCTGAAGGTGCAAGAGCAGCACCGGCAGAAGCACTTCGA
GAAGAGAAGAATGCCTGCCGCCAACCTGATTCAGGCCGCTTGGAGACTGTACAGCA
CCGACATGAGCAGAGCCTACCTGACCGCCACGTGGTATTATTACGACTCGATCCTGC
CTAGCTTCCGCGAACTGGCCCTGCTGTTTGAGCATGTGCAGAGAGCCAGAAACGGCG
GCCTCAGACCTCTGGAAGTTCGGAGAGCACCTGTGCCTGATGGCGCCCCTTCTAGAT
ATCCTCCAGTGGCCACCTGTCACAGACCCGGCAGCACATCTTTTTGCCCTGGCGAGT
CTAGCCGGATGGGCATCAAGGACAGAATCAGAATGGGCAGCAGCCAGCGGAGAAC
AGGCCCTTCTAAACAGCATCTGGCCCCTCCAACCATGCCTACAAGCCCTAGCTCTGA
GCAAGTGGGCGAAGCCACCTCTCCTACCAAGGTGCAGAAGTCCTGGTCCTTCAACGA
CCGGACCAGATTCAGAGCCAGCCTGAGACTGAAGCCCAGAACCTCTGCCGAGGATG
CCCCTTCTGAAGAGGTGGCCGAAGAGAAGTCCTACCAGTGCGAGCTGACCGTGGAC
GACATCATGCCAGCCGTGAAAACCGTGATACGGTCTATCCGGATCCTGAAGTTCCTG
GTGGCCAAGCGGAAGTTCAAAGAGACACTGCGGCCCTACGACGTGAAGGACGTGAT
CGAGCAGTATTCTGCCGGCCACCTGGACATGCTGGGCAGAATCAAGAGCCTGCAGA
CCAGAGTGGACCAGATCGTTGGAAGAGGCCCAGGCGACAGAAAGGCCAGAGAGAA
GGGCGATAAGGGCCCATCTGATGCCGAGGTTGTCGACGAGATATCAATGATGGGCA
GAGTGGTCAAGGTGGAAAAACAGGTGCAGAGCATCGAGCACAAGCTGGACCTGCTG
CTGGGATTCTACAGCCGGTGTCTGAGAAGCGGCACATCTGCATCTCTGGGCGCTGTG
CAGGTCCCACTGTTCGACCCTGATATCACCAGCGACTATCACAGCCCCGTGGACCAC
GAGGACATCTCCGTTTCTGCTCAGACCCTGAGCATCAGCAGATCCGTGTCCACCAAC
ATGGACGGATCCCGGGCTGACTACAAAGACCATGACGGTGATTATAAAGATCATGA
CATCGACTACAAGGATGACGATGACAAGTAATAAGAGCTCGCTGATCAGCCTCGAC
TGTGCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTATGttaattCG
GACCGCTAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGGC
CTCAGTGAGCGAGCGAGCGCGCAGCTGCCTGCAGG

TABLE 10
Construct Components (SEQ ID NO: 30)

| Component (5′ to | Position |
|---|---|
| 3′ orientation) | (nucleotides) |
| 5′ ITR | 12-130 |
| PLBD1 promoter | 145-1128 |
| 5′ UTR | 1129-1418 |
| KCNQ4 | 1436-3520 |
| coding region | |
| 3x Flag | 3533-3601 |
| polyA | 3626-3849 |
| 3′ ITR | 3865-3994 |

In some aspects, the PLBD1 promoter comprises a nucleic acid sequence that comprises at least 80%, at least 85%, at least 90%, at least 95%, at least 96%, at least 97%, at least 98% at least 99%, or 100% identity to nucleotides 145-1128 of SEQ ID NO: 30.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 31. In some aspects, the construct comprises the nucleic acid sequence of SEQ ID NO: 31.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4426 of SEQ ID NO: 31. In some aspects, the construct comprises nucleotides 12-4426 of SEQ ID NO: 31.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 31. In some aspects, the construct rAAVAnc80 particle the nucleic acid sequence of SEQ ID NO: 31.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a RORB promoter comprising the nucleic acid sequence of SEQ ID NO: 8, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a RORB promoter comprising the nucleic acid sequence of SEQ ID NO: 8, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 31)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTCCAATAAATGTTGGCTCTTGTTTTTTC
TGACCTGTATGTTTTGTCTTTGTTCCAAAGCTAGCCTTACCTCTCCCACATACTGGGG
TTAATTCATGCTTTGGCCCTTATCACCTTTTCCAATTTATTTCAAAATTACATGCTCTA
TTTTAATATTTGCTTTCTTTTTTTTATTTTGAAAACTTATTGAACTTGCATCTACACTTT
AAAATGAAGCAGAACTTAAAGAACTCAAAGATTATGAAGAAGACTCAGTACCTGGG
AATAAAATTGAGAATAGGTTCCTTTTATGACTATATAACCAATCTCAACCATTATTTT
TTGCTTCCCCAAATTAGGAGAGTTTAAAATGCAGATTCTCCCCACTCTCCTCTTCCCA
TTCAATAGAAACTGAGAAAGAAGGATCTTATTCAGGTCTTCACTCCATTTGTGATTC
ATATTCAGTGGCTGAAAGGTTAGAAAGCATTCACTCCACCAATAATGATCAAGCACC
CATAAAGTACCAGGAGCTCTTACAAACTCTAGGGAAATCCTGGCTCCTGTTGTCATG
AATTTTGCATTCTCAGGTAGGAAATGTGGCTCTGATGCCTGCTGGGGCAGTGTACAC
TTAGAGCTACAGAGGATCTTGGAGGTAATCTAAAACCTTTCTAAAGAGCACCCTGCA
ATCACACCTTCTAGCAACAGCCATTTCTCTTGAATTAGTAAGGTGGCTACACCGCCA
ATTTGAGCTGTTCTCCTTCAGTCCTGTAGTCCATCGCCAGGGGAGTCTCCAAATGCTA
ATAAAAATCAATTTCCCAGACAAAAGAACATAGAGGGTCAGGGAGCATCTGACGGA
CGTTTTTAAAGGAAGGGGACAGCTACTTCCATGGGACTGCATTTTAGTTGTGCTAAA
AGTGATGAAAGTGGGTTTGCATTATTCTACCACCAACACCCAAACCACCTGCCCACG
GAAACCCCCGCCGGAGACCGAAGTTTACCCAAATAGCGCTCGGCAAAGCGCTGCCA
TAAATTCAAAACTAACTCTGCCGGGCCCGCGGGGGTTGCGAGACAGGGACCGAACG
TGAAACCCGGGGAGCCCCGCGTCTCTTGCCTCCGAAGGTTTTCCGTGATCAGTGTCC
CCTTCTCTGCTGGAGTCGGAAGTGCCTGTCACCTGCGGATCTGCCCGACTCTCCCGGT
CGGCCCTTCTTCTCTGCCCAGTTCGGACAGTCTCGAATTCCCCGTCGCAGCCCCGGCC
ACCTCGGACTCCCTGGTCCCCAGCCCCCGCCCCACCCCCCGCCTCCACCACGTCCCCT
CCCCGCGGTCCCAGCCTCTCCAGGCGCTGCTGGGCTCTGATTGGCGGCTGCGCTGAC
AGCAGGCGGGGCCTGGAAGTCGCGGCCAAGCCCGCCCTCGCGTATAAGCCCCTCTC
AGCGCTCTCTCTCCCGCCGGTGGCAGGTGGAAAGGCGAGCGGCATGGAGCGCGTAA
TAAGAGAGTTGGAGTCGGAAAGAGCAGCCCCAGTCGCCGGGGAAGCGGGAGGTCA
GTGCGGGCTCCGGCGGCCCCCAGGCTCCGAGCGCCCGCCCGCGGCCCCGGCCCGGC
CCCTAGCCCCCGCCGCCCGCGCCCGCCCCGGGTCGCCCCTCTGGCCCCGGGTCCGAG
CCATGCGTCTCTGAGCGCCCCGAGCGCGCCCCCGCCCCGGACCGTGCCCGGGCCCCG
GCGCCCCCAGCCCGGCGCCGCCCACCGGTCGCTAGCCACCATGGCTGAAGCCCCTCC
TAGAAGGCTTGGACTGGGACCTCCTCCTGGGGATGCTCCTAGAGCTGAACTGGTGGC
TCTGACAGCCGTGCAGTCTGAACAAGGCGAAGCTGGTGGCGGCGGATCTCCACGTA
GACTTGGACTGCTGGGAAGCCCTCTTCCTCCTGGTGCTCCACTTCCTGGACCTGGCAG
TGGATCTGGATCTGCCTGTGGCCAGAGAAGCTCTGCCGCTCACAAGAGATACCGGCG
GCTGCAGAACTGGGTGTACAACGTGCTGGAAAGACCCAGAGGCTGGGCCTTCGTGT
ACCACGTGTTCATCTTTCTGCTGGTGTTCAGCTGCCTGGTGCTGTCCGTGCTGAGCAC
CATCCAAGAACATCAAGAGCTGGCTAACGAGTGCCTGTTAATACTGGAGTTTGTGAT
GATTGTGGTGTTCGGCCTCGAGTACATCGTCCGCGTTTGGAGCGCCGGCTGCTGCTG
CAGATATAGAGGTTGGCAAGGCAGATTCCGCTTCGCCAGAAAGCCCTTCTGCGTGAT
CGACTTCATCGTGTTCGTGGCCAGCGTGGCCGTGATTGCTGCTGGCACACAGGGCAA
CATCTTCGCCACAAGCGCCCTGCGGAGCATGCGGTTTCTGCAGATCCTGAGAATGGT
CCGAATGGACAGAAGAGGCGGCACCTGGAAGCTGCTGGGCTCTGTGGTGTACGCCC
ACAGCAAAGAGCTGATCACCGCCTGGTACATCGGATTTCTGGTGCTGATCTTCGCCT
CCTTCCTGGTGTACCTGGCCGAGAAGGACGCCAACAGCGACTTTAGCAGCTACGCCG
ACTCTCTTTGGTGGGGCACCATCACACTGACCACCATCGGCTACGGCGACAAGACCC
CTCACACATGGCTGGGAAGAGTGCTGGCCGCTGGATTTGCTCTGCTGGGCATCAGCT
TTTTCGCCCTGCCTGCCGGAATCCTCGGATCTGGCTTTGCCCTGAAGGTGCAAGAGC
AGCACCGGCAGAAGCACTTCGAGAAGAGAAGAATGCCTGCCGCCAACCTGATTCAG
GCCGCTTGGAGACTGTACAGCACCGACATGAGCAGAGCCTACCTGACCGCCACGTG
GTATTATTACGACTCGATCCTGCCTAGCTTCCGCGAACTGGCCCTGCTGTTTGAGCAT
GTGCAGAGAGCCAGAAACGGCGGCCTCAGACCTCTGGAAGTTCGGAGAGCACCTGT
GCCTGATGGCGCCCCTTCTAGATATCCTCCAGTGGCCACCTGTCACAGACCCGGCAG
CACATCTTTTTGCCCTGGCGAGTCTAGCCGGATGGGCATCAAGGACAGAATCAGAAT
GGGCAGCAGCCAGCGGAGAACAGGCCCTTCTAAACAGCATCTGGCCCCTCCAACCA
TGCCTACAAGCCCTAGCTCTGAGCAAGTGGGCGAAGCCACCTCTCCTACCAAGGTGC
AGAAGTCCTGGTCCTTCAACGACCGGACCAGATTCAGAGCCAGCCTGAGACTGAAG
CCCAGAACCTCTGCCGAGGATGCCCCTTCTGAAGAGGTGGCCGAAGAGAAGTCCTA
CCAGTGCGAGCTGACCGTGGACGACATCATGCCAGCCGTGAAAACCGTGATACGGT
CTATCCGGATCCTGAAGTTCCTGGTGGCCAAGCGGAAGTTCAAAGAGACACTGCGGC
CCTACGACGTGAAGGACGTGATCGAGCAGTATTCTGCCGGCCACCTGGACATGCTGG
GCAGAATCAAGAGCCTGCAGACCAGAGTGGACCAGATCGTTGGAAGAGGCCCAGGC
GACAGAAAGGCCAGAGAGAAGGGCGATAAGGGCCCATCTGATGCCGAGGTTGTCGA
CGAGATATCAATGATGGGCAGAGTGGTCAAGGTGGAAAAACAGGTGCAGAGCATCG
AGCACAAGCTGGACCTGCTGCTGGGATTCTACAGCCGGTGTCTGAGAAGCGGCACAT
CTGCATCTCTGGGCGCTGTGCAGGTCCCACTGTTCGACCCTGATATCACCAGCGACT
ATCACAGCCCCGTGGACCACGAGGACATCTCCGTTTCTGCTCAGACCCTGAGCATCA
GCAGATCCGTGTCCACCAACATGGACGGATCCCGGGCTGACTACAAAGACCATGAC
GGTGATTATAAAGATCATGACATCGACTACAAGGATGACGATGACAAGTAATAAGA
GCTCGCTGATCAGCCTCGACTGTGCCTTCTAGTTGCCAGCCATCTGTTGTTTGCCCCT
CCCCCGTGCCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGGGTGGGGT
GGGGCAGGACAGCAAGGGGGAGGATTGGGAAGACAATAGCAGGCATGCTGGGGAT
GCGGTGGGCTCTATGttaattCGGACCGCTAGGAACCCCTAGTGATGGAGTTGGCCACTC
CCTCTCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAGCTGCCTGCAGG

TABLE 11
Construct Components (SEQ ID NO: 31)

| Component (5′ to | Position |
|---|---|
| 3′ orientation) | (nucleotides) |
| 5′ ITR | 12-130 |
| RORB promoter | 145-1560 |
| 5′ UTR | 1561-1850 |
| KCNQ4 | 1868-3952 |
| coding region | |
| 3x Flag | 3965-4033 |
| polyA | 4058-4281 |
| 3′ ITR | 4297-4426 |

In some aspects, the RORB promoter comprises a nucleic acid sequence that comprises at least 80%, at least 85%, at least 90%, at least 95%, at least 96%, at least 97%, at least 98% at least 99%, or 100% identity to nucleotides 145-1560 of SEQ ID NO: 31.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 32. In some aspects, the construct comprises the nucleic acid sequence of SEQ ID NO: 32.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4307 of SEQ ID NO: 32. In some aspects, the construct comprises nucleotides 12-4307 of SEQ ID NO: 32.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 32. In some aspects, the rAAVAnc80 particle comprises the nucleic acid sequence of SEQ ID NO: 32.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a STRIP2 promoter comprising the nucleic acid sequence of SEQ ID NO: 9, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a STRIP2 promoter comprising the nucleic acid sequence of SEQ ID NO: 9, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 32)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTCCTTGAATATTTATGTCCATTTTAACA
CTTCCTGGTTGCAAGAGGGATGTGCCTCCATTATTTCCTCCACAGTTTTGGTATTTGT
CAGACATTTGTTCTGCTGTCTTTCTAATCCAGCCAACGTCTGCTCAGGAAGTGGGGCC
AGCTCCACTGGGACCCATAGTTTTACTTCCTTGTCATTTGATTGGATAGTTTCCAAGG
AAGCCCCTCCAGATTGGCACTATCTCAGAAAAGGAGAGCTTGTTGTGAAACACTGCT
TCCTGAAACTTCCTGCTATTGCCTAAAGCTACGTCTGAAACTGAGTAGGGAAAGGCA
TACTTTTCCAGGGACTTAGGGGGATAGGCTTTGAGGTCTCTCCTCGTGTTGACTCTAT
GCAATCTTCATAGCACCAGTTTTACACATTCCTTCTCTGAAATTAAAGCCAGATGGA
GCCTCTAGGCTTAGCAAGTGGCTTTAGATAGCCACCAGAGGGGACTTGCAAGCTGTC
CTCTATCCTACTCCCAAGATCAGTCTGCCCTTTCCCCTAGGAATAGGCAGGAAAAGA
ATAAAGGAAAGAAAGGACTGGCGAGCAGGTGAGGGTGGGGGCTTGCTCTACCCTCA
ACATTTACACACCATGAGGAAGAGGCCCCCTACAGCAGAGAAGGGCAGATGACAGG
AGCAGCCCTCGAGGGCACCACCAATTTCAGTGATGGAAAAACTCCCCCATCCCACCC
TTAGACCTCCAGTCTCCCAGCCAAGCCCTAGCTCCGGGCGAGATGCGTTCTCTTCAG
AAAAACGCTGAGAATTCTCAGCTTCCAGAGACAGCGAGTCCCTCGTTTCGGGCGATG
TCCCTGGCCACCTGGCGGTGCCATCCCTCCCCTGAGACTAAGCGGGATATGGGACGT
GTGCAGGAGCCGGGATATGGGGGGCCGGGTCGGTGGTAACAGGGAAACGGAGACT
GCTGTGGAGCAGTAGGCGGAGACTAGAGCTCCGGAAAAGGTCGCTACAGGGACGGG
GGTGAGAGCTGAGAGACACCGAGTGAGGAGCACAGAGATAACCCGCCTGATCTCAA
GGCCCAGCTTTCGCGAGGTGTGGAGCCTGTAGCTAACCTAGGAGTCTCCGTCCGCCA
GCAATGCCGCAGGACTAAAAAGATCCCCTCAAAAATCTCTTCATTGAGCCCCCACCT
CCTCGAGTCCCGCTCCGGCCGGTCGAGCAGCCAATCGCCTCGCGGGGCGGGGTTGCG
GCGAGCTGCCGTAACCAATAGAGGTGGAGGGGGCGGGGCCTGGCTCCCGGCGCGCG
GCGGTAGGGTCGCCTCCGGCGCCGGTGGCAGGTGGAAAGGCGAGCGGCATGGAGCG
CGTAATAAGAGAGTTGGAGTCGGAAAGAGCAGCCCCAGTCGCCGGGGAAGCGGGA
GGTCAGTGCGGGCTCCGGCGGCCCCCAGGCTCCGAGCGCCCGCCCGCGGCCCCGGC
CCGGCCCCTAGCCCCCGCCGCCCGCGCCCGCCCCGGGTCGCCCCTCTGGCCCCGGGT
CCGAGCCATGCGTCTCTGAGCGCCCCGAGCGCGCCCCCGCCCCGGACCGTGCCCGGG
CCCCGGCGCCCCCAGCCCGGCGCCGCCCACCGGTCGCTAGCCACCATGGCTGAAGCC
CCTCCTAGAAGGCTTGGACTGGGACCTCCTCCTGGGGATGCTCCTAGAGCTGAACTG
GTGGCTCTGACAGCCGTGCAGTCTGAACAAGGCGAAGCTGGTGGCGGCGGATCTCC
ACGTAGACTTGGACTGCTGGGAAGCCCTCTTCCTCCTGGTGCTCCACTTCCTGGACCT
GGCAGTGGATCTGGATCTGCCTGTGGCCAGAGAAGCTCTGCCGCTCACAAGAGATAC
CGGCGGCTGCAGAACTGGGTGTACAACGTGCTGGAAAGACCCAGAGGCTGGGCCTT
CGTGTACCACGTGTTCATCTTTCTGCTGGTGTTCAGCTGCCTGGTGCTGTCCGTGCTG
AGCACCATCCAAGAACATCAAGAGCTGGCTAACGAGTGCCTGTTAATACTGGAGTTT
GTGATGATTGTGGTGTTCGGCCTCGAGTACATCGTCCGCGTTTGGAGCGCCGGCTGC
TGCTGCAGATATAGAGGTTGGCAAGGCAGATTCCGCTTCGCCAGAAAGCCCTTCTGC
GTGATCGACTTCATCGTGTTCGTGGCCAGCGTGGCCGTGATTGCTGCTGGCACACAG
GGCAACATCTTCGCCACAAGCGCCCTGCGGAGCATGCGGTTTCTGCAGATCCTGAGA
ATGGTCCGAATGGACAGAAGAGGCGGCACCTGGAAGCTGCTGGGCTCTGTGGTGTA
CGCCCACAGCAAAGAGCTGATCACCGCCTGGTACATCGGATTTCTGGTGCTGATCTT
CGCCTCCTTCCTGGTGTACCTGGCCGAGAAGGACGCCAACAGCGACTTTAGCAGCTA
CGCCGACTCTCTTTGGTGGGGCACCATCACACTGACCACCATCGGCTACGGCGACAA
GACCCCTCACACATGGCTGGGAAGAGTGCTGGCCGCTGGATTTGCTCTGCTGGGCAT
CAGCTTTTTCGCCCTGCCTGCCGGAATCCTCGGATCTGGCTTTGCCCTGAAGGTGCAA
GAGCAGCACCGGCAGAAGCACTTCGAGAAGAGAAGAATGCCTGCCGCCAACCTGAT
TCAGGCCGCTTGGAGACTGTACAGCACCGACATGAGCAGAGCCTACCTGACCGCCA
CGTGGTATTATTACGACTCGATCCTGCCTAGCTTCCGCGAACTGGCCCTGCTGTTTGA
GCATGTGCAGAGAGCCAGAAACGGCGGCCTCAGACCTCTGGAAGTTCGGAGAGCAC
CTGTGCCTGATGGCGCCCCTTCTAGATATCCTCCAGTGGCCACCTGTCACAGACCCG
GCAGCACATCTTTTTGCCCTGGCGAGTCTAGCCGGATGGGCATCAAGGACAGAATCA
GAATGGGCAGCAGCCAGCGGAGAACAGGCCCTTCTAAACAGCATCTGGCCCCTCCA
ACCATGCCTACAAGCCCTAGCTCTGAGCAAGTGGGCGAAGCCACCTCTCCTACCAAG
GTGCAGAAGTCCTGGTCCTTCAACGACCGGACCAGATTCAGAGCCAGCCTGAGACTG
AAGCCCAGAACCTCTGCCGAGGATGCCCCTTCTGAAGAGGTGGCCGAAGAGAAGTC
CTACCAGTGCGAGCTGACCGTGGACGACATCATGCCAGCCGTGAAAACCGTGATAC
GGTCTATCCGGATCCTGAAGTTCCTGGTGGCCAAGCGGAAGTTCAAAGAGACACTGC
GGCCCTACGACGTGAAGGACGTGATCGAGCAGTATTCTGCCGGCCACCTGGACATGC
TGGGCAGAATCAAGAGCCTGCAGACCAGAGTGGACCAGATCGTTGGAAGAGGCCCA
GGCGACAGAAAGGCCAGAGAGAAGGGCGATAAGGGCCCATCTGATGCCGAGGTTGT
CGACGAGATATCAATGATGGGCAGAGTGGTCAAGGTGGAAAAACAGGTGCAGAGCA
TCGAGCACAAGCTGGACCTGCTGCTGGGATTCTACAGCCGGTGTCTGAGAAGCGGCA
CATCTGCATCTCTGGGCGCTGTGCAGGTCCCACTGTTCGACCCTGATATCACCAGCG
ACTATCACAGCCCCGTGGACCACGAGGACATCTCCGTTTCTGCTCAGACCCTGAGCA
TCAGCAGATCCGTGTCCACCAACATGGACGGATCCCGGGCTGACTACAAAGACCAT
GACGGTGATTATAAAGATCATGACATCGACTACAAGGATGACGATGACAAGTAATA
AGAGCTCGCTGATCAGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAATA
AAATGAGGAAATTGCATCGCATTGTCTGAGTAGGTGTCATTCTATTCTGGGGGGTGG
GGTGGGGCAGGACAGCAAGGGGGAGGATTGGGAAGACAATAGCAGGCATGCTGGG
GATGCGGTGGGCTCTATGttaattCGGACCGCTAGGAACCCCTAGTGATGGAGTTGGCCA
CTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGCTGCCTGCAG
G

TABLE 12
Construct Components (SEQ ID NO: 32)

| Component (5′ to | Position |
|---|---|
| 3′ orientation) | (nucleotides) |
| 5′ ITR | 12-130 |
| STRIP2 promoter | 145-1441 |
| 5′ UTR | 1442-1731 |
| KCNQ4 | 1749-3833 |
| coding region | |
| 3x Flag | 3846-3914 |
| polyA | 3939-4162 |
| 3′ ITR | 4178-4307 |

In some aspects, the STRIP2 promoter comprises a nucleic acid sequence that comprises at least 80%, at least 85%, at least 90%, at least 95%, at least 96%, at least 97%, at least 98% at least 99%, or 100% identity to nucleotides 145-1441 of SEQ ID NO: 32.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 33. In some aspects, the construct comprises the nucleic acid sequence of SEQ ID NO: 33.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4293 of SEQ ID NO: 33. In some aspects, the construct comprises nucleotides 12-4293 of SEQ ID NO: 33.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 33. In some aspects, the rAAVAnc80 particle comprises the nucleic acid sequence of SEQ ID NO: 33.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a AQP11 promoter comprising the nucleic acid sequence of SEQ ID NO: 10, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a AQP11 promoter comprising the nucleic acid sequence of SEQ ID NO: 10, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 33)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTAGGCATGAGCCACTATGCCCAAATGA
GAAATAATTTTGTATGAAAAATAATCTTGTATGGTAAATTTAGACCAAGAATAAAAT
GAGTGGTTGTATAAGAAAGAAAGATGTTCAGAACAAACCAAAAAGTCCAAGCATGT
CACGAATGGTCTGTGTAAGTCATAATAAAAGGATTTATCTAAAAAAACCAAAAACTT
TTATATGATCAAGTCGTCTATAATTAAAGGAAAATTATAATGGGTTTTTCTAGACATT
GGGTGTGATGTAATGAAACGTACACACTAAAGAATTCATTACAAGGCTTTCATGTTT
TGTTTTTTGTTTGTTTGACTGGTTTGTTGTTGTTGTTGTTGTTGTTGTTGTTGTTTTTGA
GACGGAGTTTCGCTCTTGTTGCCCAGGCTGGAGTACAATGGCGCGATCTAGGCTCAC
CACAACCTCTGCCTCCCGGGTTCAAGCGATTCTCCTGCATCATCCTCCCGAGTAGCTG
GGATGACAGGCATGCGCCACCATGCCCGGCTAATTTTGTATTTTTAGTAGAGACGGG
GGTTTCTCATGTTGGTCAGGCTAGTCTCGAACTCCCGACCTCCGGTGATCCGCCCGCC
TCGGCCTCCCAAAGTGCTGGGATTATAGGCGTGAGCCACCGCGCCCAGCCGCGCCCG
GTTTTTGTTGTTGTTTTTGTTTCTAAAAACAGCGTCTCGCTCTGTGGCCCAGGCAGGG
GTGCAGTGGCGCGATCTCAGCTCACTGCAGCCTGGAACTCCTGGGGTCAAGCGGTCT
TCCCACCTAAGCCTCTCCGTGCTGGGACTCCGGACGCGCTCCACCTCACGCAGCCGT
ATTCCTGCTTTCAAAGCAGATGGAAGAGGTGCGCCAGGACCCCCAGTTCTTGGAAAC
AGACCTCTCCAGTTACCTGTTGTTTCCTCTTCACGAAGAGTGCATGTAACAGTAAGA
CACAACTGTTTCATATTATACGTAAAGAGTTCATGCCAAAGGTTATAGACAGTCACA
TGCTAAAACTAGGCTACACTTTGAAGAATCACCGCTCAAGTTCTGGAAAAAAGAGGT
GACTGTTGAACAACACTGTGAGGGTAATCGATGCCACTGAAATATACACTTAAATTG
ATTAAAGTGGCGAATTTTATCTGGCATATATTACCACCATTTTTAGAAATGTTTTTTG
GCAGGTGAAGAAAAGCAAGGCTCCAGGAGGCCCTGCGCACCGGTCTACGCCCACTA
ACTCACCCGCCCCCTGCGCCGCGTCTCCCCTCTCAATTTCAGTCGCCCATTGATCGCC
GGTGGCAGGTGGAAAGGCGAGCGGCATGGAGCGCGTAATAAGAGAGTTGGAGTCG
GAAAGAGCAGCCCCAGTCGCCGGGGAAGCGGGAGGTCAGTGCGGGCTCCGGCGGCC
CCCAGGCTCCGAGCGCCCGCCCGCGGCCCCGGCCCGGCCCCTAGCCCCCGCCGCCCG
CGCCCGCCCCGGGTCGCCCCTCTGGCCCCGGGTCCGAGCCATGCGTCTCTGAGCGCC
CCGAGCGCGCCCCCGCCCCGGACCGTGCCCGGGCCCCGGCGCCCCCAGCCCGGCGC
CGCCCACCGGTCGCTAGCCACCATGGCTGAAGCCCCTCCTAGAAGGCTTGGACTGGG
ACCTCCTCCTGGGGATGCTCCTAGAGCTGAACTGGTGGCTCTGACAGCCGTGCAGTC
TGAACAAGGCGAAGCTGGTGGCGGCGGATCTCCACGTAGACTTGGACTGCTGGGAA
GCCCTCTTCCTCCTGGTGCTCCACTTCCTGGACCTGGCAGTGGATCTGGATCTGCCTG
TGGCCAGAGAAGCTCTGCCGCTCACAAGAGATACCGGCGGCTGCAGAACTGGGTGT
ACAACGTGCTGGAAAGACCCAGAGGCTGGGCCTTCGTGTACCACGTGTTCATCTTTC
TGCTGGTGTTCAGCTGCCTGGTGCTGTCCGTGCTGAGCACCATCCAAGAACATCAAG
AGCTGGCTAACGAGTGCCTGTTAATACTGGAGTTTGTGATGATTGTGGTGTTCGGCC
TCGAGTACATCGTCCGCGTTTGGAGCGCCGGCTGCTGCTGCAGATATAGAGGTTGGC
AAGGCAGATTCCGCTTCGCCAGAAAGCCCTTCTGCGTGATCGACTTCATCGTGTTCG
TGGCCAGCGTGGCCGTGATTGCTGCTGGCACACAGGGCAACATCTTCGCCACAAGCG
CCCTGCGGAGCATGCGGTTTCTGCAGATCCTGAGAATGGTCCGAATGGACAGAAGA
GGCGGCACCTGGAAGCTGCTGGGCTCTGTGGTGTACGCCCACAGCAAAGAGCTGAT
CACCGCCTGGTACATCGGATTTCTGGTGCTGATCTTCGCCTCCTTCCTGGTGTACCTG
GCCGAGAAGGACGCCAACAGCGACTTTAGCAGCTACGCCGACTCTCTTTGGTGGGGC
ACCATCACACTGACCACCATCGGCTACGGCGACAAGACCCCTCACACATGGCTGGG
AAGAGTGCTGGCCGCTGGATTTGCTCTGCTGGGCATCAGCTTTTTCGCCCTGCCTGCC
GGAATCCTCGGATCTGGCTTTGCCCTGAAGGTGCAAGAGCAGCACCGGCAGAAGCA
CTTCGAGAAGAGAAGAATGCCTGCCGCCAACCTGATTCAGGCCGCTTGGAGACTGTA
CAGCACCGACATGAGCAGAGCCTACCTGACCGCCACGTGGTATTATTACGACTCGAT
CCTGCCTAGCTTCCGCGAACTGGCCCTGCTGTTTGAGCATGTGCAGAGAGCCAGAAA
CGGCGGCCTCAGACCTCTGGAAGTTCGGAGAGCACCTGTGCCTGATGGCGCCCCTTC
TAGATATCCTCCAGTGGCCACCTGTCACAGACCCGGCAGCACATCTTTTTGCCCTGG
CGAGTCTAGCCGGATGGGCATCAAGGACAGAATCAGAATGGGCAGCAGCCAGCGGA
GAACAGGCCCTTCTAAACAGCATCTGGCCCCTCCAACCATGCCTACAAGCCCTAGCT
CTGAGCAAGTGGGCGAAGCCACCTCTCCTACCAAGGTGCAGAAGTCCTGGTCCTTCA
ACGACCGGACCAGATTCAGAGCCAGCCTGAGACTGAAGCCCAGAACCTCTGCCGAG
GATGCCCCTTCTGAAGAGGTGGCCGAAGAGAAGTCCTACCAGTGCGAGCTGACCGT
GGACGACATCATGCCAGCCGTGAAAACCGTGATACGGTCTATCCGGATCCTGAAGTT
CCTGGTGGCCAAGCGGAAGTTCAAAGAGACACTGCGGCCCTACGACGTGAAGGACG
TGATCGAGCAGTATTCTGCCGGCCACCTGGACATGCTGGGCAGAATCAAGAGCCTGC
AGACCAGAGTGGACCAGATCGTTGGAAGAGGCCCAGGCGACAGAAAGGCCAGAGA
GAAGGGCGATAAGGGCCCATCTGATGCCGAGGTTGTCGACGAGATATCAATGATGG
GCAGAGTGGTCAAGGTGGAAAAACAGGTGCAGAGCATCGAGCACAAGCTGGACCTG
CTGCTGGGATTCTACAGCCGGTGTCTGAGAAGCGGCACATCTGCATCTCTGGGCGCT
GTGCAGGTCCCACTGTTCGACCCTGATATCACCAGCGACTATCACAGCCCCGTGGAC
CACGAGGACATCTCCGTTTCTGCTCAGACCCTGAGCATCAGCAGATCCGTGTCCACC
AACATGGACGGATCCCGGGCTGACTACAAAGACCATGACGGTGATTATAAAGATCA
TGACATCGACTACAAGGATGACGATGACAAGTAATAAGAGCTCGCTGATCAGCCTC
GACTGTGCCTTCTAGTTGCCAGCCATCTGTTGTTTGCCCCTCCCCCGTGCCTTCCTTG
ACCCTGGAAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGACAGCAAG
GGGGAGGATTGGGAAGACAATAGCAGGCATGCTGGGGATGCGGTGGGCTCTATGttaa
ttCGGACCGCTAGGAACCCCTAGTGATGGAGTTGGCCACTCCCTCTCTGCGCGCTCGC
TCGCTCACTGAGGCCGGGCGACCAAAGGTCGCCCGACGCCCGGGCTTTGCCCGGGC
GGCCTCAGTGAGCGAGCGAGCGCGCAGCTGCCTGCAGG

TABLE 13
Construct Components (SEQ ID NO: 33)

| Component (5′ to | Position |
|---|---|
| 3′ orientation) | (nucleotides) |
| 5′ ITR | 12-130 |
| AQP11 promoter | 145-1427 |
| 5′ UTR | 1428-1717 |
| KCNQ4 | 1735-3819 |
| coding region | |
| 3x Flag | 3832-3900 |
| polyA | 3925-4148 |
| 3′ ITR | 4164-4293 |

In some aspects, the AQP11 promoter comprises a nucleic acid sequence that comprises at least 80%, at least 85%, at least 90%, at least 95%, at least 96%, at least 97%, at least 98% at least 99%, or 100% identity to nucleotides 145-1427 of SEQ ID NO: 33.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 34. In some aspects, the construct comprises the nucleic acid sequence of SEQ ID NO: 34.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4565 of SEQ ID NO: 34. In some aspects, the construct comprises nucleotides 12-4565 of SEQ ID NO: 34.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 34. In some aspects, the rAAVAnc80 particle comprises the nucleic acid sequence of SEQ ID NO: 34.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a KCNQ4 promoter comprising the nucleic acid sequence of SEQ ID NO: 11, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a KCNQ4 promoter comprising the nucleic acid sequence of SEQ ID NO: 11, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 34)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTAGGGCCCATCCTGGTGTAAACAAACC
CTTTGGCGCAGCCCAAGAGAGCCCCTATCTAATACCCAGCACGATCCCCTTACATCC
GGAGCACTCTTTAAACATTTTTCCTAGCTGATCTTCACAGTGACCCTGCAGGGGAGA
CAGGAAGAGGTATCATGATCCCTGTGTTAGCGTGGGAGGGCTAGTGAAGTGCAGTG
ACTTGCCCAAGGTCACTCCATGAATTGAGGGTGGAATTGAAACCAAAACACTGATCT
CCTGACCCCCTGTGCATACACAGTTGCTCTTGGAGATTGGAGACCCCTGGAATCTGG
AGCAGACAATCTGGCTGGCTTCCTTGCAGCTCAGGTCTGCGGAGGCCACAAGGGGG
CAGCATGCAGCCCTCACCTGTGTCTCTGGGACCTTTGAAGGGAGGGTCCTCCCTAGG
ATAACAGTGAGAGCTGGAAACTCTACCCTCTCCAGAGTATTGCCTCAAGATCCCTGA
ACTTAGCTCCATGTTTTCAGAATGTGCTAGCTACAATTCCTGAAATGCCCTTTTACTT
CCCTTTTCACTTATTGAGCTCCTATACATCCATCAAGGCCCAATTTAAATGGCCCTTT
CAGCAGCTATTTCTTTGGCACCTTCTGTGTGTCAGACGTTGTTTTAAACATTGTGAAT
ACAGCTTAAAACAAGTCTGACGGGTGGAAAGGAAACTGCTGAGGGTGGGGTCAGGG
GAACAGGTGGGAGAGGGACCAGTCCCCTCCAGCAGAGGGGCCAATTGAGGGAGCCT
GAGACAGCTGTTTGCTCAGAAAAGTGTCTTAGTCACTAAAGGTTGTGGTGGGGAAAG
TCCGTCCTCCCAGTCATGTCCTGGGAATCCGGATGGCGCAGGAAGGCCACCCGGTGA
CCCTAAGAGTGGCCACCTGTCCTCTCTGAACTGGACTTTCTCTTCTGGCCCTTCCCCT
CCCTCCCTCCCTCACTGGCGCTCAGCAGATCAATGCTGCCTTTGCTGACAGCTGAGA
ATCGAGCTCGCCTTCCCGCCCCTTCCCCCGCCCCTCCCGCTCGGCTTCGTCCCTCGAG
ATCCTCCCGGAGGAACCGGGAAGAGTTTGCTGCGGAAGGCTCACCCTGGGGCAGGG
CCTGCGGAGGGAGCGGCTGGTGTGGCCGCAGCTTTCCGTGGAGGAAGAGGGAAAGA
GGATCGGGAAACCCAAGTTACCAACCCTGTGCAGGGGAGATGGAGGTCGGGGACTA
AGAAAAACTGCTGCCCACCCAGCCACACACAGCACTGGGCACACTTTAAGCACCCG
CACCAGGCACACAGTGCTCGACCCCAACGGACACACCTCATCCTGCCGCCCGCGGCC
ACAACTCCACATTCACTTGCACGCGTCCGGCTTCCCGGCCCCGCGCGCTGCCCCCGC
CACGCGGTTCGGCCCAGGCACCAACTCGGCCGCCCGTGCGCCCTGCCCCGCCGCCTG
CTCCGCGCGTTCCCTCCCTCCGCCTCGCCTCGCTTGCTCGCTCGCTCCCTCCCGATTTG
GGAAGGCGGCCGCGGGGGGGGCGGGGGAGGGGCGGGGCGGGGGAGGGTCGCCGGT
GGCAGGTGGAAAGGCGAGCGGCATGGAGCGCGTAATAAGAGAGTTGGAGTCGGAA
AGAGCAGCCCCAGTCGCCGGGGAAGCGGGAGGTCAGTGCGGGCTCCGGCGGCCCCC
AGGCTCCGAGCGCCCGCCCGCGGCCCCGGCCCGGCCCCTAGCCCCCGCCGCCCGCGC
CCGCCCCGGGTCGCCCCTCTGGCCCCGGGTCCGAGCCATGCGTCTCTGAGCGCCCCG
AGCGCGCCCCCGCCCCGGACCGTGCCCGGGCCCCGGCGCCCCCAGCCCGGCGCCGC
CCACCGGTCGCTAGCCACCATGGCTGAAGCCCCTCCTAGAAGGCTTGGACTGGGACC
TCCTCCTGGGGATGCTCCTAGAGCTGAACTGGTGGCTCTGACAGCCGTGCAGTCTGA
ACAAGGCGAAGCTGGTGGCGGCGGATCTCCACGTAGACTTGGACTGCTGGGAAGCC
CTCTTCCTCCTGGTGCTCCACTTCCTGGACCTGGCAGTGGATCTGGATCTGCCTGTGG
CCAGAGAAGCTCTGCCGCTCACAAGAGATACCGGCGGCTGCAGAACTGGGTGTACA
ACGTGCTGGAAAGACCCAGAGGCTGGGCCTTCGTGTACCACGTGTTCATCTTTCTGC
TGGTGTTCAGCTGCCTGGTGCTGTCCGTGCTGAGCACCATCCAAGAACATCAAGAGC
TGGCTAACGAGTGCCTGTTAATACTGGAGTTTGTGATGATTGTGGTGTTCGGCCTCG
AGTACATCGTCCGCGTTTGGAGCGCCGGCTGCTGCTGCAGATATAGAGGTTGGCAAG
GCAGATTCCGCTTCGCCAGAAAGCCCTTCTGCGTGATCGACTTCATCGTGTTCGTGGC
CAGCGTGGCCGTGATTGCTGCTGGCACACAGGGCAACATCTTCGCCACAAGCGCCCT
GCGGAGCATGCGGTTTCTGCAGATCCTGAGAATGGTCCGAATGGACAGAAGAGGCG
GCACCTGGAAGCTGCTGGGCTCTGTGGTGTACGCCCACAGCAAAGAGCTGATCACCG
CCTGGTACATCGGATTTCTGGTGCTGATCTTCGCCTCCTTCCTGGTGTACCTGGCCGA
GAAGGACGCCAACAGCGACTTTAGCAGCTACGCCGACTCTCTTTGGTGGGGCACCAT
CACACTGACCACCATCGGCTACGGCGACAAGACCCCTCACACATGGCTGGGAAGAG
TGCTGGCCGCTGGATTTGCTCTGCTGGGCATCAGCTTTTTCGCCCTGCCTGCCGGAAT
CCTCGGATCTGGCTTTGCCCTGAAGGTGCAAGAGCAGCACCGGCAGAAGCACTTCGA
GAAGAGAAGAATGCCTGCCGCCAACCTGATTCAGGCCGCTTGGAGACTGTACAGCA
CCGACATGAGCAGAGCCTACCTGACCGCCACGTGGTATTATTACGACTCGATCCTGC
CTAGCTTCCGCGAACTGGCCCTGCTGTTTGAGCATGTGCAGAGAGCCAGAAACGGCG
GCCTCAGACCTCTGGAAGTTCGGAGAGCACCTGTGCCTGATGGCGCCCCTTCTAGAT
ATCCTCCAGTGGCCACCTGTCACAGACCCGGCAGCACATCTTTTTGCCCTGGCGAGT
CTAGCCGGATGGGCATCAAGGACAGAATCAGAATGGGCAGCAGCCAGCGGAGAAC
AGGCCCTTCTAAACAGCATCTGGCCCCTCCAACCATGCCTACAAGCCCTAGCTCTGA
GCAAGTGGGCGAAGCCACCTCTCCTACCAAGGTGCAGAAGTCCTGGTCCTTCAACGA
CCGGACCAGATTCAGAGCCAGCCTGAGACTGAAGCCCAGAACCTCTGCCGAGGATG
CCCCTTCTGAAGAGGTGGCCGAAGAGAAGTCCTACCAGTGCGAGCTGACCGTGGAC
GACATCATGCCAGCCGTGAAAACCGTGATACGGTCTATCCGGATCCTGAAGTTCCTG
GTGGCCAAGCGGAAGTTCAAAGAGACACTGCGGCCCTACGACGTGAAGGACGTGAT
CGAGCAGTATTCTGCCGGCCACCTGGACATGCTGGGCAGAATCAAGAGCCTGCAGA
CCAGAGTGGACCAGATCGTTGGAAGAGGCCCAGGCGACAGAAAGGCCAGAGAGAA
GGGCGATAAGGGCCCATCTGATGCCGAGGTTGTCGACGAGATATCAATGATGGGCA
GAGTGGTCAAGGTGGAAAAACAGGTGCAGAGCATCGAGCACAAGCTGGACCTGCTG
CTGGGATTCTACAGCCGGTGTCTGAGAAGCGGCACATCTGCATCTCTGGGCGCTGTG
CAGGTCCCACTGTTCGACCCTGATATCACCAGCGACTATCACAGCCCCGTGGACCAC
GAGGACATCTCCGTTTCTGCTCAGACCCTGAGCATCAGCAGATCCGTGTCCACCAAC
ATGGACGGATCCCGGGCTGACTACAAAGACCATGACGGTGATTATAAAGATCATGA
CATCGACTACAAGGATGACGATGACAAGTAATAAGAGCTCGCTGATCAGCCTCGAC
TGTGCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTATGttaattCG
GACCGCTAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGGC
CTCAGTGAGCGAGCGAGCGCGCAGCTGCCTGCAGG

TABLE 14
Construct Components (SEQ ID NO: 34)

| Component (5′ to | Position |
|---|---|
| 3′ orientation) | nucleotides |
| 5′ ITR | 12-130 |
| KCNQ4 promoter | 145-1699 |
| 5′ UTR | 1700-1989 |
| KCNQ4 | 2007-4091 |
| coding region | |
| 3x Flag | 4104-4172 |
| polyA | 4197-4420 |
| 3′ ITR | 4436-4565 |

In some aspects, the KCNQ4 promoter comprises a nucleic acid sequence that comprises at least 80%, at least 85%, at least 90%, at least 95%, at least 96%, at least 97%, at least 98% at least 99%, or 100% identity to nucleotides 145-1699 of SEQ ID NO: 34.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 35. In some aspects, the construct comprises the nucleic acid sequence of SEQ ID NO: 35.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4224 of SEQ ID NO: 35. In some aspects, the construct comprises nucleotides 12-4224 of SEQ ID NO: 35.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 35. In some aspects, the construct rAAVAnc80 particle the nucleic acid sequence of SEQ ID NO: 35.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a LBH promoter comprising the nucleic acid sequence of SEQ ID NO: 12, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a LBH promoter comprising the nucleic acid sequence of SEQ ID NO: 12, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 35)
CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCGCACGCGTGTGAAT
TCGATGATGTGCTTGTGTGGACATGTGGAGGTCTCAAGAACAAAAGAAGA
GCTGGGCCTGGCACACAGTGGGTGCTAATGCCTGTTAGAATTGTTGTTGA
GAGGGCAGGAGGGTGTAACATGGACCCAGCTATCTGATCCTGAGGCTGGG
CGCCATGTGGGTGTGAAGTACACCAGGGGCTCCAACCAGCAAGTGCTAGC
TCAGGTTACAGTCAGCTGCCCCTGGAGGAAGCTAGCAGACATCCTGTGTA
CTTGAAAAGAAAACTGAAAGTGCTATCTGCATCCTGGTGATAGTAACCTC
TCTTTTCTGGCTGTTGAAGTGCATTCCTGTGCGGATGTGGAAAGAGAGAA
AGCAAGATACAGCCAGGGCTAGGACAGGAATGTGAGTATTTCCTTAATTG
GACATGAGAGCCTTGAACTGATTCCAGTTGGAGTGTTTTCTTTTAGGGCC
TGGACCCTAAAGATTTCATACAGTTTTCTTTGTCAGAAAAATCCCTTTGG
TTCAAAGGCCCCTCGATAGAAATAAAGAAAAAGCCAGGGCTGAATTTCTT
TGATATGTGGGAAGGCAAGAGTTTATGAGCTGCCAGATCTCAGGCTTCTT
TTGGGGTGGAGGATTTTGTCTGGTGGGTTCGGGTTGCTTTGTGTTGTTGA
CTGCTAATTCACTGATGACCAAGTTTCTCAAATACCTTAAAAACAAGCCC
TACGTCTGCTCAGTGCTTTCCAATTTACCAAGTGTTTTCATAACATTTCT
TATGTACGCAAATGAGTTTCACCGAAAAATTGGCTAGAAACTTCCCTTCT
CCTACTCACGTTCATAGTGTAGCTGTGAAAACAAACAAAACCACAGAGGC
ATGGTAAGTGTGGTATGGTGGGGAAAACAAAGCCATTTTACAGGCGTGAT
TGAAGCGGAGGCCACAGAGCGGCAGCGCTGGGTCCCGAGTGAGACTCCCA
TCATGTGGCTCAATGGAAAAATCCTACCCAGGACGACACCACATCCTTGC
TCCCACAAATAAAACCTTCCACGGAACTCAGGGCTGCAGACGCAGAGCCG
AGCGCGCCCCCGAGCCGCCGCCCGCCGGAGCTGCGAGCGCTGAAGCCATT
CATGATTTTGGTGACGTTATTCCAGGAGTGGGCGAGGGAGGGCGGGGCCT
CTCGGGGCCAAGCCCCGCCCCCGCCCCTATAAATACGGCTTCCCGGGCTC
TTTGTGGGCGCCGGTGGCAGGTGGAAAGGCGAGCGGCATGGAGCGCGTAA
TAAGAGAGTTGGAGTCGGAAAGAGCAGCCCCAGTCGCCGGGGAAGCGGGA
GGTCAGTGCGGGCTCCGGCGGCCCCCAGGCTCCGAGCGCCCGCCCGCGGC
CCCGGCCCGGCCCCTAGCCCCCGCCGCCCGCGCCCGCCCCGGGTCGCCCC
TCTGGCCCCGGGTCCGAGCCATGCGTCTCTGAGCGCCCCGAGCGCGCCCC
CGCCCCGGACCGTGCCCGGGCCCCGGCGCCCCCAGCCCGGCGCCGCCCAC
CGGTCGCTAGCCACCATGGCTGAAGCCCCTCCTAGAAGGCTTGGACTGGG
ACCTCCTCCTGGGGATGCTCCTAGAGCTGAACTGGTGGCTCTGACAGCCG
TGCAGTCTGAACAAGGCGAAGCTGGTGGCGGCGGATCTCCACGTAGACTT
GGACTGCTGGGAAGCCCTCTTCCTCCTGGTGCTCCACTTCCTGGACCTGG
CAGTGGATCTGGATCTGCCTGTGGCCAGAGAAGCTCTGCCGCTCACAAGA
GATACCGGCGGCTGCAGAACTGGGTGTACAACGTGCTGGAAAGACCCAGA
GGCTGGGCCTTCGTGTACCACGTGTTCATCTTTCTGCTGGTGTTCAGCTG
CCTGGTGCTGTCCGTGCTGAGCACCATCCAAGAACATCAAGAGCTGGCTA
ACGAGTGCCTGTTAATACTGGAGTTTGTGATGATTGTGGTGTTCGGCCTC
GAGTACATCGTCCGCGTTTGGAGCGCCGGCTGCTGCTGCAGATATAGAGG
TTGGCAAGGCAGATTCCGCTTCGCCAGAAAGCCCTTCTGCGTGATCGACT
TCATCGTGTTCGTGGCCAGCGTGGCCGTGATTGCTGCTGGCACACAGGGC
AACATCTTCGCCACAAGCGCCCTGCGGAGCATGCGGTTTCTGCAGATCCT
GAGAATGGTCCGAATGGACAGAAGAGGCGGCACCTGGAAGCTGCTGGGCT
CTGTGGTGTACGCCCACAGCAAAGAGCTGATCACCGCCTGGTACATCGGA
TTTCTGGTGCTGATCTTCGCCTCCTTCCTGGTGTACCTGGCCGAGAAGGA
CGCCAACAGCGACTTTAGCAGCTACGCCGACTCTCTTTGGTGGGGCACCA
TCACACTGACCACCATCGGCTACGGCGACAAGACCCCTCACACATGGCTG
GGAAGAGTGCTGGCCGCTGGATTTGCTCTGCTGGGCATCAGCTTTTTCGC
CCTGCCTGCCGGAATCCTCGGATCTGGCTTTGCCCTGAAGGTGCAAGAGC
AGCACCGGCAGAAGCACTTCGAGAAGAGAAGAATGCCTGCCGCCAACCTG
ATTCAGGCCGCTTGGAGACTGTACAGCACCGACATGAGCAGAGCCTACCT
GACCGCCACGTGGTATTATTACGACTCGATCCTGCCTAGCTTCCGCGAAC
TGGCCCTGCTGTTTGAGCATGTGCAGAGAGCCAGAAACGGCGGCCTCAGA
CCTCTGGAAGTTCGGAGAGCACCTGTGCCTGATGGCGCCCCTTCTAGATA
TCCTCCAGTGGCCACCTGTCACAGACCCGGCAGCACATCTTTTTGCCCTG
GCGAGTCTAGCCGGATGGGCATCAAGGACAGAATCAGAATGGGCAGCAGC
CAGCGGAGAACAGGCCCTTCTAAACAGCATCTGGCCCCTCCAACCATGCC
TACAAGCCCTAGCTCTGAGCAAGTGGGCGAAGCCACCTCTCCTACCAAGG
TGCAGAAGTCCTGGTCCTTCAACGACCGGACCAGATTCAGAGCCAGCCTG
AGACTGAAGCCCAGAACCTCTGCCGAGGATGCCCCTTCTGAAGAGGTGGC
CGAAGAGAAGTCCTACCAGTGCGAGCTGACCGTGGACGACATCATGCCAG
CCGTGAAAACCGTGATACGGTCTATCCGGATCCTGAAGTTCCTGGTGGCC
AAGCGGAAGTTCAAAGAGACACTGCGGCCCTACGACGTGAAGGACGTGAT
CGAGCAGTATTCTGCCGGCCACCTGGACATGCTGGGCAGAATCAAGAGCC
TGCAGACCAGAGTGGACCAGATCGTTGGAAGAGGCCCAGGCGACAGAAAG
GCCAGAGAGAAGGGCGATAAGGGCCCATCTGATGCCGAGGTTGTCGACGA
GATATCAATGATGGGCAGAGTGGTCAAGGTGGAAAAACAGGTGCAGAGCA
TCGAGCACAAGCTGGACCTGCTGCTGGGATTCTACAGCCGGTGTCTGAGA
AGCGGCACATCTGCATCTCTGGGCGCTGTGCAGGTCCCACTGTTCGACCC
TGATATCACCAGCGACTATCACAGCCCCGTGGACCACGAGGACATCTCCG
TTTCTGCTCAGACCCTGAGCATCAGCAGATCCGTGTCCACCAACATGGAC
GGATCCCGGGCTGACTACAAAGACCATGACGGTGATTATAAAGATCATGA
CATCGACTACAAGGATGACGATGACAAGTAATAAGAGCTCGCTGATCAGC
CTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAA
AATGAGGAAATTGCATCGCATTGTCTGAGTAGGTGTCATTCTATTCTGGG
GGGTGGGGGGGGCAGGACAGCAAGGGGGAGGATTGGGAAGACAATAGCAG
GCATGCTGGGGATGCGGTGGGCTCTATGttaattCGGACCGCTAGGAACC
CCTAGTGATGGAGTTGGCCACTCCCTCTCTGCGCGCTCGCTCGCTCACTG
AGGCCGGGCGACCAAAGGTCGCCCGACGCCCGGGCTTTGCCCGGGCGGCC
TCAGTGAGCGAGCGAGCGCGCAGCTGCCTGCAGG

TABLE 15
Construct Components (SEQ ID NO: 35)

| Component (5′ to | Position |
|---|---|
| 3′ orientation) | nucleotides |
| 5′ ITR | 12-130 |
| LBH promoter | 145-1358 |
| 5′ UTR | 1359-1648 |
| KCNQ4 | 1666-3750 |
| coding region | |
| 3x Flag | 3763-3831 |
| polyA | 3856-4079 |
| 3′ ITR | 4095-4224 |

In some aspects, the LBH promoter comprises a nucleic acid sequence that comprises at least 80%, at least 85%, at least 90%, at least 95%, at least 96%, at least 97%, at least 98% at least 99%, or 100% identity to nucleotides 145-1358 of SEQ ID NO: 35.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 36. In some aspects, the construct comprises the nucleic acid sequence of SEQ ID NO: 36.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4140 of SEQ ID NO: 36. In some aspects, the construct comprises nucleotides 12-4140 of SEQ ID NO: 36.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 36. In some aspects, the rAAVAnc80 particle comprises the nucleic acid sequence of SEQ ID NO: 36.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a STRC promoter comprising the nucleic acid sequence of SEQ ID NO: 13, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a STRC promoter comprising the nucleic acid sequence of SEQ ID NO: 13, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 36)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTCACTGCCATCTCAACATGTGGTTTCT
AGGTTGCCTCAACAGGGAAGAGATTGTTGGAGGCTCATTTGCTAGCTCTTAAATTCT
TTGGTCAACCACAGTCCATTGAACAGAACTAATCACCTGACTGCAAGAGATCTGGGA
AATGTGAGAAACACCTAGATATCTAGTAAGCAATAAATATTTCAGTTACCAAAGCCA
AACCAAAAAAAGAGAAAAATAATTGTACTTTACAAAGGGAGGCATCTGGGTCCTGT
GGGAGTTTTGGGGAGTGAGGATGTTTCAGAGTTCTCAACTCCTGTGGCTATCCATTTC
ATTTTAGCAGGACATATGATTAATTTCTTGTTCTGGACCTTTGTAATTTAAAGTCTGA
ATCCTTAGCGGCAAGAGAATTGCTTAATCAATGGCTTACAACAGCAGAACGTGGACT
GCCAGGAAAATTTCCATCCTGAGTTAAGAAAGAAGGATAATTTATTATAAGAGGGTT
GTTACAGAATGAAGGGCAGAAATTCAGAAGGATTACAGGATGGGCTGGAACCACAA
AGCACTGTCTGCTTTTTAGACTAGGTGTGGTATCCTTGATGGGCAAAGGGAATATTG
GTAAAATTATTTGTGACCTGGGTTAAGTCATTCCATTTCTCTGGGCTTCAATTCCCCT
GTCTATAAAATGTTTGAGGGAGAGAATGGGGAAGGGTTCTAGGGAAAGAAGGACAG
AATAAAAGTTTGGGTATATGAATTACTATTTAGAGTTGGTATAAAGTGAAGGCCTTT
GGGGAGATATACCCTGACCAGACCAGATTATTTTGAATGAAATCTCTTTCTCTGTTCC
ATGAGCAGTTCTGTGTGTAGGGAGAACATTTGAATGGCCTAATGAGCAAATCACATT
TCTCTGGGTCTGTTTCCTTATCCATAAGTTCTGCATCACTGGCTCCTAACTCAAGCAA
TCTCCTTGGGTTTCTCTGAGGGGCCCCTGGGATCCCCTATCATTAGTCCCTCTCACAG
AAGCATACCCTTCTCCAGAGCTAAAGGATCAGATATTCAGCGGCTCAGGTAACAAAC
CTGCTGTCAGGTTACACATATTGTTTCCTGAAAGACCACACTACAGTGTCAGTGGAG
CCTCAGGTTGCCTGCAGTCGCCGGTGGCAGGTGGAAAGGCGAGCGGCATGGAGCGC
GTAATAAGAGAGTTGGAGTCGGAAAGAGCAGCCCCAGTCGCCGGGGAAGCGGGAG
GTCAGTGCGGGCTCCGGCGGCCCCCAGGCTCCGAGCGCCCGCCCGCGGCCCCGGCCC
GGCCCCTAGCCCCCGCCGCCCGCGCCCGCCCCGGGTCGCCCCTCTGGCCCCGGGTCC
GAGCCATGCGTCTCTGAGCGCCCCGAGCGCGCCCCCGCCCCGGACCGTGCCCGGGCC
CCGGCGCCCCCAGCCCGGCGCCGCCCACCGGTCGCTAGCCACCATGGCTGAAGCCCC
TCCTAGAAGGCTTGGACTGGGACCTCCTCCTGGGGATGCTCCTAGAGCTGAACTGGT
GGCTCTGACAGCCGTGCAGTCTGAACAAGGCGAAGCTGGTGGCGGCGGATCTCCAC
GTAGACTTGGACTGCTGGGAAGCCCTCTTCCTCCTGGTGCTCCACTTCCTGGACCTGG
CAGTGGATCTGGATCTGCCTGTGGCCAGAGAAGCTCTGCCGCTCACAAGAGATACCG
GCGGCTGCAGAACTGGGTGTACAACGTGCTGGAAAGACCCAGAGGCTGGGCCTTCG
TGTACCACGTGTTCATCTTTCTGCTGGTGTTCAGCTGCCTGGTGCTGTCCGTGCTGAG
CACCATCCAAGAACATCAAGAGCTGGCTAACGAGTGCCTGTTAATACTGGAGTTTGT
GATGATTGTGGTGTTCGGCCTCGAGTACATCGTCCGCGTTTGGAGCGCCGGCTGCTG
CTGCAGATATAGAGGTTGGCAAGGCAGATTCCGCTTCGCCAGAAAGCCCTTCTGCGT
GATCGACTTCATCGTGTTCGTGGCCAGCGTGGCCGTGATTGCTGCTGGCACACAGGG
CAACATCTTCGCCACAAGCGCCCTGCGGAGCATGCGGTTTCTGCAGATCCTGAGAAT
GGTCCGAATGGACAGAAGAGGCGGCACCTGGAAGCTGCTGGGCTCTGTGGTGTACG
CCCACAGCAAAGAGCTGATCACCGCCTGGTACATCGGATTTCTGGTGCTGATCTTCG
CCTCCTTCCTGGTGTACCTGGCCGAGAAGGACGCCAACAGCGACTTTAGCAGCTACG
CCGACTCTCTTTGGTGGGGCACCATCACACTGACCACCATCGGCTACGGCGACAAGA
CCCCTCACACATGGCTGGGAAGAGTGCTGGCCGCTGGATTTGCTCTGCTGGGCATCA
GCTTTTTCGCCCTGCCTGCCGGAATCCTCGGATCTGGCTTTGCCCTGAAGGTGCAAGA
GCAGCACCGGCAGAAGCACTTCGAGAAGAGAAGAATGCCTGCCGCCAACCTGATTC
AGGCCGCTTGGAGACTGTACAGCACCGACATGAGCAGAGCCTACCTGACCGCCACG
TGGTATTATTACGACTCGATCCTGCCTAGCTTCCGCGAACTGGCCCTGCTGTTTGAGC
ATGTGCAGAGAGCCAGAAACGGCGGCCTCAGACCTCTGGAAGTTCGGAGAGCACCT
GTGCCTGATGGCGCCCCTTCTAGATATCCTCCAGTGGCCACCTGTCACAGACCCGGC
AGCACATCTTTTTGCCCTGGCGAGTCTAGCCGGATGGGCATCAAGGACAGAATCAGA
ATGGGCAGCAGCCAGCGGAGAACAGGCCCTTCTAAACAGCATCTGGCCCCTCCAAC
CATGCCTACAAGCCCTAGCTCTGAGCAAGTGGGCGAAGCCACCTCTCCTACCAAGGT
GCAGAAGTCCTGGTCCTTCAACGACCGGACCAGATTCAGAGCCAGCCTGAGACTGA
AGCCCAGAACCTCTGCCGAGGATGCCCCTTCTGAAGAGGTGGCCGAAGAGAAGTCC
TACCAGTGCGAGCTGACCGTGGACGACATCATGCCAGCCGTGAAAACCGTGATACG
GTCTATCCGGATCCTGAAGTTCCTGGTGGCCAAGCGGAAGTTCAAAGAGACACTGCG
GCCCTACGACGTGAAGGACGTGATCGAGCAGTATTCTGCCGGCCACCTGGACATGCT
GGGCAGAATCAAGAGCCTGCAGACCAGAGTGGACCAGATCGTTGGAAGAGGCCCAG
GCGACAGAAAGGCCAGAGAGAAGGGCGATAAGGGCCCATCTGATGCCGAGGTTGTC
GACGAGATATCAATGATGGGCAGAGTGGTCAAGGTGGAAAAACAGGTGCAGAGCAT
CGAGCACAAGCTGGACCTGCTGCTGGGATTCTACAGCCGGTGTCTGAGAAGCGGCA
CATCTGCATCTCTGGGCGCTGTGCAGGTCCCACTGTTCGACCCTGATATCACCAGCG
ACTATCACAGCCCCGTGGACCACGAGGACATCTCCGTTTCTGCTCAGACCCTGAGCA
TCAGCAGATCCGTGTCCACCAACATGGACGGATCCCGGGCTGACTACAAAGACCAT
GACGGTGATTATAAAGATCATGACATCGACTACAAGGATGACGATGACAAGTAATA
AGAGCTCGCTGATCAGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAATA
AAATGAGGAAATTGCATCGCATTGTCTGAGTAGGTGTCATTCTATTCTGGGGGGTGG
GGTGGGGCAGGACAGCAAGGGGGAGGATTGGGAAGACAATAGCAGGCATGCTGGG
GATGCGGTGGGCTCTATGttaattCGGACCGCTAGGAACCCCTAGTGATGGAGTTGGCCA
CTCCCTCTCTGCGCGCTCGCTCGCTCACTGAGGCCGGGCGACCAAAGGTCGCCCGAC
GCCCGGGCTTTGCCCGGGCGGCCTCAGTGAGCGAGCGAGCGCGCAGCTGCCTGCAG
G

TABLE 16
Construct Components (SEQ ID NO: 36)

| Component (5′ to | Position |
|---|---|
| 3′ orientation) | (nucleotides) |
| 5′ ITR | 12-130 |
| STRC promoter | 145-1274 |
| 5′ UTR | 1275-1564 |
| KCNQ4 | 1582-3666 |
| coding region | |
| 3x Flag | 3679-3747 |
| polyA | 3772-3995 |
| 3′ ITR | 4011-4140 |

In some aspects, the STRC promoter comprises a nucleic acid sequence that comprises at least 80%, at least 85%, at least 90%, at least 95%, at least 96%, at least 97%, at least 98% at least 99%, or 100% identity to nucleotides 145-1274 of SEQ ID NO: 36.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 37. In some aspects, the construct comprises the nucleic acid sequence of SEQ ID NO: 37.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4816 of SEQ ID NO: 37. In some aspects, the construct comprises nucleotides 12-4816 of SEQ ID NO: 37.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 37. In some aspects, the rAAVAnc80 particle comprises the nucleic acid sequence of SEQ ID NO: 37.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a TUBA8 promoter comprising the nucleic acid sequence of SEQ ID NO: 14, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a TUBA8 promoter comprising the nucleic acid sequence of SEQ ID NO: 14, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 37)
CCTGCAGGCAGCTGCGCGCTCGCTCGCTCACTGAGGCCGCCCGGGCGTCGGGCGACC
TTTGGTCGCCCGGCCTCAGTGAGCGAGCGAGCGCGCAGAGAGGGAGTGGCCAACTC
CATCACTAGGGGTTCCTGCGGCCGCACGCGTGAAGACATAGTTCCAGTCTGAGTCTG
AAGCCTGGGACCCATGAGAGCTGAAGACGTGGTCCCAGTCTGAGTCTGAAGCCTGA
GACCCAGGAGAGCTGAAGACGTGGTTCCAGTCTGAGTCTGAAGCCTGAGATCCAGG
AGAGCTAAGGACATGGTTCTAGTCTGAGTCTGAAGCCTGAGACCCAGGAGAGCTGA
TGGTGTGGTTCCAGTCTGAGTCTGAAGCCTGAGACCCAGGAGAGCTGAATACGTAGT
TCCAGTCTGAGTCTGAAGCCTGAGTTCCAGGAGAGCTGAGGACATGGTTCCAGTCTG
AATCTGAAGCCTGAGACCCAGGAGAGCTGATGGTGTGGTCTGAAGACGTAGTTCCA
GTCTGAGTCTGAAGCCTGAGACCCAGGAGAGCTGAAGATGTGGTTTCAGTCTGTCTG
AAGCCTGAGACCCAGGAGAGCTGATGGTGTGGTTCCAGTCTGAGTCTGAAGTCTGAG
ACCCAGGAGAGCTGAAGATGTGGTTCCAGTCTGAGTCTGAAGCCTGAGACCCAGCA
GAGCTGAAGACATGGTTCCAGTCTGAGTCTGAAGCCTGAGACCCAGGAGAGCTGAA
GATGTGGTTTCAGTCTGTCTGAAGCCTGAGACCCGGGAGAGCTGAAGACGTAGTTCC
AGTCTGAGTCTGAAGCCTGAGAGACCCAGGAGAGCTGATGGTGTGGTTCCAGTCTGA
GTCTGAAGCCTGAGACCCAGGAGAGCTGAAGATGTGGTTTCAGTCTGTCTGAAGCTT
GAGACCCAGGGGAGCTGAAGATGTAGTTCCAGTCTGAGTCTGAAGCCTGAGACCCA
GGAGAGGTGAAGACGTGGTTTCAGTCTGAGTCAAGGCCTGAGAACCAGGAGAGCTG
CTGGTGAAAGTTCTAGTGCAAGGGCAGAAGACCAATGTCCTACCTAGCTCAACAGTC
AGGCAGGCAGAAGTTCCCTGTTTCTCAGCCTTTTTGTTCTATTCTGTTCTTCAGTTGGT
TGGATGAGGCCCCTGCACATTAAGGATAGACAAAAATTCAACGCATGCTTTACTAAG
TACCGTTTGTATCAGTGGGTAAAGCACTGTGTTTGGTACTCTCTCAAATGCAAAGAT
GATTACGACACATGTACTATCGTTTATGAATGGGTGGCCAACAGAACAGATTGCCGC
ATAGGTAAGCAGAAATCTGCTCTCATTCTCTATTGGCCACAAGCAGGCATGTCTTAG
GAGCAGAAGGGTAGGAAGATCTCTAACTGTGCTTGGAAACTTGGGGAGTTACCACG
TCTGGCTAAAGTGGTATTGTCTTAAGGAAAACCTCTTACTACTGGGCAGAGGCAGGG
GAACCCTGGTATGAGTTCTGGATTACATAGGAGATGTGACTTGGACACGTTTGGGGC
TTAAAAGTAGGAAGGGATCAAGGGGGGAGATTTGAAAATCCCGGTGGAGGTGCGAG
GTATCCGGGGAGAGGTGGGAGCAGAGGCCCTGCAGCTTGCCAAGCACACACGGCCC
TAGGGCGCCCAGCTGAGACGGCACCTTGGCACCCGGGCCCGCTGCAGCCCGCTCCG
GTCAGCTGCACCCCAGTCAGGAGCCTTTCCAGCGGGTCGGAGGAGAACGGAAGTTT
GGGGAGACCCGCGCGATTCGCCTGGCTGCATTTTACATTTCTTTCTCCGGCAGCTGG
GGTCACGAAGGCTGCTCTCGCCGGCGGTGTTGGAACGTGGACACGTGCGCTTTGGTA
ATAGGGCAGCCTCCCCCGCGGGCGCAGTCCCCGCTGCGAGCGCCCCCGGCTGCTGAG
GCGGGACCGAGGACCCGGAGATTTCGCCGGTGGCAGGTGGAAAGGCGAGCGGCATG
GAGCGCGTAATAAGAGAGTTGGAGTCGGAAAGAGCAGCCCCAGTCGCCGGGGAAGC
GGGAGGTCAGTGCGGGCTCCGGCGGCCCCCAGGCTCCGAGCGCCCGCCCGCGGCCC
CGGCCCGGCCCCTAGCCCCCGCCGCCCGCGCCCGCCCCGGGTCGCCCCTCTGGCCCC
GGGTCCGAGCCATGCGTCTCTGAGCGCCCCGAGCGCGCCCCCGCCCCGGACCGTGCC
CGGGCCCCGGCGCCCCCAGCCCGGCGCCGCCCACCGGTCGCTAGCCACCATGGCTGA
AGCCCCTCCTAGAAGGCTTGGACTGGGACCTCCTCCTGGGGATGCTCCTAGAGCTGA
ACTGGTGGCTCTGACAGCCGTGCAGTCTGAACAAGGCGAAGCTGGTGGCGGCGGAT
CTCCACGTAGACTTGGACTGCTGGGAAGCCCTCTTCCTCCTGGTGCTCCACTTCCTGG
ACCTGGCAGTGGATCTGGATCTGCCTGTGGCCAGAGAAGCTCTGCCGCTCACAAGAG
ATACCGGCGGCTGCAGAACTGGGTGTACAACGTGCTGGAAAGACCCAGAGGCTGGG
CCTTCGTGTACCACGTGTTCATCTTTCTGCTGGTGTTCAGCTGCCTGGTGCTGTCCGT
GCTGAGCACCATCCAAGAACATCAAGAGCTGGCTAACGAGTGCCTGTTAATACTGG
AGTTTGTGATGATTGTGGTGTTCGGCCTCGAGTACATCGTCCGCGTTTGGAGCGCCG
GCTGCTGCTGCAGATATAGAGGTTGGCAAGGCAGATTCCGCTTCGCCAGAAAGCCCT
TCTGCGTGATCGACTTCATCGTGTTCGTGGCCAGCGTGGCCGTGATTGCTGCTGGCAC
ACAGGGCAACATCTTCGCCACAAGCGCCCTGCGGAGCATGCGGTTTCTGCAGATCCT
GAGAATGGTCCGAATGGACAGAAGAGGCGGCACCTGGAAGCTGCTGGGCTCTGTGG
TGTACGCCCACAGCAAAGAGCTGATCACCGCCTGGTACATCGGATTTCTGGTGCTGA
TCTTCGCCTCCTTCCTGGTGTACCTGGCCGAGAAGGACGCCAACAGCGACTTTAGCA
GCTACGCCGACTCTCTTTGGTGGGGCACCATCACACTGACCACCATCGGCTACGGCG
ACAAGACCCCTCACACATGGCTGGGAAGAGTGCTGGCCGCTGGATTTGCTCTGCTGG
GCATCAGCTTTTTCGCCCTGCCTGCCGGAATCCTCGGATCTGGCTTTGCCCTGAAGGT
GCAAGAGCAGCACCGGCAGAAGCACTTCGAGAAGAGAAGAATGCCTGCCGCCAACC
TGATTCAGGCCGCTTGGAGACTGTACAGCACCGACATGAGCAGAGCCTACCTGACCG
CCACGTGGTATTATTACGACTCGATCCTGCCTAGCTTCCGCGAACTGGCCCTGCTGTT
TGAGCATGTGCAGAGAGCCAGAAACGGCGGCCTCAGACCTCTGGAAGTTCGGAGAG
CACCTGTGCCTGATGGCGCCCCTTCTAGATATCCTCCAGTGGCCACCTGTCACAGAC
CCGGCAGCACATCTTTTTGCCCTGGCGAGTCTAGCCGGATGGGCATCAAGGACAGAA
TCAGAATGGGCAGCAGCCAGCGGAGAACAGGCCCTTCTAAACAGCATCTGGCCCCT
CCAACCATGCCTACAAGCCCTAGCTCTGAGCAAGTGGGCGAAGCCACCTCTCCTACC
AAGGTGCAGAAGTCCTGGTCCTTCAACGACCGGACCAGATTCAGAGCCAGCCTGAG
ACTGAAGCCCAGAACCTCTGCCGAGGATGCCCCTTCTGAAGAGGTGGCCGAAGAGA
AGTCCTACCAGTGCGAGCTGACCGTGGACGACATCATGCCAGCCGTGAAAACCGTG
ATACGGTCTATCCGGATCCTGAAGTTCCTGGTGGCCAAGCGGAAGTTCAAAGAGACA
CTGCGGCCCTACGACGTGAAGGACGTGATCGAGCAGTATTCTGCCGGCCACCTGGAC
ATGCTGGGCAGAATCAAGAGCCTGCAGACCAGAGTGGACCAGATCGTTGGAAGAGG
CCCAGGCGACAGAAAGGCCAGAGAGAAGGGCGATAAGGGCCCATCTGATGCCGAG
GTTGTCGACGAGATATCAATGATGGGCAGAGTGGTCAAGGTGGAAAAACAGGTGCA
GAGCATCGAGCACAAGCTGGACCTGCTGCTGGGATTCTACAGCCGGTGTCTGAGAA
GCGGCACATCTGCATCTCTGGGCGCTGTGCAGGTCCCACTGTTCGACCCTGATATCA
CCAGCGACTATCACAGCCCCGTGGACCACGAGGACATCTCCGTTTCTGCTCAGACCC
TGAGCATCAGCAGATCCGTGTCCACCAACATGGACGGATCCCGGGCTGACTACAAA
GACCATGACGGTGATTATAAAGATCATGACATCGACTACAAGGATGACGATGACAA
GTAATAAGAGCTCGCTGATCAGCCTCGACTGTGCCTTCTAGTTGCCAGCCATCTGTTG
TTTGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTGGG
GGGTGGGGTGGGGCAGGACAGCAAGGGGGAGGATTGGGAAGACAATAGCAGGCAT
GCTGGGGATGCGGTGGGCTCTATGttaattCGGACCGCTAGGAACCCCTAGTGATGGAGT
TGGCCACTCCCTCTCTGCGCGCTCGCTCGCTCACTGAGGCCGGGCGACCAAAGGTCG
CCCGACGCCCGGGCTTTGCCCGGGCGGCCTCAGTGAGCGAGCGAGCGCGCAGCTGC
CTGCAGG

TABLE 17
Construct Components (SEQ ID NO: 37)

| Component (5′ to | Position |
|---|---|
| 3′ orientation) | (nucleotides) |
| 5′ ITR | 12-130 |
| TUBA8 promoter | 145-1950 |
| 5′ UTR | 1951-2240 |
| KCNQ4 | 2258-4342 |
| coding region | |
| 3x Flag | 4355-4423 |
| polyA | 4448-4671 |
| 3′ ITR | 4687-4816 |

In some aspects, the TUBA8 promoter comprises a nucleic acid sequence that comprises at least 80%, at least 85%, at least 90%, at least 95%, at least 96%, at least 97%, at least 98% at least 99%, or 100% identity to nucleotides 145-1950 of SEQ ID NO: 37.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 38. In some aspects, the construct comprises the nucleic acid sequence of SEQ ID NO: 38.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4915 of SEQ ID NO: 38. In some aspects, the construct comprises nucleotides 12-4915 of SEQ ID NO: 38.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 38. In some aspects, the rAAVAnc80 particle comprises the nucleic acid sequence of SEQ ID NO: 38.

In some aspects, the construct comprises (i) a 5′ ITR comprising the nucleic acid sequence of SEQ ID NO: 16, (ii) a CMV enhancer comprising the nucleic acid sequence of SEQ ID NO: 18, (iii) a prestin promoter comprising the nucleic acid sequence of SEQ ID NO: 15, (iv) a 5′ UTR comprising the nucleic acid sequence of SEQ ID NO: 19, (v) a KCNQ4 coding region comprising the nucleic acid sequence of SEQ ID NO: 20, (vi) optionally, a 3× FLAG tag comprising the nucleic acid sequence of SEQ ID NO: 39, (vii) a polyA sequence comprising the nucleic acid sequence of SEQ ID NO: 22, and (viii) a 3′ ITR comprising the nucleic acid sequence of SEQ ID NO: 17.

In some aspects, the construct comprises (i) a 5′ ITR comprising the nucleic acid sequence of SEQ ID NO: 16, (ii) a prestin promoter comprising the nucleic acid sequence of SEQ ID NO: 15, (iii) a 5′ UTR comprising the nucleic acid sequence of SEQ ID NO: 19, (iv) a KCNQ4 coding region comprising the nucleic acid sequence of SEQ ID NO: 20, (v) optionally, a 3× FLAG tag comprising the nucleic acid sequence of SEQ ID NO: 39, (vi) a polyA sequence comprising the nucleic acid sequence of SEQ ID NO: 22, and (vii) a 3′ ITR comprising the nucleic acid sequence of SEQ ID NO: 17.

Exemplary Construct
(SEQ ID NO: 38)
CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCGCACGCGTTAAACA
CTGAACAGGTGTTAGCAACATTGCCATTATTGTGTTAGTATATTAGGTAC
CTGGTGCTACCGGCAAAACCAGTTTATCATCCAACTGTCTCCAGTGTTGC
TACTCAAAGTTTGGTCCTCCAGTAGCCTATCAGGATCACCCAGGGGCCTG
TTAGAAAGGCACATCTCAGACCCCACCCCAGACCTACTGAATCAGAATCT
GCGTTTTTAACGGGATCCGCAGGTGATTCCTATGCACATTAAAGTGTAAG
AAGTACTGGGCTACAGACAGGTATGTGACAAAATAATTTCATAGGATGGC
AAAGGCCAAGTGGCAAATGAAGGACACCAGAAATGCACGTCCCAGGAGCC
CAACTCCTCCTTAGTAAATTACCCTATTAAGATTTGTTTAGAGATGTTCA
AAAGCGTGGAGAAAAGCAAATTTGGTTTCCTCAGCTAGGGACGCGGAGAG
TGGTCTGGTGCCCTTGAAGAGATCGCCCTCGTGTGGAGTAGGGAGGGAAT
CTCTAGCCTTTCCTCTCGGATGAAGAACAGCACCAGCGCTCCCAGCCAAA
GGCCTGGCCCAGGTTCTGGAGGTGGGGTCTCCTTGGCAGAAGCCTCTGGT
GTCTGCAGGCGTGCATTTACAGCTTTAAGACCAAACAGCTAGTCCGCCAC
GTGTCACTACAGTGTGCACGCGCAGAAATGCACAAAGCAAAAAAAAAAAA
AAAGATGCTCTTAATGAACCAACTATAATCCTTGCTAAGGCATAAAGCCA
GAGGGAAGTATGTATCTGAAATCATTTTCTACCCCTCACCCTCTTGGAGC
CCGGCACTCTGGCTGCGGTGCTCTCTTGTATCCCAGTTGCTAGATGCAAA
ACAAGCTATTTCCTATCTAATTTTTTTTTTTAAGAGACGGAGTCTCGCTT
TGTTGCCCAGGCTGGTCTCAAACTCCTGGACTCAAGCAATTCTCCCAGCT
TGGGGTAACGTGTTACATTATTCTACTTAATAAAAAGCAAAAGTTGTTTT
ATAAATTCTAACTTAAATGCCCAGAAAATAACTTATCATGCATTGCCTTG
TCGTGCAATAGTCAATATTTGCAAACCAAGTGTTAACCAAAGGCAGTTCA
TCAAAGATTTTTGAAAATTAAAAAAAAAAAAAAACTCATACTCACATTGT
CCTCAGGATTTCCTGTTTTCGAAATGTTCCTGTACGAATCGGAGTCTCTA
TAATGATTGTAATTGAAAAGATAAGTCAGGTTTTTTGTGTTTTTTTTTCA
TTTTAAAATCATAATACGCAATGTTTTCCACTTGAACGCTATACCTTGTG
TATTGTGCTTGCTTCAGCCTCGAGCCTCTACTGATGTTCCACCTCAAGGC
GACAGGAATGCCACCTGGAGAAACTCCTGGGCGGTATGGGAAGAAAGCCG
GTCTCATCAGAGTATATTTGCGGGGATCGACGACCAAGGTGTTAAATTCC
AAGCACGCTTTGGAAAGTTCTAGGTGCTTGGGAAGAGATCCGTAGGCGGC
AGGGATGCCCGCGCCCCGGCGTCCCAGCGCGGAGGGTGGCGGCGGGGCCT
GGCCCTAGCGGGGCGGGGCGGGCTCGGGTTACCGGGAGTCGCGGGGCGCG
GCCGGCACTGCCCGCGGCGCCTCCTCCTAGAGCCGCACCTGGAGGCAGCG
CGCGCGTCGAAGAGGCAGCGGCTGTGGAGCGCGGCGGGGCGGCTCCGCCC
AGGGCAGCCCGGGCTGGGCCAAGGAGCGAGCTCTCCCTTCTCCTGCTCTC
AGCCTCAGTGATCAAGGCTTCAGTGAACTGCACTGGAGCTCCCAGCGGGG
GATCTTGTCCCCTGTCCCGACTTTTGTGCTGCACATTGGATCTGGTGACA
CTCAGGAAATTGCTTGTCTCCGGCTGTTAAGGAATAATTTCAGAGTACTC
GCCGGTGGCAGGTGGAAAGGCGAGCGGCATGGAGCGCGTAATAAGAGAGT
TGGAGTCGGAAAGAGCAGCCCCAGTCGCCGGGGAAGCGGGAGGTCAGTGC
GGGCTCCGGCGGCCCCCAGGCTCCGAGCGCCCGCCCGCGGCCCCGGCCCG
GCCCCTAGCCCCCGCCGCCCGCGCCCGCCCCGGGTCGCCCCTCTGGCCCC
GGGTCCGAGCCATGCGTCTCTGAGCGCCCCGAGCGCGCCCCCGCCCCGGA
CCGTGCCCGGGCCCCGGCGCCCCCAGCCCGGCGCCGCCCACCGGTCGCTA
GCCACCATGGCTGAAGCCCCTCCTAGAAGGCTTGGACTGGGACCTCCTCC
TGGGGATGCTCCTAGAGCTGAACTGGTGGCTCTGACAGCCGTGCAGTCTG
AACAAGGCGAAGCTGGTGGCGGCGGATCTCCACGTAGACTTGGACTGCTG
GGAAGCCCTCTTCCTCCTGGTGCTCCACTTCCTGGACCTGGCAGTGGATC
TGGATCTGCCTGTGGCCAGAGAAGCTCTGCCGCTCACAAGAGATACCGGC
GGCTGCAGAACTGGGTGTACAACGTGCTGGAAAGACCCAGAGGCTGGGCC
TTCGTGTACCACGTGTTCATCTTTCTGCTGGTGTTCAGCTGCCTGGTGCT
GTCCGTGCTGAGCACCATCCAAGAACATCAAGAGCTGGCTAACGAGTGCC
TGTTAATACTGGAGTTTGTGATGATTGTGGTGTTCGGCCTCGAGTACATC
GTCCGCGTTTGGAGCGCCGGCTGCTGCTGCAGATATAGAGGTTGGCAAGG
CAGATTCCGCTTCGCCAGAAAGCCCTTCTGCGTGATCGACTTCATCGTGT
TCGTGGCCAGCGTGGCCGTGATTGCTGCTGGCACACAGGGCAACATCTTC
GCCACAAGCGCCCTGCGGAGCATGCGGTTTCTGCAGATCCTGAGAATGGT
CCGAATGGACAGAAGAGGCGGCACCTGGAAGCTGCTGGGCTCTGTGGTGT
ACGCCCACAGCAAAGAGCTGATCACCGCCTGGTACATCGGATTTCTGGTG
CTGATCTTCGCCTCCTTCCTGGTGTACCTGGCCGAGAAGGACGCCAACAG
CGACTTTAGCAGCTACGCCGACTCTCTTTGGTGGGGCACCATCACACTGA
CCACCATCGGCTACGGCGACAAGACCCCTCACACATGGCTGGGAAGAGTG
CTGGCCGCTGGATTTGCTCTGCTGGGCATCAGCTTTTTCGCCCTGCCTGC
CGGAATCCTCGGATCTGGCTTTGCCCTGAAGGTGCAAGAGCAGCACCGGC
AGAAGCACTTCGAGAAGAGAAGAATGCCTGCCGCCAACCTGATTCAGGCC
GCTTGGAGACTGTACAGCACCGACATGAGCAGAGCCTACCTGACCGCCAC
GTGGTATTATTACGACTCGATCCTGCCTAGCTTCCGCGAACTGGCCCTGC
TGTTTGAGCATGTGCAGAGAGCCAGAAACGGCGGCCTCAGACCTCTGGAA
GTTCGGAGAGCACCTGTGCCTGATGGCGCCCCTTCTAGATATCCTCCAGT
GGCCACCTGTCACAGACCCGGCAGCACATCTTTTTGCCCTGGCGAGTCTA
GCCGGATGGGCATCAAGGACAGAATCAGAATGGGCAGCAGCCAGCGGAGA
ACAGGCCCTTCTAAACAGCATCTGGCCCCTCCAACCATGCCTACAAGCCC
TAGCTCTGAGCAAGTGGGCGAAGCCACCTCTCCTACCAAGGTGCAGAAGT
CCTGGTCCTTCAACGACCGGACCAGATTCAGAGCCAGCCTGAGACTGAAG
CCCAGAACCTCTGCCGAGGATGCCCCTTCTGAAGAGGTGGCCGAAGAGAA
GTCCTACCAGTGCGAGCTGACCGTGGACGACATCATGCCAGCCGTGAAAA
CCGTGATACGGTCTATCCGGATCCTGAAGTTCCTGGTGGCCAAGCGGAAG
TTCAAAGAGACACTGCGGCCCTACGACGTGAAGGACGTGATCGAGCAGTA
TTCTGCCGGCCACCTGGACATGCTGGGCAGAATCAAGAGCCTGCAGACCA
GAGTGGACCAGATCGTTGGAAGAGGCCCAGGCGACAGAAAGGCCAGAGAG
AAGGGCGATAAGGGCCCATCTGATGCCGAGGTTGTCGACGAGATATCAAT
GATGGGCAGAGTGGTCAAGGTGGAAAAACAGGTGCAGAGCATCGAGCACA
AGCTGGACCTGCTGCTGGGATTCTACAGCCGGTGTCTGAGAAGCGGCACA
TCTGCATCTCTGGGCGCTGTGCAGGTCCCACTGTTCGACCCTGATATCAC
CAGCGACTATCACAGCCCCGTGGACCACGAGGACATCTCCGTTTCTGCTC
AGACCCTGAGCATCAGCAGATCCGTGTCCACCAACATGGACGGATCCCGG
GCTGACTACAAAGACCATGACGGTGATTATAAAGATCATGACATCGACTA
CAAGGATGACGATGACAAGTAATAAGAGCTCGCTGATCAGCCTCGACTGT
GCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGT
GGGGCAGGACAGCAAGGGGGAGGATTGGGAAGACAATAGCAGGCATGCTG
GGGATGCGGTGGGCTCTATGTTAATTCGGACCGCTAGGAACCCCTAGTGA
TGGAGTTGGCCACTCCCTCTCTGCGCGCTCGCTCGCTCACTGAGGCCGGG
CGACCAAAGGTCGCCCGACGCCCGGGCTTTGCCCGGGCGGCCTCAGTGAG
CGAGCGAGCGCGCAGCTGCCTGCAGG

TABLE 18
Construct Components (SEQ ID NO: 38)

| Component (5′ to | Position |
|---|---|
| 3′ orientation) | nucleotides |
| 5′ ITR | 1-130 |
| Prestin promoter | 145-2049 |
| 5′ UTR | 2050-2339 |
| KCNQ4 | 2357-4441 |
| coding region | |
| 3x Flag | 4454-4519 |
| polyA | 4547-4770 |
| 3′ ITR | 4786-4915 |

In some aspects, the prestin promoter comprises a nucleic acid sequence that comprises at least 80%, at least 85%, at least 90%, at least 95%, at least 96%, at least 97%, at least 98% at least 99%, or 100% identity to nucleotides 145-2049 of SEQ ID NO: 38.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 49. In some aspects, the construct comprises the nucleic acid sequence of SEQ ID NO: 49.

In some aspects, the construct comprises a nucleic acid sequence that comprises at least 80%, at least 85%, at least 90%, at least 95%, at least 96%, at least 97%, at least 98% at least 99%, or 100% identity to nucleotides 12-4070 of SEQ ID NO: 49. In some aspects, the construct comprises nucleotides 12-4070 of SEQ ID NO: 49.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 49. In some aspects, the rAAVAnc80 particle comprises the nucleic acid sequence of SEQ ID NO: 49.

Exemplary Construct
(SEQ ID NO: 49)
CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTGCGGCCGCACGCGTCTAGAT
CCCATATATGGAGTTCCGCGTTACATAACTTACGGTAAATGGCCCGCCTG
GCTGACCGCCCAACGACCCCCGCCCATTGACGTCAATAATGACGTATGTT
CCCATAGTAACGCCAATAGGGACTTTCCATTGACGTCAATGGGTGGAGTA
TTTACGGTAAACTGCCCACTTGGCAGTACATCAAGTGTATCATATGCCAA
GTACGCCCCCTATTGACGTCAATGACGGTAAATGGCCCGCCTGGCATTAT
GCCCAGTACATGACCTTATGGGACTTTCCTACTTGGCAGTACATCTACGT
ATTAGTCATCGCTATTACCATGGTGATGCGGTTTTGGCAGTACATCAATG
GGCGTGGATAGCGGTTTGACTCACGGGGATTTCCAAGTCTCCACCCCATT
GACGTCAATGGGAGTTTGTTTTGGCACCAAAATCAACGGGACTTTCCAAA
ATGTCGTAACAACTCCGCCCCATTGACGCAAATGGGCGGTAGGCGTGTAC
GGTGGGAGGTCTATATAAGCAGAGCTCGTTTAGTGAACCGTCAGATCGCC
TGGAGACGCACCGGTGCCACCATGGCCGAGGCCCCCCCGCGCCGCCTCGG
CCTGGGTCCCCCGCCCGGGGACGCCCCCCGCGCGGAGCTAGTGGCGCTCA
CGGCCGTGCAGAGCGAACAGGGCGAGGCGGGCGGGGGCGGCTCCCCGCGC
CGCCTCGGCCTCCTGGGCAGCCCCCTGCCGCCGGGCGCGCCCCTCCCTGG
GCCGGGCTCCGGCTCGGGCTCCGCCTGCGGCCAGCGCTCCTCGGCCGCGC
ACAAGCGCTACCGCCGCCTGCAGAACTGGGTCTACAACGTGCTGGAGCGG
CCCCGCGGCTGGGCCTTCGTCTACCACGTCTTCATATTTTTGCTGGTCTT
CAGCTGCCTGGTGCTGTCTGTGCTGTCCACTATCCAGGAGCACCAGGAAC
TTGCCAACGAGTGTCTCCTCATCTTGGAATTCGTGATGATCGTGGTTTTC
GGCTTGGAGTACATCGTCCGGGTCTGGTCCGCCGGATGCTGCTGCCGCTA
CCGAGGATGGCAGGGTCGCTTCCGCTTTGCCAGAAAGCCCTTCTGTGTCA
TCGACTTCATCGTGTTCGTGGCCTCGGTGGCCGTCATCGCCGCGGGTACC
CAGGGCAACATCTTCGCCACGTCCGCGCTGCGCAGCATGCGCTTCCTGCA
GATCCTGCGCATGGTGCGCATGGACCGCCGCGGCGGCACCTGGAAGCTGC
TGGGCTCAGTGGTCTACGCGCATAGCAAGGAGCTGATCACCGCCTGGTAC
ATCGGGTTCCTGGTGCTCATCTTCGCCTCCTTCCTGGTCTACCTGGCTGA
GAAGGACGCCAACTCCGACTTCTCCTCCTACGCCGACTCGCTCTGGTGGG
GGACGATTACATTGACAACCATCGGCTATGGTGACAAGACACCGCACACA
TGGCTGGGCAGGGTCCTGGCTGCTGGCTTCGCCTTACTGGGCATCTCTTT
CTTTGCCCTGCCTGCCGGCATCCTAGGCTCCGGCTTTGCCCTGAAGGTCC
AGGAGCAGCACCGGCAGAAGCACTTCGAGAAGCGGAGGATGCCGGCAGCC
AACCTCATCCAGGCTGCCTGGCGCCTGTACTCCACCGATATGAGCCGGGC
CTACCTGACAGCCACCTGGTACTACTATGACAGTATCCTCCCATCCTTCA
GAGAGCTGGCCCTCTTGTTTGAGCACGTGCAACGGGCCCGCAATGGGGGC
CTACGGCCCCTGGAGGTGCGGCGGGCGCCGGTACCCGACGGAGCACCCTC
CCGTTACCCGCCCGTTGCCACCTGCCACCGGCCGGGCAGCACCTCCTTCT
GCCCTGGGGAAAGCAGCCGGATGGGCATCAAAGACCGCATCCGCATGGGC
AGCTCCCAGCGGCGGACGGGTCCTTCCAAGCAGCATCTGGCACCTCCAAC
AATGCCCACCTCCCCAAGCAGCGAGCAGGTGGGTGAGGCCACCAGCCCCA
CCAAGGTGCAAAAGAGCTGGAGCTTCAATGACCGCACCCGCTTCCGGGCA
TCTCTGAGACTCAAACCCCGCACCTCTGCTGAGGATGCCCCCTCAGAGGA
AGTAGCAGAGGAGAAGAGCTACCAGTGTGAGCTCACGGTGGACGACATCA
TGCCTGCTGTGAAGACAGTCATCCGCTCCATCAGGATTCTCAAGTTCCTG
GTGGCCAAAAGGAAATTCAAGGAGACACTGCGACCGTACGACGTGAAGGA
CGTCATTGAGCAGTACTCAGCAGGCCACCTGGACATGCTGGGCCGGATCA
AGAGCCTGCAAACTCGGGTGGACCAAATTGTGGGTCGGGGGCCCGGGGAC
AGGAAGGCCCGGGAGAAGGGCGACAAGGGGCCCTCCGACGCGGAGGTGGT
GGATGAAATCAGCATGATGGGACGCGTGGTCAAGGTGGAGAAGCAGGTGC
AGTCCATCGAGCACAAGCTGGACCTGCTGTTGGGCTTCTATTCGCGCTGC
CTGCGCTCTGGCACCTCGGCCAGCCTGGGCGCCGTGCAAGTGCCGCTGTT
CGACCCCGACATCACCTCCGACTACCACAGCCCTGTGGACCACGAGGACA
TCTCCGTCTCCGCACAGACGCTCAGCATCTCCCGCTCGGTCAGCACCAAC
ATGGACGGATCCCGGGCTGACTACAAAGACCATGACGGTGATTATAAAGA
TCATGACATCGACTACAAGGATGACGATGACAAGGGCTCCGGAGAGGGCA
GAGGAAGTCTGCTAACATGCGGTGACGTCGAGGAGAATCCTGGCCCAATG
GTGAGCAAGGGCGAGGCAGTGATCAAGGAGTTCATGCGGTTCAAGGTGCA
CATGGAGGGCTCCATGAACGGCCACGAGTTCGAGATCGAGGGCGAGGGCG
AGGGCCGCCCCTACGAGGGCACCCAGACCGCCAAGCTGAAGGTGACCAAG
GGTGGCCCCCTGCCCTTCTCCTGGGACATCCTGTCCCCTCAGTTCATGTA
CGGCTCCAGGGCCTTCATCAAGCACCCCGCCGACATCCCCGACTACTATA
AGCAGTCCTTCCCCGAGGGCTTCAAGTGGGAGCGCGTGATGAACTTCGAG
GACGGCGGCGCCGTGACCGTGACCCAGGACACCTCCCTGGAGGACGGCAC
CCTGATCTACAAGGTGAAGCTCCGCGGCACCAACTTCCCTCCTGACGGCC
CCGTAATGCAGAAGAAGACAATGGGCTGGGAAGCGTCCACCGAGCGGTTG
TACCCCGAGGACGGCGTGCTGAAGGGCGACATTAAGATGGCCCTGCGCCT
GAAGGACGGCGGCCGCTACCTGGCGGACTTCAAGACCACCTACAAGGCCA
AGAAGCCCGTGCAGATGCCCGGCGCCTACAACGTCGACCGCAAGTTGGAC
ATCACCTCCCACAACGAGGACTACACCGTGGTGGAACAGTACGAACGCTC
CGAGGGCCGCCACTCCACCGGCGGCATGGACGAGCTGTACAAGTAATAAG
AGCTCGCTGATCAGCCTCGACTGTGCCTTCTAGTTGCCAGCCATCTGTTG
TTTGCCCCTCCCCCGTGCCTTCCTTGACCCTGGAAGGTGCCACTCCCACT
GTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATT
GGGAAGACAATAGCAGGCATGCTGGGGATGCGGTGGGCTCTATGGAAGCT
TGAATTCAGCTGACGTGCCTCGGACCGCTAGGAACCCCTAGTGATGGAGT
TGGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGCG
AGCGCGCAGCTGCCTGCAGGGGCGCCTGATGCGGTATTTTCTCCTTACGC
ATCTGTGCGGTATTTCACACCGCATACGTCAAAGCAACCATAGTACGCGC
CCTGTAGCGGCGCATTAAGCGCGGCGGGTGTGGTGGTTACGCGCAGCGTG
ACCGCTACACTTGCCAGCGCCCTAGCGCCCGCTCCTTTCGCTTTCTTCCC
TTCCTTTCTCGCCACGTTCGCCGGCTTTCCCCGTCAAGCTCTAAATCGGG
GGCTCCCTTTAGGGTTCCGATTTAGTGCTTTACGGCACCTCGACCCCAAA
AAACTTGATTTGGGTGATGGTTCACGTAGTGGGCCATCGCCCTGATAGAC
GGTTTTTCGCCCTTTGACGTTGGAGTCCACGTTCTTTAATAGTGGACTCT
TGTTCCAAACTGGAACAACACTCAACCCTATCTCGGGCTATTCTTTTGAT
TTATAAGGGATTTTGCCGATTTCGGCCTATTGGTTAAAAAATGAGCTGAT
TTAACAAAAATTTAACGCGAATTTTAACAAAATATTAACGTTTACAATTT
TATGGTGCACTCTCAGTACAATCTGCTCTGATGCCGCATAGTTAAGCCAG
CCCCGACACCCGCCAACACCCGCTGACGCGCCCTGACGGGCTTGTCTGCT
CCCGGCATCCGCTTACAGACAAGCTGTGACCGTCTCCGGGAGCTGCATGT
GTCAGAGGTTTTCACCGTCATCACCGAAACGCGCGAGACGAAAGGGCCTC
GTGATACGCCTATTTTTATAGGTTAATGTCATGAACAATAAAACTGTCTG
CTTACATAAACAGTAATACAAGGGGTGTTATGAGCCATATTCAACGGGAA
ACGTCGAGGCCGCGATTAAATTCCAACATGGATGCTGATTTATATGGGTA
TAAATGGGCTCGCGATAATGTCGGGCAATCAGGTGCGACAATCTATCGCT
TGTATGGGAAGCCCGATGCGCCAGAGTTGTTTCTGAAACATGGCAAAGGT
AGCGTTGCCAATGATGTTACAGATGAGATGGTCAGACTAAACTGGCTGAC
GGAATTTATGCCTCTTCCGACCATCAAGCATTTTATCCGTACTCCTGATG
ATGCATGGTTACTCACCACTGCGATCCCCGGAAAAACAGCATTCCAGGTA
TTAGAAGAATATCCTGATTCAGGTGAAAATATTGTTGATGCGCTGGCAGT
GTTCCTGCGCCGGTTGCATTCGATTCCTGTTTGTAATTGTCCTTTTAACA
GCGATCGCGTATTTCGTCTCGCTCAGGCGCAATCACGAATGAATAACGGT
TTGGTTGATGCGAGTGATTTTGATGACGAGCGTAATGGCTGGCCTGTTGA
ACAAGTCTGGAAAGAAATGCATAAACTTTTGCCATTCTCACCGGATTCAG
TCGTCACTCATGGTGATTTCTCACTTGATAACCTTATTTTTGACGAGGGG
AAATTAATAGGTTGTATTGATGTTGGACGAGTCGGAATCGCAGACCGATA
CCAGGATCTTGCCATCCTATGGAACTGCCTCGGTGAGTTTTCTCCTTCAT
TACAGAAACGGCTTTTTCAAAAATATGGTATTGATAATCCTGATATGAAT
AAATTGCAGTTTCATTTGATGCTCGATGAGTTTTTCTAATCTCATGACCA
AAATCCCTTAACGTGAGTTTTCGTTCCACTGAGCGTCAGACCCCGTAGAA
AAGATCAAAGGATCTTCTTGAGATCCTTTTTTTCTGCGCGTAATCTGCTG
CTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAG
ATACCAAATACTGTCCTTCTAGTGTAGCCGTAGTTAGGCCACCACTTCAA
GAACTCTGTAGCACCGCCTACATACCTCGCTCTGCTAATCCTGTTACCAG
TGGCTGCTGCCAGTGGCGATAAGTCGTGTCTTACCGGGTTGGACTCAAGA
CGATAGTTACCGGATAAGGCGCAGCGGTCGGGCTGAACGGGGGGTTCGTG
CACACAGCCCAGCTTGGAGCGAACGACCTACACCGAACTGAGATACCTAC
AGCGTGAGCTATGAGAAAGCGCCACGCTTCCCGAAGGGAGAAAGGCGGAC
AGGTATCCGGTAAGCGGCAGGGTCGGAACAGGAGAGCGCACGAGGGAGCT
TCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTG
GCCTTTTGCTCACATGT

TABLE 18
Construct Components (SEQ ID NO: 49)

| Component (5′ to | Position |
|---|---|
| 3′ orientation) | (nucleotides) |
| 5′ ITR | 12-130 |
| CMV enhancer | 169-472 |
| CMV promoter | 473-676 |
| KCNQ4 | 722-2806 |
| coding region | |
| 3x Flag | 2819-2884 |
| T2A | 2894-2947 |
| mScarlet | 2951-3643 |
| polyA | 3671-3895 |
| 3′ ITR | 3930-4070 |

In some aspects, the CMV promoter comprises a nucleic acid sequence that comprises at least 80%, at least 85%, at least 90%, at least 95%, at least 96%, at least 97%, at least 98% at least 99%, or 100% identity to nucleotides 473-676 of SEQ ID NO: 49.

In some aspects, the construct comprises a nucleic acid sequence that comprises at least 80%, at least 85%, at least 90%, at least 95%, at least 96%, at least 97%, at least 98% at least 99%, or 100% identity to SEQ ID NO: 50. In some aspects, the construct comprises the nucleic acid sequence of SEQ ID NO: 50.

In some aspects, the rAAVAnc80 particle comprises a nucleic acid sequence that comprises at least 80%, at least 85%, at least 90%, at least 95%, at least 96%, at least 97%, at least 98% at least 99%, or 100% identity to SEQ ID NO: 50. In some aspects, the rAAVAnc80 particle comprises the nucleic acid sequence of SEQ ID NO: 50.

Exemplary Construct
(SEQ ID NO: 50)
TCGCAGTGGTGAGTAACCATGCATCATCAGGAGTACGGATAAAATGCTTG
ATGGTCGGAAGAGGCATAAATTCCGTCAGCCAGTTTAGTCTGACCATCTC
ATCTGTAACATCATTGGCAACGCTACCTTTGCCATGTTTCAGAAACAACT
CTGGCGCATCGGGCTTCCCATACAATCGATAGATTGTCGCACCTGATTGC
CCGACATTATCGCGAGCCCATTTATACCCATATAAATCAGCATCCATGTT
GGAATTTAATCGCGGCCTAGAGCAAGACGTTTCCCGTTGAATATGGCTCA
TACTCTTCCTTTTTCAATATTATTGAAGCATTTATCAGGGTTATTGTCTC
ATGAGCGGATACATATTTGAATGTATTTAGAAAAATAAACAAATAGGGGT
TCCGCGCACATTTCCCCGAAAAGTGCCACCTGACGTCTAAGAAACCATTA
TTATCATGACATTAACCTATAAAAATAGGCGTATCACGAGGCCCTTTCGT
CTCGCGCGTTTCGGTGATGACGGTGAAAACCTCTGACACATGCAGCTCCC
GGAGACGGTCACAGCTTGTCTGTAAGCGGATGCCGGGAGCAGACAAGCCC
GTCAGGGCGCGTCAGCGGGTGTTGGCGGGTGTCGGGGCTGGCTTAACTAT
GCGGCATCAGAGCAGATTGTACTGAGAGTGCACCATATGTTGGCCACTCC
CTCTCTGCGCGCTCGCTCGCTCACTGAGGCCGCCCGGGCAAAGCCCGGGC
GTCGGGCGACCTTTGGTCGCCCGGCCTCAGTGAGCGAGCGAGCGCGCAGA
GAGGGAGTGGCCAACTCCATCACTAGGGGTTCCTTTGTCGACGCGGCCGC
ACGCGTGACATTGATTATTGACTAGTTATTAATAGTAATCAATTACGGGG
TCATTAGTTCATAGCCCATATATGGAGTTCCGCGTTACATAACTTACGGT
AAATGGCCCGCCTGGCTGACCGCCCAACGACCCCCGCCCATTGACGTCAA
TAATGACGTATGTTCCCATAGTAACGCCAATAGGGACTTTCCATTGACGT
CAATGGGTGGACTATTTACGGTAAACTGCCCACTTGGCAGTACATCAAGT
GTATCATATGCCAAGTACGCCCCCTATTGACGTCAATGACGGTAAATGGC
CCGCCTGGCATTATGCCCAGTACATGACCTTATGGGACTTTCCTACTTGG
CAGTACATCTACGTATTAGTCATCGCTATTACCATGGGTCGAGGTGAGCC
CCACGTTCTGCTTCACTCTCCCCATCTCCCCCCCCTCCCCACCCCCAATT
TTGTATTTATTTATTTTTTAATTATTTTGTGCAGCGATGGGGGCGGGGGG
GGGGGGGGGGCGCGCGCCAGGCGGGGCGGGGCGGGGCGAGGGGCGGGGCG
GGGCGAGGCGGAGAGGTGCGGCGGCAGCCAATCAGAGCGGCGCGCTCCGA
AAGTTTCCTTTTATGGCGAGGCGGCGGCGGCGGCGGCCCTATAAAAAGCG
AAGCGCGCGGCGGGCGGGAGTCGCTGCGTTGCCTTCGCCCCGTGCCCCGC
TCCGCGCCGCCTCGCGCCGCCCGCCCCGGCTCTGACTGACCGCGTTACTC
CCACAGGTGAGCGGGCGGGACGGCCCTTCTCCTCCGGGCTGTAATTAGCG
CTTGGTTTAATGACGGCTCGTTTCTTTTCTGTGGCTGCGTGAAAGCCTTA
AAGGGCTCCGGGAGGGCCCTTTGTGCGGGGGGGAGCGGCTCGGGGGGTGC
GTGCGTGTGTGTGTGCGTGGGGAGCGCCGCGTGCGGCCCGCGCTGCCCGG
CGGCTGTGAGCGCTGCGGGCGCGGCGCGGGGCTTTGTGCGCTCCGCGTGT
GCGCGAGGGGAGCGCGGCCGGGGGCGGTGCCCCGCGGTGGGGGGGGCTGC
GAGGGGAACAAAGGCTGCGTGCGGGGTGTGTGCGTGGGGGGGTGAGCAGG
GGGTGTGGGCGCGGCGGTCGGGCTGTAACCCCCCCCTGCACCCCCCTCCC
CGAGTTGCTGAGCACGGCCCGGCTTCGGGTGCGGGGCTCCGTGCGGGGCG
TGGCGCGGGGCTCGCCGTGCCGGGCGGGGGGTGGCGGCAGGTGGGGGTGC
CGGGCGGGGCGGGGCCGCCTCGGGCCGGGGAGGGCTCGGGGGAGGGGCGC
GGCGGCCCCCGGAGCGCCGGCGGCTGTCGAGGCGCGGCGAGCCGCAGCCA
TTGCCTTTTATGGTAATCGTGCGAGAGGGCGCAGGGACTTCCTTTGTCCC
AAATCTGTGCGGAGCCGAAATCTGGGAGGCGCCGCCGCACCCCCTCTAGC
GGGCGCGGGGCGAAGCGGTGCGGCGCCGGCAGGAAGGAAATGGGCGGGGA
GGGCCTTCGTGCGTCGCCGCGCCGCCGTCCCCTTCTCCCTCTCCAGCCTC
GGGGCTGTCCGCGGGGGGACGGCTGCCTTCGGGGGGGACGGGGCAGGGCG
GGGTTCGGCTTCTGGCGTGTGACCGGCGGCTCTAGAGCCTCTGCTAACCA
TGTTCATGCCTTCTTCTTTTTCCTACAGCTCCTGGGCAACGTGCTGGTTA
TTGTGACCGGTCGCTAGCCACCATGGTGAGCAAGGGCGAGGAGCTGTTCA
CCGGGGTGGTGCCCATCCTGGTCGAGCTGGACGGCGACGTAAACGGCCAC
AAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCA
CCCTCGTGACCACCCTGACCTACGGCGTGCAGTGCTTCAGCCGCTACCCC
GACCACATGAAGCAGCACGACTTCTTCAAGTCCGCCATGCCCGAAGGCTA
CGTCCAGGAGCGCACCATCTTCTTCAAGGACGACGGCAACTACAAGACCC
GCGCCGAGGTGAAGTTCGAGGGCGACACCCTGGTGAACCGCATCGAGCTG
AAGGGCATCGACTTCAAGGAGGACGGCAACATCCTGGGGCACAAGCTGGA
GTACAACTACAACAGCCACAACGTCTATATCATGGCCGACAAGCAGAAGA
ACGGCATCAAGGTGAACTTCAAGATCCGCCACAACATCGAGGACGGCAGC
GTGCAGCTCGCCGACCACTACCAGCAGAACACCCCCATCGGCGACGGCCC
CGTGCTGCTGCCCGACAACCACTACCTGAGCACCCAGTCCGCCCTGAGCA
AAGACCCCAACGAGAAGCGCGATCACATGGTCCTGCTGGAGTTCGTGACC
GCCGCCGGGATCACTCTCGGCATGGACGAGCTGTACAAGTAAGAGCTCGC
TGATCAGCCTCGACTGTGCCTTCTAGTTGCCAGCCATCTGTTGTTTGCCC
CTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGA
CAATAGCAGGCATGCTGGGGATGCGGTGGGCTCTATGGAAGCTTGAATTC
AGCTGACGTGCCTCGGACCGTCCTAGGAGGAACCCCTAGTGATGGAGTTG
GCCACTCCCTCTCTGCGCGCTCGCTCGCTCACTGAGGCCGGGCGACCAAA
GGTCGCCCGACGCCCGGGCTTTGCCCGGGCGGCCTCAGTGAGCGAGCGAG
CGCGCAGAGAGGGAGTGGCCAACATAGGCGCGCCTCGGGCCGTGCTTGTA
ATCCTGAGGAAATTGTAAACGTTAATATTTTGTTAAAATTCGCGTTAAAT
TTTTGTTAAATCAGCTCATTTTTTAACCAATAGGCCGAAATCGGCAAAAT
CCCTTATAAATCAAAAGAATAGCCCGAGATAGGGTTGAGTGTTGTTCCAG
TTTGGAACAAGAGTCCACTATTAAAGAACGTGGACTCCAACGTCAAAGGG
CGAAAAACCGTCTATCAGGGCGATGGCCCACTACGTGAACCATCACCCAA
ATCAAGTTTTTTGGGGTCGAGGTGCCGTAAAGCACTAAATCGGAACCCTA
AAGGGAGCCCCCGATTTAGAGCTTGACGGGGAAAGCCGGCGAACGTGGCG
AGAAAGGAAGGGAAGAAAGCGAAAGGAGCGGGCGCTAGGGCGCTGGCAAG
TGTAGCGGTCACGCTGCGCGTAACCACCACACCCGCCGCGCTTAATGCGC
CGCTACAGGGCGCGTACTAACATGTGAGCAAAAGGCCAGCAAAAGGCCAG
GAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCG
ACAGGACTATAAAGATACCAGGCGTTTCCCCCTGGAAGCTCCCTCGTGCG
CTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGT
TCGGTGTAGGTCGTTCGCTCCAAGCTGGGCTGTGTGCACGAACCCCCCGT
TCAGCCCGACCGCTGCGCCTTATCCGGTAACTATCGTCTTGATTCCAACC
CGGTAAGACACGACTTATCGCCACTGGCAGCAGCCACTGGTAACAGGATT
AGCAGAGCGAGGTATGTAGGCGGTGCTACAGAGTTCTTGAAGTGGTGGCC
TAACTACGGCTACACTAGAAGAACAGTATTTGGTATCTGCGCTCTGCTGA
AGCCAGTTACCTTCGGAAAAAGAGTTGGTAGCTCTTGATCCGGCAAACAA
ACCACCGCTGGTAGCGGTGGTTTTTTTGTTTGCAAGCAGCAGATTACGCG
CAGAAAAAAAGGATCTCAAGAAGATCCTTTGATCTTTTCTACGGGGTCTG
ACGCTCAGTGGAACGAAAACTCACGTTAAGGGATTTTGGTCATGAGATTA
TCAAAAAGGATCTTCACCTAGATCCTTTTAAATTAAAAATGAAGTTTTAA
ATCAATCTAAAGTATATATGAGTAAACTTGGTCTGACAGTTAGAAAAACT
CATCGAGCATCAAATGAAACTGCAATTTATTCATATCAGGATTATCAATA
CCATATTTTTGAAAAAGCCGTTTCTGTAATGAAGGAGAAAACTCACCGAG
GCAGTTCCATAGGATGGCAAGATCCTGGTATCGGTCTGCGATTCCGACCC
GTCCAACATCAATACAACCTATTAATTTCCCCTCGTCAAAAATAAGGTTA
TCAAGTGAGAAATCACCATGAGTGACGACTGAATCCGGTGAGAATGGCAA
AAGTTTATGCATTTCTTTCCAGACTTGTTCAACAGGCCAGCCATTACGCT
CGTCATCAAAATCACTCGCATCAACCAAACCGTTATTCATTCGTGATTGC
GCCTGAGCGAGACGAAATACGCGATCGCTGTTAAAAGGACAATTACAAAC
AGGAATCGAATGCAACCGGCGCAGGAACACTGCCAGCGCATCAACAATAT
TTTCACCTGAATCAGGATATTCTTCTAATACCTGGAATGCTGTTTTCCCA
GGGA

TABLE 18
Construct Components (SEQ ID NO: 50)

| Component (5′ to | Position |
|---|---|
| 3′ orientation) | (nucleotides) |
| 5′ ITR | 690-834 |
| CMV enhancer | 857-1236 |
| CBA promoter | 1239-1516 |
| Chimeric | 1517-2529 |
| intron | |
| EGFP | 2574-3293 |
| polyA | 3315-3539 |
| 3′ ITR | 3579-3723 |

In some aspects, the CBA promoter comprises a nucleic acid sequence that comprises at least 80%, at least 85%, at least 90%, at least 95%, at least 96%, at least 97%, at least 98% at least 99%, or 100% identity to nucleotides 1239-1516 of SEQ ID NO: 23.

### Pharmaceutical Compositions

Among other things, the present disclosure provides pharmaceutical compositions. In some aspects, compositions provided herein are suitable for administration to an animal for the amelioration of symptoms associated with syndromic and/or nonsyndromic hearing loss.

In some aspects, pharmaceutical compositions of the present disclosure may comprise, e.g., a polynucleotide, e.g., one or more constructs, as described herein. In some aspects, a pharmaceutical composition may comprise one or more AAV particles, e.g., one or more rAAV construct encapsidated by one or more AAV serotype capsids, as described herein.

In some aspects, a pharmaceutical composition comprises one or more pharmaceutically or physiologically acceptable carriers, diluents or excipients. As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein. Such compositions may include one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives. In some aspects, formulations are in a dosage forms, such as injectable solutions, injectable gels, drug-release capsules, and the like.

In some aspects, compositions of the present disclosure are formulated for intravenous administration. In some aspects compositions of the present disclosure are formulated for intra-cochlear administration. In some aspects, a therapeutic composition is formulated to comprise a lipid nanoparticle, a polymeric nanoparticle, a mini-circle DNA and/or a CELiD DNA. In some aspects, any of the compositions of the present disclosure are formulated for administration into or through the round window membrane of an inner ear of a subject. In some aspects, any of the compositions of the present disclosure are formulated for administration into perilymph fluid of an inner ear.

In some aspects, a therapeutic composition is formulated to comprise a synthetic perilymph solution. For example, in some aspects, a synthetic perilymph solution includes 20-200 mM NaCl; 1-5 mM KCl; 0.1-10 mM CaCl₂); 1-10 mM glucose; and 2-50 mM HEPES, with a pH between about 6 and about 9. In some aspects, a therapeutic composition is formulated to comprise a physiologically suitable solution. For example, in some aspects, a physiologically suitable solution comprises commercially available 1×PBS with pluronic acid F68, prepared to a final concentration of: 8.10 mM Sodium Phosphate Dibasic, 1.5 mM Monopotassium Phosphate, 2.7 mM Potassium Chloride, 172 mM Sodium Chloride, and 0.001% Pluronic Acid F68). In some aspects, alternative pluronic acids are utilized. In some aspects, alternative ion concentrations are utilized.

In some aspects, any of the pharmaceutical compositions described herein may further comprise one or more agents that promote the entry of a nucleic acid or any of the constructs described herein into a mammalian cell (e.g., a liposome or cationic lipid). In some aspects, any of the constructs described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Mirus Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007, each of which is incorporated in its entirety herein by reference).

In some aspects, a composition includes a pharmaceutically acceptable carrier (e.g., phosphate buffered saline, saline, or bacteriostatic water). Upon formulation, solutions will be administered in a manner compatible with a dosage formulation and in such amount as is therapeutically effective. Formulations are easily administered in a variety of dosage forms such as injectable solutions, injectable gels, drug-release capsules, and the like.

In some aspects, a composition provided herein can be, e.g., formulated to be compatible with their intended route of administration. A non-limiting example of an intended route of administration is local administration (e.g., intra-cochlear administration). In some aspects, a provided composition comprises one nucleic acid construct. In some aspects, a provided composition comprises two or more different constructs. In some aspects, a composition that include a single nucleic acid construct comprising a coding sequence that encodes a polypeptide and/or a functional characteristic portion thereof. In some aspects, compositions comprise a single nucleic acid construct comprising a coding sequence that encodes a polypeptide and/or a functional characteristic portion thereof, which, when introduced into a mammalian cell, that coding sequence is integrated into the genome of the mammalian cell.

Also provided are kits including any of the compositions described herein. In some aspects, a kit can include a solid composition (e.g., a lyophilized composition including the at least two different constructs described herein) and a liquid for solubilizing the lyophilized composition. In some aspects, a kit can include a pre-loaded syringe including any of the compositions described herein.

In some aspects, the kit includes a vial comprising any of the compositions described herein (e.g., formulated as an aqueous composition, e.g., an aqueous pharmaceutical composition).

In some aspects, the kits can include instructions for performing any of the methods described herein.

### Genetically Modified Cells

The present disclosure also provides a cell (e.g., an animal cell, e.g., a mammalian cell, e.g., a primate cell, e.g., a human cell) that includes any of the nucleic acids, constructs or compositions described herein. In some aspects, an animal cell is a human cell (e.g., a human hair cell or a human outer hair cell). In other aspects, an animal cell is a non-human mammal (e.g., Simian cell, Felidae cell, Canidae cell etc.). A person skilled in the art will appreciate that the nucleic acids and constructs described herein can be introduced into any animal cell (e.g., the outer hair cells of any animal suitable for veterinary intervention). Non-limiting examples of constructs and methods for introducing constructs into animal cells are described herein.

In some aspects, an animal cell can be any cell of the inner ear, including outer hair cells.

In some aspects, an animal cell is a specialized cell of the cochlea. In some aspects, an animal cell is a hair cell. In some aspects, an animal cell is a cochlear inner hair cell or a cochlear outer hair cell. In some aspects, an animal cell is a cochlear inner hair cell. In some aspects, an animal cell is a cochlear outer hair cell. In some aspects, an animal cell is in vitro. In some aspects, an animal cell is of a cell type which is endogenously present in an animal, e.g., in a primate and/or human. In some aspects, an animal cell is an autologous cell obtained from an animal and cultured ex vivo. In some aspects, the ex vivo cell is an inner ear cell. In some aspects, the ex vivo cell is an inner ear outer hair cell.

### Methods

Among other things, the present disclosure provides methods. In some aspects, a method comprises introducing a construct, vector, AAV particle, composition, or cell as described herein into the inner ear (e.g., a cochlea) of a subject. For example, provided herein are methods that in some aspects include administering to an inner ear (e.g., cochlea) of a subject (e.g., an animal, e.g., a mammal, e.g., a primate, e.g., a human) a therapeutically effective amount of any construct, vector, AAV particle, composition, or cell described herein. In some embodiments, administration of any compositions of the present disclosure may be carried out by administration into or through the round window membrane of an inner ear of a subject. In some embodiments, administration of any compositions of the present disclosure may be carried out by administration into perilymph fluid of an inner ear. In some aspects of any of these methods, the subject has been previously identified as having a defective inner ear cell target gene (e.g., a outer hair cell and/or hearing cell target gene having a mutation that results in a decrease in the expression and/or activity of a hearing cell target protein encoded by the gene). In some aspects of these methods, the subject has been previously identified as having a defective potassium voltage-gated channel, KQT-like subfamily, member 4 (KCNQ4) gene.

Some aspects of any of these methods further include, prior to the introducing or administering step, determining that the subject has a defective inner ear cell target gene (e.g., a outer hair cell and/or hearing cell target gene having a mutation that results in a decrease in the expression and/or activity of a hearing cell target protein encoded by the gene). Some aspects of these methods further include determining that the subject has a defective potassium voltage-gated channel, KQT-like subfamily, member 4 (KCNQ4) gene.

Some aspects of any of these methods can further include detecting a mutation in an inner ear cell target gene in a subject (e.g., an outer hair cell and/or hearing cell target gene having a mutation that results in a decrease in the expression and/or activity of a hearing cell target protein encoded by the gene). Some aspects of these methods can further include detecting a mutation in a potassium voltage-gated channel, KQT-like subfamily, member 4 (KCNQ4) gene in a subject.

Some aspects of any of the methods can further include identifying or diagnosing a subject as having nonsyndromic or syndromic sensorineural hearing loss. Some aspects of these methods can further include identifying or diagnosing a subject as having nonsyndromic or syndromic sensorineural hearing loss caused by a mutation in a potassium voltage-gated channel, KQT-like subfamily, member 4 (KCNQ4) gene. Some aspects of these methods can further include identifying or diagnosing a subject as having DFNA2 hearing loss.

In some aspects, provided herein are methods of correcting an inner ear cell target gene defect in an inner ear of a subject, e.g., an animal, e.g., a mammal, e.g., a primate, e.g., a human. In some aspects, methods include administering to the inner ear of a subject a therapeutically effective amount of any of the constructs, vectors, AAV particles, compositions, or cells described herein, where the administering repairs and or ameliorates the inner ear cell target gene defect in any cell subset of the inner ear of a subject. In some aspects, the inner ear target cell may be a sensory cell, e.g., a outer hair cell, and/or a non-sensory cell, and/or all or any subset of inner ear cells. In some aspects, the inner ear cell target gene defect is a mutation in a potassium voltage-gated channel, KQT-like subfamily, member 4 (KCNQ4) gene.

Also provided herein are methods of increasing the expression level of an inner ear cell target protein in the inner ear cells of a subject (e.g., an animal, e.g., a mammal, e.g., a primate, e.g., a human) that include: administering to the inner ear of the subject a therapeutically effective amount of any of the constructs, vectors, AAV particles, compositions, or cells described herein, where the administering results in an increase in the expression level of the inner ear cell target protein (e.g., a polypeptide) in cells the inner ear of a subject (e.g., a outer hair cell). In some aspects, the inner ear target cell may be an outer hair cell. In some aspects, the increased expression is relative to the endogenous polypeptide expression in the inner ear cells. In some aspects, the inner ear cell target protein is KQT-like subfamily, member 4 (KCNQ4).

Also provided herein are methods of treating hearing loss, e.g., nonsyndromic sensorineural hearing loss or syndromic sensorineural hearing loss, in a subject (e.g., an animal, e.g., a mammal, e.g., a primate, e.g., a human) identified as having a defective inner ear cell target gene that include: administering to the inner ear of the subject a therapeutically effective amount of any of the constructs, vectors, AAV particles, compositions, or cells described herein. In some aspects the defective inner ear cell target gene is a KQT-like subfamily, member 4 (KCNQ4) gene.

Also provided herein are methods comprising transducing a cell with any of the constructs or vectors described herein and one or more helper plasmids collectively comprising an AAV Rep gene, AAV Cap gene, AAV VA gene, AAV E2a gene, and AAV E4 gene.

Also provided herein are methods of expressing the polypeptide in an outer hair cell of a subject in need thereof, comprising administering the constructs, vectors, AAV particles, compositions, or cells described herein. In some aspects, the subject has been previously identified as having a defective potassium voltage-gated channel, KQT-like subfamily, member 4 (KCNQ4) gene.

Also provided herein are methods of increasing expression of the polypeptide in an outer hair cell of a subject in need thereof, comprising administering the constructs, vectors, AAV particles, compositions, or cells described herein. In some aspects, the increased expression is relative to the endogenous polypeptide expression in the outer hair cell of the subject.

Also provided herein are methods of treating hearing loss in a subject suffering from or at risk of hearing loss, comprising administering comprising administering the constructs, vectors, AAV particles, compositions, or cells described herein.

In some aspects, the administration is to the inner ear of the subject. In some aspects, the administration is to the cochlea of the subject. In some aspects, the administration is via a round window membrane injection.

Also provided herein are methods of expressing a polypeptide in an outer hair cell of a subject in need thereof.

In some aspects, administration is to the inner ear of the subject. In some aspects, the administration is to the cochlea of the subject. In some aspects, the administration is via a round window membrane injection.

Also provided herein are surgical methods for treatment of hearing loss (e.g., nonsyndromic sensorineural hearing loss or syndromic sensorineural hearing loss). In some aspects, the methods include the steps of: introducing into a cochlea of a subject a first incision at a first incision point; and administering intra-cochlearly a therapeutically effective amount of any of the compositions provided herein. In some aspects, the composition is administered to the subject at the first incision point. In some aspects, the composition is administered to the subject into or through the first incision.

In some aspects of any of the methods described herein, any composition described herein is administered to the subject into or through the cochlea oval window membrane. In some aspects of any of the methods described herein, any of the compositions described herein is administered to the subject into or through the cochlea round window membrane. In some aspects of any of the methods described herein, the composition is administered using a medical device capable of creating a plurality of incisions in the round window membrane. In some aspects, the medical device includes a plurality of micro-needles. In some aspects, the medical device includes a plurality of micro-needles including a generally circular first aspect, where each micro-needle has a diameter of at least about 10 microns. In some aspects, the medical device includes a base and/or a reservoir capable of holding the composition. In some aspects, the medical device includes a plurality of hollow micro-needles individually including a lumen capable of transferring the composition. In some aspects, the medical device includes a means for generating at least a partial vacuum.

In some aspects, technologies of the present disclosure are used to treat subjects with or at risk of hearing loss. In some such aspects, a pathogenic variant causes or is at risk of causing hearing loss.

In some aspects, a subject experiencing hearing loss will be evaluated to determine if and where one or more mutations may exist that may cause hearing loss. In some aspects of any of the methods described herein, the subject or animal is a mammal, in some aspects the mammal is a domestic animal, a farm animal, a zoo animal, a non-human primate, or a human. In some aspects of any of the methods described herein, the animal, subject, or mammal is an adult, a teenager, a juvenile, a child, a toddler, an infant, or a newborn. In some aspects of any of the methods described herein, the animal, subject, or mammal is 1-5, 1-10, 1-20, 1-30, 1-40, 1-50, 1-60, 1-70, 1-80, 1-90, 1-100, 1-110, 2-5, 2-10, 10-20, 20-30, 30-40, 40-50, 50-60, 60-70, 70-80, 80-90, 90-100, 100-110, 10-30, 10-40, 10-50, 10-60, 10-70, 10-80, 10-90, 10-100, 10-110, 20-40, 20-50, 20-60, 20-70, 20-80, 20-90, 20-100, 20-110, 30-50, 30-60, 30-70, 30-80, 30-90, 30-100, 40-60, 40-70, 40-80, 40-90, 40-100, 50-70, 50-80, 50-90, 50-100, 60-80, 60-90, 60-100, 70-90, 70-100, 70-110, 80-100, 80-110, or 90-110 years of age. In some aspects of any of the methods described herein, the subject or mammal is 1, 2, 3, 4, 5, 6, 7, 8, 9, 10, or 11 months of age.

In some aspects of any of the methods described herein, the methods result in improvement in hearing (e.g., any of the metrics for determining improvement in hearing described herein) in a subject in need thereof for at least 10 days, at least 15 days, at least 20 days, at least 25 days, at least 30 days, at least 35 days, at least 40 days, at least 45 days, at least 50 days, at least 55 days, at least 60 days, at least 65 days, at least 70 days, at least 75 days, at least 80 days, at least 85 days, at least 100 days, at least 105 days, at least 110 days, at least 115 days, at least 120 days, at least 5 months, at least 6 months, at least 7 months, at least 8 months, at least 9 months, at least 10 months, at least 11 months, or at least 12 months.

In some aspects a subject (e.g., an animal, e.g., a mammal, e.g., a human) has or is at risk of developing syndromic or nonsyndromic sensorineural hearing loss. In some aspects, a subject has or is at risk of developing KCNQ4-related hearing loss.

In some aspects, a subject (e.g., an animal, e.g., a mammal, e.g., a human) has been identified as having syndromic or nonsyndromic sensorineural hearing loss. In some aspects, a subject has been identified as having KCNQ4-related hearing loss.

In some aspects, a subject (e.g., an animal, e.g., a mammal, e.g., a human) has been identified as being at risk of hearing loss (e.g., at risk of being a carrier of a gene mutation,). In some such aspects, a subject (e.g., an animal, e.g., a mammal, e.g., a human) may have certain risk factors of hearing loss or risk of hearing loss (e.g., known parental carrier, afflicted sibling, or symptoms of hearing loss). In some such aspects, a subject (e.g., an animal, e.g., a mammal, e.g., a human) has been identified as being a carrier of a mutation in a gene (e.g., via genetic testing) that has not previously been identified ( ). In some such aspects, identified mutations may be novel (i.e., not previously described in the literature), and methods of treatment for a subject suffering from or susceptible to hearing loss will be personalized to the mutation(s) of the particular patient.

In some aspects, successful treatment of syndromic or nonsyndromic sensorineural hearing loss can be determined in a subject using any of the conventional functional hearing tests known in the art. Non-limiting examples of functional hearing tests are various types of audiometric assays (e.g., pure-tone testing, speech testing, test of the middle ear, auditory brainstem response, and otoacoustic emissions).

In some aspects of any method provided herein, two or more doses of any composition described herein are introduced or administered into a cochlea of a subject. Some aspects of any of these methods can include introducing or administering a first dose of a composition into a cochlea of a subject, assessing hearing function of the subject following introduction or administration of a first dose, and administering an additional dose of a composition into the cochlea of the subject found not to have a hearing function within a normal range (e.g., as determined using any test for hearing known in the art).

In some aspects of any method provided herein, the composition can be formulated for intra-cochlear administration. In some aspects of any of the methods described herein, the compositions described herein can be administered via intra-cochlear administration or local administration. In some aspects of any of the methods described herein, the compositions are administered through the use of a medical device (e.g., any of the exemplary medical devices described herein).

In some aspects, intra-cochlear administration can be performed using any of the methods described herein or known in the art. For example, in some aspects, a composition can be administered or introduced into the cochlea using the following surgical technique: first using visualization with a 0 degree, 2.5-mm rigid endoscope, the external auditory canal is cleared and a round knife is used to sharply delineate an approximately 5-mm tympanomeatal flap. The tympanomeatal flap is then elevated and the middle ear is entered posteriorly. The chorda tympani nerve is identified and divided, and a curette is used to remove the scutal bone, exposing the round window membrane. To enhance apical distribution of the administered or introduced composition, a surgical laser may be used to make a small 2-mm fenestration in the oval window to allow for perilymph displacement during trans-round window membrane infusion of the composition. The microinfusion device is then primed and brought into the surgical field. The device is maneuvered to the round window, and the tip is seated within the bony round window overhang to allow for penetration of the membrane by the microneedle(s). The footpedal is engaged to allow for a measured, steady infusion of the composition. The device is then withdrawn and the round window and stapes foot plate are sealed with a gelfoam patch.

In some aspects of any method provided herein, a subject has or is at risk of developing syndromic or nonsyndromic sensorineural hearing loss. In some aspects of any method provided herein, a subject has been previously identified as having a mutation in an inner ear cell target gene, a gene which may be expressed in outer hair cells.

In some aspects of any method provided herein, a subject has been identified as being a carrier of a mutation in an inner ear cell target gene (e.g., via genetic testing). In some such aspects, the subject has been identified as being a carrier of a mutation in a KQT-like subfamily, member 4 (KCNQ4) gene. In some aspects of any method provided herein, a subject has been identified as having a mutation in an inner ear cell target gene and has been diagnosed with hearing loss (e.g., nonsyndromic sensorineural hearing loss or syndromic sensorineural hearing loss, e.g., DFNB1, DFNA3, DFNA2). Bart-Pumphrey syndrome, hystrix-like ichthyosis with deafness (HID), palmoplantar keratoderma with deafness, keratitis-ichthyosis-deafness (KID) syndrome, or Vohwinkel syndrome, respectively). In some aspects of any of the methods described herein, the subject has been identified as having hearing loss (e.g., nonsyndromic sensorineural hearing loss or syndromic sensorineural hearing loss). In some aspects, successful treatment of hearing loss (e.g., nonsyndromic sensorineural hearing loss or syndromic sensorineural hearing loss) can be determined in a subject using any of the conventional functional hearing tests known in the art. Non-limiting examples of functional hearing tests include various types of audiometric assays (e.g., pure-tone testing, speech testing, test of the middle ear, auditory brainstem response, and otoacoustic emissions).

In some aspects, a subject cell has previously been determined to have a defective inner ear cell target gene. In some such aspects, the defective inner ear cell target gene is a KQT-like subfamily, member 4 (KCNQ4) gene. In some aspects, a subject cell has previously been determined to have a defective hair cell target gene. In some such aspects, the defective inner ear cell target gene is a KQT-like subfamily, member 4 (KCNQ4) gene.

In some aspects of these methods, following treatment e.g., one or two or more administrations of compositions described herein, there is an increase in expression of an active inner ear cell target protein (e.g., a polypeptide). In some such aspects, the active inner ear cell target protein is KQT-like subfamily, member 4 (KCNQ4). In some aspects, an increase in expression of an active inner ear target protein as described herein (e.g., a polypeptide) is relative to a control level, e.g., as compared to the level of expression of an inner ear cell target protein prior to introduction of the compositions comprising any construct(s) as described herein. In some such aspects, the active inner ear cell target protein is KQT-like subfamily, member 4 (KCNQ4).

Methods of detecting expression and/or activity of a target protein (e.g., a polypeptide) are known in the art. In some aspects, a level of expression of an inner ear cell target protein can be detected directly (e.g., detecting inner ear cell target protein or target mRNA. Non-limiting examples of techniques that can be used to detect expression and/or activity of a target RNA or protein (e.g., a polypeptide) directly include: real-time PCR, Western blotting, immunoprecipitation, immunohistochemistry, mass spectrometry, or immunofluorescence. In some aspects, expression of an inner ear cell target protein can be detected indirectly (e.g., through functional hearing tests).

### Devices, Administration, and Surgical Methods

Provided herein are therapeutic delivery systems for treating hearing loss (e.g., nonsyndromic sensorineural hearing loss or syndromic sensorineural hearing loss). In one aspect, a therapeutic delivery system includes: i) a medical device capable of creating one or a plurality of incisions in a round window membrane of an inner ear of a subject in need thereof, and ii) an effective dose of a composition (e.g., any of the compositions described herein). In some aspects, a medical device includes a plurality of micro-needles.

Also provided herein are surgical methods for treatment of hearing loss (e.g., nonsyndromic sensorineural hearing loss or syndromic sensorineural hearing loss). In some aspects, a method the steps of: introducing into a cochlea of a subject a first incision at a first incision point; and administering intra-cochlearly a therapeutically effective amount of any of the compositions provided herein. In some aspects, a composition is administered to a subject at the first incision point. In some aspects, a composition is administered to a subject into or through the first incision.

In some aspects of any method provided herein, any of the compositions described herein is administered to the subject into or through the cochlea oval window membrane. In some aspects of any method provided herein, any of the compositions described herein is administered to the subject into or through the cochlea round window membrane. In some aspects of any method provided herein, the composition is administered using a medical device capable of creating a plurality of incisions in the round window membrane. In some aspects, a medical device includes a plurality of micro-needles. In some aspects, a medical device includes a plurality of micro-needles including a generally circular first aspect, where each micro-needle has a diameter of at least about 10 microns. In some aspects, a medical device includes a base and/or a reservoir capable of holding a composition. In some aspects, a medical device includes a plurality of hollow micro-needles individually including a lumen capable of transferring a composition. In some aspects, a medical device includes a means for generating at least a partial vacuum.

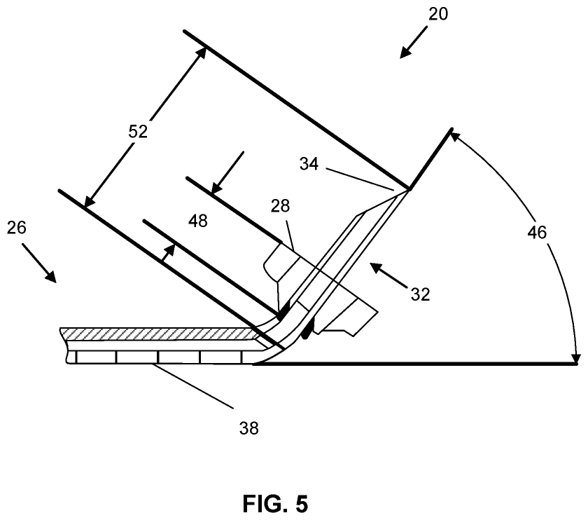

In some aspects, the present disclosure describes a delivery approach that utilizes a minimally invasive, well-accepted surgical technique for accessing the middle ear and/or inner ear through the external auditory canal. The procedure includes opening one of the physical barriers between the middle and inner ear at the oval window, and subsequently using a device disclosed herein, e.g., as shown in FIGS. 5-8 (or microcatheter) to deliver a composition disclosed herein at a controlled flow rate and in a fixed volume, via the round window membrane.

In some aspects, surgical procedures for mammals (e.g., rodents (e.g., mice, rats, hamsters, or rabbits), primates (e.g., NHP (e.g., macaque, chimpanzees, monkeys, or apes) or humans) may include venting to increase AAV vector transduction rates along the length of the cochlea. In some aspects, absence of venting during surgery may result in lower AAV vector cochlear cell transduction rates when compared to AAV vector cochlear cell transduction rates following surgeries performed with venting. In some aspects, venting facilitates transduction rates of about 75-100% of IHCs throughout the cochlea. In some aspects, venting permits IHC transduction rates of about 50-70%, about 60-80%, about 70-90%, or about 80-100% at the base of the cochlea. In some aspects, venting permits IHC transduction rates of about 50-70%, about 60-80%, about 70-90%, or about 80-100% at the apex of the cochlea.

A delivery device described herein may be placed in a sterile field of an operating room and the end of a tubing may be removed from the sterile field and connected to a syringe that has been loaded with a composition disclosed herein (e.g., one or more AAV vectors) and mounted in the pump. After appropriate priming of the system in order to remove any air, a needle may then be passed through the middle ear under visualization (surgical microscope, endoscope, and/or distal tip camera). A needle (or microneedle) may be used to puncture the RWM. The needle may be inserted until a stopper contacts the RWM. The device may then be held in that position while a composition disclosed herein is delivered at a controlled flow rate to the inner ear, for a selected duration of time. In some aspects, the flow rate (or infusion rate) may include a rate of about 30 μL/min, or from about 25 μL/min to about 35 μL/min, or from about 20 μL/min to about 40 μL/min, or from about 20 μL/min to about 70 μL/min, or from about 20 μL/min to about 90 μL/min, or from about 20 μL/min to about 100 μL/min. In some aspects, the flow rate is about 20 μL/min, about 30 μL/min, about 40 μL/min, about 50 μL/min, about 60 μL/min, about 70 μL/min, about 80 μL/min, about 90 μL/min or about 100 μL/min. In some aspects, the selected duration of time (that is, the time during which a composition disclosed herein is flowing) may be about 3 minutes, or from about 2.5 minutes to about 3.5 minutes, or from about 2 minutes to about 4 minutes, or from about 1.5 minutes to about 4.5 minutes, or from about 1 minute to about 5 minutes. In some aspects, the total volume of a composition disclosed herein that flows to the inner ear may be about 0.09 mL, or from about 0.08 mL to about 0.10 mL, or from about 0.07 mL to about 0.11 mL. In some aspects, the total volume of a composition disclosed herein equates to from about 40% to about 50% of the volume of the inner ear.

Once the delivery has been completed, the device may be removed. In some aspects, a device described herein, may be configured as a single-use disposable product. In other aspects, a device described herein may be configured as a multi-use, sterilizable product, for example, with a replaceable and/or sterilizable needle sub-assembly. Single use devices may be appropriately discarded (for example, in a biohazard sharps container) after administration is complete.

In some aspects, a composition disclosed herein comprises one or a plurality of rAAV constructs. In some aspects, when more than one rAAV construct is included in the composition, the rAAV constructs are each different. In some aspects, an rAAV construct comprises an anti-VEGF coding region, e.g., as described herein. In some aspects, a composition comprises an rAAV particle comprising an AAV construct described herein. In some aspects, the rAAV particle is encapsidated by an Anc80 capsid (e.g., an Anc80L65 capsid). In some aspects, the Anc80 capsid comprises a polypeptide of SEQ ID NO: 44.

In some aspects, a composition disclosed herein can be administered to a subject with a surgical procedure. In some aspects, administration, e.g., via a surgical procedure, comprises injecting a composition disclosed herein via a delivery device as described herein into the inner ear. In some aspects, a surgical procedure disclosed herein comprises performing a transcanal tympanotomy; performing a laser-assisted micro-stapedotomy; and injecting a composition disclosed herein via a delivery device as described herein into the inner ear.

In some aspects, a surgical procedure comprises performing a transcanal tympanotomy; performing a laser-assisted micro-stapedotomy; injecting a composition disclosed herein via a delivery device as described herein into the inner ear; applying sealant around the round window and/or an oval window of the subject; and lowering a tympanomeatal flap of the subject to the anatomical position.

In some aspects, a surgical procedure comprises performing a transcanal tympanotomy; preparing a round window of the subject; performing a laser-assisted micro-stapedotomy; preparing both a delivery device as described herein and a composition disclosed herein for delivery to the inner ear; injecting a composition disclosed herein via the delivery device into the inner ear; applying sealant around the round window and/or an oval window of the subject; and lowering a tympanomeatal flap of the subject to the anatomical position.

In some aspects, performing a laser-assisted micro-stapedotomy includes using a KTP otologic laser and/or a C02 otologic laser.

As another example, a composition disclosed herein is administered using a device and/or system specifically designed for intracochlear route of administration. In some aspects, design elements of a device described herein may include: maintenance of sterility of injected fluid; minimization of air bubbles introduced to the inner ear; ability to precisely deliver small volumes at a controlled rate; delivery through the external auditory canal by the surgeon; minimization of damage to the round window membrane (RWM), or to inner ear, e.g., cochlear structures beyond the RWM; and/or minimization of injected fluid leaking back out through the RWM.

The devices, systems, and methods provided herein also describe the potential for delivering a composition safely and efficiently into the inner ear, in order to treat conditions and disorders that would benefit from delivery of a composition disclosed herein to the inner ear, including, but not limited to, hearing disorders, e.g., as described herein. As another example, by placing a vent in the stapes footplate and injecting through the RWM, a composition disclosed herein is dispersed throughout the cochlea with minimal dilution at the site of action. The development of the described devices allows the surgical administration procedure to be performed through the external auditory canal in humans. The described devices can be removed from the ear following infusion of an amount of fluid into the perilymph of the cochlea. In subjects, the device may be advanced through the external auditory canal, either under surgical microscopic control or along with an endoscope.

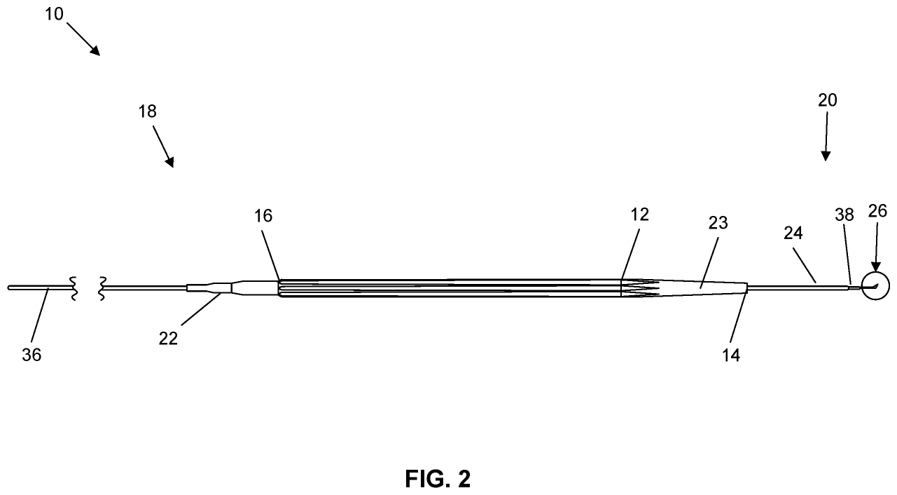

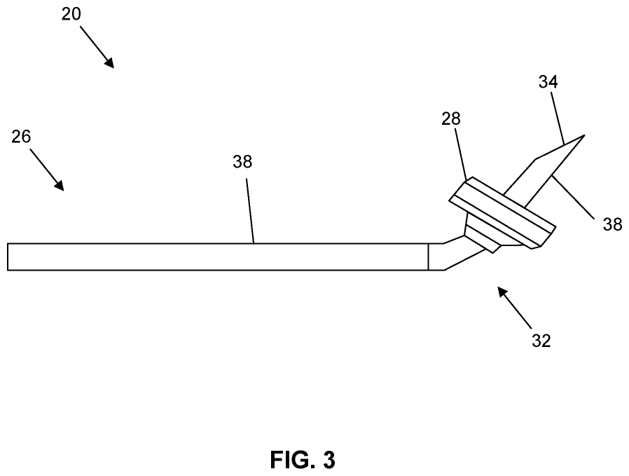

An exemplary device for use in any of the methods disclosed herein is described in FIGS. 2-5. FIG. 2 illustrates an exemplary device 10 for delivering fluid to an inner ear. Device 10 includes a knurled handle 12, and a distal handle adhesive 14 (for example, an epoxy such as Loctite 4014) that couples to a telescoping hypotube needle support 24. The knurled handle 12 (or handle portion) may include kurling features and/or grooves to enhance the grip. The knurled handle 12 (or handle portion) may be from about 5 mm to about 15 mm thick or from about 5 mm to about 12 mm thick, or from about 6 mm to about 10 mm thick, or from about 6 mm to about 9 mm thick, or from about 7 mm to about 8 mm thick. The knurled handle 12 (or handle portion) may be hollow such that fluid may pass through the device 10 during use. The device 10 may also include a proximal handle adhesive 16 at a proximal end 18 of the knurled handle 12, a needle sub-assembly 26 (shown in FIG. 2) with stopper 28 (shown in FIG. 3) at a distal end 20 of the device 10, and a strain relief feature 22. Strain relief feature 22 may be composed of a Santoprene material, a Pebax material, a polyurethane material, a silicone material, a nylon material, and/or a thermoplastic elastomer. The telescoping hypotube needle support 24 surrounds and supports a bent needle 38 (shown in FIG. 2) disposed therewithin.

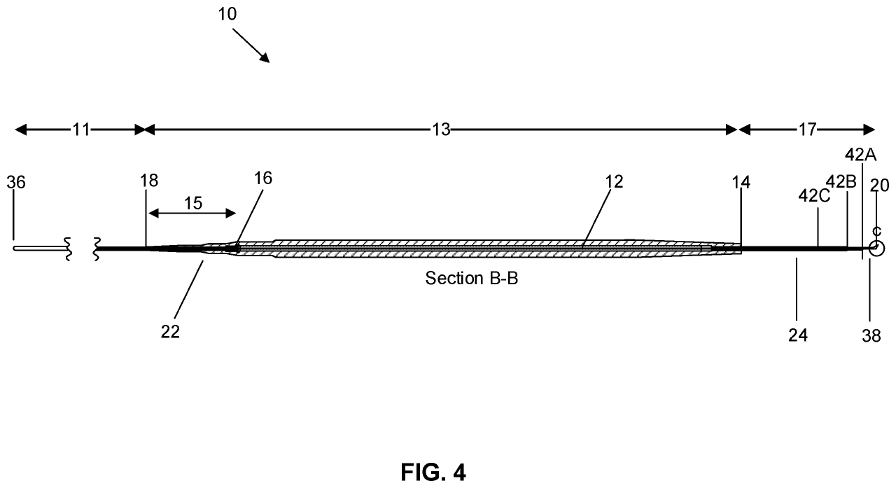

Referring still to FIGS. 2-3, the stopper 28 (shown in FIG. 3) may be composed of a thermoplastic material or plastic polymer (such as a UV-cured polymer), as well as other suitable materials, and may be used to prevent the bent needle 38 from being inserted too far into the ear canal (for example, to prevent insertion of bent needle 38 into the lateral wall or other inner ear structure). Device 10 also may include a tapered portion 23 disposed between the knurled handle 12 and the distal handle adhesive 14 that is coupled to the telescoping hypotube needle support 24. The knurled handle 12 (or handle portion) may include the tapered portion 23 at the distal end of the handle portion 12. Device 10 may also include tubing 36 fluidly connected to the proximal end 16 the device 10 and acts as a fluid inlet line connecting the device to upstream components (for example, a pump, a syringe, and/or upstream components which, in some aspects, may be coupled to a control system and/or power supply (not shown)). In some aspects, the bent needle 38 (shown in FIG. 3) extends from the distal end 20, through the telescoping hypotube needle support 24, through the tapered portion 23, through the knurled handle 12, and through the strain relief feature 22 and fluidly connects directly to the tubing 36. In other aspects, the bent needle 38 fluidly connects with the hollow interior of the knurled handle (for example, via the telescoping hypotube needle support 24) which in turn fluidly connects at a proximal end 16 with tubing 36. In aspects where the bent needle 38 does not extend all the way through the interior of the device 10, the contact area (for example, between overlapping nested hypotubes 42A-C(shown in FIG. 4), the tolerances, and/or sealants between interfacing components must be sufficient to prevent therapeutic fluid from leaking out of the device 10 (which operates at a relatively low pressure (for example, from about 1 Pascal to about 50 Pa, or from about 2 Pa to about 20 Pa, or from about 3 Pa to about 10 Pa)).

FIG. 3 illustrates a sideview of the bent needle sub-assembly 26, according to aspects of the present disclosed aspects. Bent needle sub-assembly 26 includes a needle 38 that has a bent portion 32. Bent needle sub-assembly 26 may also include a stopper 28 coupled to the bent portion 32. The bent portion 32 includes an angled tip 34 at the distal end 20 of the device 10 for piercing a membrane of the ear (for example, the RWM). The needle 38, bent portion 32, and angled top 34 are hollow such that fluid may flow therethrough. The angle 46 (as shown in FIG. 5) of the bent portion 32 may vary. A stopper 28 geometry may be cylindrical, disk-shaped, annulus-shaped, dome-shaped, and/or other suitable shapes. Stopper 28 may be molded into place onto bent portion 32. For example, stopper 28 may be positioned concentrically around the bent portion 32 using adhesives or compression fitting. Examples of adhesives include an UV cure adhesive (such as Dymax 203A-CTH-F-T), elastomer adhesives, thermoset adhesives (such as epoxy or polyurethane), or emulsion adhesives (such as polyvinyl acetate). Stopper 28 fits concentrically around the bent portion 32 such that angled tip 34 is inserted into the ear at a desired insertion depth. The bent needle 38 may be formed from a straight needle using incremental forming, as well as other suitable techniques.

FIG. 4 illustrates a perspective view of exemplary device 10 for delivering fluid to an inner ear. Tubing 36 may be from about 1300 mm in length (dimension 11 in FIG. 4) to about 1600 mm, or from about 1400 mm to about 1500 mm, or from about 1430 mm to about 1450 mm. Strain release feature 22 may be from about 25 mm to about 30 mm in length (dimension 15 in FIG. 4), or from about 20 mm to about 35 mm in length. Handle 12 may be about 155.4 mm in length (dimension 13 in FIG. 4), or from about 150 mm to about 160 mm, or from about 140 mm to about 170 mm. The telescoping hypotube needle support 24 may have two or more nested hypotubes, for example three nested hypotubes 42A, 42B, and 42C, or four nested hypotubes 42A, 42B, 42C, and 42D. The total length of hypotubes 42A, 42B, 42C and tip assembly 26 (dimension 17 in FIG. 4) may be from about 25 mm to about 45 mm, or from about 30 mm to about 40 mm, or about 35 mm. In addition, telescoping hypotube needle support 24 may have a length of about 36 mm, or from about 25 mm to about 45 mm, or form about 30 mm to about 40 mm. The three nested hypotubes 42A, 42B, and 42C each may have a length of 3.5 mm, 8.0 mm, and 19.8 mm, respectively, plus or minus about 20%. The inner-most nested hypotube (or most narrow portion) of the telescoping hypotube needle support 24 may be concentrically disposed around needle 38.

FIG. 5 illustrates a perspective view of bent needle sub-assembly 26 coupled to the distal end 20 of device 10, according to aspects of the present disclosed aspects. As shown in FIG. 5, bent needle sub-assembly 26 may include a needle 38 coupled to a bent portion 32. In other aspects, the bent needle 38 may be a single needle (for example, a straight needle that is then bent such that it includes the desired angle 46). Needle 38 may be a 33-gauge needle, or may include a gauge from about 32 to about 34, or from about 31 to 35. At finer gauges, care must be taken to ensure tubing 36 is not kinked or damaged. Needle 38 may be attached to handle 12 for safe and accurate placement of needle 38 into the inner ear. As shown in FIG. 5, bent needle sub-assembly 26 may also include a stopper 28 disposed around bent portion 32. FIG. 5 also shows that bent portion 32 may include an angled tip 34 for piercing a membrane of the ear (for example, the RWM). Stopper 28 may have a height 48 of about 0.5 mm, or from about 0.4 mm to about 0.6 mm, or from about 0.3 mm to about 0.7 mm. Bent portion 32 may have a length 52 of about 1.45 mm, or from about 1.35 mm to about 1.55 mm, or from about 1.2 mm to about 1.7 mm. In other aspects, the bent portion 32 may have a length greater than 2.0 mm such that the distance between the distal end of the stopper 28 and the distal end of the angled tip 34 is from about 0.5 mm to about 1.7 mm, or from about 0.6 mm to about 1.5 mm, or from about 0.7 mm to about 1.3 mm, or from about 0.8 mm to about 1.2 mm. FIG. 5 shows that stopper 28 may have a geometry that is cylindrical, disk-shaped, and/or dome-shaped. A person of ordinary skill will appreciate that other geometries could be used.

### Evaluating Hearing Loss and Recovery

In some aspects, hearing function is determined using auditory brainstem response measurements (ABR). In some aspects, hearing is tested by measuring distortion product optoacoustic emissions (DPOAEs). In some such aspects, measurements are taken from one or both ears of a subject. In some such aspects, recordings are compared to prior recordings for the same subject and/or known thresholds on such response measurements used to define, e.g., hearing loss versus acceptable hearing ranges to be defined as normal hearing. In some aspects, a subject has ABR and/or DPOAE measurements recorded prior to receiving any treatment. In some aspects, a subject treated with one or more technologies described herein will have improvements on ABR and/or DPOAE measurements after treatment as compared to before treatment. In some aspects, ABR and/or DPOAE measurements are taken after treatment is administered and at regular follow-up intervals post-treatment.

In some aspects, hearing function is determined using speech pattern recognition or is determined by a speech therapist. In some aspects, hearing function is determined by pure tone testing. In some aspects, hearing function is determined by bone conduction testing. In some aspects, hearing function is determined by acoustic reflex testing. In some aspects hearing function is determined by tympanometry. In some aspects, hearing function is determined by any combination of hearing analysis known in the art. In some such aspects, measurements are taken holistically, and/or from one or both ears of a subject. In some such aspects, recordings and/or professional analysis are compared to prior recordings and/or analysis for the same subject and/or known thresholds on such response measurements used to define, e.g., hearing loss versus acceptable hearing ranges to be defined as normal hearing. In some aspects, a subject has speech pattern recognition, pure tone testing, bone conduction testing, acoustic reflex testing and/or tympanometry measurements and/or analysis conducted prior to receiving any treatment. In some aspects a subject treated with one or more technologies described herein will have improvements on speech pattern recognition, pure tone testing, bone conduction testing, acoustic reflex testing and/or tympanometry measurements after treatment as compared to before treatment. In some aspects, speech pattern recognition, pure tone testing, bone conduction testing, acoustic reflex testing and/or tympanometry measurements are taken after treatment is administered and at regular follow-up intervals post-treatment.

### Production Methods

AAV systems are generally well known in the art (see, e.g., Kelleher and Vos, Biotechniques, 17(6):1110-17 (1994); Cotten et al., P.N.A.S. U.S.A., 89(13):6094-98 (1992); Curiel, Nat Immun, 13(2-3):141-64 (1994); Muzyczka, Curr Top Microbiol Immunol, 158:97-129 (1992); and Asokan A, et al., Mol. Ther., 20(4):699-708 (2012), each of which is incorporated in its entirety herein by reference). Methods for generating and using AAV constructs are described, for example, in U.S. Pat. Nos. 5,139,941, 4,797,368 and PCT filing application US2019/060328, each of which is incorporated in its entirety herein by reference.

Methods for obtaining viral constructs are known in the art. For example, to produce AAV constructs, the methods typically involve culturing a host cell which contains a nucleic acid sequence encoding an AAV capsid protein or fragment thereof, a functional rep gene; a recombinant AAV construct composed of AAV inverted terminal repeats (ITRs) and a coding sequence; and/or sufficient helper functions to permit packaging of the recombinant AAV construct into the AAV capsid proteins.

In some aspects, components to be cultured in a host cell to package an AAV construct in an AAV capsid may be provided to the host cell in trans. Alternatively, any one or more components (e.g., recombinant AAV construct, rep sequences, cap sequences, and/or helper functions) may be provided by a stable host cell that has been engineered to contain one or more such components using methods known to those of skill in the art. In some aspects, such a stable host cell contains such component(s) under the control of an inducible promoter. In some aspects, such component(s) may be under the control of a constitutive promoter. In some aspects, a selected stable host cell may contain selected component(s) under the control of a constitutive promoter and other selected component(s) under the control of one or more inducible promoters. For example, a stable host cell may be generated that is derived from HEK293 cells (which contain E1 helper functions under the control of a constitutive promoter), but that contain the rep and/or cap proteins under the control of inducible promoters. Other stable host cells may be generated by one of skill in the art using routine methods.

Recombinant AAV construct, rep sequences, cap sequences, and helper functions required for producing an AAV of the disclosure may be delivered to a packaging host cell using any appropriate genetic element (e.g., construct). A selected genetic element may be delivered by any suitable method known in the art, e.g., to those with skill in nucleic acid manipulation and include genetic engineering, recombinant engineering, and synthetic techniques (see, e.g., Sambrook et al., Molecular Cloning: A Laboratory Manual, Cold Spring Harbor Press, Cold Spring Harbor, N.Y., which is incorporated in its entirety herein by reference). Similarly, methods of generating AAV particles are well known and any suitable method can be used with the present disclosure (see, e.g., K. Fisher et al., J. Virol., 70:520-532 (1993) and U.S. Pat. No. 5,478,745, which are incorporated in their entirety herein by reference).

In some aspects, recombinant AAVs may be produced using a triple transfection method (e.g., as described in U.S. Pat. No. 6,001,650, which is incorporated in its entirety herein by reference). In some aspects, recombinant AAVs are produced by transfecting a host cell with a recombinant AAV construct (comprising a coding sequence) to be packaged into AAV particles, an AAV helper function construct, and an accessory function construct. An AAV helper function construct encodes “AAV helper function” sequences (i.e., rep and cap), which function in trans for productive AAV replication and encapsidation. In some aspects, the AAV helper function construct supports efficient AAV construct production without generating any detectable wild-type AAV particles (i.e., AAV particles containing functional rep and cap genes). Non-limiting examples of constructs suitable for use with the present disclosure include pHLP19 (see, e.g., U.S. Pat. No. 6,001,650, which is incorporated in its entirety herein by reference) and pRep6cap6 construct (see, e.g., U.S. Pat. No. 6,156,303, which is incorporated in its entirety herein by reference). An accessory function construct encodes nucleotide sequences for non-AAV derived viral and/or cellular functions upon which AAV is dependent for replication (i.e., “accessory functions”). Accessory functions may include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

Additional methods for generating and isolating AAV viral constructs suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAVs are separated from contaminating virus. Other systems do not require infection with helper virus to recover the AAV--the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In some aspects, viral construct titers post-purification are determined. In some aspects, titers are determined using quantitative PCR. In certain aspects, a TaqMan probe specific to a construct is utilized to determine construct levels. In certain aspects, the TaqMan probe is represented by SEQ ID NO: 42, while forward and reverse amplifying primers are exemplified by SEQ ID NO: 43 and 44 respectively.

Exemplary Taqman probe for quantification of
constructs
(SEQ ID NO: 42)
/56-FAM/TCTGGCTCA/ZEN/CCGTCCTCTTCATTT/3IABkFQ/
Exemplary forward qPCR primer for quantification
of constructs
(SEQ ID NO: 43)
CAAACACTCCACCAGCATTG
Exemplary reverse qPCR primer for quantification
of constructs
(SEQ ID NO: 44)
CAGCCACAACGAGGATCATA

As described herein, in some aspects, a viral construct of the present disclosure is an adeno-associated virus (AAV) construct. Several AAV serotypes have been characterized, including AAV1, AAV2, AAV3 (e.g., AAV3B), AAV4, AAV5, AAV6, AAV7, AAV8, AAV9, AAV10, AAV 11, and AAV Anc80, as well as variants thereof. In some aspects, an AAV particle is an AAV2/6, AAV2/8, AAV2/9, or AAV2/Anc80 particle (e.g., with AAV6, AAV8, AAV9, or Anc80 capsid (e.g., an Anc80L65 capsid) and construct with AAV2 ITR). Other AAV particles and constructs are described in, e.g., Sharma et al., Brain Res Bull. 2010 Feb. 15; 81(2-3): 273, which is incorporated in its entirety herein by reference. Generally, any AAV serotype may be used to deliver a coding sequence described herein. However, the serotypes have different tropisms, e.g., they preferentially infect different tissues. In some aspects, an AAV construct is a self-complementary AAV construct.

The present disclosure provides, among other things, methods of making AAV-based constructs. In some aspects, such methods include use of host cells. In some aspects, a host cell is a mammalian cell. A host cell may be used as a recipient of an AAV helper construct, an AAV minigene plasmid, an accessory function construct, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV particles suitable for delivery to a subject are described in, e.g., U.S. Pat. Nos. 7,790,449; 7,282,199; WO 2003/042397; WO 2005/033321, WO 2006/110689; and U.S. Pat. No. 7,588,772, each of which is incorporated in its entirety herein by reference. In one system, a producer cell line is transiently transfected with a construct that encodes a coding sequence flanked by ITRs and a construct(s) that encodes rep and cap. In another system, a packaging cell line that stably supplies rep and cap is transiently transfected with a construct encoding a coding sequence flanked by ITRs. In each of these systems, AAV particles are produced in response to infection with helper adenovirus or herpesvirus, and AAV particles are separated from contaminating virus. Other systems do not require infection with helper virus to recover the AAV particles—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, a coding sequence flanked by ITRs and rep/cap genes are introduced into insect host cells by infection with baculovirus-based constructs. Such production systems are known in the art (see generally, e.g., Zhang et al., 2009, Human Gene Therapy 20:922-929, which is incorporated in its entirety herein by reference). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065, each of which is incorporated in its entirety herein by reference.

### EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather should be construed to encompass any and all variations that become evident as a result of the teaching provided herein.

It is believed that one or ordinary skill in the art can, using the preceding description and following Examples, as well as what is known in the art, make and utilize technologies of the present disclosure.

### Example 1: In Vitro Demonstration of Protein Production

This example relates to the introduction, regulation, and expression analysis of plasmids expressing a hKCNQ4 gene in mammalian cells grown in vitro.

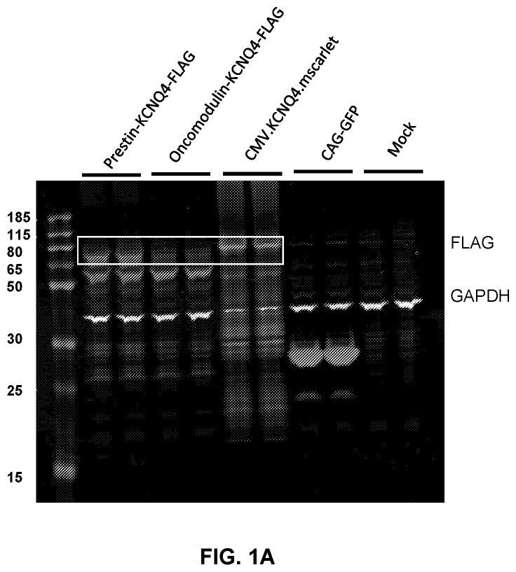

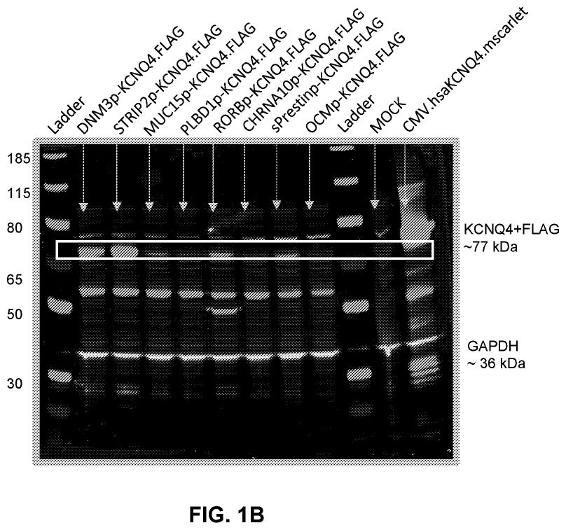

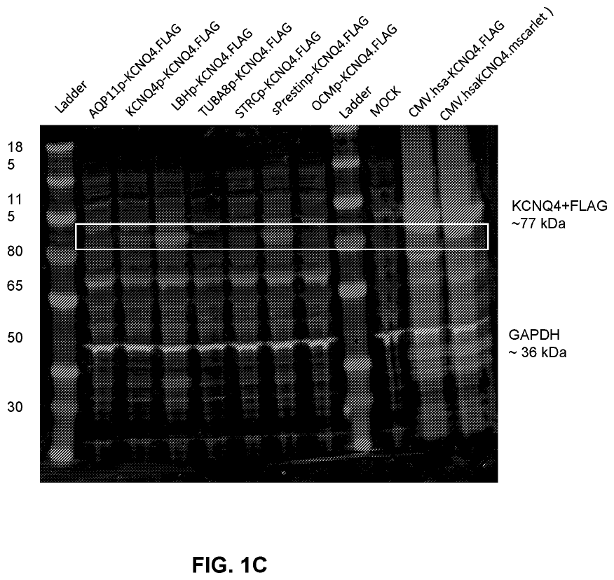

Experiments were conducted to demonstrate KCNQ4 protein expression from plasmids transfected into HEK293 cells. 500 ng of plasmids comprising prestin-KCNQ4-FLAG (comprising the promoter of SEQ ID NO: 3), oncomodulin-KCNQ4-FLAG (SEQ ID NO: 24), CMV.KCNQ4.mscarlet (SEQ ID NO:49), or CAG-GFP (SEQ ID NO: 50). Cells were harvested after 48 hours and western blot analysis shows that each construct was able to express KCNQ4 as evidenced by FLAG staining (FIG. 1A).

Next, 400 ng of plasmids comprising DNM3p-KCNQ4.FLAG (SEQ ID NO: 28), STRIP2p-KCNQ4.FLAG (SEQ ID NO: 32), MUC15p-KCNQ4.FLAG (SEQ ID NO: 29), PLBD1p-KCNQ4.FLAG (SEQ ID NO: 30), RORBp-KCNQ4.FLAG (SEQ ID NO: 31), CHRNA10p-KCNQ4.FLAG (SEQ ID NO: 27), sPrestinp-KCNQ4.FLAG (SEQ ID NO: 26), OCMp-KCNQ4.FLAG (SEQ ID NO: 24), or CMV.hsaKCNQ4.mscarlet (SEQ ID NO: 49). Cells were harvested after 48 hours and western blot analysis shows that each construct was able to express KCNQ4 as evidenced by FLAG staining (FIG. 1B).

400 ng of plasmids comprising AQP11p.KCNQ4.FLAG (SEQ ID NO: 33), KCNQ4p-KCNQ4.FLAG (SEQ ID NO: 34), LBHp-KCNQ4.FLAG (SEQ ID NO: 35), TUBA8p-KCNQ4.FLAG (SEQ ID NO: 37), STRCp-KCNQ4.FLAG (SEQ ID NO: 36), sPrestinp-KCNQ4.FLAG (SEQ ID NO: 26), OCMp-KCNQ4.FLAG (SEQ ID NO: 24), CMV.hsa-KCNQ4.FLAG, or CMV.hsa.KCNQ4.mscarlet (SEQ ID NO: 49). Cells were harvested after 48 hours and western blot analysis shows that each construct was able to express KCNQ4 as evidenced by FLAG staining (FIG. 1C).

### Example 2: In Vivo Demonstration of KCNQ4 Expression in Mutant Mice

This example relates to the introduction and expression analysis of rAAV constructs encoding KCNQ4 protein under the control of an outer hair cell specific promoter.

Experiments were conducted to demonstrate preservation of hair cells and auditory function in neonatal Kcnq4^{dn/+} (knock-in [KI]) mice, which carry a dominant negative mutation in Kcnq4 that mimics a known pathogenic variant in humans. KCNQ4 protein is essentially undectable in both heterozygous and homozygous KI mice. The AAVAnc80 vectors are intended to deliver a gene that encodes human codon modified KCNQ4 (hKCNQ4CM), under the control of an outer hair cell specific promoter (e.g., prestin or oncomodulin). Exemplary contructs can comprise a 5′ ITR, outer hair-cell specific promoter (e.g., sPrestin), a 5′ UTR, KCNQ4 coding region, epitope tag (e.g., 3× FLAG), a polyadenylation signal (e.g., BGH), and a 3′ ITR.

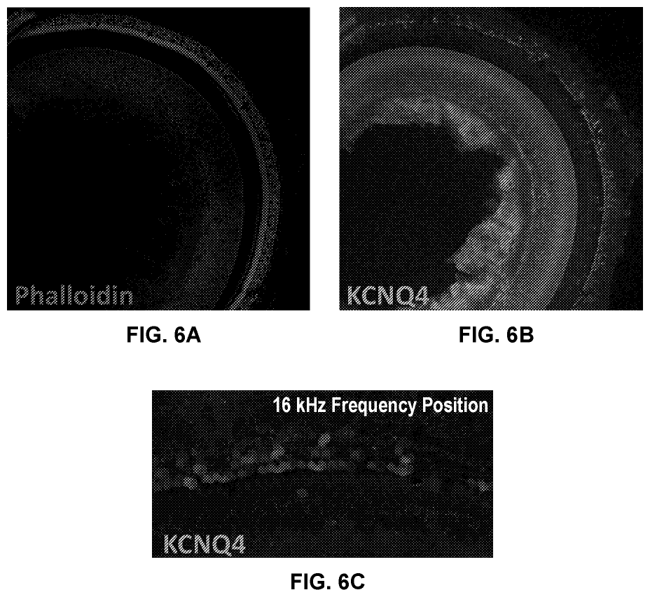

rAAVAnc80 particles, comprising a construct of SEQ ID NO: 26, were administered to the cochlea of KI postnatal day 2 neonatal mice at 8.0E9 vg/cochlea. Samples were harvested at postnatal day 30 and stained with phalloidin (FIG. 6A) and an antibody against KCNQ4 (FIG. 6B), which demonstrated KCNQ4 staining in OHCs. FIG. 6C shows a magnified view of a region of the cochlea receptive to the 16 kilohertz frequency, wherein KCNQ4 staining is further demonstrated in OHCs.

1. A construct comprising a polynucleotide encoding a polypeptide operably linked to a promoter which expresses the polynucleotide in an outer hair cell, wherein the promoter is selected the group consisting of an oncomodulin (OCM) promoter, a prestin promoter, a cholinergic receptor nicotinic alpha 10 (CHRNA10) promoter, a dynamin 3 (DNM3) promoter, a mucin 14 (MUC15) promoter, a phospholipase D (PLDB1) promoter, a RAR related orphan receptor B (RORB) promoter, a striatin interacting protein 2 (STRIP2) promoter, an aquaporin 11 (AQP11) promoter, a potassium voltage-gated channel subfamily Q member 4 (KCNQ4) promoter, a LBH promoter, a stereocilin (STRC) promoter, a tubulin alpha 8 (TUBA8) promoter, and any combination thereof, wherein the promoter is heterologous to the polynucleotide.

2-3. (canceled)

4. A construct comprising a polynucleotide encoding a polypeptide operably linked to a promoter which expresses the polynucleotide in an outer hair cell, wherein the promoter comprises a nucleic acid sequence having at least 85%, at least 90%, at least 95%, at least 96%, at least 97%, at least 98%, at least 99%, or 100% identity to any one of SEQ ID NOs: 1-15.

5-9. (canceled)

10. The construct of claim 4, wherein the promoter is heterologous to the polynucleotide.

11. The construct of claim 4, wherein the polypeptide is an outer hair cell polypeptide, therapeutic polypeptide, or a reporter polypeptide.

12. The construct of claim 11, wherein the polynucleotide encoding an outer hair cell polypeptide comprises a gene selected from actin gamma 1 (ACTG1), adenylate cyclase type 1 (ADCY1), calcium binding protein 2 (CABP2), coiled-coil domain-containing 50 (CCDC50), cadherin-related 23 (CDH23), carcinoembryonic antigen-related cell adhesion molecule 16 (CEACAM16), chromodomain helicase DNA-binding protein 7 (CHD7), calcium- and integrin-binding family member 2 (CIB2), claudin 14 (CLDN14), chloride intracellular channel 5 (CLIC5), caseinolytic mitochondrial matrix peptidase proteolytic subunit (CLPP), clarin 1 (CLRN1), pejvakin (DFNB59), endothelin 3 (EDN3), ELMO domain-containing protein 3 (ELMOD3), epidermal growth factor receptor kinase substrate 8 (EPS8), espin (ESPN), estrogen-related receptor beta (ESRRB), eyes absent homolog 1 (EYA1), GIPC PDZ domain-containing family, member 3 (GIPC3), G protein-coupled receptor 98 (GPR98), G-protein signaling modulator 2 (GPSM2), glutaredoxin, cysteine-rich 1 (GRXCR1), glutaredoxin, cysteine-rich 2 (GRXCR2), immunoglobulin-like domain-containing receptor 1 (ILDR1), lysyl-tRNA synthetase (KARS), Potassium voltage-gated channel, KQT-like subfamily, member 4 (KCNQ4), lipoma HMGIC fusion partner-like 5 (LHFPL5), leucine-rich transmembrane and O-methyltransferase domain-containing (LRTOMT1 COMT2), tricellulin (MARVELD2), micro-ma 96 (MIR96), methionine sulfoxide reductase B3 (MSRB3), myosin, heavy chain 9, non-muscle (MYH9), myosin, heavy chain 14, non-muscle (MYH14), unconventional myosin IIIA (MYO3A), unconventional myosin VI (MYO6), unconventional myosin VIIA (MYO7A), unconventional myosin XVA (MYOl5A), otoferlin (OTOF), otogelin-like protein (OTOGL), purinergic receptor P2X, ligand-gated ion channel, 2 (P2RX2), protocadherin 15 (PCDH15), PDZ domain-containing 7 (PDZD7), polyribonucleotide nucleotidyltransferase 1, mitochondrial (PNPT1), POU domain, class 4, transcription factor 3 (POU4F3), phosphoribosyl pyrophosphate synthetase 1 (PRPS1), protein tyrosine phosphatase, receptor type Q (PTPRQ), radixin (RDX), scaffold-containing ankyrin repeats and SAM domain (SANS), serpin peptidase inhibitor, clade B, member 6 (SERPINB6), SIX homeobox 1 (SIX1), SIX homeobox 5 (SIX5), prestin (SLC26A5), second mitochondrial-derived activator of caspase (SMAC/DIABLO), small muscle protein, x-linked (SMPX), stereocilin (STRC), nesprin-4 (SYNE4), TBC1 domain family, member 24 (TBC/D24), tight junction protein XO 2 (TJP2), transmembrane channel-like protein 1 (TMC1), transmembrane inner ear-expressed protein (TMIE), transmembrane protease, serine 3 (TMPRSS3), taperin (TPRN), TRIO and F-actin-binding protein (TRIOBP), Thrombospondin-type laminin G domain and EAR repeats (TSPEAR), harmonin (USH1C), usherin (USH2A), wolframin (WFS1), and whirlin (WHRN), or any combination thereof.

13. (canceled)

14. The construct of claim 11, wherein the outer hair cell polypeptide comprises KQT-like subfamily, member 4 (KCNQ4).

15-20. (canceled)

21. The construct of claim 4, wherein the construct further comprises an enhancer, optionally, wherein the enhancer is a CMV enhancer.

22-23. (canceled)

24. The construct of claim 4, wherein the construct further comprises a 5′ UTR and a 3′ UTR.

25. (canceled)

26. The construct of any of the preceding claims, wherein the construct further comprises a polyA tail, wherein the polyA tail is a bovine growth hormone, mouse-β-globin, mouse-α-globin, human collagen, polyoma virus, the Herpes simplex virus thymidine kinase gene (HSV TK), Ig heavy-chain gene, human growth hormone, or a SV40 late and early poly(A).

27-28. (canceled)

29. The construct of claim 4, wherein the construct comprises a nucleic acid sequence comprising any one of nucleotides 12-4396 of SEQ ID NO: 23, 12-4464 of SEQ ID NO: 24, nucleotides 12-4016 of SEQ ID NO: 25, nucleotides 12-4521 of SEQ ID NO: 26, nucleotides 12-3750 of SEQ ID NO: 27, nucleotides 12-3928 of SEQ ID NO: 28, nucleotides 12-4641 of SEQ ID NO: 29, nucleotides 12-3994 of SEQ ID NO: 30, nucleotides 12-4426 of SEQ ID NO: 31, nucleotides 12-4307 of SEQ ID NO: 32, nucleotides 12-4293 of SEQ ID NO: 33, nucleotides 12-4565 of SEQ ID NO: 34, nucleotides 12-4224 of SEQ ID NO: 35, nucleotides 12-4140 of SEQ ID NO: 36, nucleotides 12-4816 of SEQ ID NO: 37, or nucleotides 12-4915 of SEQ ID NO: 38.

30. (canceled)

31. The construct of claim 4, wherein the construct is an expression cassette.

32. A vector comprising the construct of claim 4, wherein the vector is a mammalian vector or a viral vector.

33. (canceled)

34. The vector of claim 32, wherein the vector is a viral vector, and wherein the viral vector is selected from the group consisting of an adeno-associated viral (AAV), adenovirus, or lentiviral vector.

35. (canceled)

36. The vector of claim 34, wherein the viral vector is an AAV vector, wherein the AAV vector further comprises a 5′ inverted terminal repeat (ITR) and a 3′ ITR.

37. (canceled)

38. The vector of claim 36, wherein the 5′ ITR and the 3′ ITR are AAV ITRs derived from a serotype selected from AAV1, AAV2, AAV3, AAV4, AAV5, AAV6, AAV7, AAV8, AAV9, AAV10, AAV11, and AAV Anc80 ITRs.

39. The vector of claim 38, wherein the AAV ITRs are serotype AAV2 or derived from serotype AAV2.

40.-41. (canceled)

42. An AAV particle comprising the construct of claim 4.

43. The AAV particle of claim 42, comprising an AAV capsid, wherein the AAV capsid is or is derived from an AAV2, AAV3, AAV4, AAV5, AAV6, AAV7, AAV8, AAV9, AAV10, AAV-rh8, AAV-rh10, AAV-rh39, AAV-rh43 or AAV Anc80 capsid.

44. The AAV particle of claim 43, wherein the AAV capsid is an AAV Anc80 capsid.

45. A composition comprising the construct of claim 4.

46. The composition of claim 45, wherein the composition is a pharmaceutical composition further comprising a pharmaceutically acceptable carrier.

47. (canceled)

48. A cell comprising the construct of claim 4.

49.-50. (canceled)

51. A method comprising, transducing a cell with:
a. the construct of claim 4; and
b. one or more helper plasmids collectively comprising an AAV Rep gene, AAV Cap gene, AAV VA gene, AAV E2a gene, and AAV E4 gene.

52.-53. (canceled)

54. A method of expressing the polypeptide in an outer hair cell of a subject in need thereof, comprising administering the construct of claim 4 to the subject.

55. A method of increasing expression of the polypeptide in an outer hair cell of a subject in need thereof, comprising administering the construct of claim 4 to the subject.

56. (canceled)

57. A method of treating hearing loss in a subject suffering from or at risk of hearing loss, comprising administering the construct of claim 4 to the subject.

58.-69. (canceled)

70. A kit comprising the construct of claim 4.

71.-77. (canceled)

78. A viral vector comprising: (i) a 5′ inverted terminal repeat (ITR), (ii) a polynucleotide encoding a polypeptide operably linked to a promoter which expresses the polynucleotide in an outer hair cell, and (iii) a 3′ ITR, wherein the promoter is heterologous to the polynucleotide, and wherein the promoter comprises a nucleic acid sequence having at least 85%, at least 90%, at least 95%, at least 96%, at least 97%, at least 98%, at least 99%, or 100% identity to any one of SEQ ID NOs: 1-15.
